(19) United States
(12) Patent Application Publication
(10) Pub. No.: US 2026/0275346 A1
(43) Pub. Date: Sep. 17, 2026

(54) ANTISENSE OLIGOMERS FOR TREATMENT OF CONDITIONS AND DISEASES

(71) Applicant: Stoke Therapeutics, Inc., Bedford, MA (US)

(72) Inventor: Isabel Aznarez, Jamaica Plain, MA (US)

(21) Appl. No.: 19/542,296

(22) Filed: Feb. 17, 2026

Related U.S. Application Data
(62) Division of application No. 17/412,664, filed on Aug. 26, 2021, now Pat. No. 12,577,561.
Application No. 17/412,664 is a continuation of application No. PCT/US2020/020175, filed on Feb. 27, 2020.
(60) Provisional application No. 62/811,511, filed on Feb. 27, 2019.

(51) Int. Cl. C12N 15/113

(52) U.S. Cl. CPC C12N 15/113; C12N 2310/11; C12N 2310/315; C12N 2310/321; C12N 2320/11; C12N 2320/33

(57) ABSTRACT

Alternative splicing events in SCN1A gene can lead to non-productive mRNA transcripts which in turn can lead to aberrant protein expression, and therapeutic agents which can target the alternative splicing events in SCN1A gene can modulate the expression level of functional proteins in Dravet Syndrome patients and/or inhibit aberrant protein expression. Such therapeutic agents can be used to treat a condition caused by SCN1A, SCN8A or SCN5A protein deficiency.

### CROSS-REFERENCE

This application is a divisional of U.S. application Ser. No. 17/412,664, filed Aug. 26, 2021, which is a continuation of international patent application no. PCT/US2020/020175, filed Feb. 27, 2020, which claims the benefit of U.S. Provisional Application No. 62/811,511, filed on Feb. 27, 2019, each of which is incorporated herein by reference in its entirety.

### SEQUENCE LISTING

The instant application contains a Sequence Listing which has been submitted electronically in XML format and is hereby incorporated by reference in its entirety. Said XML file, created on May 14, 2026, is named 47991-724_401_SL.xml and is 674,290 bytes in size. The sequence listing electronically contains no new matter.

### BACKGROUND

Nervous system disorders are often associated with channelopathy, characterized by the disturbed function of ion channels that mediate neuronal excitability, neuronal interactions, and brain functions at large. Mutations in the SCN1A gene, which is part of the SCN1A-SCN2A-SCN3A gene cluster that encodes alpha-pore forming subunits of the neuronal voltage gated sodium channel, are associated with development of disease number of diseases and conditions, such as Dravet Syndrome (DS) (Miller, et al., 1993-2015, GeneReviews, Eds. Pagon R A, et al. Seattle (WA): University of Washington, Seattle, Bookshelf ID: NBK1318, and Mulley, et al., 2005, Hum. Mutat. 25:535-542).

### SUMMARY

Disclosed herein, in certain embodiments, is a method of modulating expression of SCN1A protein in a cell having an mRNA that contains a non-sense mediated RNA decay-inducing exon (NMD exon mRNA) and encodes SCN1A protein, the method comprising contacting a therapeutic agent to the cell, whereby the therapeutic agent modulates splicing of the NMD exon from the NMD exon mRNA encoding SCN1A protein, thereby modulating the level of processed mRNA encoding SCN1A protein, and modulating expression of SCN1A protein in the cell, wherein the therapeutic agent binds to a targeted portion of the NMD exon mRNA encoding SCN1A, and wherein the targeted portion is: from about 1000 nucleotides upstream from the 5′ end of an NMD-inducing exon (NIE) to about 100 nucleotides upstream from the 5′ end of the NIE; or from about 100 nucleotides downstream of the 3′ end of the NIE to about 1000 nucleotides downstream of the 3′ end of the NIE. In some embodiments, the therapeutic agent interferes with binding of a factor involved in splicing of the NMD exon from a region of the targeted portion. In some embodiments, the targeted portion is at most about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of 5′ end of the NIE. In some embodiments, the targeted portion is at least about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides upstream of 5′ end of the NIE. In some embodiments, the targeted portion is at most about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of 3′ end of the NIE. In some embodiments, the targeted portion is at least about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides downstream of 3′ end of the NIE. In some embodiments, the therapeutic agent is an antisense oligomer (ASO). In some embodiments, the ASO comprises a sequence that is at least about 80%, 85%, 90%, 95%, 97%, or 100% identity to any one of SEQ ID NOs: 12-731. In some embodiments, the therapeutic agent promotes exclusion of the NMD exon from the processed mRNA encoding SCN1A protein. In some embodiments, exclusion of the NMD exon from the processed mRNA encoding SCN1A protein in the cell contacted with the therapeutic agent is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to exclusion of the NMD exon from the processed mRNA encoding SCN1A protein in a control cell. In some embodiments, the therapeutic agent increases level of the processed mRNA encoding SCN1A protein in the cell. In some embodiments, an amount of the processed mRNA encoding SCN1A protein in the cell contacted with the therapeutic agent is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to an total amount of the processed mRNA encoding SCN1A protein in a control cell.

Disclosed herein, in certain embodiments, is a method of treating a disease or condition in a subject in need thereof by modulating expression of SCN1A protein in a cell of the subject, comprising: contacting the cell of the subject with a therapeutic agent that modulates splicing of a non-sense mediated mRNA decay-inducing exon (NMD exon) from an mRNA in the cell that contains the NMD exon and encodes SCN1A, thereby modulating the level of processed mRNA encoding the SCN1A protein, and modulating expression of SCN1A protein in the cell of the subject; wherein the therapeutic agent binds to a targeted portion of the NMD exon mRNA encoding SCN1A, and wherein the targeted portion is: from about 1000 nucleotides upstream from the 5′ end of an NMD-inducing exon (NIE) to about 100 nucleotides upstream from the 5′ end of the NIE; or from about 100 nucleotides downstream of the 3′ end of the NIE to about 1000 nucleotides downstream of the 3′ end of the NIE. In some embodiments, the therapeutic agent interferes with binding of a factor involved in splicing of the NMD exon from a region of the targeted portion. In some embodiments, the targeted portion is at most about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of 5′ end of the NIE. In some embodiments, the targeted portion is at least about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides upstream of 5′ end of the NIE. In some embodiments, the targeted portion is at most about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of 3′ end of the NIE. In some embodiments, the targeted portion is at least about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides downstream of 3′ end of the NIE. In some embodiments, the therapeutic agent is an antisense oligomer (ASO). In some embodiments, the ASO comprises a sequence that is at least about 80%, 85%, 90%, 95%, 97%, or 100% identity to any one of SEQ ID NOs: 12-731. In some embodiments, the therapeutic agent promotes exclusion of the NMD exon from the processed mRNA encoding SCN1A protein. In some embodiments, exclusion of the NMD exon from the processed mRNA encoding SCN1A protein in the cell contacted with the therapeutic agent is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to exclusion of the NMD exon from the processed mRNA encoding SCN1A protein in a control cell. In some embodiments, the therapeutic agent increases level of the processed mRNA encoding SCN1A protein in the cell. In some embodiments, an amount of the processed mRNA encoding SCN1A protein in the cell contacted with the therapeutic agent is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to an total amount of the processed mRNA encoding SCN1A protein in a control cell. In some embodiments, the disease or condition is induced by a loss-of-function mutation in Naᵥ1.1. In some embodiments, the disease or condition is associated with haploinsufficiency of the SCN1A gene, and wherein the subject has a first allele encoding a functional SCN1A, and a second allele from which SCN1A is not produced or produced at a reduced level, or a second allele encoding a nonfunctional SCN1A or a partially functional SCN1A. In some embodiments, the disease or condition is encephalopathy. In some embodiments, the encephalopathy is epileptic encephalopathy. In some embodiments, the disease or condition is Dravet Syndrome (DS); severe myoclonic epilepsy of infancy (SMEI)-borderland (SMEB); Febrile seizure (FS); epilepsy, generalized, with febrile seizures plus (GEFS+); epileptic encephalopathy, early infantile, 13; cryptogenic generalized epilepsy; cryptogenic focal epilepsy; myoclonic-astatic epilepsy; Lennox-Gastaut syndrome; West syndrome; idiopathic spasms; early myoclonic encephalopathy; progressive myoclonic epilepsy; alternating hemiplegia of childhood; unclassified epileptic encephalopathy; sudden unexpected death in epilepsy (SUDEP); sick sinus syndrome 1; autism; or malignant migrating partial seizures of infancy. In some embodiments, GEFS+ is epilepsy, generalized, with febrile seizures plus, type 2. In some embodiments, the Febrile seizure is Febrile seizures, familial, 3A. In some embodiments, SMEB is SMEB without generalized spike wave (SMEB-SW), SMEB without myoclonic seizures (SMEB-M), SMEB lacking more than one feature of SMEI (SMEB-O), or intractable childhood epilepsy with generalized tonic-clonic seizures (ICEGTC).

Disclosed herein, in certain embodiments, is a method of modulating expression of SCN1A protein in a cell having an mRNA that contains a non-sense mediated RNA decay-inducing exon (NMD exon mRNA) and encodes SCN1A protein, the method comprising contacting a therapeutic agent to the cell, whereby the therapeutic agent modulates splicing of the NMD exon from the NMD exon mRNA encoding SCN1A protein, thereby modulating the level of processed mRNA encoding SCN1A protein, and modulating expression of SCN1A protein in the cell, wherein the therapeutic agent binds to a targeted portion of the NMD exon mRNA encoding SCN1A, and wherein the targeted portion is from about 1000 nucleotides upstream from the 5′ end of an NMD-inducing exon (NIE) to about 1000 nucleotides downstream of the 3′ end of the NIE.

Disclosed herein, in certain embodiments, is a method of treating a disease or condition in a subject in need thereof by modulating expression of SCN1A protein in a cell of the subject, comprising: contacting the cell of the subject with a therapeutic agent that modulates splicing of a non-sense mediated mRNA decay-inducing exon (NMD exon) from an mRNA in the cell that contains the NMD exon and encodes SCN1A, thereby modulating the level of processed mRNA encoding the SCN1A protein, and modulating expression of SCN1A protein in the cell of the subject; wherein the therapeutic agent binds to a targeted portion of the NMD exon mRNA encoding SCN1A, and wherein the targeted portion is from about 1000 nucleotides upstream from the 5′ end of an NMD-inducing exon (NIE) to about 1000 nucleotides downstream of the 3′ end of the NIE.

Disclosed herein, in certain embodiments, is an antisense oligomer (ASO) comprising a sequence that is at least about 80%, 85%, 90%, 95%, 97%, or 100% identity to any one of SEQ ID NOs: 12-731.

Disclosed herein, in certain embodiments, is an antisense oligomer (ASO) consisting of a sequence selected from SEQ ID NOs: 12-731.

Disclosed herein, in certain embodiments, is a method of treating a disease or condition in a subject in need thereof by modulating expression of SCN1A protein in a cell of the subject, comprising: contacting the cell of the subject with an ASO comprising a sequence that is at least about 80%, 85%, 90%, 95%, 97%, or 100% identity to any one of SEQ ID NOs: 12-731; or an ASO consisting of a sequence selected from SEQ ID NOs: 12-731.

Disclosed herein, in certain embodiments, is a kit comprising an ASO comprising a sequence that is at least about 80%, 85%, 90%, 95%, 97%, or 100% identity to any one of SEQ ID NOs: 12-731; or an ASO consisting of a sequence selected from SEQ ID NOs: 12-731.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a schematic representation of a target mRNA that contains a non-sense mediated RNA decay-inducing exon (NMD exon mRNA) and therapeutic agent-mediated exclusion of the nonsense-mediated mRNA decay-inducing exon to increase expression of the full-length target protein or functional RNA. FIG. 1A shows a cell divided into nuclear and cytoplasmic compartments. In the nucleus, a pre-mRNA transcript of a target gene undergoes splicing to generate mRNA, and this mRNA is exported to the cytoplasm and translated into target protein. For this target gene, some fraction of the mRNA contains a nonsense-mediated mRNA decay-inducing exon (NMD exon mRNA) that is degraded in the cytoplasm, thus leading to no target protein production. FIG. 1B shows an example of the same cell divided into nuclear and cytoplasmic compartments. Treatment with a therapeutic agent, such as an antisense oligomer (ASO), promotes the exclusion of the nonsense-mediated mRNA decay-inducing exon and results in an increase in mRNA, which is in turn translated into higher levels of target protein. FIG. 1C is a schematic representation of therapeutic ASO-mediated exclusion of a nonsense-mediated mRNA decay-inducing exon, which decreases non-productive mRNA and increases productive mRNA and increases expression of the full-length target protein from the productive mRNA.

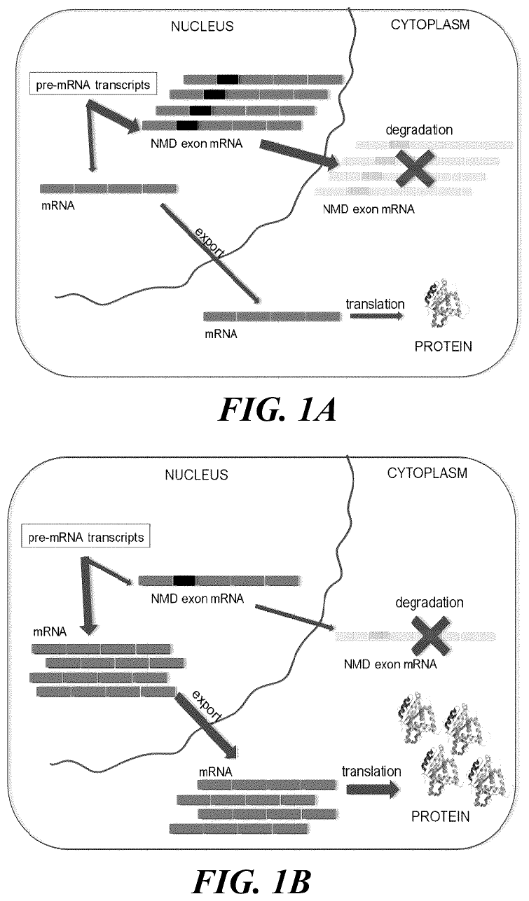

FIG. 2 depicts identification of an exemplary nonsense-mediated mRNA decay (NMD)-inducing exon in the SCN1A gene. The identification of the NMD-inducing exon in the SCN1A gene using comparative genomics is shown, visualized in the UCSC genome browser. The upper panel shows a graphic representation of the SCN1A gene to scale. The conservation level across 100 vertebrate species is shown as peaks. The highest peaks correspond to exons (black boxes), while no peaks are observed for the majority of the introns (lines with arrow heads). Peaks of conservation were identified in intron 20 (NM_006920), shown in the middle panel. Inspection of the conserved sequences identified an exon-like sequence of 64 bp (bottom panel, sequence highlighted in grey) flanked by 3′ and 5′ splice sites (underlined sequence), which we refer to as exon 20x. Inclusion of this exon leads to a frameshift and the introduction of a premature termination codon in exon 21 rendering the transcript a target of NMD. FIG. 2 discloses SEQ ID NO: 732.

FIG. 3A depicts confirmation of NMD-inducing exon via cycloheximide treatment. RT-PCR analysis using cytoplasmic RNA from DMSO-treated (CHX−) or cycloheximide-treated (CHX+) Neuro 2A (mouse neural progenitor cells) and primers in exon 21 and a downstream exon confirmed the presence of a band corresponding to the NMD-inducing exon (21x). The identity of the product was confirmed by sequencing. Densitometry analysis of the bands was performed to calculate percent exon 21x inclusion of total SCN1A transcript. Treatment of Neuro 2A with cycloheximide (CHX+) to inhibit NMD led to a 2-fold increase of the product corresponding to the NMD-inducing exon 21x in the cytoplasmic fraction (cf. light grey bar, CHX−, to dark grey bar, CHX+).

FIG. 3B depicts confirmation of NMD-inducing exon via cycloheximide treatment. RT-PCR analysis using cytoplasmic RNA from DMSO-treated (CHX−) or cycloheximide-treated (CHX+) RenCell VM (human neural progenitor cells) and primers in exon 20 and exon 23 confirmed the presence of a band corresponding to the NMD-inducing exon (20x). The identity of the product was confirmed by sequencing. Densitometry analysis of the bands was performed to calculate percent exon 20x inclusion of total SCN1A transcript. Treatment of RenCell VM with cycloheximide (CHX+) to inhibit NMD led to a 2-fold increase of the product corresponding to the NMD-inducing exon 20x in the cytoplasmic fraction (cf. light grey bar, CHX−, to dark grey bar, CHX+).

FIG. 4. depicts an exemplary graphic representation of an ASO walk performed for SCN1A exon 20x region targeting two indicated regions (region 1 and region 2) upstream of the 3′ splice site of exon 20x and two indicated regions (region 3 and region 4) downstream of the 5′ splice site of exon 20x. ASOs were designed to cover these regions by shifting 5 nucleotides at a time.

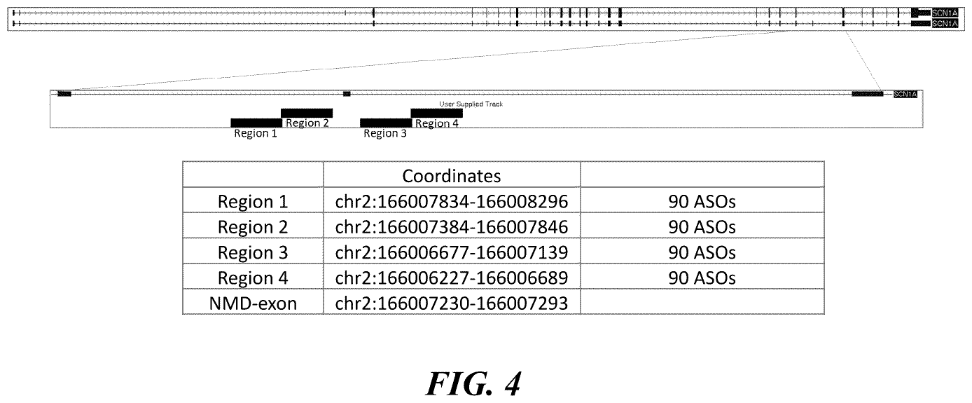

FIG. 5A depicts SCN1A exon 20x region ASOs selected from an extended ASO walk evaluated by RT-PCR. A representative PAGE shows SYBR-safe-stained RT-PCR products of SCN1A mock-treated, control ASO treated (NT), SCN1A exon 20x region ASOs from an extended walk in RenCells via nucleofection at 1 μM for 24 hrs. Mock=No ASO; control NT=non-targeting control; Posctrl=positive control.

FIG. 5B depicts a graph plotting the percent exon 20x inclusion from the data in FIG. 5A.

FIG. 5C depicts a graph of qPCR results of an extended ASO walk using the samples of FIG. 5A normalized to RPL32 internal control and the fold-change of SCN1A mRNA relative to mock is plotted.

### DETAILED DESCRIPTION OF THE INVENTION

Splicing and Nonsense-Mediated mRNA Decay

Intervening sequences or introns are removed by a large and highly dynamic RNA-protein complex termed the spliceosome, which orchestrates complex interactions between primary transcripts, small nuclear RNAs (snRNAs) and a large number of proteins. Spliceosomes assemble ad hoc on each intron in an ordered manner, starting with recognition of the 5′ splice site (5′ss) by U1 snRNA or the 3′-splice site (3′ss) by the U2 pathway, which involves binding of the U2 auxiliary factor (U2AF) to the 3′ss region to facilitate U2 binding to the branch point sequence (BPS). U2AF is a stable heterodimer composed of a U2AF2-encoded 65-kD subunit (U2AF65), which binds the polypyrimidine tract (PPT), and a U2AF1-encoded 35-kD subunit (U2AF35), which interacts with highly conserved AG dinucleotides at 3′ss and stabilizes U2AF65 binding. In addition to the BPS/PPT unit and 3′ss/5′ss, accurate splicing requires auxiliary sequences or structures that activate or repress splice site recognition, known as intronic or exonic splicing enhancers or silencers. These elements allow genuine splice sites to be recognized among a vast excess of cryptic or pseudo-sites in the genome of higher eukaryotes, which have the same sequences but outnumber authentic sites by an order of magnitude. Although they often have a regulatory function, the exact mechanisms of their activation or repression are poorly understood.

The decision of whether to splice or not to splice can be typically modeled as a stochastic rather than deterministic process, such that even the most defined splicing signals can sometimes splice incorrectly. However, under normal conditions, pre-mRNA splicing proceeds at surprisingly high fidelity. This is attributed in part to the activity of adjacent cis-acting auxiliary exonic and intronic splicing regulatory elements (ESRs or ISRs). Typically, these functional elements are classified as either exonic or intronic splicing enhancers (ESEs or ISEs) or silencers (ESSs or ISSs) based on their ability to stimulate or inhibit splicing, respectively. Although there is now evidence that some auxiliary cis-acting elements may act by influencing the kinetics of spliceosome assembly, such as the arrangement of the complex between U1 snRNP and the 5′ss, it seems very likely that many elements function in concert with trans-acting RNA-binding proteins (RBPs). For example, the serine- and arginine-rich family of RBPs (SR proteins) is a conserved family of proteins that have a key role in defining exons. SR proteins promote exon recognition by recruiting components of the pre-spliceosome to adjacent splice sites or by antagonizing the effects of ESSs in the vicinity. The repressive effects of ESSs can be mediated by members of the heterogeneous nuclear ribonucleoprotein (hnRNP) family and can alter recruitment of core splicing factors to adjacent splice sites. In addition to their roles in splicing regulation, silencer elements are suggested to have a role in repression of pseudo-exons, sets of decoy intronic splice sites with the typical spacing of an exon but without a functional open reading frame. ESEs and ESSs, in cooperation with their cognate trans-acting RBPs, represent important components in a set of splicing controls that specify how, where and when mRNAs are assembled from their precursors.

The sequences marking the exon-intron boundaries are degenerate signals of varying strengths that can occur at high frequency within human genes. In multi-exon genes, different pairs of splice sites can be linked together in many different combinations, creating a diverse array of transcripts from a single gene. This is commonly referred to as alternative pre-mRNA splicing. Although most mRNA isoforms produced by alternative splicing can be exported from the nucleus and translated into functional polypeptides, different mRNA isoforms from a single gene can vary greatly in their translation efficiency. Those mRNA isoforms with premature termination codons (PTCs) at least 50 bp upstream of an exon junction complex are likely to be targeted for degradation by the nonsense-mediated mRNA decay (NMD) pathway. Mutations in traditional (BPS/PPT/3′ss/5′ss) and auxiliary splicing motifs can cause aberrant splicing, such as exon skipping or cryptic (or pseudo-) exon inclusion or splice-site activation, and contribute significantly to human morbidity and mortality. Both aberrant and alternative splicing patterns can be influenced by natural DNA variants in exons and introns.

Given that exon-intron boundaries can occur at any of the three positions of a codon, it is clear that only a subset of alternative splicing events can maintain the canonical open reading frame. For example, only exons that are evenly divisible by 3 can be skipped or included in the mRNA without any alteration of reading frame. Splicing events that do not have compatible phases will induce a frame-shift. Unless reversed by downstream events, frame-shifts can certainly lead to one or more PTCs, probably resulting in subsequent degradation by NMD. NMD is a translation-coupled mechanism that eliminates mRNAs containing PTCs. NMD can function as a surveillance pathway that exists in all eukaryotes. NMD can reduce errors in gene expression by eliminating mRNA transcripts that contain premature stop codons. Translation of these aberrant mRNAs could, in some cases, lead to deleterious gain-of-function or dominant-negative activity of the resulting proteins. NMD targets not only transcripts with PTCs but also a broad array of mRNA isoforms expressed from many endogenous genes, suggesting that NMD is a master regulator that drives both fine and coarse adjustments in steady-state RNA levels in the cell.

A NMD-inducing exon (NIE) is an exon or a pseudo-exon that is a region within an intron and can activate the NMD pathway if included in a mature RNA transcript. In the constitutive splicing events, the intron containing an NIE is usually spliced out, but the intron or a portion thereof (e.g. NIE) can be retained during alternative or aberrant splicing events. Mature mRNA transcripts containing such an NIE can be non-productive due to frame shift which induce NMD pathway. Inclusion of a NIE in mature RNA transcripts can downregulate gene expression. mRNA transcripts containing an NIE can be referred as “NIE containing mRNA” or “NMD exon mRNA” in the current disclosure.

Cryptic (or pseudo-splice sites) have the same splicing recognition sequences as genuine splice sites but are not used in the splicing reactions. They outnumber genuine splice sites in the human genome by an order of a magnitude and are normally repressed by thus far poorly understood molecular mechanisms. Cryptic 5′ splice sites have the consensus NNN/GUNNNN or NNN/GCNNNN where N is any nucleotide and/is the exon-intron boundary. Cryptic 3′ splice sites have the consensus NAG/N. Their activation is positively influenced by surrounding nucleotides that make them more similar to the optimal consensus of authentic splice sites, namely MAG/GURAGU and YAG/G, respectively, where Mis C or A, R is G or A, and Y is C or U.

Splice sites and their regulatory sequences can be readily identified by a skilled person using suitable algorithms publicly available, listed for example in Kralovicova, J. and Vorechovsky, I. (2007) Global control of aberrant splice site activation by auxiliary splicing sequences: evidence for a gradient in exon and intron definition. Nucleic Acids Res., 35, 6399-6413, (http://www.ncbi.nlm.nih.gov/pmc/articles/PMC2095810/pdf/gkm680.pdf)

The cryptic splice sites or splicing regulatory sequences may compete for RNA-binding proteins such as U2AF with a splice site of the NIE. In one embodiment, an agent may bind to the cryptic splice site or splicing regulatory sequences to prevent the binding of RNA-binding proteins and thereby favoring utilization of the NIE splice sites.

In one embodiment, the cryptic splice site may not comprise the 5′ or 3′ splice site of the NIE. The cryptic splice site may be at least 10 nucleotides upstream of the NIE 5′ splice site. The cryptic splice site may be at least 20 nucleotides upstream of the NIE 5′ splice site. The cryptic splice site may be at least 50 nucleotides upstream of the NIE 5′ splice site. The cryptic splice site may be at least 100 nucleotides upstream of the NIE 5′ splice site. The cryptic splice site may be at least 200 nucleotides upstream of the NIE 5′ splice site.

The cryptic splice site may be at least 10 nucleotides downstream of the NIE 3′ splice site. The cryptic splice site may be at least 20 nucleotides downstream of the NIE 3′ splice site. The cryptic splice site may be at least 50 nucleotides downstream of the NIE 3′ splice site. The cryptic splice site may be at least 100 nucleotides downstream of the NIE 3′ splice site. The cryptic splice site may be at least 200 nucleotides downstream of the NIE 3′ splice site.

### Target Transcripts

In some embodiments, the methods of the present disclosure exploit the presence of NIE in the pre-mRNA transcribed from the SCN1A gene. Splicing of the identified SCN1A NIE pre-mRNA species to produce functional mature SCN1A mRNA can be induced using a therapeutic agent such as an ASO that stimulates exon skipping of an NIE. Induction of exon skipping can result in inhibition of an NMD pathway. The resulting mature SCN1A mRNA can be translated normally without activating NMD pathway, thereby increasing the amount of SCN1A protein in the patient's cells and alleviating symptoms of a condition associated with SCN1A deficiency, such as Dravet Syndrome (DS); Epilepsy, generalized, with febrile seizures plus, type 2; Febrile seizures, familial, 3A; Autism; Epileptic encephalopathy, early infantile, 13; Sick sinus syndrome 1; Alzheimer's disease; or SUDEP.

In various embodiments, the present disclosure provides a therapeutic agent which can target SCN1A mRNA transcripts to modulate, e.g., enhance or inhibit, splicing or protein expression level. The therapeutic agent can be a small molecule, polynucleotide, or polypeptide. In some embodiments, the therapeutic agent is an ASO. Various regions or sequences on the SCN1A pre-mRNA can be targeted by a therapeutic agent, such as an ASO. In some embodiments, the ASO targets a SCN1A pre-mRNA transcript containing an NIE. In some embodiments, the ASO targets a sequence within an NIE of a SCN1A pre-mRNA transcript. In some embodiments, the ASO targets a sequence upstream (or 5′) from the 5′ end of an NIE (3′ss) of a SCN1A pre-mRNA transcript. In some embodiments, the ASO targets a sequence downstream (or 3′) from the 3′ end of an NIE (5′ss) of a SCN1A pre-mRNA transcript. In some embodiments, the ASO targets a sequence that is within an intron flanking on the 5′ end of the NIE of a SCN1A pre-mRNA transcript. In some embodiments, the ASO targets a sequence that is within an intron flanking the 3′ end of the NIE of a SCN1A pre-mRNA transcript. In some embodiments, the ASO targets a sequence comprising an NIE-intron boundary of a SCN1A pre-mRNA transcript. An NIE-intron boundary can refer to the junction of an intron sequence and an NIE region. The intron sequence can flank the 5′ end of the NIE, or the 3′ end of the NIE. In some embodiments, the ASO targets a sequence within an exon of a SCN1A pre-mRNA transcript. In some embodiments, the ASO targets a sequence within an intron of a SCN1A pre-mRNA transcript. In some embodiments, the ASO targets a sequence comprising both a portion of an intron and a portion of an exon.

In some embodiments, a therapeutic agent described herein modulates binding of a factor involved in splicing of the NMD exon mRNA.

In some embodiments, a therapeutic agent described herein interferes with binding of a factor involved in splicing of the NMD exon mRNA.

In some embodiments, a therapeutic agent described herein prevents binding of a factor involved in splicing of the NMD exon mRNA.

In some embodiments, a therapeutic agent targets a targeted portion located in an intronic region between two canonical exonic regions of the NMD exon mRNA encoding SCN1A, and wherein the intronic region contains the NMD exon.

In some embodiments, a therapeutic agent targets a targeted portion at least partially overlaps with the NMD exon.

In some embodiments, a therapeutic agent targets a targeted portion that is at least partially overlaps with an intron upstream of the NMD exon.

In some embodiments, a therapeutic agent targets a targeted portion within the NMD exon.

In some embodiments, a therapeutic agent targets a targeted portion comprising at least about 5, 6, 7, 8, 9, 10, 11, 12, 13, 14, 15, 16, 17, 18, 19, 20, 21, 22, 23, 24, 25, 26, 27, 28, 29, 30, or more consecutive nucleotides of the NMD exon. In some embodiments, a therapeutic agent targets a targeted portion comprising at most about 5, 6, 7, 8, 9, 10, 11, 12, 13, 14, 15, 16, 17, 18, 19, 20, 21, 22, 23, 24, 25, 26, 27, 28, 29, 30, or more consecutive nucleotides of the NMD exon. In some embodiments, a therapeutic agent targets a targeted portion comprising about 5, 6, 7, 8, 9, 10, 11, 12, 13, 14, 15, 16, 17, 18, 19, 20, 21, 22, 23, 24, 25, 26, 27, 28, 29, 30, or more consecutive nucleotides of the NMD exon.

In some embodiments, a therapeutic agent targets a targeted portion proximal to the NMD exon.

In some embodiments, the ASO targets a sequence from about 1 to about 5000 nucleotides downstream from the 5′ end of the intron comprising the NIE. In some embodiments, the ASO targets a sequence from about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, about 250 to about 300, about 250 to about 300 nucleotides, about 350 to about 400 nucleotides, about 450 to about 500 nucleotides, about 550 to about 600 nucleotides, about 650 to about 700 nucleotides, about 750 to about 800 nucleotides, about 850 to about 900 nucleotides, about 950 to about 1000 nucleotides, about 1050 to about 1100 nucleotides, about 1150 to about 1200 nucleotides, about 1250 to about 1300 nucleotides, about 1350 to about 1400 nucleotides, about 1450 to about 1500 nucleotides, about 1550 to about 1600 nucleotides, about 1650 to about 1700 nucleotides, about 1750 to about 1800 nucleotides, about 1850 to about 1900 nucleotides, about 1950 to about 2000 nucleotides, about 2000 to about 3000 nucleotides, about 3000 to about 4000 nucleotides, or about 4000 to about 5000 nucleotides, downstream from the 5′ end of the intron comprising the NIE. In some embodiments, the ASO targets a sequence from about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, about 250 to about 300, about 250 to about 300 nucleotides, about 350 to about 400 nucleotides, about 450 to about 500 nucleotides, about 550 to about 600 nucleotides, about 650 to about 700 nucleotides, about 750 to about 800 nucleotides, about 850 to about 900 nucleotides, or about 950 to about 1000 nucleotides downstream from the 5′ end of the intron comprising the NIE.

In some embodiments, the ASO targets a sequence at least about 1 nucleotide, at least about 10 nucleotides, at least about 20 nucleotides, at least about 50 nucleotides, at least about 80 nucleotides, at least about 85 nucleotides, at least about 90 nucleotides, at least about 95 nucleotides, at least about 96 nucleotides, at least about 97 nucleotides, at least about 98 nucleotides, at least about 99 nucleotides, at least about 100 nucleotides, at least about 101 nucleotides, at least about 102 nucleotides, at least about 103 nucleotides, at least about 104 nucleotides, at least about 105 nucleotides, at least about 110 nucleotides, at least about 120 nucleotides, at least about 150 nucleotides, at least about 200 nucleotides, at least about 300 nucleotides, at least about 400 nucleotides, at least about 500 nucleotides, at least about 600 nucleotides, at least about 700 nucleotides, at least about 800 nucleotides, at least about 900 nucleotides, at least about 1000 nucleotides, at least about 1200 nucleotides, at least about 1400 nucleotides, at least about 1500 nucleotides, at least about 1600 nucleotides, at least about 1800 nucleotides, at least about 2000 nucleotides, at least about 3000 nucleotides, at least about 4000 nucleotides, or at least about 5000 nucleotides downstream from the 5′ end of the intron comprising the NIE.

In some embodiments, the ASO targets a sequence from about 1 to about 2000 nucleotides upstream (or 5′) from the 5′ end of the NIE. In some embodiments, the ASO targets a sequence from about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, about 250 to about 300, about 250 to about 300 nucleotides, about 350 to about 400 nucleotides, about 450 to about 500 nucleotides, about 550 to about 600 nucleotides, about 650 to about 700 nucleotides, about 750 to about 800 nucleotides, about 850 to about 900 nucleotides, about 950 to about 1000 nucleotides, about 1050 to about 1100 nucleotides, about 1150 to about 1200 nucleotides, about 1250 to about 1300 nucleotides, about 1350 to about 1400 nucleotides, about 1450 to about 1500 nucleotides, about 1550 to about 1600 nucleotides, about 1650 to about 1700 nucleotides, about 1750 to about 1800 nucleotides, about 1850 to about 1900 nucleotides, or about 1950 to about 2000 nucleotides upstream (or 5′) from the 5′ end of the NIE. In some embodiments, the ASO targets a sequence from about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, about 250 to about 300, about 250 to about 300 nucleotides, about 350 to about 400 nucleotides, about 450 to about 500 nucleotides, about 550 to about 600 nucleotides, about 650 to about 700 nucleotides, about 750 to about 800 nucleotides, about 850 to about 900 nucleotides, or about 950 to about 1000 nucleotides upstream (or 5′) from the 5′ end of the NIE.

In some embodiments, the ASO targets a sequence at least about 1 nucleotide, at least about 10 nucleotides, at least about 20 nucleotides, at least about 50 nucleotides, at least about 80 nucleotides, at least about 85 nucleotides, at least about 90 nucleotides, at least about 95 nucleotides, at least about 96 nucleotides, at least about 97 nucleotides, at least about 98 nucleotides, at least about 99 nucleotides, at least about 100 nucleotides, at least about 101 nucleotides, at least about 102 nucleotides, at least about 103 nucleotides, at least about 104 nucleotides, at least about 105 nucleotides, at least about 110 nucleotides, at least about 120 nucleotides, at least about 150 nucleotides, at least about 200 nucleotides, at least about 300 nucleotides, at least about 400 nucleotides, at least about 500 nucleotides, at least about 600 nucleotides, at least about 700 nucleotides, at least about 800 nucleotides, at least about 900 nucleotides, at least about 1000 nucleotides, at least about 1200 nucleotides, at least about 1400 nucleotides, at least about 1500 nucleotides, at least about 1600 nucleotides, at least about 1800 nucleotides, or at least about 2000 nucleotides upstream (or 5′) from the 5′ end of the NIE.

In some embodiments, the ASO targets a sequence from about 1 to about 500 nucleotides downstream from the 5′ end of the NIE. In some embodiments, the ASO targets a sequence at least about 1 nucleotide, at least about 10 nucleotides, at least about 20 nucleotides, at least about 50 nucleotides, at least about 80 nucleotides, at least about 85 nucleotides, at least about 90 nucleotides, at least about 95 nucleotides, at least about 96 nucleotides, at least about 97 nucleotides, at least about 98 nucleotides, at least about 99 nucleotides, at least about 100 nucleotides, at least about 101 nucleotides, at least about 102 nucleotides, at least about 103 nucleotides, at least about 104 nucleotides, at least about 105 nucleotides, at least about 110 nucleotides, at least about 120 nucleotides, at least about 150 nucleotides, at least about 200 nucleotides, at least about 300 nucleotides, at least about 400 nucleotides, or at least about 500 nucleotides downstream from the 5′ end of the NIE region.

In some embodiments, the ASO targets a sequence from about 1 to about 500 nucleotides upstream from the 3′ end of the NIE. In some embodiments, the ASO targets a sequence at least about 1 nucleotide, at least about 10 nucleotides, at least about 20 nucleotides, at least about 50 nucleotides, at least about 80 nucleotides, at least about 85 nucleotides, at least about 90 nucleotides, at least about 95 nucleotides, at least about 96 nucleotides, at least about 97 nucleotides, at least about 98 nucleotides, at least about 99 nucleotides, at least about 100 nucleotides, at least about 101 nucleotides, at least about 102 nucleotides, at least about 103 nucleotides, at least about 104 nucleotides, at least about 105 nucleotides, at least about 110 nucleotides, at least about 120 nucleotides, at least about 150 nucleotides, at least about 200 nucleotides, at least about 300 nucleotides, at least about 400 nucleotides, or at least about 500 nucleotides upstream from the 3′ end of the NIE region.

In some embodiments, the ASO targets a sequence from about 1 to about 2000 nucleotides downstream (or 3′) from the 3′ end of the NIE. In some embodiments, the ASO targets a sequence from about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, about 250 to about 300, about 250 to about 300 nucleotides, about 350 to about 400 nucleotides, about 450 to about 500 nucleotides, about 550 to about 600 nucleotides, about 650 to about 700 nucleotides, about 750 to about 800 nucleotides, about 850 to about 900 nucleotides, about 950 to about 1000 nucleotides, about 1050 to about 1100 nucleotides, about 1150 to about 1200 nucleotides, about 1250 to about 1300 nucleotides, about 1350 to about 1400 nucleotides, about 1450 to about 1500 nucleotides, about 1550 to about 1600 nucleotides, about 1650 to about 1700 nucleotides, about 1750 to about 1800 nucleotides, about 1850 to about 1900 nucleotides, or about 1950 to about 2000 nucleotides downstream (or 3′) from the 3′ end of the NIE. In some embodiments, the ASO targets a sequence from about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, about 250 to about 300, about 250 to about 300 nucleotides, about 350 to about 400 nucleotides, about 450 to about 500 nucleotides, about 550 to about 600 nucleotides, about 650 to about 700 nucleotides, about 750 to about 800 nucleotides, about 850 to about 900 nucleotides, or about 950 to about 1000 nucleotides downstream (or 3′) from the 3′ end of the NIE.

In some embodiments, the ASO targets a sequence at least about 1 nucleotide, at least about 10 nucleotides, at least about 20 nucleotides, at least about 50 nucleotides, at least about 80 nucleotides, at least about 85 nucleotides, at least about 90 nucleotides, at least about 95 nucleotides, at least about 96 nucleotides, at least about 97 nucleotides, at least about 98 nucleotides, at least about 99 nucleotides, at least about 100 nucleotides, at least about 101 nucleotides, at least about 102 nucleotides, at least about 103 nucleotides, at least about 104 nucleotides, at least about 105 nucleotides, at least about 110 nucleotides, at least about 120 nucleotides, at least about 150 nucleotides, at least about 200 nucleotides, at least about 300 nucleotides, at least about 400 nucleotides, at least about 500 nucleotides, at least about 600 nucleotides, at least about 700 nucleotides, at least about 800 nucleotides, at least about 900 nucleotides, at least about 1000 nucleotides, at least about 1200 nucleotides, at least about 1400 nucleotides, at least about 1500 nucleotides, at least about 1600 nucleotides, at least about 1800 nucleotides, or at least about 2000 nucleotides downstream (or 3′) from the 3′ end of the NIE.

In some embodiments, the ASO targets a sequence from about 1 to about 5000 nucleotides upstream from the 3′ end of the intron comprising the NIE. In some embodiments, the ASO targets a sequence from about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, about 250 to about 300, about 250 to about 300 nucleotides, about 350 to about 400 nucleotides, about 450 to about 500 nucleotides, about 550 to about 600 nucleotides, about 650 to about 700 nucleotides, about 750 to about 800 nucleotides, about 850 to about 900 nucleotides, about 950 to about 1000 nucleotides, about 1050 to about 1100 nucleotides, about 1150 to about 1200 nucleotides, about 1250 to about 1300 nucleotides, about 1350 to about 1400 nucleotides, about 1450 to about 1500 nucleotides, about 1550 to about 1600 nucleotides, about 1650 to about 1700 nucleotides, about 1750 to about 1800 nucleotides, about 1850 to about 1900 nucleotides, about 1950 to about 2000 nucleotides, about 2000 to about 3000 nucleotides, about 3000 to about 4000 nucleotides, or about 4000 to about 5000 nucleotides, upstream from the 3′ end of the intron comprising the NIE. In some embodiments, the ASO targets a sequence from about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, about 250 to about 300, about 250 to about 300 nucleotides, about 350 to about 400 nucleotides, about 450 to about 500 nucleotides, about 550 to about 600 nucleotides, about 650 to about 700 nucleotides, about 750 to about 800 nucleotides, about 850 to about 900 nucleotides, or about 950 to about 1000 nucleotides upstream from the 3′ end of the intron comprising the NIE.

In some embodiments, the ASO targets a sequence at least about 1 nucleotide, at least about 10 nucleotides, at least about 20 nucleotides, at least about 50 nucleotides, at least about 80 nucleotides, at least about 85 nucleotides, at least about 90 nucleotides, at least about 95 nucleotides, at least about 96 nucleotides, at least about 97 nucleotides, at least about 98 nucleotides, at least about 99 nucleotides, at least about 100 nucleotides, at least about 101 nucleotides, at least about 102 nucleotides, at least about 103 nucleotides, at least about 104 nucleotides, at least about 105 nucleotides, at least about 110 nucleotides, at least about 120 nucleotides, at least about 150 nucleotides, at least about 200 nucleotides, at least about 300 nucleotides, at least about 400 nucleotides, at least about 500 nucleotides, at least about 600 nucleotides, at least about 700 nucleotides, at least about 800 nucleotides, at least about 900 nucleotides, at least about 1000 nucleotides, at least about 1200 nucleotides, at least about 1400 nucleotides, at least about 1500 nucleotides, at least about 1600 nucleotides, at least about 1800 nucleotides, at least about 2000 nucleotides, at least about 3000 nucleotides, at least about 4000 nucleotides, or at least about 5000 nucleotides upstream from the 3′ end of the intron comprising the NIE.

In some embodiments, the ASO targets a sequence from about 4 to about 300 nucleotides upstream (or 5′) from the 5′ end of the NIE. In some embodiments, the ASO targets a sequence from about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, about 250 to about 300, about 250 to about 300 nucleotides, about 350 to about 400 nucleotides, about 450 to about 500 nucleotides, about 550 to about 600 nucleotides, about 650 to about 700 nucleotides, about 750 to about 800 nucleotides, about 850 to about 900 nucleotides, about 950 to about 1000 nucleotides, about 1050 to about 1100 nucleotides, about 1150 to about 1200 nucleotides, about 1250 to about 1300 nucleotides, about 1350 to about 1400 nucleotides, or about 1450 to about 1500 nucleotides upstream (or 5′) from the 5′ end of the NIE region. In some embodiments, the ASO may target a sequence more than 300 nucleotides upstream from the 5′ end of the NIE. In some embodiments, the ASO targets a sequence from about 4 to about 300 nucleotides downstream (or 3′) from the 3′ end of the NIE. In some embodiments, the ASO targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, about 250 to about 300 nucleotides, about 350 to about 400 nucleotides, about 450 to about 500 nucleotides, about 550 to about 600 nucleotides, about 650 to about 700 nucleotides, about 750 to about 800 nucleotides, about 850 to about 900 nucleotides, about 950 to about 1000 nucleotides, about 1050 to about 1100 nucleotides, about 1150 to about 1200 nucleotides, about 1250 to about 1300 nucleotides, about 1350 to about 1400 nucleotides, or about 1450 to about 1500 nucleotides downstream from the 3′ end of the NIE. In some embodiments, the ASO targets a sequence more than 300 nucleotides downstream from the 3′ end of the NIE.

In some embodiments, the ASO targets a sequence from about 4 to about 300 nucleotides upstream (or 5′) from the 5′ end of the NIE. In some embodiments, the ASO targets a sequence at least about 1 nucleotide, at least about 10 nucleotides, at least about 20 nucleotides, at least about 50 nucleotides, at least about 80 nucleotides, at least about 85 nucleotides, at least about 90 nucleotides, at least about 95 nucleotides, at least about 96 nucleotides, at least about 97 nucleotides, at least about 98 nucleotides, at least about 99 nucleotides, at least about 100 nucleotides, at least about 101 nucleotides, at least about 102 nucleotides, at least about 103 nucleotides, at least about 104 nucleotides, at least about 105 nucleotides, at least about 110 nucleotides, at least about 120 nucleotides, at least about 150 nucleotides, at least about 200 nucleotides, at least about 300 nucleotides, at least about 400 nucleotides, at least about 500 nucleotides, at least about 600 nucleotides, at least about 700 nucleotides, at least about 800 nucleotides, at least about 900 nucleotides, or at least about 1000 nucleotides upstream (or 5′) from the 5′ end of the NIE region. In some embodiments, the ASO targets a sequence about 4 to about 300 nucleotides downstream (or 3′) from the 3′ end of the NIE. In some embodiments, the ASO targets a sequence at least about 1 nucleotide, at least about 10 nucleotides, at least about 20 nucleotides, at least about 50 nucleotides, at least about 80 nucleotides, at least about 85 nucleotides, at least about 90 nucleotides, at least about 95 nucleotides, at least about 96 nucleotides, at least about 97 nucleotides, at least about 98 nucleotides, at least about 99 nucleotides, at least about 100 nucleotides, at least about 101 nucleotides, at least about 102 nucleotides, at least about 103 nucleotides, at least about 104 nucleotides, at least about 105 nucleotides, at least about 110 nucleotides, at least about 120 nucleotides, at least about 150 nucleotides, at least about 200 nucleotides, at least about 300 nucleotides, at least about 400 nucleotides, at least about 500 nucleotides, at least about 600 nucleotides, at least about 700 nucleotides, at least about 800 nucleotides, at least about 900 nucleotides, or at least about 1000 nucleotides downstream from the 3′ end of the NIE. In some embodiments, the ASO targets a sequence more than 300 nucleotides downstream from the 3′ end of the NIE.

In some embodiments, the ASO targets a sequence from about 4 to about 300 nucleotides upstream (or 5′) from the 5′ end of the NIE. In some embodiments, the ASO targets a sequence at most about 10 nucleotides, at most about 20 nucleotides, at most about 50 nucleotides, at most about 80 nucleotides, at most about 85 nucleotides, at most about 90 nucleotides, at most about 95 nucleotides, at most about 96 nucleotides, at most about 97 nucleotides, at most about 98 nucleotides, at most about 99 nucleotides, at most about 100 nucleotides, at most about 101 nucleotides, at most about 102 nucleotides, at most about 103 nucleotides, at most about 104 nucleotides, at most about 105 nucleotides, at most about 110 nucleotides, at most about 120 nucleotides, at most about 150 nucleotides, at most about 200 nucleotides, at most about 300 nucleotides, at most about 400 nucleotides, at most about 500 nucleotides, at most about 600 nucleotides, at most about 700 nucleotides, at most about 800 nucleotides, at most about 900 nucleotides, at most about 1000 nucleotides, at most about 1100 nucleotides, at most about 1200 nucleotides, at most about 1300 nucleotides, at most about 1400 nucleotides, or at most about 1500 nucleotides upstream (or 5′) from the 5′ end of the NIE region. In some embodiments, the ASO targets a sequence about 4 to about 300 nucleotides downstream (or 3′) from the 3′ end of the NIE. In some embodiments, the ASO targets a sequence at most about 10 nucleotides, at most about 20 nucleotides, at most about 50 nucleotides, at most about 80 nucleotides, at most about 85 nucleotides, at most about 90 nucleotides, at most about 95 nucleotides, at most about 96 nucleotides, at most about 97 nucleotides, at most about 98 nucleotides, at most about 99 nucleotides, at most about 100 nucleotides, at most about 101 nucleotides, at most about 102 nucleotides, at most about 103 nucleotides, at most about 104 nucleotides, at most about 105 nucleotides, at most about 110 nucleotides, at most about 120 nucleotides, at most about 150 nucleotides, at most about 200 nucleotides, at most about 300 nucleotides, at most about 400 nucleotides, at most about 500 nucleotides, at most about 600 nucleotides, at most about 700 nucleotides, at most about 800 nucleotides, at most about 900 nucleotides, or at most about 1000 nucleotides, at most about 1100 nucleotides, at most about 1200 nucleotides, at most about 1300 nucleotides, at most about 1400 nucleotides, or at most about 1500 nucleotides downstream from the 3′ end of the NIE. In some embodiments, the ASO targets a sequence more than 300 nucleotides downstream from the 3′ end of the NIE.

In some embodiments, the NIE (exon 23) as described herein is located between GRCh38/hg38: chr2: 166007230 and chr2: 166007293. In some embodiments, the 5′ end of the NIE is located at GRCh38/hg38: chr2: 166007230. In some embodiments, the 3′ end of the NIE is located at GRCh38/hg38: chr2: 166007293.

In some embodiments, the ASO targets a sequence from about 1 to about 2000 nucleotides upstream (or 5′) from genomic site GRCh38/hg38: chr2: 166007230. In some embodiments, the ASO targets a sequence from about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, about 250 to about 300, about 250 to about 300 nucleotides, about 350 to about 400 nucleotides, about 450 to about 500 nucleotides, about 550 to about 600 nucleotides, about 650 to about 700 nucleotides, about 750 to about 800 nucleotides, about 850 to about 900 nucleotides, about 950 to about 1000 nucleotides, about 1050 to about 1100 nucleotides, about 1150 to about 1200 nucleotides, about 1250 to about 1300 nucleotides, about 1350 to about 1400 nucleotides, about 1450 to about 1500 nucleotides, about 1550 to about 1600 nucleotides, about 1650 to about 1700 nucleotides, about 1750 to about 1800 nucleotides, about 1850 to about 1900 nucleotides, or about 1950 to about 2000 nucleotides upstream (or 5′) from genomic site GRCh38/hg38: chr2: 166007230. In some embodiments, the ASO targets a sequence from about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, about 250 to about 300, about 250 to about 300 nucleotides, about 350 to about 400 nucleotides, about 450 to about 500 nucleotides, about 550 to about 600 nucleotides, about 650 to about 700 nucleotides, about 750 to about 800 nucleotides, about 850 to about 900 nucleotides, or about 950 to about 1000 nucleotides upstream (or 5′) from genomic site GRCh38/hg38: chr2: 166007230.

In some embodiments, the ASO targets a sequence at least about 1 nucleotide, at least about 10 nucleotides, at least about 20 nucleotides, at least about 50 nucleotides, at least about 80 nucleotides, at least about 85 nucleotides, at least about 90 nucleotides, at least about 95 nucleotides, at least about 96 nucleotides, at least about 97 nucleotides, at least about 98 nucleotides, at least about 99 nucleotides, at least about 100 nucleotides, at least about 101 nucleotides, at least about 102 nucleotides, at least about 103 nucleotides, at least about 104 nucleotides, at least about 105 nucleotides, at least about 110 nucleotides, at least about 120 nucleotides, at least about 150 nucleotides, at least about 200 nucleotides, at least about 300 nucleotides, at least about 400 nucleotides, at least about 500 nucleotides, at least about 600 nucleotides, at least about 700 nucleotides, at least about 800 nucleotides, at least about 900 nucleotides, at least about 1000 nucleotides, at least about 1200 nucleotides, at least about 1400 nucleotides, at least about 1500 nucleotides, at least about 1600 nucleotides, at least about 1800 nucleotides, or at least about 2000 nucleotides upstream (or 5′) from genomic site GRCh38/hg38: chr2: 166007230.

In some embodiments, the ASO targets a sequence from about 1 to about 500 nucleotides downstream from genomic site GRCh38/hg38: chr2: 166007230. In some embodiments, the ASO targets a sequence at least about 1 nucleotide, at least about 10 nucleotides, at least about 20 nucleotides, at least about 50 nucleotides, at least about 80 nucleotides, at least about 85 nucleotides, at least about 90 nucleotides, at least about 95 nucleotides, at least about 96 nucleotides, at least about 97 nucleotides, at least about 98 nucleotides, at least about 99 nucleotides, at least about 100 nucleotides, at least about 101 nucleotides, at least about 102 nucleotides, at least about 103 nucleotides, at least about 104 nucleotides, at least about 105 nucleotides, at least about 110 nucleotides, at least about 120 nucleotides, at least about 150 nucleotides, at least about 200 nucleotides, at least about 300 nucleotides, at least about 400 nucleotides, or at least about 500 nucleotides downstream from genomic site GRCh38/hg38: chr2: 166007230.

In some embodiments, the ASO targets a sequence from about 1 to about 500 nucleotides upstream from genomic site GRCh38/hg38: chr2: 166007293. In some embodiments, the ASO targets a sequence at least about 1 nucleotide, at least about 10 nucleotides, at least about 20 nucleotides, at least about 50 nucleotides, at least about 80 nucleotides, at least about 85 nucleotides, at least about 90 nucleotides, at least about 95 nucleotides, at least about 96 nucleotides, at least about 97 nucleotides, at least about 98 nucleotides, at least about 99 nucleotides, at least about 100 nucleotides, at least about 101 nucleotides, at least about 102 nucleotides, at least about 103 nucleotides, at least about 104 nucleotides, at least about 105 nucleotides, at least about 110 nucleotides, at least about 120 nucleotides, at least about 150 nucleotides, at least about 200 nucleotides, at least about 300 nucleotides, at least about 400 nucleotides, or at least about 500 nucleotides upstream from genomic site GRCh38/hg38: chr2: 166007293.

In some embodiments, the ASO targets a sequence from about 1 to about 2000 nucleotides downstream (or 3′) from genomic site GRCh38/hg38: chr2: 166007293. In some embodiments, the ASO targets a sequence from about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, about 250 to about 300, about 250 to about 300 nucleotides, about 350 to about 400 nucleotides, about 450 to about 500 nucleotides, about 550 to about 600 nucleotides, about 650 to about 700 nucleotides, about 750 to about 800 nucleotides, about 850 to about 900 nucleotides, about 950 to about 1000 nucleotides, about 1050 to about 1100 nucleotides, about 1150 to about 1200 nucleotides, about 1250 to about 1300 nucleotides, about 1350 to about 1400 nucleotides, about 1450 to about 1500 nucleotides, about 1550 to about 1600 nucleotides, about 1650 to about 1700 nucleotides, about 1750 to about 1800 nucleotides, about 1850 to about 1900 nucleotides, or about 1950 to about 2000 nucleotides downstream (or 3′) from genomic site GRCh38/hg38: chr2: 166007293. In some embodiments, the ASO targets a sequence from about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, about 250 to about 300, about 250 to about 300 nucleotides, about 350 to about 400 nucleotides, about 450 to about 500 nucleotides, about 550 to about 600 nucleotides, about 650 to about 700 nucleotides, about 750 to about 800 nucleotides, about 850 to about 900 nucleotides, or about 950 to about 1000 nucleotides downstream (or 3′) from genomic site GRCh38/hg38: chr2: 166007293.

In some embodiments, the ASO targets a sequence at least about 1 nucleotide, at least about 10 nucleotides, at least about 20 nucleotides, at least about 50 nucleotides, at least about 80 nucleotides, at least about 85 nucleotides, at least about 90 nucleotides, at least about 95 nucleotides, at least about 96 nucleotides, at least about 97 nucleotides, at least about 98 nucleotides, at least about 99 nucleotides, at least about 100 nucleotides, at least about 101 nucleotides, at least about 102 nucleotides, at least about 103 nucleotides, at least about 104 nucleotides, at least about 105 nucleotides, at least about 110 nucleotides, at least about 120 nucleotides, at least about 150 nucleotides, at least about 200 nucleotides, at least about 300 nucleotides, at least about 400 nucleotides, at least about 500 nucleotides, at least about 600 nucleotides, at least about 700 nucleotides, at least about 800 nucleotides, at least about 900 nucleotides, at least about 1000 nucleotides, at least about 1200 nucleotides, at least about 1400 nucleotides, at least about 1500 nucleotides, at least about 1600 nucleotides, at least about 1800 nucleotides, or at least about 2000 nucleotides downstream (or 3′) from genomic site GRCh38/hg38: chr2: 166007293.

In some embodiments, the intron comprising the NIE is located between GRCh38/hg38: chr2: 166002754 and chr2: 166009718. In some embodiments, the 5′ end of the intron comprising the NIE is located at GRCh38/hg38: chr2: 166002754. In some embodiments, the 3′ end of the intron comprising the NIE is located at GRCh38/hg38: chr2: 166009718.

In some embodiments, the ASO targets a sequence from about 1 to about 5000 nucleotides downstream from genomic site GRCh38/hg38: chr2: 166002754. In some embodiments, the ASO targets a sequence from about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, about 250 to about 300, about 250 to about 300 nucleotides, about 350 to about 400 nucleotides, about 450 to about 500 nucleotides, about 550 to about 600 nucleotides, about 650 to about 700 nucleotides, about 750 to about 800 nucleotides, about 850 to about 900 nucleotides, about 950 to about 1000 nucleotides, about 1050 to about 1100 nucleotides, about 1150 to about 1200 nucleotides, about 1250 to about 1300 nucleotides, about 1350 to about 1400 nucleotides, about 1450 to about 1500 nucleotides, about 1550 to about 1600 nucleotides, about 1650 to about 1700 nucleotides, about 1750 to about 1800 nucleotides, about 1850 to about 1900 nucleotides, about 1950 to about 2000 nucleotides, about 2000 to about 3000 nucleotides, about 3000 to about 4000 nucleotides, or about 4000 to about 5000 nucleotides, downstream from genomic site GRCh38/hg38: chr2: 166002754. In some embodiments, the ASO targets a sequence from about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, about 250 to about 300, about 250 to about 300 nucleotides, about 350 to about 400 nucleotides, about 450 to about 500 nucleotides, about 550 to about 600 nucleotides, about 650 to about 700 nucleotides, about 750 to about 800 nucleotides, about 850 to about 900 nucleotides, or about 950 to about 1000 nucleotides downstream from genomic site GRCh38/hg38: chr2: 166002754.

In some embodiments, the ASO targets a sequence at least about 1 nucleotide, at least about 10 nucleotides, at least about 20 nucleotides, at least about 50 nucleotides, at least about 80 nucleotides, at least about 85 nucleotides, at least about 90 nucleotides, at least about 95 nucleotides, at least about 96 nucleotides, at least about 97 nucleotides, at least about 98 nucleotides, at least about 99 nucleotides, at least about 100 nucleotides, at least about 101 nucleotides, at least about 102 nucleotides, at least about 103 nucleotides, at least about 104 nucleotides, at least about 105 nucleotides, at least about 110 nucleotides, at least about 120 nucleotides, at least about 150 nucleotides, at least about 200 nucleotides, at least about 300 nucleotides, at least about 400 nucleotides, at least about 500 nucleotides, at least about 600 nucleotides, at least about 700 nucleotides, at least about 800 nucleotides, at least about 900 nucleotides, at least about 1000 nucleotides, at least about 1200 nucleotides, at least about 1400 nucleotides, at least about 1500 nucleotides, at least about 1600 nucleotides, at least about 1800 nucleotides, at least about 2000 nucleotides, at least about 3000 nucleotides, at least about 4000 nucleotides, or at least about 5000 nucleotides downstream from genomic site GRCh38/hg38: chr2: 166002754.

In some embodiments, the ASO targets a sequence from about 1 to about 5000 nucleotides upstream from genomic site GRCh38/hg38: chr2: 166007229. In some embodiments, the ASO targets a sequence from about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, about 250 to about 300, about 250 to about 300 nucleotides, about 350 to about 400 nucleotides, about 450 to about 500 nucleotides, about 550 to about 600 nucleotides, about 650 to about 700 nucleotides, about 750 to about 800 nucleotides, about 850 to about 900 nucleotides, about 950 to about 1000 nucleotides, about 1050 to about 1100 nucleotides, about 1150 to about 1200 nucleotides, about 1250 to about 1300 nucleotides, about 1350 to about 1400 nucleotides, about 1450 to about 1500 nucleotides, about 1550 to about 1600 nucleotides, about 1650 to about 1700 nucleotides, about 1750 to about 1800 nucleotides, about 1850 to about 1900 nucleotides, about 1950 to about 2000 nucleotides, about 2000 to about 3000 nucleotides, about 3000 to about 4000 nucleotides, or about 4000 to about 5000 nucleotides, upstream from genomic site GRCh38/hg38: chr2: 166007229. In some embodiments, the ASO targets a sequence from about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, about 250 to about 300, about 250 to about 300 nucleotides, about 350 to about 400 nucleotides, about 450 to about 500 nucleotides, about 550 to about 600 nucleotides, about 650 to about 700 nucleotides, about 750 to about 800 nucleotides, about 850 to about 900 nucleotides, or about 950 to about 1000 nucleotides upstream from genomic site GRCh38/hg38: chr2: 166007229.

In some embodiments, the ASO targets a sequence at least about 1 nucleotide, at least about 10 nucleotides, at least about 20 nucleotides, at least about 50 nucleotides, at least about 80 nucleotides, at least about 85 nucleotides, at least about 90 nucleotides, at least about 95 nucleotides, at least about 96 nucleotides, at least about 97 nucleotides, at least about 98 nucleotides, at least about 99 nucleotides, at least about 100 nucleotides, at least about 101 nucleotides, at least about 102 nucleotides, at least about 103 nucleotides, at least about 104 nucleotides, at least about 105 nucleotides, at least about 110 nucleotides, at least about 120 nucleotides, at least about 150 nucleotides, at least about 200 nucleotides, at least about 300 nucleotides, at least about 400 nucleotides, at least about 500 nucleotides, at least about 600 nucleotides, at least about 700 nucleotides, at least about 800 nucleotides, at least about 900 nucleotides, at least about 1000 nucleotides, at least about 1200 nucleotides, at least about 1400 nucleotides, at least about 1500 nucleotides, at least about 1600 nucleotides, at least about 1800 nucleotides, at least about 2000 nucleotides, at least about 3000 nucleotides, at least about 4000 nucleotides, or at least about 5000 nucleotides upstream from genomic site GRCh38/hg38: chr2: 166007229.

In some embodiments, the ASO targets a sequence from about 1 to about 5000 nucleotides downstream from genomic site GRCh38/hg38: chr2: 166007294. In some embodiments, the ASO targets a sequence from about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, about 250 to about 300, about 250 to about 300 nucleotides, about 350 to about 400 nucleotides, about 450 to about 500 nucleotides, about 550 to about 600 nucleotides, about 650 to about 700 nucleotides, about 750 to about 800 nucleotides, about 850 to about 900 nucleotides, about 950 to about 1000 nucleotides, about 1050 to about 1100 nucleotides, about 1150 to about 1200 nucleotides, about 1250 to about 1300 nucleotides, about 1350 to about 1400 nucleotides, about 1450 to about 1500 nucleotides, about 1550 to about 1600 nucleotides, about 1650 to about 1700 nucleotides, about 1750 to about 1800 nucleotides, about 1850 to about 1900 nucleotides, about 1950 to about 2000 nucleotides, about 2000 to about 3000 nucleotides, about 3000 to about 4000 nucleotides, or about 4000 to about 5000 nucleotides, downstream from genomic site GRCh38/hg38: chr2: 166007294. In some embodiments, the ASO targets a sequence from about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, about 250 to about 300, about 250 to about 300 nucleotides, about 350 to about 400 nucleotides, about 450 to about 500 nucleotides, about 550 to about 600 nucleotides, about 650 to about 700 nucleotides, about 750 to about 800 nucleotides, about 850 to about 900 nucleotides, or about 950 to about 1000 nucleotides downstream from genomic site GRCh38/hg38: chr2: 166007294.

In some embodiments, the ASO targets a sequence at least about 1 nucleotide, at least about 10 nucleotides, at least about 20 nucleotides, at least about 50 nucleotides, at least about 80 nucleotides, at least about 85 nucleotides, at least about 90 nucleotides, at least about 95 nucleotides, at least about 96 nucleotides, at least about 97 nucleotides, at least about 98 nucleotides, at least about 99 nucleotides, at least about 100 nucleotides, at least about 101 nucleotides, at least about 102 nucleotides, at least about 103 nucleotides, at least about 104 nucleotides, at least about 105 nucleotides, at least about 110 nucleotides, at least about 120 nucleotides, at least about 150 nucleotides, at least about 200 nucleotides, at least about 300 nucleotides, at least about 400 nucleotides, at least about 500 nucleotides, at least about 600 nucleotides, at least about 700 nucleotides, at least about 800 nucleotides, at least about 900 nucleotides, at least about 1000 nucleotides, at least about 1200 nucleotides, at least about 1400 nucleotides, at least about 1500 nucleotides, at least about 1600 nucleotides, at least about 1800 nucleotides, at least about 2000 nucleotides, at least about 3000 nucleotides, at least about 4000 nucleotides, or at least about 5000 nucleotides downstream from genomic site GRCh38/hg38: chr2: 166007294.

In some embodiments, the ASO targets a sequence from about 1 to about 5000 nucleotides upstream from genomic site GRCh38/hg38: chr2: 166009718. In some embodiments, the ASO targets a sequence from about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, about 250 to about 300, about 250 to about 300 nucleotides, about 350 to about 400 nucleotides, about 450 to about 500 nucleotides, about 550 to about 600 nucleotides, about 650 to about 700 nucleotides, about 750 to about 800 nucleotides, about 850 to about 900 nucleotides, about 950 to about 1000 nucleotides, about 1050 to about 1100 nucleotides, about 1150 to about 1200 nucleotides, about 1250 to about 1300 nucleotides, about 1350 to about 1400 nucleotides, about 1450 to about 1500 nucleotides, about 1550 to about 1600 nucleotides, about 1650 to about 1700 nucleotides, about 1750 to about 1800 nucleotides, about 1850 to about 1900 nucleotides, about 1950 to about 2000 nucleotides, about 2000 to about 3000 nucleotides, about 3000 to about 4000 nucleotides, or about 4000 to about 5000 nucleotides, upstream from genomic site GRCh38/hg38: chr2: 166009718. In some embodiments, the ASO targets a sequence from about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, about 250 to about 300, about 250 to about 300 nucleotides, about 350 to about 400 nucleotides, about 450 to about 500 nucleotides, about 550 to about 600 nucleotides, about 650 to about 700 nucleotides, about 750 to about 800 nucleotides, about 850 to about 900 nucleotides, or about 950 to about 1000 nucleotides upstream from genomic site GRCh38/hg38: chr2: 166009718.

In some embodiments, the ASO targets a sequence at least about 1 nucleotide, at least about 10 nucleotides, at least about 20 nucleotides, at least about 50 nucleotides, at least about 80 nucleotides, at least about 85 nucleotides, at least about 90 nucleotides, at least about 95 nucleotides, at least about 96 nucleotides, at least about 97 nucleotides, at least about 98 nucleotides, at least about 99 nucleotides, at least about 100 nucleotides, at least about 101 nucleotides, at least about 102 nucleotides, at least about 103 nucleotides, at least about 104 nucleotides, at least about 105 nucleotides, at least about 110 nucleotides, at least about 120 nucleotides, at least about 150 nucleotides, at least about 200 nucleotides, at least about 300 nucleotides, at least about 400 nucleotides, at least about 500 nucleotides, at least about 600 nucleotides, at least about 700 nucleotides, at least about 800 nucleotides, at least about 900 nucleotides, at least about 1000 nucleotides, at least about 1200 nucleotides, at least about 1400 nucleotides, at least about 1500 nucleotides, at least about 1600 nucleotides, at least about 1800 nucleotides, at least about 2000 nucleotides, at least about 3000 nucleotides, at least about 4000 nucleotides, or at least about 5000 nucleotides upstream from genomic site GRCh38/hg38: chr2: 166009718.

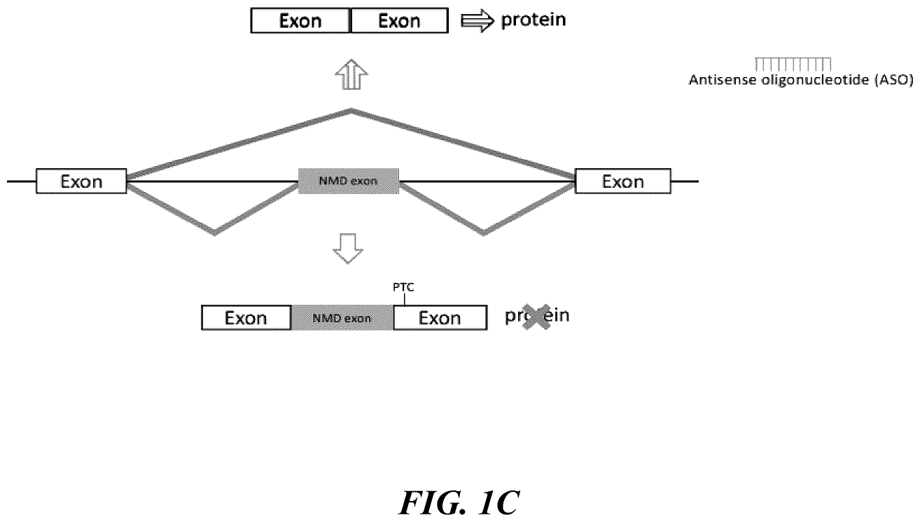

In some embodiments, the NIE as described herein is located between GRCh37/hg19: chr2: 166,863,740 and GRCh37/hg19: chr2: 166,863,803, as depicted in FIG. 2. In some embodiments, the 5′ end of the NIE is located at GRCh37/hg19: chr2: 166,863,803. In some embodiments, the 3′ end of the NIE is located at GRCh37/hg19: chr2: 166,863,740.

In some embodiments, In some embodiments, the ASO targets a sequence from about 4 to about 300 nucleotides upstream (or 5′) from genomic site GRCh37/hg19: chr2: 166,863,803. In some embodiments, the ASO targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, about 250 to about 300, about 250 to about 300 nucleotides, about 350 to about 400 nucleotides, about 450 to about 500 nucleotides, about 550 to about 600 nucleotides, about 650 to about 700 nucleotides, about 750 to about 800 nucleotides, about 850 to about 900 nucleotides, about 950 to about 1000 nucleotides, about 1050 to about 1100 nucleotides, about 1150 to about 1200 nucleotides, about 1250 to about 1300 nucleotides, about 1350 to about 1400 nucleotides, or about 1450 to about 1500 nucleotides upstream (or 5′) from genomic site GRCh37/hg19: chr2: 166,863,803. In some embodiments, the ASO may target a sequence more than 300 nucleotides upstream from genomic site GRCh37/hg19: chr2: 166,863,803. In some embodiments, the ASO targets a sequence from about 4 to about 300 nucleotides downstream (or 3′) from GRCh37/hg19: chr2: 166,863,740. In some embodiments, the ASO targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, about 250 to about 300 nucleotides, about 350 to about 400 nucleotides, about 450 to about 500 nucleotides, about 550 to about 600 nucleotides, about 650 to about 700 nucleotides, about 750 to about 800 nucleotides, about 850 to about 900 nucleotides, about 950 to about 1000 nucleotides, about 1050 to about 1100 nucleotides, about 1150 to about 1200 nucleotides, about 1250 to about 1300 nucleotides, about 1350 to about 1400 nucleotides, or about 1450 to about 1500 nucleotides downstream from GRCh37/hg19: chr2: 166,863,740. In some embodiments, the ASO targets a sequence more than 300 nucleotides downstream from GRCh37/hg19: chr2: 166,863,740.

In some embodiments, the ASO targets a sequence from about 4 to about 300 nucleotides upstream (or 5′) from genomic site GRCh37/hg19: chr2: 166,863,803. In some embodiments, the ASO targets a sequence at least about 1 nucleotide, at least about 10 nucleotides, at least about 20 nucleotides, at least about 50 nucleotides, at least about 80 nucleotides, at least about 85 nucleotides, at least about 90 nucleotides, at least about 95 nucleotides, at least about 96 nucleotides, at least about 97 nucleotides, at least about 98 nucleotides, at least about 99 nucleotides, at least about 100 nucleotides, at least about 101 nucleotides, at least about 102 nucleotides, at least about 103 nucleotides, at least about 104 nucleotides, at least about 105 nucleotides, at least about 110 nucleotides, at least about 120 nucleotides, at least about 150 nucleotides, at least about 200 nucleotides, at least about 300 nucleotides, at least about 400 nucleotides, at least about 500 nucleotides, at least about 600 nucleotides, at least about 700 nucleotides, at least about 800 nucleotides, at least about 900 nucleotides, or at least about 1000 nucleotides upstream (or 5′) from genomic site GRCh37/hg19: chr2: 166,863,803. In some embodiments, the ASO targets a sequence from about 4 to about 300 nucleotides downstream (or 3′) from GRCh37/hg19: chr2: 166,863,740. In some embodiments, the ASO targets a sequence at least about 1 nucleotide, at least about 10 nucleotides, at least about 20 nucleotides, at least about 50 nucleotides, at least about 80 nucleotides, at least about 85 nucleotides, at least about 90 nucleotides, at least about 95 nucleotides, at least about 96 nucleotides, at least about 97 nucleotides, at least about 98 nucleotides, at least about 99 nucleotides, at least about 100 nucleotides, at least about 101 nucleotides, at least about 102 nucleotides, at least about 103 nucleotides, at least about 104 nucleotides, at least about 105 nucleotides, at least about 110 nucleotides, at least about 120 nucleotides, at least about 150 nucleotides, at least about 200 nucleotides, at least about 300 nucleotides, at least about 400 nucleotides, at least about 500 nucleotides, at least about 600 nucleotides, at least about 700 nucleotides, at least about 800 nucleotides, at least about 900 nucleotides, or at least about 1000 nucleotides downstream from GRCh37/hg19: chr2: 166,863,740. In some embodiments, the ASO targets a sequence more than 300 nucleotides downstream from GRCh37/hg19: chr2: 166,863,740.

In some embodiments, the ASO targets a sequence from about 4 to about 300 nucleotides upstream (or 5′) from genomic site GRCh37/hg19: chr2: 166,863,803. In some embodiments, the ASO targets a sequence at most about 10 nucleotides, at most about 20 nucleotides, at most about 50 nucleotides, at most about 80 nucleotides, at most about 85 nucleotides, at most about 90 nucleotides, at most about 95 nucleotides, at most about 96 nucleotides, at most about 97 nucleotides, at most about 98 nucleotides, at most about 99 nucleotides, at most about 100 nucleotides, at most about 101 nucleotides, at most about 102 nucleotides, at most about 103 nucleotides, at most about 104 nucleotides, at most about 105 nucleotides, at most about 110 nucleotides, at most about 120 nucleotides, at most about 150 nucleotides, at most about 200 nucleotides, at most about 300 nucleotides, at most about 400 nucleotides, at most about 500 nucleotides, at most about 600 nucleotides, at most about 700 nucleotides, at most about 800 nucleotides, at most about 900 nucleotides, at most about 1000 nucleotides, at most about 1100 nucleotides, at most about 1200 nucleotides, at most about 1300 nucleotides, at most about 1400 nucleotides, or at most about 1500 nucleotides upstream (or 5′) from genomic site GRCh37/hg19: chr2: 166,863,803. In some embodiments, the ASO targets a sequence from about 4 to about 300 nucleotides downstream (or 3′) from GRCh37/hg19: chr2: 166,863,740. In some embodiments, the ASO targets a sequence at most about 10 nucleotides, at most about 20 nucleotides, at most about 50 nucleotides, at most about 80 nucleotides, at most about 85 nucleotides, at most about 90 nucleotides, at most about 95 nucleotides, at most about 96 nucleotides, at most about 97 nucleotides, at most about 98 nucleotides, at most about 99 nucleotides, at most about 100 nucleotides, at most about 101 nucleotides, at most about 102 nucleotides, at most about 103 nucleotides, at most about 104 nucleotides, at most about 105 nucleotides, at most about 110 nucleotides, at most about 120 nucleotides, at most about 150 nucleotides, at most about 200 nucleotides, at most about 300 nucleotides, at most about 400 nucleotides, at most about 500 nucleotides, at most about 600 nucleotides, at most about 700 nucleotides, at most about 800 nucleotides, at most about 900 nucleotides, or at most about 1000 nucleotides, at most about 1100 nucleotides, at most about 1200 nucleotides, at most about 1300 nucleotides, at most about 1400 nucleotides, or at most about 1500 nucleotides downstream from GRCh37/hg19: chr2: 166,863,740. In some embodiments, the ASO targets a sequence more than 300 nucleotides downstream from GRCh37/hg19: chr2: 166,863,740.

As described herein in the Examples, the SCN1A gene (SEQ ID NO. 1) was analyzed for NIE and inclusion of a portion of intron 20 (see SEQ ID NO. 6 which encodes the Intron 20 pre-mRNA) (this portion is referred as Exon 23 or Exon 20x throughout the present disclosure) was observed. In some embodiments, the ASOs disclosed herein target a NIE containing pre-mRNA (SEQ ID NO. 2) transcribed from a SCN1A genomic sequence. In some embodiments, the ASO targets a NIE containing pre-mRNA transcript from a SCN1A genomic sequence comprising a portion of intron 20. In some embodiments, the ASO targets a NIE containing pre-mRNA transcript from a SCN1A genomic sequence comprising exon 23 (or exon 20x) (SEQ ID NO. 4). In some embodiments, the ASO targets a NIE containing pre-mRNA transcript of SEQ ID NO. 2 or 9. In some embodiments, the ASO targets a NIE containing pre-mRNA transcript of SEQ ID NO. 2 or 9 comprising an NIE. In some embodiments, the ASO targets a NIE containing pre-mRNA transcript of SEQ ID NO. 2 comprising exon 23 (or exon 20x) (SEQ ID NO. 7). In some embodiments, the ASOs disclosed herein target a SCN1A pre-mRNA sequence (SEQ ID NO. 2 or 9). In some embodiments, the ASO targets a SCN1A pre-mRNA sequence comprising an NIE (SEQ ID NO. 7 or 11). In some embodiments, the ASO targets a SCN1A pre-mRNA sequence according to any one of SEQ ID NOs: 6, 7, 10, or 11. In some embodiments, the ASO has a sequence according to any one of SEQ ID NOs: 12-731. In some embodiments, the ASO has a sequence according to any one of SEQ ID NOs: 12-371. In some embodiments, the ASO has a sequence according to any one of SEQ ID NOs: 372-731.

In some embodiments, the SCN1A NIE containing pre-mRNA transcript is encoded by a genetic sequence with at least about 80%, 85%, 90%, 95%, 96%, 97%, 98%, 99% or 100% sequence identity to SEQ ID NO.: 1 or 8. In some embodiments, the SCN1A NIE pre-mRNA transcript comprises a sequence with at least about 80%, 85%, 90%, 95%, 96%, 97%, 98%, 99% or 100% sequence identity to any one of SEQ ID NOs: 2-7 and 9-11.

In some embodiments, the SCN1A NIE containing pre-mRNA transcript (or NMD exon mRNA) comprises a sequence with at least about 80%, 85%, 90%, 95%, 97%, or 100% sequence identity to any one of SEQ ID NOs: 2, 6, 7, 9, 10, and 12. In some embodiments, SCN1A NIE containing pre-mRNA transcript (or NMD exon mRNA) is encoded by a sequence with at least about 80%, 85%, 90%, 95%, 97%, or 100% sequence identity to SEQ ID NOs: 1 and 8. In some embodiments, the targeted portion of the NMD exon mRNA comprises a sequence with at least 80%, 85%, 90%, 95%, 97%, or 100% sequence identity to a region comprising at least 8 contiguous nucleic acids of SEQ ID NOs: 2, 6, 7, 9, 10, and 12.

In some embodiments, the ASO targets a sequence upstream from the 5′ end of an NIE. For example, ASOs targeting a sequence upstream from the 5′ end of an NIE (e.g. exon 23 (or exon 20x) in human SCN1A, or exon 21x in mouse SCN1A) can comprise a sequence with at least 80%, 85%, 90%, 95%, 97%, or 100% sequence identity to any one of SEQ ID NOs: 12-191 or 372-551. For another example, ASOs targeting a sequence upstream from the 5′ end of an NIE (e.g. exon 23 (or exon 20x) in human SCN1A, or exon 21x in mouse SCN1A) can comprise a sequence with at least 80%, 85%, 90%, 95%, 97%, or 100% sequence identity to any one of SEQ ID NOs: 12-191. For an additional example, ASOs targeting a sequence upstream from the 5′ end of an NIE (e.g. exon 23 (or exon 20x) in human SCN1A, or exon 21x in mouse SCN1A) can comprise a sequence with at least 80%, 85%, 90%, 95%, 97%, or 100% sequence identity to any one of SEQ ID NOs: 372-551.

In some embodiments, the ASO targets exon 23 (or exon 20x) in a SCN1A NIE containing pre-mRNA comprising exon 23. In some embodiments, the ASO targets an exon 23 sequence downstream (or 3′) from the 5′ end of the exon 23 of a SCN1A pre-mRNA. In some embodiments, the ASO targets an exon 23 sequence upstream (or 5′) from the 3′ end of the exon 20x of a SCN1A pre-mRNA.

In some embodiments, the ASO targets a sequence downstream from the 3′ end of an NIE. For example, ASOs targeting a sequence downstream from the 3′ end of an NIE (e.g. exon 23 (or exon 20x) in human SCN1A, or exon 21x in mouse SCN1A) can comprise a sequence with at least 80%, 85%, 90%, 95%, 97%, or 100% sequence identity to any one of SEQ ID NOs: 192-371 or 552-731. For another example, ASOs targeting a sequence downstream from the 3′ end of an NIE (e.g. exon 23 (or exon 20x) in human SCN1A, or exon 21x in mouse SCN1A) can comprise a sequence with at least 80%, 85%, 90%, 95%, 97%, or 100% sequence identity to any one of SEQ ID NOs: 192-371. For an additional example, ASOs targeting a sequence downstream from the 3′ end of an NIE (e.g. exon 23 (or exon 20x) in human SCN1A, or exon 21x in mouse SCN1A) can comprise a sequence with at least 80%, 85%, 90%, 95%, 97%, or 100% sequence identity to any one of SEQ ID NOs: 552-731.

In some embodiments, the targeted portion of the SCN1A NIE containing pre-mRNA is in intron 1, 2, 3, 4, 5, 6, 7, 8, 9, 10, 11, 12, 13, 14, 15, 16, 17, 18, 19, 20, 21, 22, 23, 24, or 25 (intron numbering corresponding to the mRNA sequence at NM_006920). In some embodiments, hybridization of an ASO to the targeted portion of the NIE pre-mRNA results in exon skipping of at least one of NIE within intron 1, 2, 3, 4, 5, 6, 7, 8, 9, 10, 11, 12, 13, 14, 15, 16, 17, 18, 19, 20, 21, 22, 23, 24, or 25, and subsequently increases SCN1A protein production. In some embodiments, hybridization of an ASO to the targeted portion of the NIE pre-mRNA inhibits or blocks exon skipping of at least one of NIE within intron 1, 2, 3, 4, 5, 6, 7, 8, 9, 10, 11, 12, 13, 14, 15, 16, 17, 18, 19, 20, 21, 22, 23, 24, or 25, and subsequently decreases SCN1A protein production. In some embodiments, the targeted portion of the SCN1A NIE containing pre-mRNA is in intron 20. One of skill in the art can determine the corresponding intron number in any isoform based on an intron sequence provided herein or using the number provided in reference to the mRNA sequence at NM_006920, NM_001202435, NM_001165964, or NM_001165963. One of skill in the art also can determine the sequences of flanking exons in any SCN1A isoform for targeting using the methods of the invention, based on an intron sequence provided herein or using the intron number provided in reference to the mRNA sequence at NM_006920, NM_001202435, NM_001165964, or NM_001165963.

In some embodiments, the methods and compositions of the present disclosure are used to modulate, e.g., increase or decrease, the expression of SCN1A by inducing or inhibiting exon skipping of a pseudo-exon of an SCN1A NIE containing pre-mRNA. In some embodiments, the pseudo-exon is a sequence within any of introns 1-25. In some embodiments, the pseudo-exon is a sequence within any of introns 2, 4, 6, 13, 14, 15, 16, 17, 18, 20, 21, 22, 23, 24, and 25. In some embodiments, the pseudo-exon is a sequence within any of introns 15, 18, and 19. In some embodiments, the pseudo-exon can be any SCN1A intron or a portion thereof. In some embodiments, the pseudo-exon is within intron 20. The SCN1A intron numbering used herein corresponds to the mRNA sequence at NM 006920. It is understood that the intron numbering may change in reference to a different SCN1A isoform sequence.

### SCN1A Protein

The SCN1A gene can encode SCN1A (sodium channel, voltage-gated, type I, alpha subunit) protein, which can also be referred to as alpha-subunit of voltage-gated sodium channel Naᵥ1.1. Also described above, SCN1A mutations in DS are spread across the entire protein. More than 100 novel mutations have been identified throughout the gene with the more debilitating arising de novo. These comprise of truncations (47%), missense (43%), deletions (3%), and splice site mutations (7%). The percentage of subjects carrying SCN1A mutations varies between 33 and 100%. The majority of mutations are novel changes (88%).

In some embodiments, the methods described herein are used to modulate, e.g., increase or decrease, the production of a functional SCN1A protein. As used herein, the term “functional” refers to the amount of activity or function of a SCN1A protein that is necessary to eliminate any one or more symptoms of a treated condition, e.g., Dravet syndrome; Epilepsy, generalized, with febrile seizures plus, type 2; Febrile seizures, familial, 3A; Autism; Epileptic encephalopathy, early infantile, 13; Sick sinus syndrome 1; Alzheimer's disease; or SUDEP. In some embodiments, the methods are used to increase the production of a partially functional SCN1A protein. As used herein, the term “partially functional” refers to any amount of activity or function of the SCN1A protein that is less than the amount of activity or function that is necessary to eliminate or prevent any one or more symptoms of a disease or condition. In some embodiments, a partially functional protein or RNA will have at least 10%, at least 20%, at least 30%, at least 40%, at least 50%, at least 60%, at least 70%, at least 75%, at least 80%, at least 85%, at least 90%, or at least 95% less activity relative to the fully functional protein or RNA.

In some embodiments, the method is a method of increasing the expression of the SCN1A protein by cells of a subject having a NIE containing pre-mRNA encoding the SCN1A protein, wherein the subject has Dravet syndrome caused by a deficient amount of activity of SCN1A protein, and wherein the deficient amount of the SCN1A protein is caused by haploinsufficiency of the SCN1A protein. In such an embodiment, the subject has a first allele encoding a functional SCN1A protein, and a second allele from which the SCN1A protein is not produced. In another such embodiment, the subject has a first allele encoding a functional SCN1A protein, and a second allele encoding a nonfunctional SCN1A protein. In another such embodiment, the subject has a first allele encoding a functional SCN1A protein, and a second allele encoding a partially functional SCN1A protein. In any of these embodiments, the antisense oligomer binds to a targeted portion of the NIE containing pre-mRNA transcribed from the second allele, thereby inducing exon skipping of the pseudo-exon from the pre-mRNA, and causing an increase in the level of mature mRNA encoding functional SCN1A protein, and an increase in the expression of the SCN1A protein in the cells of the subject.

In related embodiments, the method is a method of using an ASO to increase the expression of a protein or functional RNA. In some embodiments, an ASO is used to increase the expression of SCN1A protein in cells of a subject having a NIE containing pre-mRNA encoding SCN1A protein, wherein the subject has a deficiency, e.g., Dravet Syndrome (DS) (also known as SMEI); severe myoclonic epilepsy of infancy (SMEI)-borderland (SMEB); Febrile seizure (FS); epilepsy, generalized, with febrile seizures plus (GEFS+); epileptic encephalopathy, early infantile, 13; cryptogenic generalized epilepsy; cryptogenic focal epilepsy; myoclonic-astatic epilepsy; Lennox-Gastaut syndrome; West syndrome; idiopathic spasms; early myoclonic encephalopathy; progressive myoclonic epilepsy; alternating hemiplegia of childhood; unclassified epileptic encephalopathy; sudden unexpected death in epilepsy (SUDEP); sick sinus syndrome 1; early infantile SCN1A encephalopathy; early infantile epileptic encephalopathy (EIEE); or autism, in the amount or function of a SCN1A protein. In some embodiments, an ASO is used to increase the expression of SCN1A protein in cells of a subject, wherein the subject has a deficiency, e.g., Epileptic encephalopathy, early infantile, 13; in the amount or function of a SCN8A protein. In some embodiments, an ASO is used to increase the expression of SCN1A protein in cells of a subject, wherein the subject has a deficiency, e.g., Sick sinus syndrome 1; in the amount or function of a SCN5A protein.

In some embodiments, the NIE containing pre-mRNA transcript that encodes the protein that is causative of the disease or condition is targeted by the ASOs described herein. In some embodiments, a NIE containing pre-mRNA transcript that encodes a protein that is not causative of the disease is targeted by the ASOs. For example, a disease that is the result of a mutation or deficiency of a first protein in a particular pathway may be ameliorated by targeting a NIE containing pre-mRNA that encodes a second protein, thereby increasing production of the second protein. In some embodiments, the function of the second protein is able to compensate for the mutation or deficiency of the first protein (which is causative of the disease or condition).

In some embodiments, the subject has:
(a) a first mutant allele from which
   (i) the SCN1A protein is produced at a reduced level compared to production from a wild-type allele,
   (ii) the SCN1A protein is produced in a form having reduced function compared to an equivalent wild-type protein, or
   (iii) the SCN1A protein or functional RNA is not produced; and
(b) a second mutant allele from which
   (i) the SCN1A protein is produced at a reduced level compared to production from a wild-type allele,
   (ii) the SCN1A protein is produced in a form having reduced function compared to an equivalent wild-type protein, or
   (iii) the SCN1A protein is not produced, and
      wherein the NIE containing pre-mRNA is transcribed from the first allele and/or the second allele. In these embodiments, the ASO binds to a targeted portion of the NIE containing pre-mRNA transcribed from the first allele or the second allele, thereby inducing exon skipping of the pseudo-exon from the NIE containing pre-mRNA, and causing an increase in the level of mRNA encoding SCN1A protein and an increase in the expression of the target protein or functional RNA in the cells of the subject. In these embodiments, the target protein or functional RNA having an increase in expression level resulting from the exon skipping of the pseudo-exon from the NIE containing pre-mRNA is either in a form having reduced function compared to the equivalent wild-type protein (partially-functional), or having full function compared to the equivalent wild-type protein (fully-functional).

In some embodiments, the level of mRNA encoding SCN1A protein is increased 1.1 to 10-fold, when compared to the amount of mRNA encoding SCN1A protein that is produced in a control cell, e.g., one that is not treated with the antisense oligomer or one that is treated with an antisense oligomer that does not bind to the targeted portion of the SCN1A NIE containing pre-mRNA.

In some embodiments, a subject treated using the methods of the present disclosure expresses a partially functional SCN1A protein from one allele, wherein the partially functional SCN1A protein is caused by a frameshift mutation, a nonsense mutation, a missense mutation, or a partial gene deletion. In some embodiments, a subject treated using the methods of the invention expresses a nonfunctional SCN1A protein from one allele, wherein the nonfunctional SCN1A protein is caused by a frameshift mutation, a nonsense mutation, a missense mutation, a partial gene deletion, in one allele. In some embodiments, a subject treated using the methods of the invention has a SCN1A whole gene deletion, in one allele.

In some embodiments, the method is a method of decreasing the expression of the SCN1A protein by cells of a subject having a NIE containing pre-mRNA encoding the SCN1A protein, and wherein the subject has a gain-of-function mutation in Naᵥ1.1. In such an embodiment, the subject has an allele from which the SCN1A protein is produced in an elevated amount or an allele encoding a mutant SCN1A that induces increased activity of Naᵥ1.1 in the cell. In some embodiments, the increased activity of Naᵥ1.1 is characterized by a prolonged or near persistent sodium current mediated by the mutant Naᵥ1.1 channel, a slowing of fast inactivation, a positive shift in steady-state inactivation, higher channel availability during repetitive stimulation, increased non-inactivated depolarization-induced persistent sodium currents, delayed entry into inactivation, accelerated recovery from fast inactivation, and/or rescue of folding defects by incubation at lower temperature or co-expression of interacting proteins. In any of these embodiments, the antisense oligomer binds to a targeted portion of the NIE containing pre-mRNA transcribed from the second allele, thereby inhibiting or blocking exon skipping of the pseudo-exon from the pre-mRNA, and causing a decrease in the level of mature mRNA encoding functional SCN1A protein, and a decrease in the expression of the SCN1A protein in the cells of the subject.

In related embodiments, the method is a method of using an ASO to decrease the expression of a protein or functional RNA. In some embodiments, an ASO is used to decrease the expression of SCN1A protein in cells of a subject having a NIE containing pre-mRNA encoding SCN1A protein. In some embodiments, the subject has a gain-of-function mutation in Naᵥ1.1, e.g., migraine. In some embodiments, an ASO is used to decrease the expression of SCN1A protein in cells of a subject, the subject has a gain-of-function mutation in Naᵥ1.1, e.g., migraine, familial hemiplegic, 3.

In some embodiments, the level of mRNA encoding SCN1A protein is decreased 1.1 to 10-fold, when compared to the amount of mRNA encoding SCN1A protein that is produced in a control cell, e.g., one that is not treated with the antisense oligomer or one that is treated with an antisense oligomer that does not bind to the targeted portion of the SCN1A NIE containing pre-mRNA.

In some embodiments, a subject treated using the methods of the present disclosure expresses a mutant SCN1A protein from one allele, wherein the mutant SCN1A protein is caused by a frameshift mutation, a nonsense mutation, a missense mutation, or a partial gene deletion, and wherein the mutant SCN1A protein causes an elevated activity level of Naᵥ1.1. In some embodiments, a subject treated using the methods of the present disclosure expresses an elevated amount of SCN1A protein from one allele due to a frameshift mutation, a nonsense mutation, a missense mutation, or a partial gene deletion.

In embodiments of the present invention, a subject can have a mutation in SCN1A. Mutations in SCN1A can be spread throughout said gene. SCN1A protein can consist of four domains. Said SCN1A domains can have transmembrane segments. Mutations in said SCN1A protein may arise throughout said protein. Said SCN1A protein may consist of at least two isoforms. Mutations in SCN1A may comprise of R931C, R946C, M934I, R1648C, or R1648H. In some cases, mutations may be observed in a C-terminus of a SCN1A protein. Mutations in a SCN1A protein may also be found in loops between segments 5 and 6 of the first three domains of said SCN1A protein. In some cases, mutations may be observed in an N-terminus of a SCN1A protein. Exemplary mutations within SCN1A include, but are not limited to, R222X, R712X, I227S, R1892X, W952X, R1245X, R1407X, W1434R, c.4338+1G>A, S1516X, L1670fsX1678, or K1846fsX1856. Mutations that can be targeted with the present invention may also encode a pore of an ion channel.

In some embodiments, the methods and compositions described herein can be used to treat DS. In other embodiments, the methods and compositions described herein can be used to treat severe myoclonic epilepsy of infancy (SMEI). In other embodiments, the methods and compositions described herein can be used to treat borderline Dravet syndrome; Epilepsy, generalized, with febrile seizures plus, type 2; Febrile seizures, familial, 3A; Migraine, familial hemiplegic, 3; Autism; Epileptic encephalopathy, early infantile, 13; Sick sinus syndrome 1; Alzheimer's disease or SUDEP. The methods and compositions described herein can also be used to treat borderline SMEI. Additionally, the methods and compositions described herein can be used to treat generalized epilepsy with febrile seizures plus (GEFS+). GEFS+ may be associated with mutations in epilepsy-associated ion channel subunits such as SCN1B or GABRG2. The methods and compositions described herein can also be used to treat sodium channelopathies. Sodium channelopathies may be associated with mutations in SCN1A. Sodium channelopathies may also be associated with subunits of SCN1A, such as the beta subunit, SCN1B. In some cases, additional diseases associated with SCN1A mutations may also be treated with the present disclosure. Related SCN1A diseases associated with SCN1A mutations include, but are not limited to, atypical myotonia congenita, hyperkalemic periodic paralysis, and paramyotonia congenita.

In some embodiments, a subject having any SCN1A mutation known in the art and described in the literature referenced above (e.g., by Hamdan, et al., 2009, Mulley, et al., 2005) can be treated using the methods and compositions described herein. In some embodiments, the mutation is within any SCN1A intron or exon.

### Exon Inclusion

As used herein, a “NIE containing pre-mRNA” is a pre-mRNA transcript that contains at least one pseudo-exon. Alternative or aberrant splicing can result in inclusion of the at least one pseudo-exon in the mature mRNA transcripts. The terms “mature mRNA,” and “fully-spliced mRNA,” are used interchangeably herein to describe a fully processed mRNA. Inclusion of the at least one pseudo-exon can be non-productive mRNA and lead to NMD of the mature mRNA. NIE containing mature mRNA may sometimes lead to aberrant protein expression.

In some embodiments, the included pseudo-exon is the most abundant pseudo-exon in a population of NIE containing pre-mRNAs transcribed from the gene encoding the target protein in a cell. In some embodiments, the included pseudo-exon is the most abundant pseudo-exon in a population of NIE containing pre-mRNAs transcribed from the gene encoding the target protein in a cell, wherein the population of NIE containing pre-mRNAs comprises two or more included pseudo-exons. In some embodiments, an antisense oligomer targeted to the most abundant pseudo-exon in the population of NIE containing pre-mRNAs encoding the target protein induces exon skipping of one or two or more pseudo-exons in the population, including the pseudo-exon to which the antisense oligomer is targeted or binds. In embodiments, the targeted region is in a pseudo-exon that is the most abundant pseudo-exon in a NIE containing pre-mRNA encoding the SCN1A protein.

The degree of exon inclusion can be expressed as percent exon inclusion, e.g., the percentage of transcripts in which a given pseudo-exon is included. In brief, percent exon inclusion can be calculated as the percentage of the amount of RNA transcripts with the exon inclusion, over the sum of the average of the amount of RNA transcripts with exon inclusion plus the average of the amount of RNA transcripts with exon exclusion.

In some embodiments, an included pseudo-exon is an exon that is identified as an included pseudo-exon based on a determination of at least about 5%, at least about 10%, at least about 15%, at least about 20%, at least about 25%, at least about 30%, at least about 35%, at least about 40%, at least about 45%, or at least about 50%, inclusion. In embodiments, a included pseudo-exon is an exon that is identified as a included pseudo-exon based on a determination of about 5% to about 100%, about 5% to about 95%, about 5% to about 90%, about 5% to about 85%, about 5% to about 80%, about 5% to about 75%, about 5% to about 70%, about 5% to about 65%, about 5% to about 60%, about 5% to about 55%, about 5% to about 50%, about 5% to about 45%, about 5% to about 40%, about 5% to about 35%, about 5% to about 30%, about 5% to about 25%, about 5% to about 20%, about 5% to about 15%, about 10% to about 100%, about 10% to about 95%, about 10% to about 90%, about 10% to about 85%, about 10% to about 80%, about 10% to about 75%, about 10% to about 70%, about 10% to about 65%, about 10% to about 60%, about 10% to about 55%, about 10% to about 50%, about 10% to about 45%, about 10% to about 40%, about 10% to about 35%, about 10% to about 30%, about 10% to about 25%, about 10% to about 20%, about 15% to about 100%, about 15% to about 95%, about 15% to about 90%, about 15% to about 85%, about 15% to about 80%, about 15% to about 75%, about 15% to about 70%, about 15% to about 65%, about 15% to about 60%, about 15% to about 55%, about 15% to about 50%, about 15% to about 45%, about 15% to about 40%, about 15% to about 35%, about 15% to about 30%, about 15% to about 25%, about 20% to about 100%, about 20% to about 95%, about 20% to about 90%, about 20% to about 85%, about 20% to about 80%, about 20% to about 75%, about 20% to about 70%, about 20% to about 65%, about 20% to about 60%, about 20% to about 55%, about 20% to about 50%, about 20% to about 45%, about 20% to about 40%, about 20% to about 35%, about 20% to about 30%, about 25% to about 100%, about 25% to about 95%, about 25% to about 90%, about 25% to about 85%, about 25% to about 80%, about 25% to about 75%, about 25% to about 70%, about 25% to about 65%, about 25% to about 60%, about 25% to about 55%, about 25% to about 50%, about 25% to about 45%, about 25% to about 40%, or about 25% to about 35%, inclusion. ENCODE data (described by, e.g., Tilgner, et al., 2012, “Deep sequencing of subcellular RNA fractions shows splicing to be predominantly co-transcriptional in the human genome but inefficient for lncRNAs,” Genome Research 22(9):1616-25) can be used to aid in identifying exon inclusion.

In some embodiments, contacting cells with an ASO that is complementary to a targeted portion of a SCN1A pre-mRNA transcript results in an increase in the amount of SCN1A protein produced by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of SCN1A protein produced by the cell to which the antisense oligomer is contacted is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the amount of target protein produced by a control compound. A control compound can be, for example, an oligonucleotide that is not complementary to a targeted portion of the pre-mRNA.

In some embodiments, contacting cells with an ASO that is complementary to a targeted portion of a SCN1A pre-mRNA transcript results in a decrease in the amount of SCN1A protein produced by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of SCN1A protein produced by the cell to which the antisense oligomer is contacted is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the amount of target protein produced by a control compound. A control compound can be, for example, an oligonucleotide that is not complementary to a targeted portion of the pre-mRNA.

In some embodiments, contacting cells with an ASO that is complementary to a targeted portion of a SCN1A pre-mRNA transcript results in an increase in the amount of mRNA encoding SCN1A, including the mature mRNA encoding the target protein. In some embodiments, the amount of mRNA encoding SCN1A protein, or the mature mRNA encoding the SCN1A protein, is increased by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of the mRNA encoding SCN1A protein, or the mature mRNA encoding SCN1A protein produced in the cell to which the antisense oligomer is contacted is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold compared to the amount of mature RNA produced in an untreated cell, e.g., an untreated cell or a cell treated with a control compound. A control compound can be, for example, an oligonucleotide that is not complementary to a targeted portion of the SCN1A NIE containing pre-mRNA.

In some embodiments, contacting cells with an ASO that is complementary to a targeted portion of a SCN1A pre-mRNA transcript results in a decrease in the amount of mRNA encoding SCN1A, including the mature mRNA encoding the target protein. In some embodiments, the amount of mRNA encoding SCN1A protein, or the mature mRNA encoding the SCN1A protein, is decreased by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of the mRNA encoding SCN1A protein, or the mature mRNA encoding SCN1A protein produced in the cell to which the antisense oligomer is contacted is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold compared to the amount of mature RNA produced in an untreated cell, e.g., an untreated cell or a cell treated with a control compound. A control compound can be, for example, an oligonucleotide that is not complementary to a targeted portion of the SCN1A NIE containing pre-mRNA.

The NIE can be in any length. In some embodiments, the NIE comprises a full sequence of an intron, in which case, it can be referred to as intron retention. In some embodiments, the NIE can be a portion of the intron. In some embodiments, the NIE can be a 5′ end portion of an intron including a 5′ss sequence. In some embodiments, the NIE can be a 3′ end portion of an intron including a 3′ss sequence. In some embodiments, the NIE can be a portion within an intron without inclusion of a 5′ss sequence. In some embodiments, the NIE can be a portion within an intron without inclusion of a 3′ss sequence. In some embodiments, the NIE can be a portion within an intron without inclusion of either a 5′ss or a 3′ss sequence. In some embodiments, the NIE can be from 5 nucleotides to 10 nucleotides in length, from 10 nucleotides to 15 nucleotides in length, from 15 nucleotides to 20 nucleotides in length, from 20 nucleotides to 25 nucleotides in length, from 25 nucleotides to 30 nucleotides in length, from 30 nucleotides to 35 nucleotides in length, from 35 nucleotides to 40 nucleotides in length, from 40 nucleotides to 45 nucleotides in length, from 45 nucleotides to 50 nucleotides in length, from 50 nucleotides to 55 nucleotides in length, from 55 nucleotides to 60 nucleotides in length, from 60 nucleotides to 65 nucleotides in length, from 65 nucleotides to 70 nucleotides in length, from 70 nucleotides to 75 nucleotides in length, from 75 nucleotides to 80 nucleotides in length, from 80 nucleotides to 85 nucleotides in length, from 85 nucleotides to 90 nucleotides in length, from 90 nucleotides to 95 nucleotides in length, or from 95 nucleotides to 100 nucleotides in length. In some embodiments, the NIE can be at least 10 nucleotides, at least 20 nucleotides, at least 30 nucleotides, at least 40 nucleotides, at least 50 nucleotides, at least 60 nucleoids, at least 70 nucleotides, at least 80 nucleotides in length, at least 90 nucleotides, or at least 100 nucleotides in length. In some embodiments, the NIE can be from 100 to 200 nucleotides in length, from 200 to 300 nucleotides in length, from 300 to 400 nucleotides in length, from 400 to 500 nucleotides in length, from 500 to 600 nucleotides in length, from 600 to 700 nucleotides in length, from 700 to 800 nucleotides in length, from 800 to 900 nucleotides in length, from 900 to 1,000 nucleotides in length. In some embodiments, the NIE may be longer than 1,000 nucleotides in length.

Inclusion of a pseudo-exon can lead to a frameshift and the introduction of a premature termination codon (PIC) in the mature mRNA transcript rendering the transcript a target of NMD. Mature mRNA transcript containing NIE can be non-productive mRNA transcript which does not lead to protein expression. The PIC can be present in any position downstream of an NIE. In some embodiments, the PIC can be present in any exon downstream of an NIE. In some embodiments, the PIC can be present within the NIE. For example, inclusion of exon 20x in an mRNA transcript encoded by the SCN1A gene can induce a PIC in the mRNA transcript, e.g., a PIC in exon 21 of the mRNA transcript.

### Therapeutic Agents

In various embodiments of the present disclosure, compositions and methods comprising a therapeutic agent are provided to modulate protein expression level of SCN1A. In some embodiments, provided herein are compositions and methods to modulate alternative splicing of SCNA1 pre-mRNA. In some embodiments, provided herein are compositions and methods to induce exon skipping in the splicing of SCN1A pre-mRNA, e.g., to induce skipping of a pseudo-exon during splicing of SCN1A pre-mRNA. In other embodiments, therapeutic agents may be used to induce the inclusion of an exon in order to decrease the protein expression level.

In some embodiment, a therapeutic agent disclosed herein is a small molecule, a polypeptide, or a polynucleic acid polymer. In some instances, the therapeutic agent is a small molecule. In some instances, the therapeutic agent is a polypeptide. In some instances, the therapeutic agent is a polynucleic acid polymer. In some cases, the therapeutic agent is a repressor agent. In additional cases, the therapeutic agent is an enhancer agent.

A therapeutic agent disclosed herein can be a NIE repressor agent. A therapeutic agent may comprise a polynucleic acid polymer.

According to one aspect of the present disclosure, provided herein is a method of treatment or prevention of a condition associated with a functional-SCN1A protein deficiency, comprising administering a NIE repressor agent to a subject to increase levels of functional SCN1A protein, wherein the agent binds to a region of the pre-mRNA transcript to decrease inclusion of the NIE in the mature transcript. For example, provided herein is a method of treatment or prevention of a condition associated with a functional-SCN1A protein deficiency, comprising administering a NIE repressor agent to a subject to increase levels of functional SCN1A protein, wherein the agent binds to a region of an intron containing an NIE (e.g., intron 20 in human SCN1A gene) of the pre-mRNA transcript or to a NIE-activating regulatory sequence in the same intron.

Where reference is made to reducing NIE inclusion in the mature mRNA, the reduction may be complete, e.g., 100%, or may be partial. The reduction may be clinically significant. The reduction/correction may be relative to the level of NIE inclusion in the subject without treatment, or relative to the amount of NIE inclusion in a population of similar subjects. The reduction/correction may be at least 10% less NIE inclusion relative to the average subject, or the subject prior to treatment. The reduction may be at least 20% less NIE inclusion relative to an average subject, or the subject prior to treatment. The reduction may be at least 40% less NIE inclusion relative to an average subject, or the subject prior to treatment. The reduction may be at least 50% less NIE inclusion relative to an average subject, or the subject prior to treatment. The reduction may be at least 60% less NIE inclusion relative to an average subject, or the subject prior to treatment. The reduction may be at least 80% less NIE inclusion relative to an average subject, or the subject prior to treatment. The reduction may be at least 90% less NIE inclusion relative to an average subject, or the subject prior to treatment.

Where reference is made to increasing active-SCN1A protein levels, the increase may be clinically significant. The increase may be relative to the level of active-SCN1A protein in the subject without treatment, or relative to the amount of active-SCN1A protein in a population of similar subjects. The increase may be at least 10% more active-SCN1A protein relative to the average subject, or the subject prior to treatment. The increase may be at least 20% more active-SCN1A protein relative to the average subject, or the subject prior to treatment. The increase may be at least 40% more active-SCN1A protein relative to the average subject, or the subject prior to treatment. The increase may be at least 50% more active-SCN1A protein relative to the average subject, or the subject prior to treatment. The increase may be at least 80% more active-SCN1A protein relative to the average subject, or the subject prior to treatment. The increase may be at least 100% more active-SCN1A protein relative to the average subject, or the subject prior to treatment. The increase may be at least 200% more active-SCN1A protein relative to the average subject, or the subject prior to treatment. The increase may be at least 500% more active-SCN1A protein relative to the average subject, or the subject prior to treatment.

In embodiments wherein the NIE repressor agent comprises a polynucleic acid polymer, the polynucleic acid polymer may be about 50 nucleotides in length. The polynucleic acid polymer may be about 45 nucleotides in length. The polynucleic acid polymer may be about 40 nucleotides in length. The polynucleic acid polymer may be about 35 nucleotides in length. The polynucleic acid polymer may be about 30 nucleotides in length. The polynucleic acid polymer may be about 24 nucleotides in length. The polynucleic acid polymer may be about 25 nucleotides in length. The polynucleic acid polymer may be about 20 nucleotides in length. The polynucleic acid polymer may be about 19 nucleotides in length. The polynucleic acid polymer may be about 18 nucleotides in length. The polynucleic acid polymer may be about 17 nucleotides in length. The polynucleic acid polymer may be about 16 nucleotides in length. The polynucleic acid polymer may be about 15 nucleotides in length. The polynucleic acid polymer may be about 14 nucleotides in length. The polynucleic acid polymer may be about 13 nucleotides in length. The polynucleic acid polymer may be about 12 nucleotides in length. The polynucleic acid polymer may be about 11 nucleotides in length. The polynucleic acid polymer may be about 10 nucleotides in length. The polynucleic acid polymer may be between about 10 and about 50 nucleotides in length. The polynucleic acid polymer may be between about 10 and about 45 nucleotides in length. The polynucleic acid polymer may be between about 10 and about 40 nucleotides in length. The polynucleic acid polymer may be between about 10 and about 35 nucleotides in length. The polynucleic acid polymer may be between about 10 and about 30 nucleotides in length. The polynucleic acid polymer may be between about 10 and about 25 nucleotides in length. The polynucleic acid polymer may be between about 10 and about 20 nucleotides in length. The polynucleic acid polymer may be between about 15 and about 25 nucleotides in length. The polynucleic acid polymer may be between about 15 and about 30 nucleotides in length. The polynucleic acid polymer may be between about 12 and about 30 nucleotides in length.

The sequence of the polynucleic acid polymer may be at least 50%, 55%, 60%, 65%, 70%, 75%, 80%, 85%, 90%, 91%, 92%, 93%, 94%, 95%, 96%, 97%, 98%, 99%, or 99.5% complementary to a target sequence of an mRNA transcript, e.g., a partially processed mRNA transcript. The sequence of the polynucleic acid polymer may be 100% complementary to a target sequence of a pre-mRNA transcript.

The sequence of the polynucleic acid polymer may have 4 or fewer mismatches to a target sequence of the pre-mRNA transcript. The sequence of the polynucleic acid polymer may have 3 or fewer mismatches to a target sequence of the pre-mRNA transcript. The sequence of the polynucleic acid polymer may have 2 or fewer mismatches to a target sequence of the pre-mRNA transcript. The sequence of the polynucleic acid polymer may have 1 or fewer mismatches to a target sequence of the pre-mRNA transcript. The sequence of the polynucleic acid polymer may have no mismatches to a target sequence of the pre-mRNA transcript.

The polynucleic acid polymer may specifically hybridize to a target sequence of the pre-mRNA transcript. For example, the polynucleic acid polymer may have 91%, 92%, 93%, 94%, 95%, 96%, 97%, 98%, 99%, 99.5% or 100% sequence complementarity to a target sequence of the pre-mRNA transcript. The hybridization may be under high stringent hybridization conditions.

The polynucleic acid polymer may have a sequence with at least 50%, 55%, 60%, 65%, 70%, 75%, 80%, 85%, 90%, 91%, 92%, 93%, 94%, 95%, 96%, 97%, 98%, 99%, or 99.5% sequence identity to a sequence selected from the group consisting of SEQ ID NOs: 12-731. The polynucleic acid polymer may have a sequence with 100% sequence identity to a sequence selected from the group consisting of SEQ ID NOs: 12-731. In some instances, the polynucleic acid polymer may have a sequence with at least 50%, 55%, 60%, 65%, 70%, 75%, 80%, 85%, 90%, 91%, 92%, 93%, 94%, 95%, 96%, 97%, 98%, 99%, or 99.5% sequence identity to a sequence selected from the group consisting of SEQ ID NOs: 12-371. In some cases, the polynucleic acid polymer may have a sequence with 100% sequence identity to a sequence selected from the group consisting of SEQ ID NOs: 12-371. In some instances, the polynucleic acid polymer may have a sequence with at least 50%, 55%, 60%, 65%, 70%, 75%, 80%, 85%, 90%, 91%, 92%, 93%, 94%, 95%, 96%, 97%, 98%, 99%, or 99.5% sequence identity to a sequence selected from the group consisting of SEQ ID NOs: 372-731. In some cases, the polynucleic acid polymer may have a sequence with 100% sequence identity to a sequence selected from the group consisting of SEQ ID NOs: 372-731.

Where reference is made to a polynucleic acid polymer sequence, the skilled person will understand that one or more substitutions may be tolerated, optionally two substitutions may be tolerated in the sequence, such that it maintains the ability to hybridize to the target sequence; or where the substitution is in a target sequence, the ability to be recognized as the target sequence. References to sequence identity may be determined by BLAST sequence alignment using standard/default parameters. For example, the sequence may have 99% identity and still function according to the present disclosure. In other embodiments, the sequence may have 98% identity and still function according to the present disclosure. In another embodiment, the sequence may have 95% identity and still function according to the present disclosure. In another embodiment, the sequence may have 90% identity and still function according to the present disclosure.

### Antisense Oligomers

Provided herein is a composition comprising an antisense oligomer that induces exon skipping by binding to a targeted portion of a SCN1A NIE containing pre-mRNA. As used herein, the terms “ASO” and “antisense oligomer” are used interchangeably and refer to an oligomer such as a polynucleotide, comprising nucleobases that hybridizes to a target nucleic acid (e.g., a SCN1A NIE containing pre-mRNA) sequence by Watson-Crick base pairing or wobble base pairing (G-U). The ASO may have exact sequence complementary to the target sequence or near complementarity (e.g., sufficient complementarity to bind the target sequence and enhancing splicing at a splice site). ASOs are designed so that they bind (hybridize) to a target nucleic acid (e.g., a targeted portion of a pre-mRNA transcript) and remain hybridized under physiological conditions. Typically, if they hybridize to a site other than the intended (targeted) nucleic acid sequence, they hybridize to a limited number of sequences that are not a target nucleic acid (to a few sites other than a target nucleic acid). Design of an ASO can take into consideration the occurrence of the nucleic acid sequence of the targeted portion of the pre-mRNA transcript or a sufficiently similar nucleic acid sequence in other locations in the genome or cellular pre-mRNA or transcriptome, such that the likelihood the ASO will bind other sites and cause “off-target” effects is limited. Any antisense oligomers known in the art, for example in PCT Application No. PCT/US2014/054151, published as WO 2015/035091, titled “Reducing Nonsense-Mediated mRNA Decay,” incorporated by reference herein, can be used to practice the methods described herein.

In some embodiments, ASOs “specifically hybridize” to or are “specific” to a target nucleic acid or a targeted portion of a NIE containing pre-mRNA. Typically such hybridization occurs with a Tₘ substantially greater than 37° C., preferably at least 50° C., and typically between 60° C. to approximately 90° C. Such hybridization preferably corresponds to stringent hybridization conditions. At a given ionic strength and pH, the Tₘ is the temperature at which 50% of a target sequence hybridizes to a complementary oligonucleotide.

Oligomers, such as oligonucleotides, are “complementary” to one another when hybridization occurs in an antiparallel configuration between two single-stranded polynucleotides. A double-stranded polynucleotide can be “complementary” to another polynucleotide, if hybridization can occur between one of the strands of the first polynucleotide and the second. Complementarity (the degree to which one polynucleotide is complementary with another) is quantifiable in terms of the proportion (e.g., the percentage) of bases in opposing strands that are expected to form hydrogen bonds with each other, according to generally accepted base-pairing rules. The sequence of an antisense oligomer (ASO) need not be 100% complementary to that of its target nucleic acid to hybridize. In certain embodiments, ASOs can comprise at least 70%, at least 75%, at least 80%, at least 85%, at least 90%, at least 95%, at least 96%, at least 97%, at least 98%, or at least 99% sequence complementarity to a target region within the target nucleic acid sequence to which they are targeted. For example, an ASO in which 18 of 20 nucleobases of the oligomeric compound are complementary to a target region, and would therefore specifically hybridize, would represent 90 percent complementarity. In this example, the remaining non-complementary nucleobases may be clustered together or interspersed with complementary nucleobases and need not be contiguous to each other or to complementary nucleobases. Percent complementarity of an ASO with a region of a target nucleic acid can be determined routinely using BLAST programs (basic local alignment search tools) and PowerBLAST programs known in the art (Altschul, et al., J. Mol. Biol., 1990, 215, 403-410; Zhang and Madden, Genome Res., 1997, 7, 649-656).

An ASO need not hybridize to all nucleobases in a target sequence and the nucleobases to which it does hybridize may be contiguous or noncontiguous. ASOs may hybridize over one or more segments of a pre-mRNA transcript, such that intervening or adjacent segments are not involved in the hybridization event (e.g., a loop structure or hairpin structure may be formed). In certain embodiments, an ASO hybridizes to noncontiguous nucleobases in a target pre-mRNA transcript. For example, an ASO can hybridize to nucleobases in a pre-mRNA transcript that are separated by one or more nucleobase(s) to which the ASO does not hybridize.

The ASOs described herein comprise nucleobases that are complementary to nucleobases present in a targeted portion of a NIE containing pre-mRNA. The term ASO embodies oligonucleotides and any other oligomeric molecule that comprises nucleobases capable of hybridizing to a complementary nucleobase on a target mRNA but does not comprise a sugar moiety, such as a peptide nucleic acid (PNA). The ASOs may comprise naturally-occurring nucleotides, nucleotide analogs, modified nucleotides, or any combination of two or three of the preceding. The term “naturally occurring nucleotides” includes deoxyribonucleotides and ribonucleotides. The term “modified nucleotides” includes nucleotides with modified or substituted sugar groups and/or having a modified backbone. In some embodiments, all of the nucleotides of the ASO are modified nucleotides. Chemical modifications of ASOs or components of ASOs that are compatible with the methods and compositions described herein will be evident to one of skill in the art and can be found, for example, in U.S. Pat. No. 8,258,109 B2, U.S. Pat. No. 5,656,612, U.S. Patent Publication No. 2012/0190728, and Dias and Stein, Mol. Cancer Ther. 2002, 347-355, herein incorporated by reference in their entirety.

One or more nucleobases of an ASO may be any naturally occurring, unmodified nucleobase such as adenine, guanine, cytosine, thymine and uracil, or any synthetic or modified nucleobase that is sufficiently similar to an unmodified nucleobase such that it is capable of hydrogen bonding with a nucleobase present on a target pre-mRNA. Examples of modified nucleobases include, without limitation, hypoxanthine, xanthine, 7-methylguanine, 5,6-dihydrouracil, 5-methylcytosine, and 5-hydroxymethoylcytosine.

The ASOs described herein also comprise a backbone structure that connects the components of an oligomer. The term “backbone structure” and “oligomer linkages” may be used interchangeably and refer to the connection between monomers of the ASO. In naturally occurring oligonucleotides, the backbone comprises a 3′-5′ phosphodiester linkage connecting sugar moieties of the oligomer. The backbone structure or oligomer linkages of the ASOs described herein may include (but are not limited to) phosphorothioate, phosphorodithioate, phosphoroselenoate, phosphorodiselenoate, phosphoroanilothioate, phosphoraniladate, phosphoramidate, and the like. See, e.g., LaPlanche, et al., Nucleic Acids Res. 14:9081 (1986); Stec, et al., J. Am. Chem. Soc. 106:6077 (1984), Stein, et al., Nucleic Acids Res. 16:3209 (1988), Zon, et al., Anti-Cancer Drug Design 6:539 (1991); Zon, et al., Oligonucleotides and Analogues: A Practical Approach, pp. 87-108 (F. Eckstein, Ed., Oxford University Press, Oxford England (1991)); Stec, et al., U.S. Pat. No. 5,151,510; Uhlmann and Peyman, Chemical Reviews 90:543 (1990). In some embodiments, the backbone structure of the ASO does not contain phosphorous but rather contains peptide bonds, for example in a peptide nucleic acid (PNA), or linking groups including carbamate, amides, and linear and cyclic hydrocarbon groups. In some embodiments, the backbone modification is a phosphothioate linkage. In some embodiments, the backbone modification is a phosphoramidate linkage.

In embodiments, the stereochemistry at each of the phosphorus internucleotide linkages of the ASO backbone is random. In embodiments, the stereochemistry at each of the phosphorus internucleotide linkages of the ASO backbone is controlled and is not random. For example, U.S. Pat. App. Pub. No. 2014/0194610, “Methods for the Synthesis of Functionalized Nucleic Acids,” incorporated herein by reference, describes methods for independently selecting the handedness of chirality at each phosphorous atom in a nucleic acid oligomer. In embodiments, an ASO used in the methods of the invention, including, but not limited to, any of the ASOs set forth herein in Tables 5 and 6, comprises an ASO having phosphorus internucleotide linkages that are not random. In embodiments, a composition used in the methods of the invention comprises a pure diastereomeric ASO. In embodiments, a composition used in the methods of the invention comprises an ASO that has diastereomeric purity of at least about 90%, at least about 91%, at least about 92%, at least about 93%, at least about 94%, at least about 95%, at least about 96%, at least about 97%, at least about 98%, at least about 99%, about 100%, about 90% to about 100%, about 91% to about 100%, about 92% to about 100%, about 93% to about 100%, about 94% to about 100%, about 95% to about 100%, about 96% to about 100%, about 97% to about 100%, about 98% to about 100%, or about 99% to about 100%.

In embodiments, the ASO has a nonrandom mixture of Rp and Sp configurations at its phosphorus internucleotide linkages. For example, it has been suggested that a mix of Rp and Sp is required in antisense oligonucleotides to achieve a balance between good activity and nuclease stability (Wan, et al., 2014, “Synthesis, biophysical properties and biological activity of second generation antisense oligonucleotides containing chiral phosphorothioate linkages,” Nucleic Acids Research 42(22): 13456-13468, incorporated herein by reference). In embodiments, an ASO used in the methods of the invention, including, but not limited to, any of the ASOs set forth herein in SEQ ID NOs: 12-731, comprises about 5-100% Rp, at least about 5% Rp, at least about 10% Rp, at least about 15% Rp, at least about 20% Rp, at least about 25% Rp, at least about 30% Rp, at least about 35% Rp, at least about 40% Rp, at least about 45% Rp, at least about 50% Rp, at least about 55% Rp, at least about 60% Rp, at least about 65% Rp, at least about 70% Rp, at least about 75% Rp, at least about 80% Rp, at least about 85% Rp, at least about 90% Rp, or at least about 95% Rp, with the remainder Sp, or about 100% Rp. In embodiments, an ASO used in the methods of the invention, including, but not limited to, any of the ASOs set forth herein in SEQ ID NOs: 12-731, comprises about 10% to about 100% Rp, about 15% to about 100% Rp, about 20% to about 100% Rp, about 25% to about 100% Rp, about 30% to about 100% Rp, about 35% to about 100% Rp, about 40% to about 100% Rp, about 45% to about 100% Rp, about 50% to about 100% Rp, about 55% to about 100% Rp, about 60% to about 100% Rp, about 65% to about 100% Rp, about 70% to about 100% Rp, about 75% to about 100% Rp, about 80% to about 100% Rp, about 85% to about 100% Rp, about 90% to about 100% Rp, or about 95% to about 100% Rp, about 20% to about 80% Rp, about 25% to about 75% Rp, about 30% to about 70% Rp, about 40% to about 60% Rp, or about 45% to about 55% Rp, with the remainder Sp.

In embodiments, an ASO used in the methods of the invention, including, but not limited to, any of the ASOs set forth herein in SEQ ID NOs: 12-731, comprises about 5-100% Sp, at least about 5% Sp, at least about 10% Sp, at least about 15% Sp, at least about 20% Sp, at least about 25% Sp, at least about 30% Sp, at least about 35% Sp, at least about 40% Sp, at least about 45% Sp, at least about 50% Sp, at least about 55% Sp, at least about 60% Sp, at least about 65% Sp, at least about 70% Sp, at least about 75% Sp, at least about 80% Sp, at least about 85% Sp, at least about 90% Sp, or at least about 95% Sp, with the remainder Rp, or about 100% Sp. In embodiments, an ASO used in the methods of the invention, including, but not limited to, any of the ASOs set forth herein in SEQ ID NOs: 12-731, comprises about 10% to about 100% Sp, about 15% to about 100% Sp, about 20% to about 100% Sp, about 25% to about 100% Sp, about 30% to about 100% Sp, about 35% to about 100% Sp, about 40% to about 100% Sp, about 45% to about 100% Sp, about 50% to about 100% Sp, about 55% to about 100% Sp, about 60% to about 100% Sp, about 65% to about 100% Sp, about 70% to about 100% Sp, about 75% to about 100% Sp, about 80% to about 100% Sp, about 85% to about 100% Sp, about 90% to about 100% Sp, or about 95% to about 100% Sp, about 20% to about 80% Sp, about 25% to about 75% Sp, about 30% to about 70% Sp, about 40% to about 60% Sp, or about 45% to about 55% Sp, with the remainder Rp.

Any of the ASOs described herein may contain a sugar moiety that comprises ribose or deoxyribose, as present in naturally occurring nucleotides, or a modified sugar moiety or sugar analog, including a morpholine ring. Non-limiting examples of modified sugar moieties include 2′ substitutions such as 2′-O-methyl(2′-O-Me), 2′-O-methoxyethyl (2′MOE), 2′-O-aminoethyl, 2′F; N3′->P5′ phosphoramidate, 2′dimethylaminooxyethoxy, 2′dimethylaminoethoxyethoxy, 2′-guanidinium, 2′-O-guanidinium ethyl, carbamate modified sugars, and bicyclic modified sugars. In some embodiments, the sugar moiety modification is selected from 2′-O-Me, 2′F, and 2′MOE. In some embodiments, the sugar moiety modification is an extra bridge bond, such as in a locked nucleic acid (LNA). In some embodiments the sugar analog contains a morpholine ring, such as phosphorodiamidate morpholino (PMO). In some embodiments, the sugar moiety comprises a ribofuransyl or 2′deoxyribofuransyl modification. In some embodiments, the sugar moiety comprises 2′4′-constrained 2′O-methyloxyethyl (cMOE) modifications. In some embodiments, the sugar moiety comprises cEt 2′,4′ constrained 2′-O ethyl BNA modifications. In some embodiments, the sugar moiety comprises tricycloDNA (tcDNA) modifications. In some embodiments, the sugar moiety comprises ethylene nucleic acid (ENA) modifications. In some embodiments, the sugar moiety comprises MCE modifications. Modifications are known in the art and described in the literature, e.g., by Jarver, et al., 2014, “A Chemical View of Oligonucleotides for Exon Skipping and Related Drug Applications,” Nucleic Acid Therapeutics 24(1): 37-47, incorporated by reference for this purpose herein.

In some embodiments, each monomer of the ASO is modified in the same way, for example each linkage of the backbone of the ASO comprises a phosphorothioate linkage or each ribose sugar moiety comprises a 2′O-methyl modification. Such modifications that are present on each of the monomer components of an ASO are referred to as “uniform modifications.” In some examples, a combination of different modifications may be desired, for example, an ASO may comprise a combination of phosphorodiamidate linkages and sugar moieties comprising morpholine rings (morpholinos). Combinations of different modifications to an ASO are referred to as “mixed modifications” or “mixed chemistries.”

In some embodiments, the ASO comprises one or more backbone modifications. In some embodiments, the ASO comprises one or more sugar moiety modification. In some embodiments, the ASO comprises one or more backbone modifications and one or more sugar moiety modifications. In some embodiments, the ASO comprises a 2′MOE modification and a phosphorothioate backbone. In some embodiments, the ASO comprises a phosphorodiamidate morpholino (PMO). In some embodiments, the ASO comprises a peptide nucleic acid (PNA). Any of the ASOs or any component of an ASO (e.g., a nucleobase, sugar moiety, backbone) described herein may be modified in order to achieve desired properties or activities of the ASO or reduce undesired properties or activities of the ASO. For example, an ASO or one or more components of any ASO may be modified to enhance binding affinity to a target sequence on a pre-mRNA transcript; reduce binding to any non-target sequence; reduce degradation by cellular nucleases (i.e., RNase H); improve uptake of the ASO into a cell and/or into the nucleus of a cell; alter the pharmacokinetics or pharmacodynamics of the ASO; and/or modulate the half-life of the ASO.

In some embodiments, the ASOs are comprised of 2′-O-(2-methoxyethyl) (MOE) phosphorothioate-modified nucleotides. ASOs comprised of such nucleotides are especially well-suited to the methods disclosed herein; oligomers having such modifications have been shown to have significantly enhanced resistance to nuclease degradation and increased bioavailability, making them suitable, for example, for oral delivery in some embodiments described herein. See e.g., Geary, et al., J Pharmacol Exp Ther. 2001; 296(3):890-7; Geary, et al., J Pharmacol Exp Ther. 2001; 296(3):898-904.

Methods of synthesizing ASOs will be known to one of skill in the art. Alternatively or in addition, ASOs may be obtained from a commercial source.

Unless specified otherwise, the left-hand end of single-stranded nucleic acid (e.g., pre-mRNA transcript, oligonucleotide, ASO, etc.) sequences is the 5′ end and the left-hand direction of single or double-stranded nucleic acid sequences is referred to as the 5′ direction. Similarly, the right-hand end or direction of a nucleic acid sequence (single or double stranded) is the 3′ end or direction. Generally, a region or sequence that is 5′ to a reference point in a nucleic acid is referred to as “upstream,” and a region or sequence that is 3′ to a reference point in a nucleic acid is referred to as “downstream.” Generally, the 5′ direction or end of an mRNA is where the initiation or start codon is located, while the 3′ end or direction is where the termination codon is located. In some aspects, nucleotides that are upstream of a reference point in a nucleic acid may be designated by a negative number, while nucleotides that are downstream of a reference point may be designated by a positive number. For example, a reference point (e.g., an exon-exon junction in mRNA) may be designated as the “zero” site, and a nucleotide that is directly adjacent and upstream of the reference point is designated “minus one,” e.g., “−1,” while a nucleotide that is directly adjacent and downstream of the reference point is designated “plus one,” e.g., “+1.”

In some embodiments, the ASOs are complementary to (and bind to) a targeted portion of a SCN1A NIE containing pre-mRNA that is downstream (in the 3′ direction) of the 5′ splice site (or 3′ end of the NIE) of the included exon in a SCN1A NIE containing pre-mRNA (e.g., the direction designated by positive numbers relative to the 5′ splice site). In some embodiments, the ASOs are complementary to a targeted portion of the SCN1A NIE containing pre-mRNA that is within the region about +1 to about +500 relative to the 5′ splice site (or 3′ end) of the included exon. In some embodiments, the ASOs may be complementary to a targeted portion of a SCN1A NIE containing pre-mRNA that is within the region between nucleotides +6 and +496 relative to the 5′ splice site (or 3′ end) of the included exon. In some aspects, the ASOs are complementary to a targeted portion that is within the region about +1 to about +500, about +1 to about +490, about +1 to about +480, about +1 to about +470, about +1 to about +460, about +1 to about +450, about +1 to about +440, about +1 to about +430, about +1 to about +420, about +1 to about +410, about +1 to about +400, about +1 to about +390, about +1 to about +380, about +1 to about +370, about +1 to about +360, about +1 to about +350, about +1 to about +340, about +1 to about +330, about +1 to about +320, about +1 to about +310, about +1 to about +300, about +1 to about +290, about +1 to about +280, about +1 to about +270, about +1 to about +260, about +1 to about +250, about +1 to about +240, about +1 to about +230, about +1 to about +220, about +1 to about +210, about +1 to about +200, about +1 to about +190, about +1 to about +180, about +1 to about +170, about +1 to about +160, about +1 to about +150, about +1 to about +140, about +1 to about +130, about +1 to about +120, about +1 to about +110, about +1 to about +100, about +1 to about +90, about +1 to about +80, about +1 to about +70, about +1 to about +60, about +1 to about +50, about +1 to about +40, about +1 to about +30, or about +1 to about +20 relative to 5′ splice site (or 3′ end) of the included exon. In some aspects, the ASOs are complementary to a targeted portion that is within the region from about +1 to about +100, from about +100 to about +200, from about +200 to about +300, from about +300 to about +400, or from about +400 to about +500 relative to 5′ splice site (or 3′ end) of the included exon.

In some embodiments, the ASOs are complementary to (and bind to) a targeted portion of a SCN1A NIE containing pre-mRNA that is upstream (in the 5′ direction) of the 5′ splice site (or 3′ end) of the included exon in a SCN1A NIE containing pre-mRNA (e.g., the direction designated by negative numbers relative to the 5′ splice site). In some embodiments, the ASOs are complementary to a targeted portion of the SCN1A NIE containing pre-mRNA that is within the region about −4 to about −270 relative to the 5′ splice site (or 3′-end) of the included exon. In some embodiments, the ASOs may be complementary to a targeted portion of a SCN1A NIE containing pre-mRNA that is within the region between nucleotides −1 and −264 relative to the 5′ splice site (or 3′ end) of the included exon. In some aspects, the ASOs are complementary to a targeted portion that is within the region about −1 to about −270, about −1 to about −260, about −1 to about −250, about −1 to about −240, about −1 to about −230, about −1 to about −220, about −1 to about −210, about −1 to about −200, about −1 to about −190, about −1 to about −180, about −1 to about −170, about −1 to about −160, about −1 to about −150, about −1 to about −140, about −1 to about −130, about −1 to about −120, about −1 to about −110, about −1 to about −100, about −1 to about −90, about −1 to about −80, about −1 to about −70, about −1 to about −60, about −1 to about −50, about −1 to about −40, about −1 to about −30, or about −1 to about −20 relative to 5′ splice site (or 3′ end) of the included exon. In some aspects, the ASOs are complementary to a targeted portion that is within the region from about −1 to about −50, from about −50 to about −100, from about −100 to about −150, from about −150 to about −200, or from about −200 to about −250 relative to 5′ splice site (or 3′ end) of the included exon.

In some embodiments, the ASOs are complementary to a targeted region of a SCN1A NIE containing pre-mRNA that is upstream (in the 5′ direction) of the 3′ splice site (or 5′ end) of the included exon in a SCN1A NIE containing pre-mRNA (e.g., in the direction designated by negative numbers). In some embodiments, the ASOs are complementary to a targeted portion of the SCN1A NIE containing pre-mRNA that is within the region about −1 to about −500 relative to the 3′ splice site (or 5′ end) of the included exon. In some embodiments, the ASOs are complementary to a targeted portion of the SCN1A NIE containing pre-mRNA that is within the region −1 to −496 relative to the 3′ splice site of the included exon. In some aspects, the ASOs are complementary to a targeted portion that is within the region about −1 to about −500, about −1 to about −490, about −1 to about −480, about −1 to about −470, about −1 to about −460, about −1 to about −450, about −1 to about −440, about −1 to about −430, about −1 to about −420, about −1 to about −410, about −1 to about −400, about −1 to about −390, about −1 to about −380, about −1 to about −370, about −1 to about −360, about −1 to about −350, about −1 to about −340, about −1 to about −330, about −1 to about −320, about −1 to about −310, about −1 to about −300, about −1 to about −290, about −1 to about −280, about −1 to about −270, about −1 to about −260, about −1 to about −250, about −1 to about −240, about −1 to about −230, about −1 to about −220, about −1 to about −210, about −1 to about −200, about −1 to about −190, about −1 to about −180, about −1 to about −170, about −1 to about −160, about −1 to about −150, about −1 to about −140, about −1 to about −130, about −1 to about −120, about −1 to about −110, about −1 to about −100, about −1 to about −90, about −1 to about −80, about −1 to about −70, about −1 to about −60, about −1 to about −50, about −1 to about −40, or about −1 to about −30 relative to 3′ splice site of the included exon. In some aspects, the ASOs are complementary to a targeted portion that is within the region from about −1 to about −100, from about −100 to about −200, from about −200 to about −300, from about −300 to about −400, or from about −400 to about −500 relative to 3′ splice site of the included exon.

In some embodiments, the ASOs are complementary to a targeted region of a SCN1A NIE containing pre-mRNA that is downstream (in the 3′ direction) of the 3′ splice site (5′ end) of the included exon in a SCN1A NIE containing pre-mRNA (e.g., in the direction designated by positive numbers). In some embodiments, the ASOs are complementary to a targeted portion of the SCN1A NIE containing pre-mRNA that is within the region of about +1 to about +100 relative to the 3′ splice site of the included exon. In some aspects, the ASOs are complementary to a targeted portion that is within the region about +1 to about +90, about +1 to about +80, about +1 to about +70, about +1 to about +60, about +1 to about +50, about +1 to about +40, about +1 to about +30, about +1 to about +20, or about +1 to about +10 relative to 3′ splice site of the included exon.

In some embodiments, the targeted portion of the SCN1A NIE containing pre-mRNA is within the region +100 relative to the 5′ splice site (3′ end) of the included exon to −100 relative to the 3′ splice site (5′ end) of the included exon. In some embodiments, the targeted portion of the SCN1A NIE containing pre-mRNA is within the NIE. In some embodiments, the targeted portion of the SCN1A NIE containing pre-mRNA comprises a pseudo-exon and intron boundary.

The ASOs may be of any length suitable for specific binding and effective enhancement of splicing. In some embodiments, the ASOs consist of 8 to 50 nucleobases. For example, the ASO may be 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, or 50 nucleobases in length. In some embodiments, the ASOs consist of more than 50 nucleobases. In some embodiments, the ASO is from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, 12 to 15 nucleobases, 13 to 50 nucleobases, 13 to 40 nucleobases, 13 to 35 nucleobases, 13 to 30 nucleobases, 13 to 25 nucleobases, 13 to 20 nucleobases, 14 to 50 nucleobases, 14 to 40 nucleobases, 14 to 35 nucleobases, 14 to 30 nucleobases, 14 to 25 nucleobases, 14 to 20 nucleobases, 15 to 50 nucleobases, 15 to 40 nucleobases, 15 to 35 nucleobases, 15 to 30 nucleobases, 15 to 25 nucleobases, 15 to 20 nucleobases, 20 to 50 nucleobases, 20 to 40 nucleobases, 20 to 35 nucleobases, 20 to 30 nucleobases, 20 to 25 nucleobases, 25 to 50 nucleobases, 25 to 40 nucleobases, 25 to 35 nucleobases, or 25 to 30 nucleobases in length. In some embodiments, the ASOs are 18 nucleotides in length. In some embodiments, the ASOs are 15 nucleotides in length. In some embodiments, the ASOs are 25 nucleotides in length.

In some embodiments, two or more ASOs with different chemistries but complementary to the same targeted portion of the NIE containing pre-mRNA are used. In some embodiments, two or more ASOs that are complementary to different targeted portions of the NIE containing pre-mRNA are used.

In embodiments, the antisense oligonucleotides of the invention are chemically linked to one or more moieties or conjugates, e.g., a targeting moiety or other conjugate that enhances the activity or cellular uptake of the oligonucleotide. Such moieties include, but are not limited to, a lipid moiety, e.g., as a cholesterol moiety, a cholesteryl moiety, an aliphatic chain, e.g., dodecandiol or undecyl residues, a polyamine or a polyethylene glycol chain, or adamantane acetic acid. Oligonucleotides comprising lipophilic moieties and preparation methods have been described in the published literature. In embodiments, the antisense oligonucleotide is conjugated with a moiety including, but not limited to, an abasic nucleotide, a polyether, a polyamine, a polyamide, a peptides, a carbohydrate, e.g., N-acetylgalactosamine (GalNAc), N-Ac-Glucosamine (GluNAc), or mannose (e.g., mannose-6-phosphate), a lipid, or a polyhydrocarbon compound. Conjugates can be linked to one or more of any nucleotides comprising the antisense oligonucleotide at any of several positions on the sugar, base or phosphate group, as understood in the art and described in the literature, e.g., using a linker. Linkers can include a bivalent or trivalent branched linker. In embodiments, the conjugate is attached to the 3′ end of the antisense oligonucleotide. Methods of preparing oligonucleotide conjugates are described, e.g., in U.S. Pat. No. 8,450,467, “Carbohydrate conjugates as delivery agents for oligonucleotides,” incorporated by reference herein.

In some embodiments, the nucleic acid to be targeted by an ASO is a SCN1A NIE containing pre-mRNA expressed in a cell, such as a eukaryotic cell. In some embodiments, the term “cell” may refer to a population of cells. In some embodiments, the cell is in a subject. In some embodiments, the cell is isolated from a subject. In some embodiments, the cell is ex vivo. In some embodiments, the cell is a condition or disease-relevant cell or a cell line. In some embodiments, the cell is in vitro (e.g., in cell culture).

### Pharmaceutical Compositions

Pharmaceutical compositions or formulations comprising the agent, e.g., antisense oligonucleotide, of the described compositions and for use in any of the described methods can be prepared according to conventional techniques well known in the pharmaceutical industry and described in the published literature. In embodiments, a pharmaceutical composition or formulation for treating a subject comprises an effective amount of any antisense oligomer as described herein, or a pharmaceutically acceptable salt, solvate, hydrate or ester thereof. The pharmaceutical formulation comprising an antisense oligomer may further comprise a pharmaceutically acceptable excipient, diluent or carrier.

Pharmaceutically acceptable salts are suitable for use in contact with the tissues of humans and lower animals without undue toxicity, irritation, allergic response, etc., and are commensurate with a reasonable benefit/risk ratio. (See, e.g., S. M. Berge, et al., J. Pharmaceutical Sciences, 66:1-19 (1977), incorporated herein by reference for this purpose. The salts can be prepared in situ during the final isolation and purification of the compounds, or separately by reacting the free base function with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other documented methodologies such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

In embodiments, the compositions are formulated into any of many possible dosage forms such as, but not limited to, tablets, capsules, gel capsules, liquid syrups, soft gels, suppositories, and enemas. In embodiments, the compositions are formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. In embodiments, a pharmaceutical formulation or composition of the present invention includes, but is not limited to, a solution, emulsion, microemulsion, foam or liposome-containing formulation (e.g., cationic or noncationic liposomes).

The pharmaceutical composition or formulation described herein may comprise one or more penetration enhancers, carriers, excipients or other active or inactive ingredients as appropriate and well known to those of skill in the art or described in the published literature. In embodiments, liposomes also include sterically stabilized liposomes, e.g., liposomes comprising one or more specialized lipids. These specialized lipids result in liposomes with enhanced circulation lifetimes. In embodiments, a sterically stabilized liposome comprises one or more glycolipids or is derivatized with one or more hydrophilic polymers, such as a polyethylene glycol (PEG) moiety. In embodiments, a surfactant is included in the pharmaceutical formulation or compositions. The use of surfactants in drug products, formulations and emulsions is well known in the art. In embodiments, the present invention employs a penetration enhancer to effect the efficient delivery of the antisense oligonucleotide, e.g., to aid diffusion across cell membranes and/or enhance the permeability of a lipophilic drug. In embodiments, the penetration enhancers are a surfactant, fatty acid, bile salt, chelating agent, or non-chelating nonsurfactant.

In embodiments, the pharmaceutical formulation comprises multiple antisense oligonucleotides. In embodiments, the antisense oligonucleotide is administered in combination with another drug or therapeutic agent.

### Combination Therapies

In some embodiments, the ASOs disclosed in the present disclosure can be used in combination with one or more additional therapeutic agents. In some embodiments, the one or more additional therapeutic agents can comprise a small molecule. For example, the one or more additional therapeutic agents can comprise a small molecule described in WO2016128343A1, WO2017053982A1, WO2016196386A1, WO201428459A1, WO201524876A2, WO2013119916A2, and WO2014209841A2, which are incorporated by reference herein in their entirety. In some embodiments, the one or more additional therapeutic agents comprise an ASO that can be used to correct intron retention. In some embodiments, the one or more other agents are selected from the ASOs listed in Table 4.

### Treatment of Subjects

Any of the compositions provided herein may be administered to an individual. “Individual” may be used interchangeably with “subject” or “patient.” An individual may be a mammal, for example a human or animal such as a non-human primate, a rodent, a rabbit, a rat, a mouse, a horse, a donkey, a goat, a cat, a dog, a cow, a pig, or a sheep. In embodiments, the individual is a human. In embodiments, the individual is a fetus, an embryo, or a child. In other embodiments, the individual may be another eukaryotic organism, such as a plant. In some embodiments, the compositions provided herein are administered to a cell ex vivo.

In some embodiments, the compositions provided herein are administered to an individual as a method of treating a disease or disorder. In some embodiments, the individual has a genetic disease, such as any of the diseases described herein. In some embodiments, the individual is at risk of having a disease, such as any of the diseases described herein. In some embodiments, the individual is at increased risk of having a disease or disorder caused by insufficient amount of a protein or insufficient activity of a protein. If an individual is “at an increased risk” of having a disease or disorder caused insufficient amount of a protein or insufficient activity of a protein, the method involves preventative or prophylactic treatment. For example, an individual may be at an increased risk of having such a disease or disorder because of family history of the disease. Typically, individuals at an increased risk of having such a disease or disorder benefit from prophylactic treatment (e.g., by preventing or delaying the onset or progression of the disease or disorder). In embodiments, a fetus is treated in utero, e.g., by administering the ASO composition to the fetus directly or indirectly (e.g., via the mother).

Suitable routes for administration of ASOs of the present invention may vary depending on cell type to which delivery of the ASOs is desired. Multiple tissues and organs are affected by Dravet syndrome; Epilepsy, generalized, with febrile seizures plus, type 2; Febrile seizures, familial, 3A; Migraine, familial hemiplegic, 3; Autism; Epileptic encephalopathy, early infantile, 13; Sick sinus syndrome 1; Alzheimer's disease or SUDEP, with the brain being the most significantly affected tissue. The ASOs of the present invention may be administered to patients parenterally, for example, by intrathecal injection, intracerebroventricular injection, intraperitoneal injection, intramuscular injection, subcutaneous injection, intravitreal injection, or intravenous injection.

In some embodiments, the disease or condition is induced by a mutation in Naᵥ1.1 (a protein encoded by the SCN1A gene). In some instances, the mutation is a loss-of-function mutation in Naᵥ1.1. In some cases, the loss-of-function mutation in Naᵥ1.1 comprises one or more mutations that decreases or impairs the function of Naᵥ1.1 (e.g., by 10%, 20%, 30%, 40%, 50%, 60%, 70%, 80%, 90%, 95%, or more) relative to the function of a wild-type Naᵥ1.1. In some cases, the loss-of-function mutation in Naᵥ1.1 comprises one or more mutations that result in a disease phenotype. Exemplary loss-of-function mutations include, but are not limited to, R859C, T875M, V1353L, I1656M, R1657C, A1685V, M1841T, and R1916G.

In other instances, the mutation is a gain-of-function mutation in Naᵥ1.1. In such cases, the gain-of-function mutation comprises one or more mutations that prolongs activation of Naᵥ1.1 (e.g., by 10%, 20%, 30%, 40%, 50%, 60%, 70%, 80%, 90%, 95%, or more) relative to the function of a wild-type Naᵥ1.1. In such cases, the gain-of-function mutation in Naᵥ1.1 comprises one or more mutations that result in a disease phenotype. Exemplary gain-of-function mutations include, but are not limited to, D188V, W1204R, R1648H, and D1866Y.

In some embodiments, the disease or condition is an encephalopathy. In some cases, the encephalopathy is induced by a loss-of-function mutation in Naᵥ1.1.

In some embodiments, the encephalopathy is epileptic encephalopathy. Exemplary epileptic encephalopathies include, but are not limited to, Dravet Syndrome (DS) (also known as severe myoclonic epilepsy of infancy or SMEI); severe myoclonic epilepsy of infancy (SMEI)-borderland (SMEB); Febrile seizure (FS); epilepsy, generalized, with febrile seizures plus (GEFS+); epileptic encephalopathy, early infantile, 13; cryptogenic generalized epilepsy; cryptogenic focal epilepsy; myoclonic-astatic epilepsy; Lennox-Gastaut syndrome; West syndrome; idiopathic spasms; early myoclonic encephalopathy; progressive myoclonic epilepsy; alternating hemiplegia of childhood; unclassified epileptic encephalopathy; sudden unexpected death in epilepsy (SUDEP); early infantile SCN1A encephalopathy; early infantile epileptic encephalopathy (EIEE); or sick sinus syndrome 1. In some embodiments, the disease or condition is epileptic encephalopathy, optionally selected from Dravet Syndrome (DS) (also known as severe myoclonic epilepsy of infancy or SMEI); severe myoclonic epilepsy of infancy (SMEI)-borderland (SMEB); Febrile seizure (FS); epilepsy, generalized, with febrile seizures plus (GEFS+); epileptic encephalopathy, early infantile, 13; cryptogenic generalized epilepsy; cryptogenic focal epilepsy; myoclonic-astatic epilepsy; Lennox-Gastaut syndrome; West syndrome; idiopathic spasms; early myoclonic encephalopathy; progressive myoclonic epilepsy; alternating hemiplegia of childhood; unclassified epileptic encephalopathy; sudden unexpected death in epilepsy (SUDEP); and sick sinus syndrome 1.

In some instances, GEFS+ is epilepsy, generalized, with febrile seizures plus, type 2.

In some instances, the Febrile seizure is Febrile seizures, familial, 3A.

In some instances, SMEB is SMEB without generalized spike wave (SMEB-SW), SMEB without myoclonic seizures (SMEB-M), SMEB lacking more than one feature of SMEI (SMEB-O), or intractable childhood epilepsy with generalized tonic-clonic seizures (ICEGTC).

In some embodiments, the diseases or conditions induced by a loss-of-function mutation in Naᵥ1.1 include, but are not limited to, Dravet Syndrome (DS) (also known as SMEI); severe myoclonic epilepsy of infancy (SMEI)-borderland (SMEB); Febrile seizure (FS); epilepsy, generalized, with febrile seizures plus (GEFS+); epileptic encephalopathy, early infantile, 13; cryptogenic generalized epilepsy; cryptogenic focal epilepsy; myoclonic-astatic epilepsy; Lennox-Gastaut syndrome; West syndrome; idiopathic spasms; early myoclonic encephalopathy; progressive myoclonic epilepsy; alternating hemiplegia of childhood; unclassified epileptic encephalopathy; sudden unexpected death in epilepsy (SUDEP); sick sinus syndrome 1; early infantile SCN1A encephalopathy; early infantile epileptic encephalopathy (EIEE); autism; or malignant migrating partial seizures of infancy.

In some embodiments, the disease or condition is induced by a gain-of-function mutation in Naᵥ1.1. Exemplary diseases or conditions associated with a gain-of-function mutation in Naᵥ1.1 include, but are not limited to, migraine. In some instances, the disease or condition induced by a gain-of-function mutation in Naᵥ1.1 is migraine.

In some instances, the migraine is migraine, familial hemiplegic, 3.

In some embodiments, the disease or condition is a Naᵥ1.1 genetic epilepsy. The Naᵥ1.1 genetic epilepsy can include a loss-of-function mutation in Naᵥ1.1 or a gain-of-function mutation in Naᵥ1.1. In some cases, the Naᵥ1.1 genetic epilepsy includes one or more hereditary mutations. In other cases, the Naᵥ1.1 genetic epilepsy includes one or more de novo mutations. In some cases, the Naᵥ1.1 genetic epilepsy includes Dravet Syndrome (DS) (also known as severe myoclonic epilepsy of infancy or SMEI); severe myoclonic epilepsy of infancy (SMEI)-borderland (SMEB); Febrile seizure (FS); epilepsy, generalized, with febrile seizures plus (GEFS+); epileptic encephalopathy, early infantile, 13; cryptogenic generalized epilepsy; cryptogenic focal epilepsy; myoclonic-astatic epilepsy; Lennox-Gastaut syndrome; West syndrome; idiopathic spasms; early myoclonic encephalopathy; progressive myoclonic epilepsy; alternating hemiplegia of childhood; unclassified epileptic encephalopathy; early infantile SCN1A encephalopathy; early infantile epileptic encephalopathy (EIEE); sudden unexpected death in epilepsy (SUDEP); or malignant migrating partial seizures of infancy. In some cases, the Naᵥ1.1 genetic epilepsy associated with a loss-of-function mutation in Naᵥ1.1 includes Dravet Syndrome (DS) (also known as severe myoclonic epilepsy of infancy or SMEI); severe myoclonic epilepsy of infancy (SMEI)-borderland (SMEB); Febrile seizure (FS); epilepsy, generalized, with febrile seizures plus (GEFS+); epileptic encephalopathy, early infantile, 13; cryptogenic generalized epilepsy; cryptogenic focal epilepsy; myoclonic-astatic epilepsy; Lennox-Gastaut syndrome; West syndrome; idiopathic spasms; early myoclonic encephalopathy; progressive myoclonic epilepsy; alternating hemiplegia of childhood; unclassified epileptic encephalopathy; early infantile SCN1A encephalopathy; early infantile epileptic encephalopathy (EIEE); sudden unexpected death in epilepsy (SUDEP); malignant migrating partial seizures of infancy.

In some embodiments, the disease or condition is associated with a haploinsufficiency of the SCN1A gene. Exemplary diseases or conditions associated with a haploinsufficiency of the SCN1A gene include, but are not limited to, Dravet Syndrome (DS) (also known as SMEI); severe myoclonic epilepsy of infancy (SMEI)-borderland (SMEB); Febrile seizure (FS); epilepsy, generalized, with febrile seizures plus (GEFS+); epileptic encephalopathy, early infantile, 13; cryptogenic generalized epilepsy; cryptogenic focal epilepsy; myoclonic-astatic epilepsy; Lennox-Gastaut syndrome; West syndrome; idiopathic spasms; early myoclonic encephalopathy; progressive myoclonic epilepsy; alternating hemiplegia of childhood; unclassified epileptic encephalopathy; sudden unexpected death in epilepsy (SUDEP); sick sinus syndrome 1; early infantile SCN1A encephalopathy; early infantile epileptic encephalopathy (EIEE); or malignant migrating partial seizures of infancy. In some cases, the disease or condition is Dravet Syndrome (DS) (also known as SMEI); severe myoclonic epilepsy of infancy (SMEI)-borderland (SMEB); Febrile seizure (FS); epilepsy, generalized, with febrile seizures plus (GEFS+); epileptic encephalopathy, early infantile, 13; cryptogenic generalized epilepsy; cryptogenic focal epilepsy; myoclonic-astatic epilepsy; Lennox-Gastaut syndrome; West syndrome; idiopathic spasms; early myoclonic encephalopathy; progressive myoclonic epilepsy; alternating hemiplegia of childhood; unclassified epileptic encephalopathy; sudden unexpected death in epilepsy (SUDEP); sick sinus syndrome 1; early infantile SCN1A encephalopathy; early infantile epileptic encephalopathy (EIEE); or malignant migrating partial seizures of infancy.

In some cases, the disease or condition is Dravet Syndrome (DS).

Dravet syndrome (DS), otherwise known as severe myoclonic epilepsy of infancy (SMEI), is an epileptic encephalopathy presenting in the first year of life. Dravet syndrome is an increasingly recognized epileptic encephalopathy in which the clinical diagnosis is supported by the finding of sodium channel gene mutations in approximately 70-80% of patients. Mutations of ion channel genes play a major role in the pathogenesis of a range of epilepsy syndromes, resulting in some epilepsies being regarded as channelopathies. Voltage-gated sodium channels (VGSCs) play an essential role in neuronal excitability; therefore, it is not surprising that many mutations associated with DS have been identified in the gene encoding a VGSC subunit. The disease is described by, e.g., Mulley, et al., 2005, and the disease description at OMIM #607208 (Online Mendelian Inheritance in Man, Johns Hopkins University, 1966-2015), both incorporated by reference herein.

Between 70% and 80% of patients carry sodium channel al subunit gene (SCN1A) abnormalities, and truncating mutations account for about 40%, and have a significant correlation with an earlier age of seizures onset. Sequencing mutations are found in about 70% of cases and comprise truncating (40%) and missense mutations (40%) with the remaining being splice-site changes. Most mutations are de novo, but familial mutations occur in 5-10% of cases and are usually missense in nature. The remaining SCN1A mutations comprise splice-site and missense mutations, most of which fall into the pore-forming region of the sodium channel. At present, over 500 mutations have been associated with DS and are randomly distributed along the gene (Mulley, et al., Neurol. 2006, 67, 1094-1095).

The SCN1A gene is located in the cluster of sodium channel genes on human chromosome 2q24 and encodes the α-pore forming subunits known as Nav1.1 of the neuronal voltage gated sodium channel. The SCN1A gene spans approximately 100 kb of genomic DNA and comprises 26 exons. The SCN1A protein consists of four domains, each with six-transmembrane segments. Two splice variants have been identified that result in a long and short isoform that differ in the presence or absence of 11 amino acids in the cytoplasmic loop between domains 1 and 2, in exon 11 (Miller, et al., 1993-2015, and Mulley, et al., 2005, 25, 535-542, incorporated herein by reference).

Alternative splicing events in SCN1A gene can lead to non-productive mRNA transcripts which in turn can lead to aberrant protein expression, and therapeutic agents which can target the alternative splicing events in SCN1A gene can modulate the expression level of functional proteins in DS patients and/or inhibit aberrant protein expression. Such therapeutic agents can be used to treat a condition caused by SCN1A protein deficiency.

One of the alternative splicing events that can lead to non-productive mRNA transcripts is the inclusion of an extra exon in the mRNA transcript that can induce non-sense mediated mRNA decay. The present disclosure provides compositions and methods for modulating alternative splicing of SCN1A to increase the production of protein-coding mature mRNA, and thus, translated functional SCN1A protein. These compositions and methods include antisense oligomers (ASOs) that can cause exon skipping and promote constitutive splicing of SCN1A pre-mRNA. In various embodiments, functional SCN1A protein can be increased using the methods of the disclosure to treat a condition caused by SCN1A protein deficiency.

In some cases, the disease or condition is SMEB.

In some cases, the disease or condition is GEFS+.

In some cases, the disease or condition is a Febrile seizure (e.g., Febrile seizures, familial, 3A).

In some cases, the disease or condition is autism (also known as autism spectrum disorder or ASD).

In some cases, the disease or condition is migraine (e.g., migraine, familial hemiplegic, 3).

In some cases, the disease or condition is Alzheimer's disease.

In some embodiments, the disease or condition is SCN2A encephalopathy.

In some embodiments, the disease or condition is SCN8A encephalopathy.

In some embodiments, the disease or condition is SCN5A arrhythmia.

In embodiments, the antisense oligonucleotide is administered with one or more agents capable of promoting penetration of the subject antisense oligonucleotide across the blood-brain barrier by any method known in the art. For example, delivery of agents by administration of an adenovirus vector to motor neurons in muscle tissue is described in U.S. Pat. No. 6,632,427, “Adenoviral-vector-mediated gene transfer into medullary motor neurons,” incorporated herein by reference. Delivery of vectors directly to the brain, e.g., the striatum, the thalamus, the hippocampus, or the substantia nigra, is described, e.g., in U.S. Pat. No. 6,756,523, “Adenovirus vectors for the transfer of foreign genes into cells of the central nervous system particularly in brain,” incorporated herein by reference.

In embodiments, the antisense oligonucleotides are linked or conjugated with agents that provide desirable pharmaceutical or pharmacodynamic properties. In embodiments, the antisense oligonucleotide is coupled to a substance, known in the art to promote penetration or transport across the blood-brain barrier, e.g., an antibody to the transferrin receptor. In embodiments, the antisense oligonucleotide is linked with a viral vector, e.g., to render the antisense compound more effective or increase transport across the blood-brain barrier. In embodiments, osmotic blood brain barrier disruption is assisted by infusion of sugars, e.g., meso erythritol, xylitol, D(+) galactose, D(+) lactose, D(+) xylose, dulcitol, myo-inositol, L(−) fructose, D(−) mannitol, D(+) glucose, D(+) arabinose, D(−) arabinose, cellobiose, D(+) maltose, D(+) raffinose, L(+) rhamnose, D(+) melibiose, D(−) ribose, adonitol, D(+) arabitol, L(−) arabitol, D(+) fucose, L(−) fucose, D(−) lyxose, L(+) lyxose, and L(−) lyxose, or amino acids, e.g., glutamine, lysine, arginine, asparagine, aspartic acid, cysteine, glutamic acid, glycine, histidine, leucine, methionine, phenylalanine, proline, serine, threonine, tyrosine, valine, and taurine. Methods and materials for enhancing blood brain barrier penetration are described, e.g., in U.S. Pat. No. 9,193,969, “Compositions and methods for selective delivery of oligonucleotide molecules to specific neuron types,” U.S. Pat. No. 4,866,042, “Method for the delivery of genetic material across the blood brain barrier,” U.S. Pat. No. 6,294,520, “Material for passage through the blood-brain barrier,” and U.S. Pat. No. 6,936,589, “Parenteral delivery systems,” each incorporated herein by reference.

In embodiments, an ASO of the invention is coupled to a dopamine reuptake inhibitor (DRI), a selective serotonin reuptake inhibitor (SSRI), a noradrenaline reuptake inhibitor (NRI), a norepinephrine-dopamine reuptake inhibitor (NDRI), and a serotonin-norepinephrine-dopamine reuptake inhibitor (SNDRI), using methods described in, e.g., U.S. Pat. No. 9,193,969, incorporated herein by reference.

In embodiments, subjects treated using the methods and compositions are evaluated for improvement in condition using any methods known and described in the art.

Methods of Identifying Additional ASOs that Induce Exon Skipping

Also within the scope of the present disclosure are methods for identifying or determining ASOs that induce exon skipping of a SCN1A NIE containing pre-mRNA. For example, a method can comprise identifying or determining ASOs that induce pseudo-exon skipping of a SCN1A NIE containing pre-mRNA. ASOs that specifically hybridize to different nucleotides within the target region of the pre-mRNA may be screened to identify or determine ASOs that improve the rate and/or extent of splicing of the target intron. In some embodiments, the ASO may block or interfere with the binding site(s) of a splicing repressor(s)/silencer. Any method known in the art may be used to identify (determine) an ASO that when hybridized to the target region of the exon results in the desired effect (e.g., pseudo-exon skipping, protein or functional RNA production). These methods also can be used for identifying ASOs that induce exon skipping of the included exon by binding to a targeted region in an intron flanking the included exon, or in a non-included exon. An example of a method that may be used is provided below.

A round of screening, referred to as an ASO “walk” may be performed using ASOs that have been designed to hybridize to a target region of a pre-mRNA. For example, the ASOs used in the ASO walk can be tiled every 5 nucleotides from approximately 100 nucleotides upstream of the 3′ splice site of the included exon (e.g., a portion of sequence of the exon located upstream of the target/included exon) to approximately 100 nucleotides downstream of the 3′ splice site of the target/included exon and/or from approximately 100 nucleotides upstream of the 5′ splice site of the included exon to approximately 100 nucleotides downstream of the 5′ splice site of the target/included exon (e.g., a portion of sequence of the exon located downstream of the target/included exon). For example, a first ASO of 15 nucleotides in length may be designed to specifically hybridize to nucleotides +6 to +20 relative to the 3′ splice site of the target/included exon. A second ASO may be designed to specifically hybridize to nucleotides +11 to +25 relative to the 3′ splice site of the target/included exon. ASOs are designed as such spanning the target region of the pre-mRNA. In embodiments, the ASOs can be tiled more closely, e.g., every 1, 2, 3, or 4 nucleotides. Further, the ASOs can be tiled from 100 nucleotides downstream of the 5′ splice site, to 100 nucleotides upstream of the 3′ splice site. In some embodiments, the ASOs can be tiled from about 1,160 nucleotides upstream of the 3′ splice site, to about 500 nucleotides downstream of the 5′ splice site. In some embodiments, the ASOs can be tiled from about 500 nucleotides upstream of the 3′ splice site, to about 1,920 nucleotides downstream of the 3′ splice site.

One or more ASOs, or a control ASO (an ASO with a scrambled sequence, sequence that is not expected to hybridize to the target region) are delivered, for example by transfection, into a disease-relevant cell line that expresses the target pre-mRNA (e.g., a NIE containing pre-mRNA described herein). The exon skipping effects of each of the ASOs may be assessed by any method known in the art, for example by reverse transcriptase (RT)-PCR using primers that span the splice junction, as described in Example 4. A reduction or absence of a longer RT-PCR product produced using the primers spanning the region containing the included exon (e.g. including the flanking exons of the NIE) in ASO-treated cells as compared to in control ASO-treated cells indicates that splicing of the target NIE has been enhanced. In some embodiments, the exon skipping efficiency (or the splicing efficiency to splice the intron containing the NIE), the ratio of spliced to unspliced pre-mRNA, the rate of splicing, or the extent of splicing may be improved using the ASOs described herein. The amount of protein or functional RNA that is encoded by the target pre-mRNA can also be assessed to determine whether each ASO achieved the desired effect (e.g., enhanced functional protein production). Any method known in the art for assessing and/or quantifying protein production, such as Western blotting, flow cytometry, immunofluorescence microscopy, and ELISA, can be used.

A second round of screening, referred to as an ASO “micro-walk” may be performed using ASOs that have been designed to hybridize to a target region of a pre-mRNA. The ASOs used in the ASO micro-walk are tiled every 1 nucleotide to further refine the nucleotide acid sequence of the pre-mRNA that when hybridized with an ASO results in exon skipping (or enhanced splicing of NIE).

Regions defined by ASOs that promote splicing of the target intron are explored in greater detail by means of an ASO “micro-walk”, involving ASOs spaced in 1-nt steps, as well as longer ASOs, typically 18-25 nt.

As described for the ASO walk above, the ASO micro-walk is performed by delivering one or more ASOs, or a control ASO (an ASO with a scrambled sequence, sequence that is not expected to hybridize to the target region), for example by transfection, into a disease-relevant cell line that expresses the target pre-mRNA. The splicing-inducing effects of each of the ASOs may be assessed by any method known in the art, for example by reverse transcriptase (RT)-PCR using primers that span the NIE, as described herein (see, e.g., Example 4). A reduction or absence of a longer RT-PCR product produced using the primers spanning the NIE in ASO-treated cells as compared to in control ASO-treated cells indicates that exon skipping (or splicing of the target intron containing an NIE) has been enhanced. In some embodiments, the exon skipping efficiency (or the splicing efficiency to splice the intron containing the NIE), the ratio of spliced to unspliced pre-mRNA, the rate of splicing, or the extent of splicing may be improved using the ASOs described herein. The amount of protein or functional RNA that is encoded by the target pre-mRNA can also be assessed to determine whether each ASO achieved the desired effect (e.g., enhanced functional protein production). Any method known in the art for assessing and/or quantifying protein production, such as Western blotting, flow cytometry, immunofluorescence microscopy, and ELISA, can be used.

ASOs that when hybridized to a region of a pre-mRNA result in exon skipping (or enhanced splicing of the intron containing a NIE) and increased protein production may be tested in vivo using animal models, for example transgenic mouse models in which the full-length human gene has been knocked-in or in humanized mouse models of disease. Suitable routes for administration of ASOs may vary depending on the disease and/or the cell types to which delivery of the ASOs is desired. ASOs may be administered, for example, by intrathecal injection, intracerebroventricular injection, intraperitoneal injection, intramuscular injection, subcutaneous injection, intravitreal injection, or intravenous injection. Following administration, the cells, tissues, and/or organs of the model animals may be assessed to determine the effect of the ASO treatment by for example evaluating splicing (efficiency, rate, extent) and protein production by methods known in the art and described herein. The animal models may also be any phenotypic or behavioral indication of the disease or disease severity.

As described herein in various examples, exon 20x in human SCN1A gene is equivalent to exon 21x in mouse SCN1A gene.

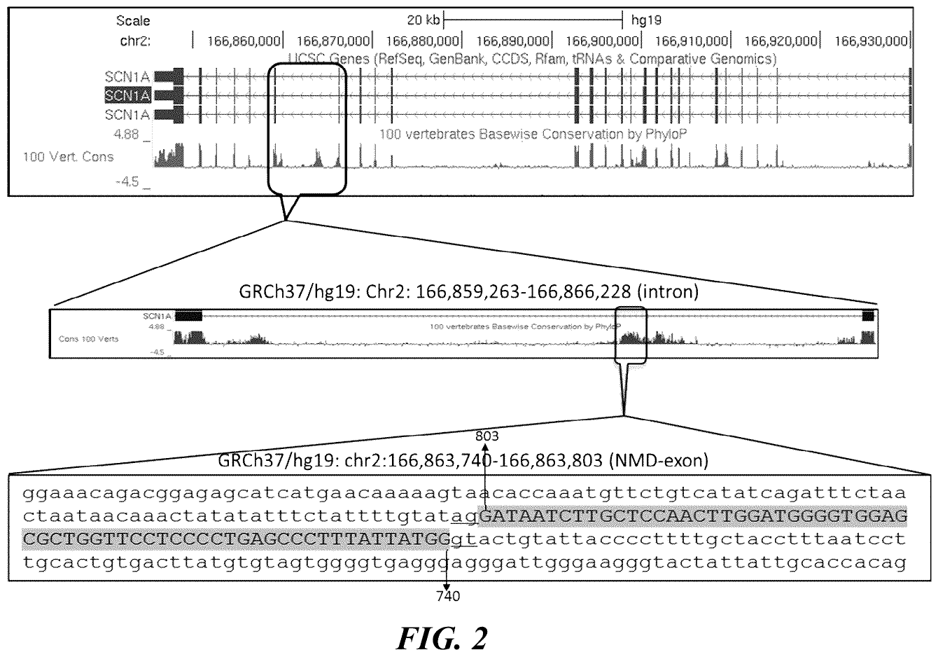

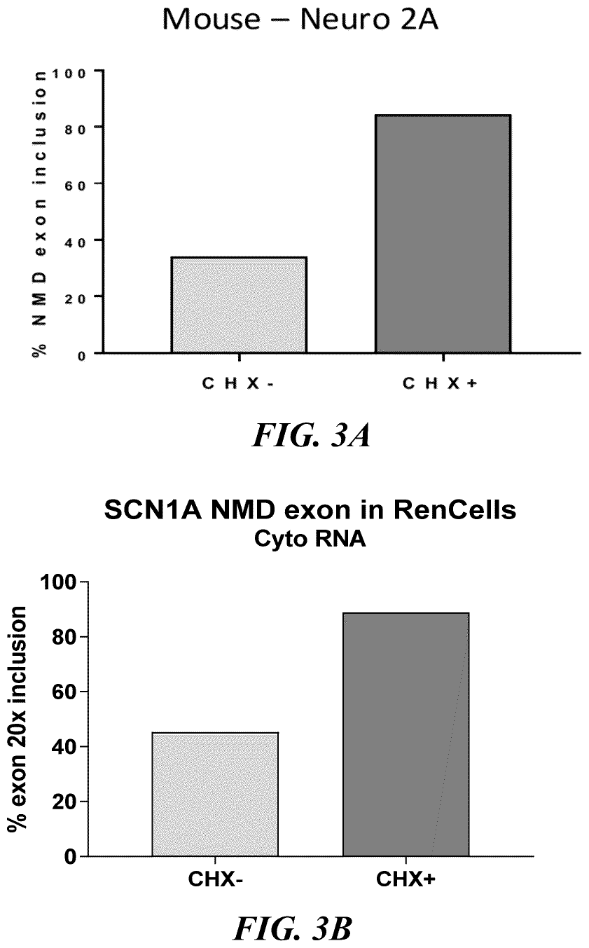

Also within the scope of the present disclosure is a method to identify or validate an NMD-inducing exon in the presence of an NMD inhibitor, for example, cycloheximide. An exemplary method is provided in FIG. 3 and Example 2.

### SPECIFIC EMBODIMENTS

Embodiment 1. A method of modulating expression of SCN1A protein in a cell having an mRNA that contains a non-sense mediated RNA decay-inducing exon (NMD exon mRNA) and encodes SCN1A protein, the method comprising contacting a therapeutic agent to the cell, whereby the therapeutic agent modulates splicing of the NMD exon from the NMD exon mRNA encoding SCN1A protein, thereby modulating the level of processed mRNA encoding SCN1A protein, and modulating expression of SCN1A protein in the cell, wherein the therapeutic agent binds to a targeted portion of the NMD exon mRNA encoding SCN1A, and wherein the targeted portion is: from about 1000 nucleotides upstream from the 5′ end of an NMD-inducing exon (NIE) to about 100 nucleotides upstream from the 5′ end of the NIE; or from about 100 nucleotides downstream of the 3′ end of the NIE to about 1000 nucleotides downstream of the 3′ end of the NIE.

Embodiment 2. A method of treating a disease or condition in a subject in need thereof by modulating expression of SCN1A protein in a cell of the subject, comprising: contacting the cell of the subject with a therapeutic agent that modulates splicing of a non-sense mediated mRNA decay-inducing exon (NMD exon) from an mRNA in the cell that contains the NMD exon and encodes SCN1A, thereby modulating the level of processed mRNA encoding the SCN1A protein, and modulating expression of SCN1A protein in the cell of the subject; wherein the therapeutic agent binds to a targeted portion of the NMD exon mRNA encoding SCN1A, and wherein the targeted portion is: from about 1000 nucleotides upstream from the 5′ end of an NMD-inducing exon (NIE) to about 100 nucleotides upstream from the 5′ end of the NIE; or from about 100 nucleotides downstream of the 3′ end of the NIE to about 1000 nucleotides downstream of the 3′ end of the NIE.

Embodiment 3. The method of embodiment 1 or 2, wherein the therapeutic agent interferes with binding of a factor involved in splicing of the NMD exon from a region of the targeted portion.

Embodiment 4. The method of embodiment 1 or 2, wherein the targeted portion is at most about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides upstream of 5′ end of the NIE.

Embodiment 5. The method of embodiment 1 or 2, wherein the targeted portion is at least about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides upstream of 5′ end of the NIE.

Embodiment 6. The method of embodiment 1 or 2, wherein the targeted portion is at most about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides downstream of 3′ end of the NIE.

Embodiment 7. The method of embodiment 1 or 2, wherein the targeted portion is at least about 800 nucleotides, about 700 nucleotides, about 600 nucleotides, about 500 nucleotides, about 400 nucleotides, about 300 nucleotides, about 200 nucleotides, about 100 nucleotides, about 80 nucleotides, about 70 nucleotides, about 60 nucleotides, about 50 nucleotides, about 40 nucleotides, about 30 nucleotides, about 20 nucleotides, about 10 nucleotides, about 5 nucleotides, about 4 nucleotides, about 2 nucleotides, about 1 nucleotides downstream of 3′ end of the NIE.

Embodiment 8. The method of any one of the embodiments 1-7, wherein the therapeutic agent is an antisense oligomer (ASO).

Embodiment 9. The method of embodiment 8, wherein the ASO comprises a sequence that is at least about 80%, 85%, 90%, 95%, 97%, or 100% identity to any one of SEQ ID NOs: 12-731.

Embodiment 10. The method of any one of the embodiments 1-9, wherein the therapeutic agent promotes exclusion of the NMD exon from the processed mRNA encoding SCN1A protein.

Embodiment 11. The method of embodiment 10, wherein exclusion of the NMD exon from the processed mRNA encoding SCN1A protein in the cell contacted with the therapeutic agent is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to exclusion of the NMD exon from the processed mRNA encoding SCN1A protein in a control cell.

Embodiment 12. The method of embodiment 10, wherein the therapeutic agent increases level of the processed mRNA encoding SCN1A protein in the cell.

Embodiment 13. The method of embodiment 10, wherein an amount of the processed mRNA encoding SCN1A protein in the cell contacted with the therapeutic agent is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to an total amount of the processed mRNA encoding SCN1A protein in a control cell.

Embodiment 14. The method of embodiment 2, wherein the disease or condition is induced by a loss-of-function mutation in Naᵥ1.1.

Embodiment 15. The method of embodiment 14, wherein the disease or condition is associated with haploinsufficiency of the SCN1A gene, and wherein the subject has a first allele encoding a functional SCN1A, and a second allele from which SCN1A is not produced or produced at a reduced level, or a second allele encoding a nonfunctional SCN1A or a partially functional SCN1A.

Embodiment 16. The method of embodiment 14, wherein the disease or condition is encephalopathy.

Embodiment 17. The method of embodiment 16, wherein the encephalopathy is epileptic encephalopathy.

Embodiment 18. The method of embodiment 14, wherein the disease or condition is Dravet Syndrome (DS); severe myoclonic epilepsy of infancy (SMEI)-borderland (SMEB); Febrile seizure (FS); epilepsy, generalized, with febrile seizures plus (GEFS+); epileptic encephalopathy, early infantile, 13; cryptogenic generalized epilepsy; cryptogenic focal epilepsy; myoclonic-astatic epilepsy; Lennox-Gastaut syndrome; West syndrome; idiopathic spasms; early myoclonic encephalopathy; progressive myoclonic epilepsy; alternating hemiplegia of childhood; unclassified epileptic encephalopathy; sudden unexpected death in epilepsy (SUDEP); sick sinus syndrome 1; autism; or malignant migrating partial seizures of infancy.

Embodiment 19. The method of embodiment 18, wherein GEFS+ is epilepsy, generalized, with febrile seizures plus, type 2.

Embodiment 20. The method of embodiment 18, wherein the Febrile seizure is Febrile seizures, familial, 3A.

Embodiment 21. The method of embodiment 18, wherein SMEB is SMEB without generalized spike wave (SMEB-SW), SMEB without myoclonic seizures (SMEB-M), SMEB lacking more than one feature of SMEI (SMEB-O), or intractable childhood epilepsy with generalized tonic-clonic seizures (ICEGTC).

Embodiment 22. The method of embodiments 1 or 2, wherein the ASO consists of a sequence selected from SEQ ID NOs: 72 or 432.

Embodiment 23. The method of embodiments 1 or 2, wherein the ASO consists of a sequence selected from SEQ ID NOs: 73 or 433.

Embodiment 24. The method of embodiments 1 or 2, wherein the ASO consists of a sequence selected from SEQ ID NOs: 76 or 436.

Embodiment 25. The method of embodiments 1 or 2, wherein the ASO consists of a sequence selected from SEQ ID NOs: 181 or 541.

Embodiment 26. The method of embodiments 1 or 2, wherein the ASO consists of a sequence selected from SEQ ID NOs: 220 or 580.

Embodiment 27. A method of modulating expression of SCN1A protein in a cell having an mRNA that contains a non-sense mediated RNA decay-inducing exon (NMD exon mRNA) and encodes SCN1A protein, the method comprising contacting a therapeutic agent to the cell, whereby the therapeutic agent modulates splicing of the NMD exon from the NMD exon mRNA encoding SCN1A protein, thereby modulating the level of processed mRNA encoding SCN1A protein, and modulating expression of SCN1A protein in the cell, wherein the therapeutic agent binds to a targeted portion of the NMD exon mRNA encoding SCN1A, and wherein the targeted portion is from about 1000 nucleotides upstream from the 5′ end of an NMD-inducing exon (NIE) to about 1000 nucleotides downstream of the 3′ end of the NIE.

Embodiment 28. A method of treating a disease or condition in a subject in need thereof by modulating expression of SCN1A protein in a cell of the subject, comprising: contacting the cell of the subject with a therapeutic agent that modulates splicing of a non-sense mediated mRNA decay-inducing exon (NMD exon) from an mRNA in the cell that contains the NMD exon and encodes SCN1A, thereby modulating the level of processed mRNA encoding the SCN1A protein, and modulating expression of SCN1A protein in the cell of the subject; wherein the therapeutic agent binds to a targeted portion of the NMD exon mRNA encoding SCN1A, and wherein the targeted portion is from about 1000 nucleotides upstream from the 5′ end of an NMD-inducing exon (NIE) to about 1000 nucleotides downstream of the 3′ end of the NIE.

Embodiment 29. An antisense oligomer (ASO) comprising a sequence that is at least about 80%, 85%, 90%, 95%, 97%, or 100% identity to any one of SEQ ID NOs: 12-731.

Embodiment 30. An antisense oligomer (ASO) consisting of a sequence selected from SEQ ID NOs: 12-731.

Embodiment 31. A method of treating a disease or condition in a subject in need thereof by modulating expression of SCN1A protein in a cell of the subject, comprising: contacting the cell of the subject with an ASO of embodiment 29 or embodiment 30.

Embodiment 32. A kit comprising an ASO of embodiment 29 or embodiment 30.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

The present invention will be more specifically illustrated by the following Examples. However, it should be understood that the present invention is not limited by these examples in any manner.

### Example 1: Identification of NMD-Inducing Exon Inclusion Events in SCN1A Transcripts by RNAseq Using Next Generation Sequencing

Whole transcriptome shotgun sequencing was carried out using next generation sequencing to reveal a snapshot of transcripts produced by the SCN1A gene to identify NIE inclusion events. For this purpose, poly A+ RNA from nuclear and cytoplasmic fractions of HCN (human cortical neurons) was isolated and cDNA libraries constructed using Illumina's TruSeq Stranded mRNA library Prep Kit. The libraries were pair-end sequenced resulting in 100-nucleotide reads that were mapped to the human genome (February 2009, GRCh37/hg19 assembly). The sequencing results for SCN1A are shown in FIG. 2. Briefly, FIG. 2 shows the mapped reads visualized using the UCSC genome browser (operated by the UCSC Genome Informatics Group (Center for Biomolecular Science & Engineering, University of California, Santa Cruz, 1156 High Street, Santa Cruz, CA 95064) and described by, e.g., Rosenbloom, et al., 2015, “The UCSC Genome Browser database: 2015 update,” Nucleic Acids Research 43, Database Issue, doi: 10.1093/nar/gku1177) and the coverage and number of reads can be inferred by the peak signals. The height of the peaks indicates the level of expression given by the density of the reads in a particular region. The upper panel shows a graphic representation of the SCN1A gene to scale. The conservation level across 100 vertebrate species is shown as peaks. The highest peaks correspond to exons (black boxes), while no peaks are observed for the majority of the introns (lines with arrow heads). Peaks of conservation were identified in intron 20 (NM_006920), shown in the middle panel. Inspection of the conserved sequences identified an exon-like sequence of 64 bp (bottom panel, sequence highlighted in grey) flanked by 3′ and 5′ splice sites (underlined sequence). Inclusion of this exon leads to a frameshift and the introduction of a premature termination codon in exon 21 rendering the transcript a target of NMD.

Exemplary SCN1A gene, pre-mRNA, exon, and intron sequences are summarized in Table 1. The sequence for each exon or intron is summarized in Table 2.

TABLE 1
List of target SCN1A gene and pre-mRNA sequences.

| Species | SEQ ID NO. | Sequence Type |
|---|---|---|
| Human | SEQ ID NO. 1 | SCN1A gene (NC_000002.12) |
| | SEQ ID NO. 2 | SCN1A pre-mRNA (encoding e.g., SCN1A |
| | | mRNA NM_006920.5) |
| | SEQ ID NO. 3 | Intron 22 gene |
| | | (GRCh38/hg38 assembly) |
| | | (coordinate: chr2 166002754 166007229) |
| | SEQ ID NO. 4 | Exon 23 (Exon 20x) gene |
| | | (GRCh38/hg38 assembly) |
| | | (coordinate: chr2 166007230 166007293) |
| | SEQ ID NO. 5 | Intron 23 gene |
| | | (GRCh38/hg38 assembly) |
| | | (coordinate: chr2 166007294 166009718) |
| | SEQ ID NO. 6 | IVS 22 + IVS 23 pre-mRNA |
| | | (pre-mRNA sequence of introns 22 and 23) |
| | SEQ ID NO. 7 | Exon 23 (Exon 20x) pre-mRNA |
| Mouse | SEQ ID NO. 8 | SCN1A gene (NC_000068.7) |
| | SEQ ID NO. 9 | SCN1A pre-mRNA (encoding e.g., SCN1A |
| | | mRNA NM_001313997.1) |
| | SEQ ID NO. 10 | Intron 21 pre-mRNA |
| | SEQ ID NO. 11 | Exon 21x pre-mRNA |

TABLE 2
Sequences of target exon or intron to SCN1A pre-mRNA transcripts.

| SEQ ID | | |
|---|---|---|
| NO. | Sequence Type | Sequence |
| 3 | Intron 22 gene | gtaagaaaaatgaaagaacctgaagtattgtatatagccaaaattaaactaaattaaatttagaaaaagga |
| | | aaatctatgcatgcaaaaggaatggcaaattcttgcaaaattgctactttattgttttatctgttgcatatttactt |
| | | ctaggtgatatgcaagagaaataggcctctcttgaaatgatataatatcatttatctgctgtgcttatttaaatg |
| | | actttatttcctaatccatcttgggagtttccttacaaatctatatacaaaaaaaagctgatgcattattaaagta |
| | | ctatgtgtaatgatataatggtaatctaaagtaaattctatatcaggtacttattctagtgatgatatactgtact |
| | | taacgagttttcctgaaaataatgtgaatcacacatgtgcctaagtatgagtgttaagaaaaaaatgaaagg |
| | | agttgttaaaacttttgtctgtataatgccaaagtttgcattatttgaatatattcaagattagatggttagatatt |
| | | aagtgttgactgaatttataaaactagtaatactaacttaaagattacatacaaatccacatcatttttataaca |
| | | ataaagtaaaacacttataatgaacagaaaatataattttgactcattactataggtaatttatacattaacctt |
| | | aacttgcatcttattggtcagagtcacacaaaatgttattttatccttttcaaagatgcaataatcattttccatc |
| | | atgcataacagattagaaattttgccattattgacttattttccatgcctttttttacggcatgaagcattagttta |
| | | tagatatataatataaaaaattagttctgcttttttttaaaaaaaaatattatcaaaacaaaacactgaattgtgt |
| | | gattccaatagaaaaacactgctctttcacctcctaaggtgtagttacttttatggaaactaagctgtattgta |
| | | gacttccatttgcactttgtagattgtaatagccttatgttctatctcaagtcttattataaatgtcactttgtaa |
| | | gaacgtaggacttgtcttcgatttccctaacatatatgaaaactttgtcctcattatcgacaactcagaacaat |
| | | ataatacaagtagtcctcttttatttctcacagagagcctcaaattttcaccaaaatgttaacagaaattatctc |
| | | tggggtgtataagaattaagtctgttaccaattaaatgtcactttgltttgtttcagactggcagtttcagttctg |
| | | gagaaaaaaaatgtcatttgtgtacattctacttgaaaacatgttgcctgaatcaaaataatatattttatatgg |
| | | cttgtgaaatctgaacaatgctaaacatttgaaaatattataaaccttttacatttgaccatttgaaagtttatta |
| | | aattcattggtcaagtgctcagatatttccatacattacacttcatttctataaaaaagctgatcttatcggtata |
| | | cttttaattttctcagaaataaccatatctataattattaatcaataatgccttttatattaaaagaggttagtttttg |
| | | aaacttggagttttagacataaaatccttataaatgctgatagtgatataactaatagtttaaatggtcagattt |
| | | atgaatatggctctattcctcataatgacaacatacacacagcactaaaatgactaatctcttcaatacgtgtt |
| | | tggcattgtagagtcaaaataacgttataattgattctattttttatacttctagtgtaggatattttattttgtaaa |
| | | aatataatcatgaatgatggtgaggttggatataagaatgatgattatgattgggaagtgagatttgaacat |
| | | gctcagaaactctcatttaattctttgccctagcagcataaaatcacaatagctgcgtcaaagcgtaactca |
| | | ggcactcattttatttttgttgttctgttattttttcaaagcatgtgcttttatgcaacattactgaataaagcatgtt |
| | | gtacagtgcttgataagaagttagaaagtaacaaataaattatcatcacgttgcactttgtgttttgcatgtttt |
| | | atgcacatttctggctgacagcttttaaacatttattgtatttcaaatttccagtccaaatttttcaacttgtaaaa |
| | | ttaaactgagtgaattgatgtcgtgaatatctagggtaaaataaaatttgtgtttaaatttgtatttttaatttcct |
| | | aacctaggaaatcttaaataccttctttttcaaaagaactcaagtcttaatggatagggaaacagacggaga |
| | | gcatcatgaacaaaaagtaacaccaaatgttctgtcatatcagatttctaactaataacaaactatatatttct |
| | | attttgtatag |
| 4 | Exon 23 (Exon | GATAATCTTGCTCCAACTTGGATGGGGTGGAGCGCTGGTTCCT |
| | 20x) gene | CCCCTGAGCCCTTTATTATGG |
| 5 | Intron 23 gene | gtactgtattaccccttttgctacctttaatccttgcactgtgacttatgtgtagtggggtgagggagggattg |
| | | ggaagggtactattattgcaccacagtagggaaaatacattatttacatcctaatcccctcttttcaattgtctt |
| | | aaatttcatttgaaaaaaaaaaaaacctttatgaatttaccctctgtggattttaaccccaatggttgatatcttt |
| | | attaagtttcattgaatatgatttagttatgtgtatatggagttatccatctttggggagattactggattggtga |
| | | gggcgggggaccctggtgtagaatgattatgtgaaaaaacaatttaacttgttaagctcatgatactgtttg |
| | | aggcatacagcccctgctgtttagtacattggtctgggtcctgaaaattaccagttagataccatcagttgat |
| | | tattgatatgtatgagcagatactagggtgcaatatttcaggtttcataagactggtattgattgtgaccactc |
| | | tcattItttattgtgtaagttcatatggggttattttcaaaatgttaacaaggcaaaaatatattaagaaatagtt |
| | | gaataagcacatgtgaattgtgttgtaaacaaaaagttagaataaaaaaatccacttatttgaattatgcaga |
| | | atagaatacatacctagaaataaaacaaaaacgtcttatcatgagtattaagataaaatttaaggcataaact |
| | | cacttcttagaataagtaactcccaactaactttctaggattttaaaacataacacagtgaaaacatacataa |
| | | acataactctacattttatttattcttaaagtttaagtgtattatacaagaagaagagtttatattcgagagaca |
| | | gaaaaagtcagaatttllgtttggatcaccaatatatcatagcttacaaaaaaactgtcttaattaaaacccac |
| | | aacataatttttttagatttttaagaaagattctattattcttctttatacttaaaaatggatgattcctactttgccc |
| | | acttttatttttattcacatagattttctttatttctattagagaagcactagaattcatgaatagtgttgatttgaa |
| | | gttcaaagtaattaattcagataaaaagacatttctgcatgtatgaaaatttctaatgtgaatttgcatatttaat |
| | | tatcaatccttcatttagtgtagacttatttttaaaaatgcaggtaatgaaccagaaatagaattggttgtgcta |
| | | gagtagagaaactttatttgatgattgltttgaaaaaaaagcttctgagaagaaacaacctctagtacagtat |
| | | taattcattaagatagctcctttctcagacatttcctttcatgtagcctgaaagttcaatttgaaatttgttcttcc |
| | | aatttattcagactaattctgcctactttcttccccccataagaaccaattactgcagctttattgagactgaaa |
| | | aaagttaatacacctccttattgctgaaccaaggaatggcttggaactcttgggaaaagacaatcttttcta |
| | | tgatctttcattgtctaatttaatacatcataaatatgactatagctttgtataataaactccccaatactgtgcc |
| | | agatgltttctaagataaagttattttatgttcacaaaaaaaataaaacttttctctgggccaaatgtatgccaa |
| | | ctttgcaaatcatatcctgaagtgcactgctgcagagtacatgcttgcgtcataaattccatagagttcgcttt |
| | | aactctaaatcaatccccagtttcaaagtaaacctctcaaacatattacctaagcacaaacttctccctgtgc |
| | | tcagttccttaattattctcatcccatattcagaaataacatttaaaaattatgctttgatcaataaatactaatct |
| | | aaactttgcttcattaacccattcatttttgtcaaccattattttattcctatattcaaagctctctggtatgttctta |
| | | tattcaagacactcaaggccctggaagattcacgaacatatgtttgcatcttaaatttttagaaaatcttacaa |
| | | tctgtcaggattacactgaactctagtacagagtaatatgggtaccagataagtgggagcaactcttccac |
| | | gtagactggaaacagcactaaatgctatttataggctactttctgaacttaacttgttttaacctcatttttctca |
| | | tatgccaaatgagaacgcaatactgaattatctgtacagttctgttcagtactagaattctgattcttgaattca |
| | | aaggggaaaacattcctctttattttggaggctaaactgggggacaaagttaggctccatgaaagaagtg |
| | | ctatttgaactaaagcctttaagaggggagagtatttcagaagaggagctattagacaaggaatttcaatgt |
| | | aaatggcatctcaatcacctggcaattatattagcacacggttattatattaattgaagtggcatgaagtata |
| | | gatgaccagggaagttaaaactggaaatatagattgtggagtgatgtgaataccaaggtaagaaaaatat |
| | | ttgttagttaccagagagccaataaataactttcaagtgggacttggggaagattaattcatcttacatagat |
| | | taaatgaaggagaaggttaggagacagatgacagtgcaagtatgaaataacagagggcagttctaggt |
| | | ggtgactgtgagaatggaaaagaggtggcaaagctgagaaacgtttcaaagaaaaaatgtgagacagg |
| | | taatgtgaaaagaaaatcgagaaataggtatagataatcagtgttctgctcatactctaaattgggtgttgaa |
| | | ggcaaaatacgtaltttaattagtactctgtgtatacacactagaaacagcattgtaatctggatagtggaca |
| | | aaatattcagaaaagaagggaaatagtaacttgatttcaatttccaaatctctaatctgaaagaaatctaattc |
| | | tattcatccatttaaaataaattatataacgagaatttatgaagtccattgtattaatgcagacagtcagatga |
| | | gataaggcaaagtgtcacgtgtcagcttggtagttgcatcggccacatcatttggttctgcctggataactc |
| | | aaccaaattaatttttcatactcatcccctccacctttgtcattactggtattcttattttctttggcccacttatca |
| | | cactgltttatgttccccagaaggcctagagttctttacaggctttaaacagggatcagaagtataagaaatt |
| | | ggctcatgtatttttttttcagacaggcagttaaaaaaaattgttctaaaaatacactggcatcaaatggcaa |
| | | atagaagatgttttgacgactacttccattggatcagactgacaagaataatacaagcacataggtggaatt |
| | | aaacttagctattaatgtccaagtttgaggcagctgccccttataagcattttagggtctglttttagcttccct |
| | | cttagccactcctgtgcagctccagtgggaggtatggaggaaaaagcaaggaagccatccctatgttgtt |
| | | tccaaacatgaacactcaagatttttaactagtggtccagaagtaaagagggggaaaacatccttctatag |
| | | aaaaaaaaaaaagtagataataattgaacacagaacttcatgtgatcacatcagatttgagaactatgtatg |
| | | gcatccctctttttcttattttcctaagaaatgatttctattatgtttcatttgaaataagtttlltgaattaaactcag |
| | | taaatgaaacaactgacatgactggagcttgaaataaacgatgtgatgatctaatgaaatacataatgcaa |
| | | attgtcttgcttcttatgcaaaaattattagtcatagcaatgcatgaataattaaagacaattatattaggtattt |
| | | aataataltttttatatttatcatctgaatttttaagttattttaaaaatatattggtcaaatcaactcaggtccaaat |
| | | gttttagttttgttctttaatatattgcctttttaaaatgagttaaacttctgtataggctttttaacttttctttattc |
| | | tgataacacaattctgacttcatctggcagcaagttcctctgattttccttttcctttaaccttttaatgcttctccct |
| | | cccttttttttaaaaacatttttgtttcatttcttggttatattgcctatagttgttttcctaagtgtattgcttaagaa |
| | | aaaaaaatgaattttaagatttttttgaaccttgcttttacatatcctagaataaatagcattgatagaaaaaaa |
| | | gaatggaaagaccagagattactaggggaattttttttctttattaacagataagaattctgacttttctttttttc |
| | | catttgtgtattag |
| 6 | IVS 22 + IVS 23 | guaagaaaaaugaaagaaccugaaguauuguauauagccaaaauuaaacuaaauuaaauuuag |
| | pre-mRNA | aaaaaggaaaaucuaugcaugcaaaaggaauggcaaauucuugcaaaauugcuacuuuauugu |
| | | uuuaucuguugcauauuuacuucuaggugauaugcaagagaaauaggccucucuugaaauga |
| | | uauaauaucauuuaucugcugugcuuauuuaaaugacuuuauuuccuaauccaucuugggag |
| | | uuuccuuacaaaucuauauacaaaaaaaagcugaugcauuauuaaaguacuauguguaaugau |
| | | auaaugguaaucuaaaguaaauucuauaucagguacuuauucuuugugaugauauacuguac |
| | | uuaacgaguuuuccugaaaauaaugugaaucacacaugugccuaaguaugaguguuaagaaa |
| | | aaaaugaaaggaguuguuaaaacuuuugucuguauaaugccaaaguuugcauuauuugaaua |
| | | uauucaagauuagaugguuagauauuaaguguugacugaauuuauaaaacuaguaauacuaa |
| | | cuuaaagauuacauacaaauccacaucauuuuuauaacaauaaaguaaaacacuuauaaugaac |
| | | agaaaauauaauuuugacucauuacuauagguaauuuauacauuaaccuuaacuugcaucuua |
| | | uuggucagagucacacaaaauguuauuuuauccuuuucaaagaugcaauaaucauuuuccau |
| | | caugcauaacagauuagaaauuuugccauuauugacuuauuuuccaugccuuuuuuuacggc |
| | | augaagcauuaguuuauagauauauaauauaaaaaauuaguucugcuuuuuuuuaaaaaaaa |
| | | auauuaucaaaacaaaacacugaauugugugauuccaauagaaaaacacugcucuuucaccuc |
| | | cuaagguguaguuacuuuuauggaaacuaagcuguauuguagacuuccauuugcacuuugua |
| | | gauuguuuauagccuuauguucucuucucaagucuuauuauaaaugucacuuuguaagaacg |
| | | uaggacuugucuucgauuucccuaacauauaugaaaacuuuguccucauuaucgacaacucag |
| | | aacaauauaauacaaguaguccucuuuuauuucucacagagagccucaaauuuucaccaaaau |
| | | guuaacagaaauuaucucugggguguauaagaauuaagucuguuuuccaauuaaaugucacu |
| | | uuguuuuguuucagacuggcaguuucaguucuggagaaaaaaaaugucauuuguguacauuc |
| | | uacuugaaaacauguugccugaaucaaaauaauauauuuuauauggcuugugaaaucugaac |
| | | aaugcuaaacauuugaaaauauuauaaaccuuuuacauuugaccauuugaaaguuuauuaaau |
| | | ucauuggucaagugcucagauauuuccauacauuacacuucauuucuauaaaaaagcugaucu |
| | | uaucgguauacuuuuaauuuucucagaaauaaccauaucuauaauuauuaaucaauaaugccu |
| | | uuuauauuaaaagagguuaguuuuugaaacuuggaguuuuagacauaaaauccuuauaaaug |
| | | cugauagugauauaacuaauaguuuaaauggucagauuuaugaauauggcucuauuccucau |
| | | aaugacaacauacacacagcacuaaaaugacuaaucucuucaauacguguuuggcauuguaga |
| | | gucaaaauaacguuauaauugauucuauuuuuuauacuucuaguguuuggauauuuuauuu |
| | | uguaaaaauauaaucaugaaugauggugagguuggauauaagaaugaugauuaugauuggga |
| | | agugagauuugaacaugcucagaaacucucauuuaauucuuugcccuagcagcauaaaaucac |
| | | aauagcugcgucaaagcguaacucaggcacucauuuuauuuuuguuguucuguuauuuuuuc |
| | | aaagcaugugcuuuuaugcaacauuacugaauaaagcauguuguacagugcuugauaagaag |
| | | uuagaaaguaacaaauaaauuaucaucacguugcacuuuguguuuugcauguuuuaugcaca |
| | | uuucuggcugacagcuuuuaaacauuuauuguauuucaaauuuccaguccaaauuuuucaac |
| | | uuguaaaauuaaacugagugaauugaugucgugaauaucuaggguaaaauaaaauuuguguu |
| | | uaaauuuguauuuuuaauuuccuaaccuaggaaaucuuaaauaccuucuuuuucaaaagaac |
| | | ucaagucuuaauggauagggaaacagacggagagcaucaugaacaaaaaguaacaccaaaugu |
| | | ucugucauaucagauuucuaacuaauaacaaacuauauauuucuauuuuguauagguacugu |
| | | auuaccccuuuugcuaccuuuaauccuugcacugugacuuauguguaguggggugagggag |
| | | ggauugggaaggguacuauuauugcaccacaguagggaaaauacauuauuuacauccuaauc |
| | | cccucuuuucaauugucuuaaauuucauuugaaaaaaaaaaaaaccuuuaugaauuuacccuc |
| | | uguggauuuuaaccccaaugguugauaucuuuauuaaguuucauugaauaugauuuaguua |
| | | uguguauauggaguuauccaucuuuggggagauuacuggauuggugagggcgggggacccu |
| | | gguguagaaugauuaugugaaaaaacaauuuaacuuguuaagcucaugauacuguuugaggc |
| | | auacagccccugcuguuuaguacauuggucuggguccugaaaauuaccaguuagauaccauc |
| | | aguugauuauugauauguaugagcagauacuagggugcaauauuucagguuucauaagacug |
| | | guauugauugugaccacucucauuuuuuauuguguaaguucauaugggguuauuuucaaaa |
| | | uguuaacaaggcaaaaauauauuaagaaauaguugaauaagcacaugugaauuguguuguaa |
| | | acaaaaaguuagaauaaaaaaauccacuuauuugaauuaugcagaauagaauacauaccuagaa |
| | | auaaaacaaaaacgucuuaucaugaguauuaagauaaaauuuaaggcauaaacucacuucuua |
| | | gaauaaguaacucccaacuaacuuucuaggauuuuaaaacauaacacagugaaaacauacauaa |
| | | acauaacucuacauuuuauuuauucuuaaaguuuaaguguauuauacaagaagaagaguuua |
| | | uauucgagagacagaaaaagucagaauuuuuguuuggaucaccaauauaucauagcuuacaaa |
| | | aaaacugucuuaauuaaaacccacaacauaauuuuuuuagauuuuuaagaaagauucuauuau |
| | | ucuucuuuauacuuaaaaauggaugauuccuacuuugcccacuuuuauuuuuauucacauag |
| | | auuuucuuuauuucuauuagagaagcacuagaauucaugaauaguguugauuugaaguucaa |
| | | aguaauuaauucagauaaaaagacauuucugcauguaugaaaauuucuaaugugaauuugca |
| | | uauuuaauuaucaauccuucauuuaguguagacuuauuuuuaaaaaugcagguaaugaacca |
| | | gaaauagaauugguugugcuagaguagagaaacuuuauuugaugauuguuuugaaaaaaaag |
| | | cuucugagaagaaacaaccucuaguacaguauuaauucauuaagauagcuccuuucucagaca |
| | | uuuccuuucauguagccugaaaguucaauuugaaauuuguucuuuccaauuuauucagacua |
| | | auucugccuacuuucuuccccccauaagaaccaauuacugcagcuuuauugagacugaaaaaa |
| | | guuaauacaccuccuucuuugcugaaccaaggaauggcuuggaacucuugggaaaagacaauc |
| | | uuuucuaugaucuuucauugucuaauuuaauacaucauaaauaugacuauagcuuuguauaa |
| | | uaaacuccccaauacugugccagauguuuucuaagauaaaguuauuuuauguucacaaaaaaa |
| | | auaaaacuuuucucugggccaaauguaugccaacuuugcaaaucauauccugaagugcacugc |
| | | ugcagaguacaugcuugcgucauaaauuccauagaguucgcuuuaacucuaaaucaaucccca |
| | | guuucaaaguaaaccucucaaacauauuaccuaagcacaaacuucucccugugcucaguuccu |
| | | uaauuauucucaucccauauucagaaauaacauuuaaaaauuaugcuuugaucaauaaauacu |
| | | aaucuaaacuuugcuucauuaacccauucauuuuugucaaccauuauuuuauuccuauauuc |
| | | aaagcucucugguauguucuuauauucaagacacucaaggcccuggaagauucacgaacauau |
| | | guuugcaucuuaaauuuuuagaaaaucuuacaaucugucaggauuacacugaacucuaguac |
| | | agaguaauauggguaccagauaagugggagcaacucuuccacguagacuggaaacagcacuaa |
| | | augcuauuuauaggcuacuuucugaacuuaacuuguuuuaaccucauuuuucucauaugcca |
| | | aaugagaacgcaauacugaauuaucuguacaguucuguucaguacuagaauucugauucuug |
| | | aauucaaaggggaaaacauuccucuuuauuuuggaggcuaaacugggggacaaaguuaggcu |
| | | ccaugaaagaagugcuauuugaacuaaagccuuuaagaggggagaguauuucagaagaggag |
| | | cuauuagacaaggaauuucaauguaaauggcaucucaaucaccuggcaauuauauuagcacac |
| | | gguuauuauauuaauugaaguggcaugaaguauagaugaccagggaaguuaaaacuggaaau |
| | | auagauuguggagugaugugaauaccaagguaagaaaaauauuuguuaguuaccagagagcc |
| | | aauaaauaacuuucaagugggacuuggggaagauuaauucaucuuacauagauuaaaugaag |
| | | gagaagguuaggagacagaugacagugcaaguaugaaauaacagagggcaguucuagguggu |
| | | gacugugagaauggaaaagagguggcaaagcugagaaacguuucaaagaaaaaaugugagaca |
| | | gguaaugugaaaagaaaaucgagaaauagguauagauaaucaguguucugcucauacucuaaa |
| | | uuggguguugaaggcaaaauacguauuuuaauuaguacucuguguauacacacuagaaacag |
| | | cauuguaaucuggauaguggacaaaauauucagaaaagaagggaaauaguaacuugauuucaa |
| | | uuuccaaaucucuaaucugaaagaaaucuaauucuauucauccauuuaaaauaaauuauauaa |
| | | cgagaauuuaugaaguccauuguauuaaugcagacagucagaugagauaaggcaaaguguca |
| | | cgugucagcuugguaguugcaucggccacaucauuugguucugccuggauaacucaaccaaa |
| | | uuaauuuuucauacucauccccuccaccuuugucauuacugguauucuuauuuucuuuggcc |
| | | cacuuaucacacuguuuuauguuccccagaaggccuagaguucuuuacaggcuuuaaacagg |
| | | gaucagaaguauaagaaauuggcucauguauuuuuuuuucagacaggcaguuaaaaaaaauu |
| | | guucuaaaaauacacuggcaucaaauggcaaauagaagauguuuugacgacuacuuccauugg |
| | | aucagacugacaagaauaauacaagcacauagguggaauuaaacuuagcuauuaauguccaag |
| | | uuugaggcagcugccccuuauaagcauuuuagggucuguuuuuagcuucccucuuagccacu |
| | | ccugugcagcuccagugggagguauggaggaaaaagcaaggaagccaucccuauguuguuuc |
| | | caaacaugaacacucaagauuuuuaacuagugguccagaaguaaagagggggaaaacauccuu |
| | | cuauagaaaaaaaaaaaaguagauaauaauugaacacagaacuucaugugaucacaucagauu |
| | | ugagaacuauguauggcaucccucuuuuucuuauuuuccuaagaaaugauuucuauuauguu |
| | | ucauuugaaauaaguuuuuugaauuaaacucaguaaaugaaacaacugacaugacuggagcu |
| | | ugaaauaaacgaugugaugaucuaaugaaauacauaaugcaaauugucuugcuucuuaugca |
| | | aaaauuauuagucauagcaaugcaugaauaauuaaagacaauuauauuagguauuuaauaaua |
| | | uuuuuuauauuuaucaucugaauuuuuaaguuauuuuaaaaauauauuggucaaaucaacuc |
| | | agguccaaauguuuuaguuuuguucuuuaauauauugccuuuuuaaaaugaguuaaacuucu |
| | | guauaggcuuuuuaacuuuucuuuauucugauaacacaauucugacuucaucuggcagcaag |
| | | uuccucugauuuuccuuuuccuuuaaccuuuuaaugcuucucccucccuuuuuuuuaaaaac |
| | | auuuuuguuucauuucuugguuauauugccuauaguuguuuuccuaaguguauugcuuaag |
| | | aaaaaaaaaugaauuuuaagauuuuuuugaaccuugcuuuuacauauccuagaauaaauagca |
| | | uugauagaaaaaaagaauggaaagaccagagauuacuaggggaauuuuuuuucuuuauuaac |
| | | agauaagaauucugacuuuucuuuuuuuccauuuguguauuag |
| 7 | Exon 23 (Exon | GAUAAUCUUGCUCCAACUUGGAUGGGGUGGAGCGCUGGUUC |
| | 20x) pre-mRNA | CUCCCCUGAGCCCUUUAUUAUGG |
| 10 | Intron 21 pre- | guaagaaaaaggaaaacucugcagcguuguauauugucaaagcuaggcugaguucaacuuaac |
| | mRNA | uaacgaaaaacacgugcaugcaaaaggaauggcaacccuuugcaaacuugcuacuuuacccuu |
| | | uucucuguugcauauuuacuucuuggugauaugcaagagaaaaucggccucuuugaaaauga |
| | | uuuaauaucauuuaucugcuuugcuaauuaaaaugaccuuaguucauaaucgaucuugggag |
| | | uuuccuuauaauuccuaauacaaagggggaggggcagauacucucuuaaagaacuaaguuga |
| | | gucauguaauaauuaccuagagauaauuuuguuucauaucguucuccucuaugacagcccau |
| | | caguacuuaagggauccuauggaaaguaaugugaaucacaaauguguaugaauacaaaggaaa |
| | | aaaugaagaauuguuaaauguuuugucuuuacaaugccaaaauucucauuauuugaauauau |
| | | ccaagggcagauauuaaccauugacuggaguauaauaauacugccucaacuguaacuaaauua |
| | | augacauugaauaaguaagacacuaauuuaauuacuauaaauacauacacaucuuaugacaau |
| | | acagcugauaaggaaaaagaacauguauuuuuauucauugccauacauggcucgucaaccuua |
| | | acuuaaaccucgguucucaguuacacagaguuuuauguugcucuuuugagcaaagcauuauu |
| | | ccccucuccauaauucaacacguaucagauuuuugcauuuauuggcucauuguuaugaugau |
| | | uauuucaaagcauuauacaaucauuuauagaagaugugccgugugaaaauuauuuuuuuauu |
| | | aagauccaaaauuuacgcucuuuaaaccaaucagaugaaauguauaaggcaaagagugcucau |
| | | uguccugacacuuacaaaccaaggcuccaacaaacaggcuccucucugcaccacauagagggcu |
| | | uucagccuguguccccccauaaaaaccuauuauaaauuuuauuauacuauacuguaagaaacc |
| | | uguccaauuuuuaauuucucuagcacauaugaaaacuucucuucaguagauccaaguaagcac |
| | | aaaggagcuuugauucucacacaagaaaucacauuuguauuaaaaauguaucauaaauuuucu |
| | | cccaauuaugcaaaacuuaaaugcuuuuccaauuaaaagagcacuuucguuucagaauagcaa |
| | | uuucaguugucaaggaaaacauuguuuuuauacauuuuauauaaaaauacaugagcuaaauu |
| | | uaaauucacauuuuucaacuuuuuaugguuuuuuuauguuucuuuuucuuucgcauuuuuu |
| | | aacaauccagccauuaccucuccucccugucccccuccuauaguuucucaucucauuccuccu |
| | | cccccuugccucugagaggaugcucccucccucacuaggccucccucuuccccagggcuucaa |
| | | guuccucaaggauuauacacaucuucucccacugagaccaggccaggcaguccucugcuucug |
| | | cccagccuguguauguuccugcuugguagcucagucucuggaagcucccuggggucugggu |
| | | uaguugggacugcuggucuuccuauggaguuacccuccccuucaacuucuucaauccuuccc |
| | | cuaauucaaccacagguauccagacuucaguccaaugguugaguguaaauauuggcaucugu |
| | | cucugucagcuguuguuaggggcucagaggacagccaugcuaggcuccugccuacaagcagc |
| | | acaccauaacaucaguaauagugucaggccuuuaaugaauaaugcuacguauauaagguugu |
| | | uagauuauacuucaacuuugaucuuuuagaauauuauuaaauccagucguuuauuuuuuaua |
| | | uguaauauugacuuuccauaacaaaugagucuauuuuccuuuuguguagaaauaacuuuauc |
| | | aauuauuguuaauaaugcuuuugucaauuauuguugauaugcuucucuuuuuuaaaacugg |
| | | agucaucaaaacaaaaauucuggguagauauuacgaagcugacuccuuggucagcuuggcaca |
| | | uagugagaccacaaauaucucaaggucacggcaauuccucaccaccaguuuggcauugugaag |
| | | ucaaaaccaaccuucuguugauucugguuuuuguauuucuaguaugagacauuuucuacuuu |
| | | guaagaguauauaacuguggauggcggcgagguugggcaugaugaugauuguaagcgggaa |
| | | gugagcuagaguaucuucagaaacucucacuuuauccuccuuggcagcauagaaccgcaaucg |
| | | cuguguccgagugucaaccaggcagucauuuuguuuuggguuuuuugucacucuuucaaag |
| | | caugugcuucuacgcaacacuaccaaacacagcaugcugcauagugcuugagaaggaguuaga |
| | | aaguaacaaacgaguuaucaucacguugcccuuuguguuuugcaugucuuaugcacacuuuu |
| | | ggcugacagcuuuugaacauuuauuguauuucaaauuuccaguccaaauuuuuuuuucaacu |
| | | ugugaaauugaacggaaugaaccgaugucgugaauaccuagggucaaauaaaacuuguauuu |
| | | aaauucguaguuuuaauuucccaagcugggaaaaucguaaaaaccuuuuccaaagaacucaag |
| | | ucuuaguugcuagggaaacaggcagugagcaucauauacaaaaaguaacaccaaauguucugu |
| | | cauaucagcuuucuaacuaauaauaaacuauauauuucuauuuuauauaggauaaucuugcu |
| | | ccaacuuggaugggguggagcggugguuccuccccucagcccuuuauuauggguacuguauu |
| | | accccuuuugcuaccuuuaauccuugcacugugacuuauguguagugggauugagggaggga |
| | | gugggaaggguacaauugcaccacaguagggacaauacaggauuuauuuccaaauccacuacu |
| | | uuuaaugagcuuaaacuucuuuugggaaaaaaaaaguuaucucugacuuaccaucuguggau |
| | | uauaaccccagaaguacauaucuuuauuacguuucacugaauaugauuuagcuauuuauacu |
| | | ucauuguccauuuauggggaaauuacucaauuggugagggugggggacccugguguaggau |
| | | gcugaugaaaacguuuucauuugucaagcucaugguagugacagagcauauaguccuuauuu |
| | | uuucaacacacugcucuggucccucaaugggccagucacauuccaucaguugaucguugaug |
| | | ugugcgagcaguggcuaaagguacaacaggccagguaucucaggguugccaaugguuaugau |
| | | cauucucaucuuuauugcauaaaaauguguuauuugcagaaaguagcaaggcaagaucccug |
| | | ugaaacaagggaauacaaaaaaaaaaaaagaugugcuuuaaguuauaaaaccaaaacaugugaa |
| | | aagucaacuucauugaaguauaaagaauaggauaugcaugaaaaacaaaaaaaucaugagcac |
| | | uaagaaauugguguauaagccaacuccuuguaagcuaccccaauuaacuucccagaaucuuag |
| | | aaggcaucacagugcaccccaaaauaaaaagccaaacugacacuucugcuuccucuuaaaaugu |
| | | aggagucuuggauaagaaagauaauuuuuauuguuuggaagaaaaaaaaauuguuuggauaa |
| | | uugaggcauuuaucuaucaaaaauauuuaucuuaauaaaauuucacaacacugauuuaguug |
| | | uuggcuuuucuaaaaauuuuuuauauuucauauuaagaacucaugauuuuuacuuuccauuu |
| | | uuuaaauucuuauucacauaugguuuuucucuauuucuuagaaaagcuauagaacccauggu |
| | | uuccggugacuuaaaaaacuaaucuaaaugucuucacuuagaugauacuuucaaaugcacuga |
| | | aauuucuaauaucaacaagaauauuugccugguccuaauuuuucacugauuuaauaaaaagua |
| | | ugaacccuaaagaagaaauagacuugaagaacugguugugugacaauacagaaauucugcugg |
| | | gagauguccuuuuaaaacauuguuagaagagacaaccucuacaauccacccauuaagcauacu |
| | | ucucucuuagacaucuccuuuuauguaccuuauaaccucaauguguucuuuccaauugacuu |
| | | agaccaacacuucccagcgcaucccacaugggagccaauuacugacucucccuagagacugcaa |
| | | agaauuaauauuguagaaccaagggaugguugggcucuugggagaggcaauccguuugugau |
| | | cuuuugcucuggauguuaaugaaaucgcaacuuuaaguggauuuucaguggcaaauccucug |
| | | auccuaugccaaauguucucuaagacaaacauccuuguaaaauaaaugucucacugggccaaa |
| | | ucuauggcaaauuugcacguuuuccugaacugcauuccuauauaguauuugccauccugaau |
| | | ucacuaaugggcauuacuuuuaauucaaaaccagucccuucuucaaaggaaaucucucccauu |
| | | uauuacauuaugcaaacugcuuucuuaugcagugguuaaauccuuagccaggcaaguaugag |
| | | gacuggaauuuggauauccagaacccucagaaaugucggaugggcauaguagcuuacaugua |
| | | auuccagagcuagaaagaugaaacuagcccuguccucaagcucugaauucaguuggugcaaua |
| | | aauaagaaagaauccccccccccgaccccuacaucucuuuccucuacaucuauacauguauuuc |
| | | ccauacagcucuaugcuuccacauauaugcucacauaugugcaugcacacuugcacacauaug |
| | | uacacuugacacauugaagcaucauaaucuaacaauuggaauauaagaaaauauuuaacuuuc |
| | | acacagagcagucagagaaaaccauaagagguugaaacucuugaaaaaacuugcaggauaaaca |
| | | gaaaagaguaugagaugccaauucuggucuacuuucugaaccuaacuuguuuuaccuucauu |
| | | agucuaauuuuccaaaugaaccccaagcaccaaauuguccuuauugcucuuuccaguacuaaa |
| | | uuauaauuccucaauucgaaggcaaacacucucaucuuuguuaaggauguguagaguuagac |
| | | ucaauaaacgacauguauauuugagcuaaagccuggaggagggagauuauuucaagggcaag |
| | | uacuuggcagggaguuucaaagaaagaaggcucucaauucucagacuaacaccuuagcgugga |
| | | gugacugucacagaggagggugaaguguaugucaccaguuagggaagcugaaaggggaaaug |
| | | uaucaggcuugugagaauccuucagcagccaagucuagcaccugagcacaauccccagaacug |
| | | accccacaugguggaaaggagaggaauaauuccugcaaauuuuccugugaccuccacccaagu |
| | | gcuauagaaaaugcaugcaugcccacaugcacacacaaauaaacauaguugcaaacuguuuug |
| | | aggaaaauaaccucacaaacugucgagugauguaaaugccaaagaaagagaaauguuuucuaa |
| | | uggcuagagaaccauuaaggaauuuuucaaaaugggacaugggauagauaaauuuaguaucc |
| | | auacugaaagaaggcaagcaaauaaaaucugauaagaauguaauucuuaguaaccgaggacag |
| | | agcgaagauagagaacagggccaauggccaagguggaaagguuugaaggaagcagcaugaaac |
| | | cuacagacuuguaccaaaauguucagugucaugaguguuaaaagugaaaagcuugcauguua |
| | | guauggauuucauauccucggcagacagagcaccucacugugagugggagaugaaguauuca |
| | | gaaaugagaaacaacuacucaauuucaguguucauaucucaaauccaauaaacaacuuuaggg |
| | | guacaauuuuuuaaaaaauuacauuaaaauguuuuuaaaucucuucuuaauaauuuaaaaau |
| | | uaaauugaaaauaacuuuaaaaaguaauauauacaggaaagccugugugcuaauuuuuuagg |
| | | gaggccauaaagggagauaguugcucauuaauuucuacacaucagccuaucuuuggcuucug |
| | | ccuugauagcgcacucugaauuaucuucuucauguucaucccucaucuuuauuguuacuggu |
| | | uucauuucccuuggccacauagcccacuauuuuguauuccccaauggauauuguuccuuaca |
| | | aaguucagccagggcucagaaguacaaggaauuggcucuuauacuucugucagacaggcaaaa |
| | | acuucuaaaauuauacuauaauaaaaaucaaagagaugauauucauaauuaaacuaacaaaagu |
| | | ggcaggcccccccucccccaacaugaguagaauuaaucugacguccauguucaagucugaaac |
| | | acacuugccaauuaagagcacauuagggccagccuuuaucucccucuuaguuacuaaugugca |
| | | guucaauggugagcuauagagaaggaagccaagacuaccauaugucaaauauaaaaaaaaaaa |
| | | aucccauuuuaaaucuguagucccgaauuaaggacaagagagagggaaauaucuuugacauua |
| | | gaaaauggagaaaauauuuuagcacaggacuuuacucagucacaucagaguugauaaguacgu |
| | | augacaucccucuuuuuccuguuuuccugagaaaaugaucucucuaguguuucauuuaagau |
| | | aaguuuauugaauuaaacucaguaaaugaaacaacugacaugacuggagcuugaaauaaacga |
| | | ugugaugaucuacugaaauacaugaugcuaaauugucuugcuucuuaugcaaaaacuacuau |
| | | uaguuauagcaaugcauggauaauuaaggccaaaaauauauuagauguuaaaaauaguuuua |
| | | uauuuauacaucugaauuuuaauuuauauuuaaaguauauugguccaaucaauucaugccca |
| | | aauguuuuaguucuauucuuugagauacuguuuuguuuugggauuuuuuuuuaugagcuaa |
| | | ucucuugccuaggaguuccuacuucucucuccuccuuuuauuuuuucuaauaaacuacacau |
| | | gugucuucauccaggagcuaacuucucccauuuugcuuuuccuuuagcaccuuuuuuauauu |
| | | agauuucuuucuuuucuccaucucuuugcauauugccuauauuucuuuuccuaagcauaaua |
| | | uuuaaaaaagacugaguuuuauguuaagauuauuucugcuuugcucuuacacagauaggaua |
| | | aguagucuugauagaaaauaaaucaaugauuccuagggggaugucuuuuugcuuuuaaucaa |
| | | uaaggauucugacuucucuuucucuccauuuguguauuag |
| 11 | Exon 21x pre- | GAUAAUCUUGCUCCAACUUGGAUGGGGUGGAGCGGUGG |
| | mRNA | UUCCUCCCCUCAGCCCUUUAUUAUGG |

### Example 2: Confirmation of NIE Via Cycloheximide Treatment

RT-PCR analysis using cytoplasmic RNA from DMSO-treated (CHX−) or cycloheximide-treated (CHX+) mouse Neuro 2A cells (FIG. 3A) and RenCell VM (human neuroprogenitor cells) (FIG. 3B) and primers in exon 20 and exon 23 confirmed the presence of a band corresponding to the NMD-inducing exon (20x). The identity of the product was confirmed by sequencing. Densitometry analysis of the bands was performed to calculate percent exon 20x inclusion of total SCN1A transcript. Treatment of RenCell VM with cycloheximide (CHX+) to inhibit NMD led to a 2-fold increase of the product corresponding to the NMD-inducing exon 20x in the cytoplasmic fraction (cf. light grey bar, CHX−, to dark grey bar, CHX+).

### Example 3: SCN1A Exon 23 (Exon 20x) Region ASO Walk

ASOs were designed to cover a region from about 1000 to about 100 nucleotides upstream of the 5′ end of the SCN1A exon 23 (exon 20x) gene by shifting 5 nucleotides at a time. ASOs were also designed to cover a second region from about 1000 to about 100 nucleotides downstream from the 3′ end of the SCN1A exon 23 (exon 20x) gene by shifting 5 nucleotides at a time. A list of ASOs targeting SCN1A is summarized in Table 3. Sequences of ASOs are summarized in Table 4.

TABLE 3
List of ASOs targeting SCN1A

| Gene | Pre-mRNA | ASOs | |
|---|---|---|---|
| SEQ ID NO. | SEQ ID NO. | SEQ ID NO. | NIE |
| SEQ ID NO. 1 | SEQ ID NO. 2 | SEQ ID NOs: | Exon 23 (Exon 20x) |
| | | 12-731 | |

TABLE 4
Sequences of ASOs targeting human SCN1A

| SEQUENCE | Chr2 | Chr2 | SEQ ID | | SEQ ID | |
|---|---|---|---|---|---|---|
| NAME | Start | End | NO: | ASO sequence | NO: | ASO sequence |
| SCN1A- | 166864788 | 166864806 | 12 | ATGAATTTAATA | 372 | AUGAAUUUAAUA |
| IVS22-986 | | | | AACTTT | | AACUUU |
| SCN1A- | 166864783 | 166864801 | 13 | GACCAATGAATT | 373 | GACCAAUGAAUU |
| IVS22-981 | | | | TAATAA | | UAAUAA |
| SCN1A- | 166864778 | 166864796 | 14 | CACTTGACCAAT | 374 | CACUUGACCAAU |
| IVS22-976 | | | | GAATTT | | GAAUUU |
| SCN1A- | 166864773 | 166864791 | 15 | CTGAGCACTTGA | 375 | CUGAGCACUUGA |
| IVS22-971 | | | | CCAATG | | CCAAUG |
| SCN1A- | 166864768 | 166864786 | 16 | AATATCTGAGCA | 376 | AAUAUCUGAGCA |
| IVS22-966 | | | | CTTGAC | | CUUGAC |
| SCN1A- | 166864763 | 166864781 | 17 | ATGGAAATATCT | 377 | AUGGAAAUAUCU |
| IVS22-961 | | | | GAGCAC | | GAGCAC |
| SCN1A- | 166864758 | 166864776 | 18 | AATGTATGGAAA | 378 | AAUGUAUGGAAA |
| IVS22-956 | | | | TATCTG | | UAUCUG |
| SCN1A- | 166864753 | 166864771 | 19 | AGTGTAATGTAT | 379 | AGUGUAAUGUAU |
| IVS22-951 | | | | GGAAAT | | GGAAAU |
| SCN1A- | 166864748 | 166864766 | 20 | AATGAAGTGTAA | 380 | AAUGAAGUGUAA |
| IVS22-946 | | | | TGTATG | | UGUAUG |
| SCN1A- | 166864743 | 166864761 | 21 | ATAGAAATGAAG | 381 | AUAGAAAUGAAG |
| IVS22-941 | | | | TGTAAT | | UGUAAU |
| SCN1A- | 166864738 | 166864756 | 22 | TTTTTATAGAAAT | 382 | UUUUUAUAGAAA |
| IVS22-936 | | | | GAAGT | | UGAAGU |
| SCN1A- | 166864733 | 166864751 | 23 | CAGCTTTTTTATA | 383 | CAGCUUUUUUAU |
| IVS22-931 | | | | GAAAT | | AGAAAU |
| SCN1A- | 166864728 | 166864746 | 24 | AAGATCAGCTTT | 384 | AAGAUCAGCUUU |
| IVS22-926 | | | | TTTATA | | UUUAUA |
| SCN1A- | 166864723 | 166864741 | 25 | CCGATAAGATCA | 385 | CCGAUAAGAUCA |
| IVS22-921 | | | | GCTTTT | | GCUUUU |
| SCN1A- | 166864718 | 166864736 | 26 | GTATACCGATAA | 386 | GUAUACCGAUAA |
| IVS22-916 | | | | GATCAG | | GAUCAG |
| SCN1A- | 166864713 | 166864731 | 27 | TAAAAGTATACC | 387 | UAAAAGUAUACC |
| IVS22-911 | | | | GATAAG | | GAUAAG |
| SCN1A- | 166864708 | 166864726 | 28 | AAAATTAAAAGT | 388 | AAAAUUAAAAGU |
| IVS22-906 | | | | ATACCG | | AUACCG |
| SCN1A- | 166864703 | 166864721 | 29 | CTGAGAAAATTA | 389 | CUGAGAAAAUUA |
| IVS22-901 | | | | AAAGTA | | AAAGUA |
| SCN1A- | 166864698 | 166864716 | 30 | TATTTCTGAGAA | 390 | UAUUUCUGAGAA |
| IVS22-896 | | | | AATTAA | | AAUUAA |
| SCN1A- | 166864693 | 166864711 | 31 | ATGGTTATTTCTG | 391 | AUGGUUAUUUCU |
| IVS22-891 | | | | AGAAA | | GAGAAA |
| SCN1A- | 166864688 | 166864706 | 32 | TAGATATGGTTA | 392 | UAGAUAUGGUUA |
| IVS22-886 | | | | TTTCTG | | UUUCUG |
| SCN1A- | 166864683 | 166864701 | 33 | AATTATAGATAT | 393 | AAUUAUAGAUAU |
| IVS22-881 | | | | GGTTAT | | GGUUAU |
| SCN1A- | 166864678 | 166864696 | 34 | TTAATAATTATA | 394 | UUAAUAAUUAUA |
| IVS22-876 | | | | GATATG | | GAUAUG |
| SCN1A- | 166864673 | 166864691 | 35 | ATTGATTAATAA | 395 | AUUGAUUAAUAA |
| IVS22-871 | | | | TTATAG | | UUAUAG |
| SCN1A- | 166864668 | 166864686 | 36 | GCATTATTGATTA | 396 | GCAUUAUUGAUU |
| IVS22-866 | | | | ATAAT | | AAUAAU |
| SCN1A- | 166864663 | 166864681 | 37 | AAAAGGCATTAT | 397 | AAAAGGCAUUAU |
| IVS22-861 | | | | TGATTA | | UGAUUA |
| SCN1A- | 166864658 | 166864676 | 38 | AATATAAAAGGC | 398 | AAUAUAAAAGGC |
| IVS22-856 | | | | ATTATT | | AUUAUU |
| SCN1A- | 166864653 | 166864671 | 39 | CTTTTAATATAAA | 399 | CUUUUAAUAUAA |
| IVS22-851 | | | | AGGCA | | AAGGCA |
| SCN1A- | 166864648 | 166864666 | 40 | AACCTCTTTTAAT | 400 | AACCUCUUUUAA |
| IVS22-846 | | | | ATAAA | | UAUAAA |
| SCN1A- | 166864643 | 166864661 | 41 | AAACTAACCTCT | 401 | AAACUAACCUCU |
| IVS22-841 | | | | TTTAAT | | UUUAAU |
| SCN1A- | 166864638 | 166864656 | 42 | TTCAAAAACTAA | 402 | UUCAAAAACUAA |
| IVS22-836 | | | | CCTCTT | | CCUCUU |
| SCN1A- | 166864633 | 166864651 | 43 | CAAGTTTCAAAA | 403 | CAAGUUUCAAAA |
| IVS22-831 | | | | ACTAAC | | ACUAAC |
| SCN1A- | 166864628 | 166864646 | 44 | AACTCCAAGTTT | 404 | AACUCCAAGUUU |
| IVS22-826 | | | | CAAAAA | | CAAAAA |
| SCN1A- | 166864623 | 166864641 | 45 | TCTAAAACTCCA | 405 | UCUAAAACUCCA |
| IVS22-821 | | | | AGTTTC | | AGUUUC |
| SCN1A- | 166864618 | 166864636 | 46 | TTATGTCTAAAA | 406 | UUAUGUCUAAAA |
| IVS22-816 | | | | CTCCAA | | CUCCAA |
| SCN1A- | 166864613 | 166864631 | 47 | GGATTTTATGTCT | 407 | GGAUUUUAUGUC |
| IVS22-811 | | | | AAAAC | | UAAAAC |
| SCN1A- | 166864608 | 166864626 | 48 | TATAAGGATTTT | 408 | UAUAAGGAUUUU |
| IVS22-806 | | | | ATGTCT | | AUGUCU |
| SCN1A- | 166864603 | 166864621 | 49 | GCATTTATAAGG | 409 | GCAUUUAUAAGG |
| IVS22-801 | | | | ATTTTA | | AUUUUA |
| SCN1A- | 166864598 | 166864616 | 50 | TATCAGCATTTAT | 410 | UAUCAGCAUUUA |
| IVS22-796 | | | | AAGGA | | UAAGGA |
| SCN1A- | 166864593 | 166864611 | 51 | ATCACTATCAGC | 411 | AUCACUAUCAGC |
| IVS22-791 | | | | ATTTAT | | AUUUAU |
| SCN1A- | 166864588 | 166864606 | 52 | GTTATATCACTAT | 412 | GUUAUAUCACUA |
| IVS22-786 | | | | CAGCA | | UCAGCA |
| SCN1A- | 166864583 | 166864601 | 53 | TATTAGTTATATC | 413 | UAUUAGUUAUAU |
| IVS22-781 | | | | ACTAT | | CACUAU |
| SCN1A- | 166864578 | 166864596 | 54 | TAAACTATTAGTT | 414 | UAAACUAUUAGU |
| IVS22-776 | | | | ATATC | | UAUAUC |
| SCN1A- | 166864573 | 166864591 | 55 | CCATTTAAACTAT | 415 | CCAUUUAAACUA |
| IVS22-771 | | | | TAGTT | | UUAGUU |
| SCN1A- | 166864568 | 166864586 | 56 | TCTGACCATTTAA | 416 | UCUGACCAUUUA |
| IVS22-766 | | | | ACTAT | | AACUAU |
| SCN1A- | 166864563 | 166864581 | 57 | ATAAATCTGACC | 417 | AUAAAUCUGACC |
| IVS22-761 | | | | ATTTAA | | AUUUAA |
| SCN1A- | 166864558 | 166864576 | 58 | TATTCATAAATCT | 418 | UAUUCAUAAAUC |
| IVS22-756 | | | | GACCA | | UGACCA |
| SCN1A- | 166864553 | 166864571 | 59 | AGCCATATTCAT | 419 | AGCCAUAUUCAU |
| IVS22-751 | | | | AAATCT | | AAAUCU |
| SCN1A- | 166864548 | 166864566 | 60 | AATAGAGCCATA | 420 | AAUAGAGCCAUA |
| IVS22-746 | | | | TTCATA | | UUCAUA |
| SCN1A- | 166864543 | 166864561 | 61 | TGAGGAATAGAG | 421 | UGAGGAAUAGAG |
| IVS22-741 | | | | CCATAT | | CCAUAU |
| SCN1A- | 166864538 | 166864556 | 62 | CATTATGAGGAA | 422 | CAUUAUGAGGAA |
| IVS22-736 | | | | TAGAGC | | UAGAGC |
| SCN1A- | 166864533 | 166864551 | 63 | GTTGTCATTATGA | 423 | GUUGUCAUUAUG |
| IVS22-731 | | | | GGAAT | | AGGAAU |
| SCN1A- | 166864528 | 166864546 | 64 | TGTATGTTGTCAT | 424 | UGUAUGUUGUCA |
| IVS22-726 | | | | TATGA | | UUAUGA |
| SCN1A- | 166864523 | 166864541 | 65 | CTGTGTGTATGTT | 425 | CUGUGUGUAUGU |
| IVS22-721 | | | | GTCAT | | UGUCAU |
| SCN1A- | 166864518 | 166864536 | 66 | TAGTGCTGTGTGT | 426 | UAGUGCUGUGUG |
| IVS22-716 | | | | ATGTT | | UAUGUU |
| SCN1A- | 166864513 | 166864531 | 67 | CATTTTAGTGCTG | 427 | CAUUUUAGUGCU |
| IVS22-711 | | | | TGTGT | | GUGUGU |
| SCN1A- | 166864508 | 166864526 | 68 | TTAGTCATTTTAG | 428 | UUAGUCAUUUUA |
| IVS22-706 | | | | TGCTG | | GUGCUG |
| SCN1A- | 166864503 | 166864521 | 69 | AGAGATTAGTCA | 429 | AGAGAUUAGUCA |
| IVS22-701 | | | | TTTTAG | | UUUUAG |
| SCN1A- | 166864498 | 166864516 | 70 | ATTGAAGAGATT | 430 | AUUGAAGAGAUU |
| IVS22-696 | | | | AGTCAT | | AGUCAU |
| SCN1A- | 166864493 | 166864511 | 71 | CACGTATTGAAG | 431 | CACGUAUUGAAG |
| IVS22-691 | | | | AGATTA | | AGAUUA |
| SCN1A- | 166864488 | 166864506 | 72 | CCAAACACGTAT | 432 | CCAAACACGUAU |
| IVS22-686 | | | | TGAAGA | | UGAAGA |
| SCN1A- | 166864483 | 166864501 | 73 | CAATGCCAAACA | 433 | CAAUGCCAAACA |
| IVS22-681 | | | | CGTATT | | CGUAUU |
| SCN1A- | 166864478 | 166864496 | 74 | CTCTACAATGCC | 434 | CUCUACAAUGCC |
| IVS22-676 | | | | AAACAC | | AAACAC |
| SCN1A- | 166864473 | 166864491 | 75 | TTTGACTCTACAA | 435 | UUUGACUCUACA |
| IVS22-671 | | | | TGCCA | | AUGCCA |
| SCN1A- | 166864468 | 166864486 | 76 | GTTATTTTGACTC | 436 | GUUAUUUUGACU |
| IVS22-666 | | | | TACAA | | CUACAA |
| SCN1A- | 166864463 | 166864481 | 77 | ATAACGTTATTTT | 437 | AUAACGUUAUUU |
| IVS22-661 | | | | GACTC | | UGACUC |
| SCN1A- | 166864458 | 166864476 | 78 | CAATTATAACGT | 438 | CAAUUAUAACGU |
| IVS22-656 | | | | TATTTT | | UAUUUU |
| SCN1A- | 166864453 | 166864471 | 79 | AGAATCAATTAT | 439 | AGAAUCAAUUAU |
| IVS22-651 | | | | AACGTT | | AACGUU |
| SCN1A- | 166864448 | 166864466 | 80 | AAAATAGAATCA | 440 | AAAAUAGAAUCA |
| IVS22-646 | | | | ATTATA | | AUUAUA |
| SCN1A- | 166864443 | 166864461 | 81 | TATAAAAAATAG | 441 | UAUAAAAAAUAG |
| IVS22-641 | | | | AATCAA | | AAUCAA |
| SCN1A- | 166864438 | 166864456 | 82 | AGAAGTATAAAA | 442 | AGAAGUAUAAAA |
| IVS22-636 | | | | AATAGA | | AAUAGA |
| SCN1A- | 166864433 | 166864451 | 83 | ACACTAGAAGTA | 443 | ACACUAGAAGUA |
| IVS22-631 | | | | TAAAAA | | UAAAAA |
| SCN1A- | 166864428 | 166864446 | 84 | TCCAAACACTAG | 444 | UCCAAACACUAG |
| IVS22-626 | | | | AAGTAT | | AAGUAU |
| SCN1A- | 166864423 | 166864441 | 85 | AAATATCCAAAC | 445 | AAAUAUCCAAAC |
| IVS22-621 | | | | ACTAGA | | ACUAGA |
| SCN1A- | 166864418 | 166864436 | 86 | AAATAAAATATC | 446 | AAAUAAAAUAUC |
| IVS22-616 | | | | CAAACA | | CAAACA |
| SCN1A- | 166864413 | 166864431 | 87 | TTACAAAATAAA | 447 | UUACAAAAUAAA |
| IVS22-611 | | | | ATATCC | | AUAUCC |
| SCN1A- | 166864408 | 166864426 | 88 | TATTTTTACAAAA | 448 | UAUUUUUACAAA |
| IVS22-606 | | | | TAAAA | | AUAAAA |
| SCN1A- | 166864403 | 166864421 | 89 | GATTATATTTTTA | 449 | GAUUAUAUUUUU |
| IVS22-601 | | | | CAAAA | | ACAAAA |
| SCN1A- | 166864398 | 166864416 | 90 | TTCATGATTATAT | 450 | UUCAUGAUUAUA |
| IVS22-596 | | | | TTTTA | | UUUUUA |
| SCN1A- | 166864393 | 166864411 | 91 | CATCATTCATGAT | 451 | CAUCAUUCAUGA |
| IVS22-591 | | | | TATAT | | UUAUAU |
| SCN1A- | 166864388 | 166864406 | 92 | CTCACCATCATTC | 452 | CUCACCAUCAUU |
| IVS22-586 | | | | ATGAT | | CAUGAU |
| SCN1A- | 166864383 | 166864401 | 93 | CCAACCTCACCA | 453 | CCAACCUCACCA |
| IVS22-581 | | | | TCATTC | | UCAUUC |
| SCN1A- | 166864378 | 166864396 | 94 | TATATCCAACCTC | 454 | UAUAUCCAACCU |
| IVS22-576 | | | | ACCAT | | CACCAU |
| SCN1A- | 166864373 | 166864391 | 95 | ATTCTTATATCCA | 455 | AUUCUUAUAUCC |
| IVS22-571 | | | | ACCTC | | AACCUC |
| SCN1A- | 166864368 | 166864386 | 96 | TCATCATTCTTAT | 456 | UCAUCAUUCUUA |
| IVS22-566 | | | | ATCCA | | UAUCCA |
| SCN1A- | 166864363 | 166864381 | 97 | CATAATCATCATT | 457 | CAUAAUCAUCAU |
| IVS22-561 | | | | CTTAT | | UCUUAU |
| SCN1A- | 166864358 | 166864376 | 98 | CCAATCATAATC | 458 | CCAAUCAUAAUC |
| IVS22-556 | | | | ATCATT | | AUCAUU |
| SCN1A- | 166864353 | 166864371 | 99 | ACTTCCCAATCAT | 459 | ACUUCCCAAUCA |
| IVS22-551 | | | | AATCA | | UAAUCA |
| SCN1A- | 166864348 | 166864366 | 100 | ATCTCACTTCCCA | 460 | AUCUCACUUCCC |
| IVS22-546 | | | | ATCAT | | AAUCAU |
| SCN1A- | 166864343 | 166864361 | 101 | TTCAAATCTCACT | 461 | UUCAAAUCUCAC |
| IVS22-541 | | | | TCCCA | | UUCCCA |
| SCN1A- | 166864338 | 166864356 | 102 | GCATGTTCAAAT | 462 | GCAUGUUCAAAU |
| IVS22-536 | | | | CTCACT | | CUCACU |
| SCN1A- | 166864333 | 166864351 | 103 | TCTGAGCATGTTC | 463 | UCUGAGCAUGUU |
| IVS22-531 | | | | AAATC | | CAAAUC |
| SCN1A- | 166864328 | 166864346 | 104 | GAGTTTCTGAGC | 464 | GAGUUUCUGAGC |
| IVS22-526 | | | | ATGTTC | | AUGUUC |
| SCN1A- | 166864323 | 166864341 | 105 | AATGAGAGTTTC | 465 | AAUGAGAGUUUC |
| IVS22-521 | | | | TGAGCA | | UGAGCA |
| SCN1A- | 166864318 | 166864336 | 106 | AATTAAATGAGA | 466 | AAUUAAAUGAGA |
| IVS22-516 | | | | GTTTCT | | GUUUCU |
| SCN1A- | 166864313 | 166864331 | 107 | CAAAGAATTAAA | 467 | CAAAGAAUUAAA |
| IVS22-511 | | | | TGAGAG | | UGAGAG |
| SCN1A- | 166864308 | 166864326 | 108 | TAGGGCAAAGAA | 468 | UAGGGCAAAGAA |
| IVS22-506 | | | | TTAAAT | | UUAAAU |
| SCN1A- | 166864303 | 166864321 | 109 | GCTGCTAGGGCA | 469 | GCUGCUAGGGCA |
| IVS22-501 | | | | AAGAAT | | AAGAAU |
| SCN1A- | 166864298 | 166864316 | 110 | TTTATGCTGCTAG | 470 | UUUAUGCUGCUA |
| IVS22-496 | | | | GGCAA | | GGGCAA |
| SCN1A- | 166864293 | 166864311 | 111 | GTGATTTTATGCT | 471 | GUGAUUUUAUGC |
| IVS22-491 | | | | GCTAG | | UGCUAG |
| SCN1A- | 166864288 | 166864306 | 112 | CTATTGTGATTTT | 472 | CUAUUGUGAUUU |
| IVS22-486 | | | | ATGCT | | UAUGCU |
| SCN1A- | 166864283 | 166864301 | 113 | CGCAGCTATTGT | 473 | CGCAGCUAUUGU |
| IVS22-481 | | | | GATTTT | | GAUUUU |
| SCN1A- | 166864278 | 166864296 | 114 | TTTGACGCAGCT | 474 | UUUGACGCAGCU |
| IVS22-476 | | | | ATTGTG | | AUUGUG |
| SCN1A- | 166864273 | 166864291 | 115 | TACGCTTTGACG | 475 | UACGCUUUGACG |
| IVS22-471 | | | | CAGCTA | | CAGCUA |
| SCN1A- | 166864268 | 166864286 | 116 | TGAGTTACGCTTT | 476 | UGAGUUACGCUU |
| IVS22-466 | | | | GACGC | | UGACGC |
| SCN1A- | 166864263 | 166864281 | 117 | GTGCCTGAGTTA | 477 | GUGCCUGAGUUA |
| IVS22-461 | | | | CGCTTT | | CGCUUU |
| SCN1A- | 166864258 | 166864276 | 118 | AATGAGTGCCTG | 478 | AAUGAGUGCCUG |
| IVS22-456 | | | | AGTTAC | | AGUUAC |
| SCN1A- | 166864253 | 166864271 | 119 | AATAAAATGAGT | 479 | AAUAAAAUGAGU |
| IVS22-451 | | | | GCCTGA | | GCCUGA |
| SCN1A- | 166864248 | 166864266 | 120 | ACAAAAATAAAA | 480 | ACAAAAAUAAAA |
| IVS22-446 | | | | TGAGTG | | UGAGUG |
| SCN1A- | 166864243 | 166864261 | 121 | GAACAACAAAAA | 481 | GAACAACAAAAA |
| IVS22-441 | | | | TAAAAT | | UAAAAU |
| SCN1A- | 166864238 | 166864256 | 122 | TAACAGAACAAC | 482 | UAACAGAACAAC |
| IVS22-436 | | | | AAAAAT | | AAAAAU |
| SCN1A- | 166864233 | 166864251 | 123 | AAAAATAACAGA | 483 | AAAAAUAACAGA |
| IVS22-431 | | | | ACAACA | | ACAACA |
| SCN1A- | 166864228 | 166864246 | 124 | TTTGAAAAAATA | 484 | UUUGAAAAAAUA |
| IVS22-426 | | | | ACAGAA | | ACAGAA |
| SCN1A- | 166864223 | 166864241 | 125 | CATGCTTTGAAA | 485 | CAUGCUUUGAAA |
| IVS22-421 | | | | AAATAA | | AAAUAA |
| SCN1A- | 166864218 | 166864236 | 126 | AAGCACATGCTT | 486 | AAGCACAUGCUU |
| IVS22-416 | | | | TGAAAA | | UGAAAA |
| SCN1A- | 166864213 | 166864231 | 127 | CATAAAAGCACA | 487 | CAUAAAAGCACA |
| IVS22-411 | | | | TGCTTT | | UGCUUU |
| SCN1A- | 166864208 | 166864226 | 128 | TGTTGCATAAAA | 488 | UGUUGCAUAAAA |
| IVS22-406 | | | | GCACAT | | GCACAU |
| SCN1A- | 166864203 | 166864221 | 129 | AGTAATGTTGCA | 489 | AGUAAUGUUGCA |
| IVS22-401 | | | | TAAAAG | | UAAAAG |
| SCN1A- | 166864198 | 166864216 | 130 | TATTCAGTAATGT | 490 | UAUUCAGUAAUG |
| IVS22-396 | | | | TGCAT | | UUGCAU |
| SCN1A- | 166864193 | 166864211 | 131 | TGCTTTATTCAGT | 491 | UGCUUUAUUCAG |
| IVS22-391 | | | | AATGT | | UAAUGU |
| SCN1A- | 166864188 | 166864206 | 132 | CAACATGCTTTAT | 492 | CAACAUGCUUUA |
| IVS22-386 | | | | TCAGT | | UUCAGU |
| SCN1A- | 166864183 | 166864201 | 133 | CTGTACAACATG | 493 | CUGUACAACAUG |
| IVS22-381 | | | | CTTTAT | | CUUUAU |
| SCN1A- | 166864178 | 166864196 | 134 | AAGCACTGTACA | 494 | AAGCACUGUACA |
| IVS22-376 | | | | ACATGC | | ACAUGC |
| SCN1A- | 166864173 | 166864191 | 135 | TTATCAAGCACT | 495 | UUAUCAAGCACU |
| IVS22-371 | | | | GTACAA | | GUACAA |
| SCN1A- | 166864168 | 166864186 | 136 | ACTTCTTATCAAG | 496 | ACUUCUUAUCAA |
| IVS22-366 | | | | CACTG | | GCACUG |
| SCN1A- | 166864163 | 166864181 | 137 | TTCTAACTTCTTA | 497 | UUCUAACUUCUU |
| IVS22-361 | | | | TCAAG | | AUCAAG |
| SCN1A- | 166864158 | 166864176 | 138 | TTACTTTCTAACT | 498 | UUACUUUCUAAC |
| IVS22-356 | | | | TCTTA | | UUCUUA |
| SCN1A- | 166864153 | 166864171 | 139 | ATTTGTTACTTTC | 499 | AUUUGUUACUUU |
| IVS22-351 | | | | TAACT | | CUAACU |
| SCN1A- | 166864148 | 166864166 | 140 | AATTTATTTGTTA | 500 | AAUUUAUUUGUU |
| IVS22-346 | | | | CTTTC | | ACUUUC |
| SCN1A- | 166864143 | 166864161 | 141 | ATGATAATTTATT | 501 | AUGAUAAUUUAU |
| IVS22-341 | | | | TGTTA | | UUGUUA |
| SCN1A- | 166864138 | 166864156 | 142 | ACGTGATGATAA | 502 | ACGUGAUGAUAA |
| IVS22-336 | | | | TTTATT | | UUUAUU |
| SCN1A- | 166864133 | 166864151 | 143 | GTGCAACGTGAT | 503 | GUGCAACGUGAU |
| IVS22-331 | | | | GATAAT | | GAUAAU |
| SCN1A- | 166864128 | 166864146 | 144 | ACAAAGTGCAAC | 504 | ACAAAGUGCAAC |
| IVS22-326 | | | | GTGATG | | GUGAUG |
| SCN1A- | 166864123 | 166864141 | 145 | AAAACACAAAGT | 505 | AAAACACAAAGU |
| IVS22-321 | | | | GCAACG | | GCAACG |
| SCN1A- | 166864118 | 166864136 | 146 | CATGCAAAACAC | 506 | CAUGCAAAACAC |
| IVS22-316 | | | | AAAGTG | | AAAGUG |
| SCN1A- | 166864113 | 166864131 | 147 | TAAAACATGCAA | 507 | UAAAACAUGCAA |
| IVS22-311 | | | | AACACA | | AACACA |
| SCN1A- | 166864108 | 166864126 | 148 | GTGCATAAAACA | 508 | GUGCAUAAAACA |
| IVS22-306 | | | | TGCAAA | | UGCAAA |
| SCN1A- | 166864103 | 166864121 | 149 | GAAATGTGCATA | 509 | GAAAUGUGCAUA |
| IVS22-301 | | | | AAACAT | | AAACAU |
| SCN1A- | 166864098 | 166864116 | 150 | AGCCAGAAATGT | 510 | AGCCAGAAAUGU |
| IVS22-296 | | | | GCATAA | | GCAUAA |
| SCN1A- | 166864093 | 166864111 | 151 | CTGTCAGCCAGA | 511 | CUGUCAGCCAGA |
| IVS22-291 | | | | AATGTG | | AAUGUG |
| SCN1A- | 166864088 | 166864106 | 152 | AAAAGCTGTCAG | 512 | AAAAGCUGUCAG |
| IVS22-286 | | | | CCAGAA | | CCAGAA |
| SCN1A- | 166864083 | 166864101 | 153 | TGTTTAAAAGCT | 513 | UGUUUAAAAGCU |
| IVS22-281 | | | | GTCAGC | | GUCAGC |
| SCN1A- | 166864078 | 166864096 | 154 | ATAAATGTTTAA | 514 | AUAAAUGUUUAA |
| IVS22-276 | | | | AAGCTG | | AAGCUG |
| SCN1A- | 166864073 | 166864091 | 155 | ATACAATAAATG | 515 | AUACAAUAAAUG |
| IVS22-271 | | | | TTTAAA | | UUUAAA |
| SCN1A- | 166864068 | 166864086 | 156 | TTGAAATACAAT | 516 | UUGAAAUACAAU |
| IVS22-266 | | | | AAATGT | | AAAUGU |
| SCN1A- | 166864063 | 166864081 | 157 | GAAATTTGAAAT | 517 | GAAAUUUGAAAU |
| IVS22-261 | | | | ACAATA | | ACAAUA |
| SCN1A- | 166864058 | 166864076 | 158 | GACTGGAAATTT | 518 | GACUGGAAAUUU |
| IVS22-256 | | | | GAAATA | | GAAAUA |
| SCN1A- | 166864053 | 166864071 | 159 | ATTTGGACTGGA | 519 | AUUUGGACUGGA |
| IVS22-251 | | | | AATTTG | | AAUUUG |
| SCN1A- | 166864048 | 166864066 | 160 | GAAAAATTTGGA | 520 | GAAAAAUUUGGA |
| IVS22-246 | | | | CTGGAA | | CUGGAA |
| SCN1A- | 166864043 | 166864061 | 161 | AAGTTGAAAAAT | 521 | AAGUUGAAAAAU |
| IVS22-241 | | | | TTGGAC | | UUGGAC |
| SCN1A- | 166864038 | 166864056 | 162 | TTTACAAGTTGA | 522 | UUUACAAGUUGA |
| IVS22-236 | | | | AAAATT | | AAAAUU |
| SCN1A- | 166864033 | 166864051 | 163 | TTAATTTTACAAG | 523 | UUAAUUUUACAA |
| IVS22-231 | | | | TTGAA | | GUUGAA |
| SCN1A- | 166864028 | 166864046 | 164 | TCAGTTTAATTTT | 524 | UCAGUUUAAUUU |
| IVS22-226 | | | | ACAAG | | UACAAG |
| SCN1A- | 166864023 | 166864041 | 165 | TTCACTCAGTTTA | 525 | UUCACUCAGUUU |
| IVS22-221 | | | | ATTTT | | AAUUUU |
| SCN1A- | 166864018 | 166864036 | 166 | ATCAATTCACTC | 526 | AUCAAUUCACUC |
| IVS22-216 | | | | AGTTTA | | AGUUUA |
| SCN1A- | 166864013 | 166864031 | 167 | ACGACATCAATT | 527 | ACGACAUCAAUU |
| IVS22-211 | | | | CACTCA | | CACUCA |
| SCN1A- | 166864008 | 166864026 | 168 | TATTCACGACAT | 528 | UAUUCACGACAU |
| IVS22-206 | | | | CAATTC | | CAAUUC |
| SCN1A- | 166864003 | 166864021 | 169 | CTAGATATTCAC | 529 | CUAGAUAUUCAC |
| IVS22-201 | | | | GACATC | | GACAUC |
| SCN1A- | 166863998 | 166864016 | 170 | TTACCCTAGATAT | 530 | UUACCCUAGAUA |
| IVS22-196 | | | | TCACG | | UUCACG |
| SCN1A- | 166863993 | 166864011 | 171 | TTATTTTACCCTA | 531 | UUAUUUUACCCU |
| IVS22-191 | | | | GATAT | | AGAUAU |
| SCN1A- | 166863988 | 166864006 | 172 | AAATTTTATTTTA | 532 | AAAUUUUAUUUU |
| IVS22-186 | | | | CCCTA | | ACCCUA |
| SCN1A- | 166863983 | 166864001 | 173 | AACACAAATTTT | 533 | AACACAAAUUUU |
| IVS22-181 | | | | ATTTTA | | AUUUUA |
| SCN1A- | 166863978 | 166863996 | 174 | ATTTAAACACAA | 534 | AUUUAAACACAA |
| IVS22-176 | | | | ATTTTA | | AUUUUA |
| SCN1A- | 166863973 | 166863991 | 175 | TACAAATTTAAA | 535 | UACAAAUUUAAA |
| IVS22-171 | | | | CACAAA | | CACAAA |
| SCN1A- | 166863968 | 166863986 | 176 | AAAAATACAAAT | 536 | AAAAAUACAAAU |
| IVS22-166 | | | | TTAAAC | | UUAAAC |
| SCN1A- | 166863963 | 166863981 | 177 | AAATTAAAAATA | 537 | AAAUUAAAAAUA |
| IVS22-161 | | | | CAAATT | | CAAAUU |
| SCN1A- | 166863958 | 166863976 | 178 | TTAGGAAATTAA | 538 | UUAGGAAAUUAA |
| IVS22-156 | | | | AAATAC | | AAAUAC |
| SCN1A- | 166863953 | 166863971 | 179 | CTAGGTTAGGAA | 539 | CUAGGUUAGGAA |
| IVS22-151 | | | | ATTAAA | | AUUAAA |
| SCN1A- | 166863948 | 166863966 | 180 | ATTTCCTAGGTTA | 540 | AUUUCCUAGGUU |
| IVS22-146 | | | | GGAAA | | AGGAAA |
| SCN1A- | 166863943 | 166863961 | 181 | TTAAGATTTCCTA | 541 | UUAAGAUUUCCU |
| IVS22-141 | | | | GGTTA | | AGGUUA |
| SCN1A- | 166863938 | 166863956 | 182 | GGTATTTAAGAT | 542 | GGUAUUUAAGAU |
| IVS22-136 | | | | TTCCTA | | UUCCUA |
| SCN1A- | 166863933 | 166863951 | 183 | AAGAAGGTATTT | 543 | AAGAAGGUAUUU |
| IVS22-131 | | | | AAGATT | | AAGAUU |
| SCN1A- | 166863928 | 166863946 | 184 | TGAAAAAGAAGG | 544 | UGAAAAAGAAGG |
| IVS22-126 | | | | TATTTA | | UAUUUA |
| SCN1A- | 166863923 | 166863941 | 185 | TCTTTTGAAAAA | 545 | UCUUUUGAAAAA |
| IVS22-121 | | | | GAAGGT | | GAAGGU |
| SCN1A- | 166863918 | 166863936 | 186 | TGAGTTCTTTTGA | 546 | UGAGUUCUUUUG |
| IVS22-116 | | | | AAAAG | | AAAAAG |
| SCN1A- | 166863913 | 166863931 | 187 | AGACTTGAGTTC | 547 | AGACUUGAGUUC |
| IVS22-111 | | | | TTTTGA | | UUUUGA |
| SCN1A- | 166863908 | 166863926 | 188 | CATTAAGACTTG | 548 | CAUUAAGACUUG |
| IVS22-106 | | | | AGTTCT | | AGUUCU |
| SCN1A- | 166863903 | 166863921 | 189 | CTATCCATTAAG | 549 | CUAUCCAUUAAG |
| IVS22-101 | | | | ACTTGA | | ACUUGA |
| SCN1A- | 166863898 | 166863916 | 190 | TTTCCCTATCCAT | 550 | UUUCCCUAUCCA |
| IVS22-096 | | | | TAAGA | | UUAAGA |
| SCN1A- | 166863893 | 166863911 | 191 | GTCTGTTTCCCTA | 551 | GUCUGUUUCCCU |
| IVS22-091 | | | | TCCAT | | AUCCAU |
| SCN1A- | 166863631 | 166863649 | 192 | TTTCCCTACTGTG | 552 | UUUCCCUACUGU |
| IVS23 + 091 | | | | GTGCA | | GGUGCA |
| SCN1A- | 166863626 | 166863644 | 193 | TGTATTTTCCCTA | 553 | UGUAUUUUCCCU |
| IVS23 + 096 | | | | CTGTG | | ACUGUG |
| SCN1A- | 166863621 | 166863639 | 194 | AATAATGTATTTT | 554 | AAUAAUGUAUUU |
| IVS23 + 101 | | | | CCCTA | | UCCCUA |
| SCN1A- | 166863616 | 166863634 | 195 | ATGTAAATAATG | 555 | AUGUAAAUAAUG |
| IVS23 + 106 | | | | TATTTT | | UAUUUU |
| SCN1A- | 166863611 | 166863629 | 196 | AGGGATTAGGAT | 556 | AGGGAUUAGGAU |
| IVS23 + 111 | | | | TAATGT | | UAAUGU |
| SCN1A- | 166863606 | 166863624 | 197 | AGGGATTAGGAT | 557 | AGGGAUUAGGAU |
| IVS23 + 116 | | | | GTAAAT | | GUAAAU |
| SCN1A- | 166863601 | 166863619 | 198 | AAAGAGGGAATT | 558 | AAAGAGGGAAUU |
| IVS23 + 121 | | | | AGGATG | | AGGAUG |
| SCN1A- | 166863596 | 166863614 | 199 | ATTGAAAAGAAG | 559 | AUUGAAAAGAAG |
| IVS23 + 126 | | | | GGATTA | | GGAUUA |
| SCN1A- | 166863591 | 166863609 | 200 | AGACAATTGAAA | 560 | AGACAAUUGAAA |
| IVS23 + 131 | | | | AGAGGG | | AGAGGG |
| SCN1A- | 166863586 | 166863604 | 201 | ATTTAAGACAAT | 561 | AUUUAAGACAAU |
| IVS23 + 136 | | | | TGAAAA | | UGAAAA |
| SCN1A- | 166863581 | 166863599 | 202 | ATGAAATTTAAG | 562 | AUGAAAUUUAAG |
| IVS23 + 141 | | | | ACAATT | | ACAAUU |
| SCN1A- | 166863576 | 166863594 | 203 | TTCAAATGAAAT | 563 | UUCAAAUGAAAU |
| IVS23 + 146 | | | | TTAAGA | | UUAAGA |
| SCN1A- | 166863571 | 166863589 | 204 | TTTTTTTCAAATG | 564 | UUUUUUUCAAAU |
| IVS23 + 151 | | | | AAATT | | GAAAUU |
| SCN1A- | 166863566 | 166863584 | 205 | TTTTTTTTTTTTC | 565 | UUUUUUUUUUUU |
| IVS23 + 156 | | | | AAATG | | CAAAUG |
| SCN1A- | 166863561 | 166863579 | 206 | AAGGTTTTTTTTT | 566 | AAGGUUUUUUUU |
| IVS23 + 161 | | | | TTTTC | | UUUUUC |
| SCN1A- | 166863556 | 166863574 | 207 | TCATAAAGGTTTT | 567 | UCAUAAAGGUUU |
| IVS23 + 166 | | | | TTTTT | | UUUUUU |
| SCN1A- | 166863551 | 166863569 | 208 | TAAATTCATAAA | 568 | UAAAUUCAUAAA |
| IVS23 + 171 | | | | GGTTTT | | GGUUUU |
| SCN1A- | 166863546 | 166863564 | 209 | GAGGGTAAATTC | 569 | GAGGGUAAAUUC |
| IVS23 + 176 | | | | ATAAAG | | AUAAAG |
| SCN1A- | 166863541 | 166863559 | 210 | CCACAGAGGGTA | 570 | CCACAGAGGGUA |
| IVS23 + 181 | | | | AATTCA | | AAUUCA |
| SCN1A- | 166863536 | 166863554 | 211 | AAAATCCACAGA | 571 | AAAAUCCACAGA |
| IVS23 + 186 | | | | GGGTAA | | GGGUAA |
| SCN1A- | 166863531 | 166863549 | 212 | GGGTTAAAATCC | 572 | GGGUUAAAAUCC |
| IVS23 + 191 | | | | ACAGAG | | ACAGAG |
| SCN1A- | 166863526 | 166863544 | 213 | CATTGGGATTAA | 573 | CAUUGGGAUUAA |
| IVS23 + 196 | | | | AATCCA | | AAUCCA |
| SCN1A- | 166863521 | 166863539 | 214 | TCAACCATTAGG | 574 | UCAACCAUUAGG |
| IVS23 + 201 | | | | GTTAAA | | GUUAAA |
| SCN1A- | 166863516 | 166863534 | 215 | AGATATCAACCA | 575 | AGAUAUCAACCA |
| IVS23 + 206 | | | | TTGGGA | | UUGGGA |
| SCN1A- | 166863511 | 166863529 | 216 | AATAAAGATATC | 576 | AAUAAAGAUAUC |
| IVS23 + 211 | | | | AACCAT | | AACCAU |
| SCN1A- | 166863506 | 166863524 | 217 | AACTTAATAAAG | 577 | AACUUAAUAAAG |
| IVS23 + 216 | | | | ATATCA | | AUAUCA |
| SCN1A- | 166863501 | 166863519 | 218 | AATGAAACTTAA | 578 | AAUGAAACUUAA |
| IVS23 + 221 | | | | TAAAGA | | UAAAGA |
| SCN1A- | 166863496 | 166863514 | 219 | TATTCAATGAAA | 579 | UAUUCAAUGAAA |
| IVS23 + 226 | | | | CTTAAT | | CUUAAU |
| SCN1A- | 166863491 | 166863509 | 220 | AATCATATTCAA | 580 | AAUCAUAUUCAA |
| IVS23 + 231 | | | | TGAAAC | | UGAAAC |
| SCN1A- | 166863486 | 166863504 | 221 | AACTAAATCATA | 581 | AACUAAAUCAUA |
| IVS23 + 236 | | | | TTCAAT | | UUCAAU |
| SCN1A- | 166863481 | 166863499 | 222 | CACATAACTAAA | 582 | CACAUAACUAAA |
| IVS23 + 241 | | | | TCATAT | | UCAUAU |
| SCN1A- | 166863476 | 166863494 | 223 | ATATACACATAA | 583 | AUAUACACAUAA |
| IVS23 + 246 | | | | CTAAAT | | CUAAAU |
| SCN1A- | 166863471 | 166863489 | 224 | ACTCCATATACA | 584 | ACUCCAUAUACA |
| IVS23 + 251 | | | | CATAAC | | CAUAAC |
| SCN1A- | 166863466 | 166863484 | 225 | GGATAACTCCAT | 585 | GGAUAACUCCAU |
| IVS23 + 256 | | | | ATACAC | | AUACAC |
| SCN1A- | 166863461 | 166863479 | 226 | AAGATGGATAAC | 586 | AAGAUGGAUAAC |
| IVS23 + 261 | | | | TCCATA | | UCCAUA |
| SCN1A- | 166863456 | 166863474 | 227 | CCCCAAAGATGG | 587 | CCCCAAAGAUGG |
| IVS23 + 266 | | | | ATAACT | | AUAACU |
| SCN1A- | 166863451 | 166863469 | 228 | AATCTCCCCAAA | 588 | AAUCUCCCCAAA |
| IVS23 + 271 | | | | GATGGA | | GAUGGA |
| SCN1A- | 166863446 | 166863464 | 229 | CCAGTAATCTCC | 589 | CCAGUAAUCUCC |
| IVS23 + 276 | | | | CCAAAG | | CCAAAG |
| SCN1A- | 166863441 | 166863459 | 230 | CCAATCCAGTAA | 590 | CCAAUCCAGUAA |
| IVS23 + 281 | | | | TCTCCC | | UCUCCC |
| SCN1A- | 166863436 | 166863454 | 231 | CCTCACCAATCC | 591 | CCUCACCAAUCC |
| IVS23 + 286 | | | | AGTAAT | | AGUAAU |
| SCN1A- | 166863431 | 166863449 | 232 | CCCGCCCTCACC | 592 | CCCGCCCUCACCA |
| IVS23 + 291 | | | | AATCCA | | AUCCA |
| SCN1A- | 166863426 | 166863444 | 233 | GGTCCCCCGCCC | 593 | GGUCCCCCGCCC |
| IVS23 + 296 | | | | TCACCA | | UCACCA |
| SCN1A- | 166863421 | 166863439 | 234 | ACCAGGGTCCCC | 594 | ACCAGGGUCCCC |
| IVS23 + 301 | | | | CGCCCT | | CGCCCU |
| SCN1A- | 166863416 | 166863434 | 235 | TCTACACCAGGG | 595 | UCUACACCAGGG |
| IVS23 + 306 | | | | TCCCCC | | UCCCCC |
| SCN1A- | 166863411 | 166863429 | 236 | ATCATTCTACACC | 596 | AUCAUUCUACAC |
| IVS23 + 311 | | | | AGGGT | | CAGGGU |
| SCN1A- | 166863406 | 166863424 | 237 | ACATAATCATTCT | 597 | ACAUAAUCAUUC |
| IVS23 + 316 | | | | ACACC | | UACACC |
| SCN1A- | 166863401 | 166863419 | 238 | TTTTCACATAATC | 598 | UUUUCACAUAAU |
| IVS23 + 321 | | | | ATTCT | | CAUUCU |
| SCN1A- | 166863396 | 166863414 | 239 | TTGTTTTTTCACA | 599 | UUGUUUUUUCAC |
| IVS23 + 326 | | | | TAATC | | AUAAUC |
| SCN1A- | 166863391 | 166863409 | 240 | TTAAATTGTTTTT | 600 | UUAAAUUGUUUU |
| IVS23 + 331 | | | | TCACA | | UUCACA |
| SCN1A- | 166863386 | 166863404 | 241 | ACAAGTTAAATT | 601 | ACAAGUUAAAUU |
| IVS23 + 336 | | | | GTTTTT | | GUUUUU |
| SCN1A- | 166863381 | 166863399 | 242 | GCTTAACAAGTT | 602 | GCUUAACAAGUU |
| IVS23 + 341 | | | | AAATTG | | AAAUUG |
| SCN1A- | 166863376 | 166863394 | 243 | CATGAGCTTAAC | 603 | CAUGAGCUUAAC |
| IVS23 + 346 | | | | AAGTTA | | AAGUUA |
| SCN1A- | 166863371 | 166863389 | 244 | AGTATCATGAGC | 604 | AGUAUCAUGAGC |
| IVS23 + 351 | | | | TTAACA | | UUAACA |
| SCN1A- | 166863366 | 166863384 | 245 | CAAACAGTATCA | 605 | CAAACAGUAUCA |
| IVS23 + 356 | | | | TGAGCT | | UGAGCU |
| SCN1A- | 166863361 | 166863379 | 246 | TGCCTCAAACAG | 606 | UGCCUCAAACAG |
| IVS23 + 361 | | | | TATCAT | | UAUCAU |
| SCN1A- | 166863356 | 166863374 | 247 | CTGTATGCCTCA | 607 | CUGUAUGCCUCA |
| IVS23 + 366 | | | | AACAGT | | AACAGU |
| SCN1A- | 166863351 | 166863369 | 248 | AGGGACTGTATG | 608 | AGGGACUGUAUG |
| IVS23 + 371 | | | | CCTCAA | | CCUCAA |
| SCN1A- | 166863346 | 166863364 | 249 | ACAGCAGGGACT | 609 | ACAGCAGGGACU |
| IVS23 + 376 | | | | GTATGC | | GUAUGC |
| SCN1A- | 166863341 | 166863359 | 250 | ACTAAACAGCAA | 610 | ACUAAACAGCAA |
| IVS23 + 381 | | | | GGGCTG | | GGGCUG |
| SCN1A- | 166863336 | 166863354 | 251 | AATGTACTAAAC | 611 | AAUGUACUAAAC |
| IVS23 + 386 | | | | AGCAGG | | AGCAGG |
| SCN1A- | 166863331 | 166863349 | 252 | AGACCAATGTAC | 612 | AGACCAAUGUAC |
| IVS23 + 391 | | | | TAAACA | | UAAACA |
| SCN1A- | 166863326 | 166863344 | 253 | GACCCAGACCAA | 613 | GACCCAGACCAA |
| IVS23 + 396 | | | | TGTACT | | UGUACU |
| SCN1A- | 166863321 | 166863339 | 254 | TTCAGGACCCAG | 614 | UUCAGGACCCAG |
| IVS23 + 401 | | | | ACCAAT | | ACCAAU |
| SCN1A- | 166863316 | 166863334 | 255 | TAATTTTCAGGA | 615 | UAAUUUUCAGGA |
| IVS23 + 406 | | | | CCCAGA | | CCCAGA |
| SCN1A- | 166863311 | 166863329 | 256 | ACTGGTAATTTTC | 616 | ACUGGUAAUUUU |
| IVS23 + 411 | | | | AGGAC | | CAGGAC |
| SCN1A- | 166863306 | 166863324 | 257 | ATCTAACTGGTA | 617 | AUCUAACUGGUA |
| IVS23 + 416 | | | | ATTTTC | | AUUUUC |
| SCN1A- | 166863301 | 166863319 | 258 | ATGGTATCTAAC | 618 | AUGGUAUCUAAC |
| IVS23 + 421 | | | | TGGTAA | | UGGUAA |
| SCN1A- | 166863296 | 166863314 | 259 | AACTGATGGTAT | 619 | AACUGAUGGUAU |
| IVS23 + 426 | | | | CTAACT | | CUAACU |
| SCN1A- | 166863291 | 166863309 | 260 | TAATCAACTGAT | 620 | UAAUCAACUGAU |
| IVS23 + 431 | | | | GGTATC | | GGUAUC |
| SCN1A- | 166863286 | 166863304 | 261 | ATCAATAATCAA | 621 | AUCAAUAAUCAA |
| IVS23 + 436 | | | | CTGATG | | CUGAUG |
| SCN1A- | 166863281 | 166863299 | 262 | TACATATCAATA | 622 | UACAUAUCAAUA |
| IVS23 + 441 | | | | ATCAAC | | AUCAAC |
| SCN1A- | 166863276 | 166863294 | 263 | GCTCATACATAT | 623 | GCUCAUACAUAU |
| IVS23 + 446 | | | | CAATAA | | CAAUAA |
| SCN1A- | 166863271 | 166863289 | 264 | TATCTGCTCATAC | 624 | UAUCUGCUCAUA |
| IVS23 + 451 | | | | ATATC | | CAUAUC |
| SCN1A- | 166863266 | 166863284 | 265 | CCTAGTATCTGCT | 625 | CCUAGUAUCUGC |
| IVS23 + 456 | | | | CATAC | | UCAUAC |
| SCN1A- | 166863261 | 166863279 | 266 | TGCACCCTAGTA | 626 | UGCACCCUAGUA |
| IVS23 + 461 | | | | TCTGCT | | UCUGCU |
| SCN1A- | 166863256 | 166863274 | 267 | AATATTGCACCC | 627 | AAUAUUGCACCC |
| IVS23 + 466 | | | | TAGTAT | | UAGUAU |
| SCN1A- | 166863251 | 166863269 | 268 | CCTGAAATATTG | 628 | CCUGAAAUAUUG |
| IVS23 + 471 | | | | CACCCT | | CACCCU |
| SCN1A- | 166863246 | 166863264 | 269 | TGAAACCTGAAA | 629 | UGAAACCUGAAA |
| IVS23 + 476 | | | | TATTGC | | UAUUGC |
| SCN1A- | 166863241 | 166863259 | 270 | TCTTATGAAACCT | 630 | UCUUAUGAAACC |
| IVS23 + 481 | | | | GAAAT | | UGAAAU |
| SCN1A- | 166863236 | 166863254 | 271 | ACCAGTCTTATG | 631 | ACCAGUCUUAUG |
| IVS23 + 486 | | | | AAACCT | | AAACCU |
| SCN1A- | 166863231 | 166863249 | 272 | TCAATACCAGTC | 632 | UCAAUACCAGUC |
| IVS23 + 491 | | | | TTATGA | | UUAUGA |
| SCN1A- | 166863226 | 166863244 | 273 | CACAATCAATAC | 633 | CACAAUCAAUAC |
| IVS23 + 496 | | | | CAGTCT | | CAGUCU |
| SCN1A- | 166863221 | 166863239 | 274 | GTGGTCACAATC | 634 | GUGGUCACAAUC |
| IVS23 + 501 | | | | AATACC | | AAUACC |
| SCN1A- | 166863216 | 166863234 | 275 | TGAGAGTGGTCA | 635 | UGAGAGUGGUCA |
| IVS23 + 506 | | | | CAATCA | | CAAUCA |
| SCN1A- | 166863211 | 166863229 | 276 | AAAAATGAGAGT | 636 | AAAAAUGAGAGU |
| IVS23 + 511 | | | | GGTCAC | | GGUCAC |
| SCN1A- | 166863206 | 166863224 | 277 | CAATAAAAAATG | 637 | CAAUAAAAAAUG |
| IVS23 + 516 | | | | AGAGTG | | AGAGUG |
| SCN1A- | 166863201 | 166863219 | 278 | TTACACAATAAA | 638 | UUACACAAUAAA |
| IVS23 + 521 | | | | AAATGA | | AAAUGA |
| SCN1A- | 166863196 | 166863214 | 279 | TGAACTTACACA | 639 | UGAACUUACACA |
| IVS23 + 526 | | | | ATAAAA | | AUAAAA |
| SCN1A- | 166863191 | 166863209 | 280 | CCATATGAACTT | 640 | CCAUAUGAACUU |
| IVS23 + 531 | | | | ACACAA | | ACACAA |
| SCN1A- | 166863186 | 166863204 | 281 | TAACCCCATATG | 641 | UAACCCCAUAUG |
| IVS23 + 536 | | | | AACTTA | | AACUUA |
| SCN1A- | 166863181 | 166863199 | 282 | GAAAATAACCCC | 642 | GAAAAUAACCCC |
| IVS23 + 541 | | | | ATATGA | | AUAUGA |
| SCN1A- | 166863176 | 166863194 | 283 | ATTTTGAAAATA | 643 | AUUUUGAAAAUA |
| IVS23 + 546 | | | | ACCCCA | | ACCCCA |
| SCN1A- | 166863171 | 166863189 | 284 | TTAACATTTTGAA | 644 | UUAACAUUUUGA |
| IVS23 + 551 | | | | AATAA | | AAAUAA |
| SCN1A- | 166863166 | 166863184 | 285 | CCTTGTTAACATT | 645 | CCUUGUUAACAU |
| IVS23 + 556 | | | | TTGAA | | UUUGAA |
| SCN1A- | 166863161 | 166863179 | 286 | TTTTGCCTTGTTA | 646 | UUUUGCCUUGUU |
| IVS23 + 561 | | | | ACATT | | AACAUU |
| SCN1A- | 166863156 | 166863174 | 287 | TATATTTTTGCCT | 647 | UAUAUUUUUGCC |
| IVS23 + 566 | | | | TGTTA | | UUGUUA |
| SCN1A- | 166863151 | 166863169 | 288 | CTTAATATATTTT | 648 | CUUAAUAUAUUU |
| IVS23 + 571 | | | | TGCCT | | UUGCCU |
| SCN1A- | 166863146 | 166863164 | 289 | TATTTCTTAATAT | 649 | UAUUUCUUAAUA |
| IVS23 + 576 | | | | ATTTT | | UAUUUU |
| SCN1A- | 166863141 | 166863159 | 290 | TCAACTATTTCTT | 650 | UCAACUAUUUCU |
| IVS23 + 581 | | | | AATAT | | UAAUAU |
| SCN1A- | 166863136 | 166863154 | 291 | CTTATTCAACTAT | 651 | CUUAUUCAACUA |
| IVS23 + 586 | | | | TTCTT | | UUUCUU |
| SCN1A- | 166863131 | 166863149 | 292 | ATGTGCTTATTCA | 652 | AUGUGCUUAUUC |
| IVS23 + 591 | | | | ACTAT | | AACUAU |
| SCN1A- | 166863126 | 166863144 | 293 | TTCACATGTGCTT | 653 | UUCACAUGUGCU |
| IVS23 + 596 | | | | ATTCA | | UAUUCA |
| SCN1A- | 166863121 | 166863139 | 294 | CACAATTCACAT | 654 | CACAAUUCACAU |
| IVS23 + 601 | | | | GTGCTT | | GUGCUU |
| SCN1A- | 166863116 | 166863134 | 295 | TACAACACAATT | 655 | UACAACACAAUU |
| IVS23 + 606 | | | | CACATG | | CACAUG |
| SCN1A- | 166863111 | 166863129 | 296 | TTGTTTACAACAC | 656 | UUGUUUACAACA |
| IVS23 + 611 | | | | AATTC | | CAAUUC |
| SCN1A- | 166863106 | 166863124 | 297 | ACTTTTTGTTTAC | 657 | ACUUUUUGUUUA |
| IVS23 + 616 | | | | AACAC | | CAACAC |
| SCN1A- | 166863101 | 166863119 | 298 | TTCTAACTTTTTG | 658 | UUCUAACUUUUU |
| IVS23 + 621 | | | | TTTAC | | GUUUAC |
| SCN1A- | 166863096 | 166863114 | 299 | TTTTATTCTAACT | 659 | UUUUAUUCUAAC |
| IVS23 + 626 | | | | TTTTG | | UUUUUG |
| SCN1A- | 166863091 | 166863109 | 300 | GATTTTTTTATTC | 660 | GAUUUUUUUAUU |
| IVS23 + 631 | | | | TAACT | | CUAACU |
| SCN1A- | 166863086 | 166863104 | 301 | AAGTGGATTTTTT | 661 | AAGUGGAUUUUU |
| IVS23 + 636 | | | | TATTC | | UUAUUC |
| SCN1A- | 166863081 | 166863099 | 302 | CAAATAAGTGGA | 662 | CAAAUAAGUGGA |
| IVS23 + 641 | | | | TTTTTT | | UUUUUU |
| SCN1A- | 166863076 | 166863094 | 303 | TAATTCAAATAA | 663 | UAAUUCAAAUAA |
| IVS23 + 646 | | | | GTGGAT | | GUGGAU |
| SCN1A- | 166863071 | 166863089 | 304 | CTGCATAATTCA | 664 | CUGCAUAAUUCA |
| IVS23 + 651 | | | | AATAAG | | AAUAAG |
| SCN1A- | 166863066 | 166863084 | 305 | CTATTCTGCATAA | 665 | CUAUUCUGCAUA |
| IVS23 + 656 | | | | TTCAA | | AUUCAA |
| SCN1A- | 166863061 | 166863079 | 306 | GTATTCTATTCTG | 666 | GUAUUCUAUUCU |
| IVS23 + 661 | | | | CATAA | | GCAUAA |
| SCN1A- | 166863056 | 166863074 | 307 | GGTATGTATTCTA | 667 | GGUAUGUAUUCU |
| IVS23 + 666 | | | | TTCTG | | AUUCUG |
| SCN1A- | 166863051 | 166863069 | 308 | TTCTAGGTATGTA | 668 | UUCUAGGUAUGU |
| IVS23 + 671 | | | | TTCTA | | AUUCUA |
| SCN1A- | 166863046 | 166863064 | 309 | TTTATTTCTAGGT | 669 | UUUAUUUCUAGG |
| IVS23 + 676 | | | | ATGTA | | UAUGUA |
| SCN1A- | 166863041 | 166863059 | 310 | TTTGTTTTATTTC | 670 | UUUGUUUUAUUU |
| IVS23 + 681 | | | | TAGGT | | CUAGGU |
| SCN1A- | 166863036 | 166863054 | 311 | ACGTTTTTGTTTT | 671 | ACGUUUUUGUUU |
| IVS23 + 686 | | | | ATTTC | | UAUUUC |
| SCN1A- | 166863031 | 166863049 | 312 | ATAAGACGTTTTT | 672 | AUAAGACGUUUU |
| IVS23 + 691 | | | | GTTTT | | UGUUUU |
| SCN1A- | 166863026 | 166863044 | 313 | TCATGATAAGAC | 673 | UCAUGAUAAGAC |
| IVS23 + 696 | | | | GTTTTT | | GUUUUU |
| SCN1A- | 166863021 | 166863039 | 314 | AATACTCATGAT | 674 | AAUACUCAUGAU |
| IVS23 + 701 | | | | AAGACG | | AAGACG |
| SCN1A- | 166863016 | 166863034 | 315 | ATCTTAATACTCA | 675 | AUCUUAAUACUC |
| IVS23 + 706 | | | | TGATA | | AUGAUA |
| SCN1A- | 166863011 | 166863029 | 316 | ATTTTATCTTAAT | 676 | AUUUUAUCUUAA |
| IVS23 + 711 | | | | ACTCA | | UACUCA |
| SCN1A- | 166863006 | 166863024 | 317 | CTTAAATTTTATC | 677 | CUUAAAUUUUAU |
| IVS23 + 716 | | | | TTAAT | | CUUAAU |
| SCN1A- | 166863001 | 166863019 | 318 | TATGCCTTAAATT | 678 | UAUGCCUUAAAU |
| IVS23 + 721 | | | | TTATC | | UUUAUC |
| SCN1A- | 166862996 | 166863014 | 319 | GAGTTTATGCCTT | 679 | GAGUUUAUGCCU |
| IVS23 + 726 | | | | AAATT | | UAAAUU |
| SCN1A- | 166862991 | 166863009 | 320 | GAAGTGAGTTTA | 680 | GAAGUGAGUUUA |
| IVS23 + 731 | | | | TGCCTT | | UGCCUU |
| SCN1A- | 166862986 | 166863004 | 321 | TCTAAGAAGTGA | 681 | UCUAAGAAGUGA |
| IVS23 + 736 | | | | GTTTAT | | GUUUAU |
| SCN1A- | 166862981 | 166862999 | 322 | CTTATTCTAAGA | 682 | CUUAUUCUAAGA |
| IVS23 + 741 | | | | AGTGAG | | AGUGAG |
| SCN1A- | 166862976 | 166862994 | 323 | AGTTACTTATTCT | 683 | AGUUACUUAUUC |
| IVS23 + 746 | | | | AAGAA | | UAAGAA |
| SCN1A- | 166862971 | 166862989 | 324 | TTGGGAGTTACTT | 684 | UUGGGAGUUACU |
| IVS23 + 751 | | | | ATTCT | | UAUUCU |
| SCN1A- | 166862966 | 166862984 | 325 | GTTAGTTGGGAG | 685 | GUUAGUUGGGAG |
| IVS23 + 756 | | | | TTACTT | | UUACUU |
| SCN1A- | 166862961 | 166862979 | 326 | AGAAAGTTAGTT | 686 | AGAAAGUUAGUU |
| IVS23 + 761 | | | | GGGAGT | | GGGAGU |
| SCN1A- | 166862956 | 166862974 | 327 | ATCCTAGAAAGT | 687 | AUCCUAGAAAGU |
| IVS23 + 766 | | | | TAGTTG | | UAGUUG |
| SCN1A- | 166862951 | 166862969 | 328 | TTAAAATCCTAG | 688 | UUAAAAUCCUAG |
| IVS23 + 771 | | | | AAAGTT | | AAAGUU |
| SCN1A- | 166862946 | 166862964 | 329 | ATGTTTTAAAATC | 689 | AUGUUUUAAAAU |
| IVS23 + 776 | | | | CTAGA | | CCUAGA |
| SCN1A- | 166862941 | 166862959 | 330 | GTGTTATGTTTTA | 690 | GUGUUAUGUUUU |
| IVS23 + 781 | | | | AAATC | | AAAAUC |
| SCN1A- | 166862936 | 166862954 | 331 | TCACTGTGTTATG | 691 | UCACUGUGUUAU |
| IVS23 + 786 | | | | TTTTA | | GUUUUA |
| SCN1A- | 166862931 | 166862949 | 332 | TGTTTTCACTGTG | 692 | UGUUUUCACUGU |
| IVS23 + 791 | | | | TTATG | | GUUAUG |
| SCN1A- | 166862926 | 166862944 | 333 | ATGTATGTTTTCA | 693 | AUGUAUGUUUUC |
| IVS23 + 796 | | | | CTGTG | | ACUGUG |
| SCN1A- | 166862921 | 166862939 | 334 | TGTTTATGTATGT | 694 | UGUUUAUGUAUG |
| IVS23 + 801 | | | | TTTCA | | UUUUCA |
| SCN1A- | 166862916 | 166862934 | 335 | AGTTATGTTTATG | 695 | AGUUAUGUUUAU |
| IVS23 + 806 | | | | TATGT | | GUAUGU |
| SCN1A- | 166862911 | 166862929 | 336 | TGTAGAGTTATG | 696 | UGUAGAGUUAUG |
| IVS23 + 811 | | | | TTTATG | | UUUAUG |
| SCN1A- | 166862906 | 166862924 | 337 | TAAAATGTAGAG | 697 | UAAAAUGUAGAG |
| IVS23 + 816 | | | | TTATGT | | UUAUGU |
| SCN1A- | 166862901 | 166862919 | 338 | ATAAATAAAATG | 698 | AUAAAUAAAAUG |
| IVS23 + 821 | | | | TAGAGT | | UAGAGU |
| SCN1A- | 166862896 | 166862914 | 339 | TAAGAATAAATA | 699 | UAAGAAUAAAUA |
| IVS23 + 826 | | | | AAATGT | | AAAUGU |
| SCN1A- | 166862891 | 166862909 | 340 | AACTTTAAGAAT | 700 | AACUUUAAGAAU |
| IVS23 + 831 | | | | AAATAA | | AAAUAA |
| SCN1A- | 166862886 | 166862904 | 341 | ACTTAAACTTTA | 701 | ACUUAAACUUUA |
| IVS23 + 836 | | | | AGAATA | | AGAAUA |
| SCN1A- | 166862881 | 166862899 | 342 | AATACACTTAAA | 702 | AAUACACUUAAA |
| IVS23 + 841 | | | | CTTTAA | | CUUUAA |
| SCN1A- | 166862876 | 166862894 | 343 | TGTATAATACAC | 703 | UGUAUAAUACAC |
| IVS23 + 846 | | | | TTAAAC | | UUAAAC |
| SCN1A- | 166862871 | 166862889 | 344 | CTTCTTGTATAAT | 704 | CUUCUUGUAUAA |
| IVS23 + 851 | | | | ACACT | | UACACU |
| SCN1A- | 166862866 | 166862884 | 345 | CTCTTCTTCTTGT | 705 | CUCUUCUUCUUG |
| IVS23 + 856 | | | | ATAAT | | UAUAAU |
| SCN1A- | 166862861 | 166862879 | 346 | ATAAACTCTTCTT | 706 | AUAAACUCUUCU |
| IVS23 + 861 | | | | CTTGT | | UCUUGU |
| SCN1A- | 166862856 | 166862874 | 347 | CGAATATAAACT | 707 | CGAAUAUAAACU |
| IVS23 + 866 | | | | CTTCTT | | CUUCUU |
| SCN1A- | 166862851 | 166862869 | 348 | TCTCTCGAATATA | 708 | UCUCUCGAAUAU |
| IVS23 + 871 | | | | AACTC | | AAACUC |
| SCN1A- | 166862846 | 166862864 | 349 | TTCTGTCTCTCGA | 709 | UUCUGUCUCUCG |
| IVS23 + 876 | | | | ATATA | | AAUAUA |
| SCN1A- | 166862841 | 166862859 | 350 | ACTTTTTCTGTCT | 710 | ACUUUUUCUGUC |
| IVS23 + 881 | | | | CTCGA | | UCUCGA |
| SCN1A- | 166862836 | 166862854 | 351 | TTCTGACTTTTTC | 711 | UUCUGACUUUUU |
| IVS23 + 886 | | | | TGTCT | | CUGUCU |
| SCN1A- | 166862831 | 166862849 | 352 | AAAAATTCTGAC | 712 | AAAAAUUCUGAC |
| IVS23 + 891 | | | | TTTTTC | | UUUUUC |
| SCN1A- | 166862826 | 166862844 | 353 | CAAACAAAAATT | 713 | CAAACAAAAAUU |
| IVS23 + 896 | | | | CTGACT | | CUGACU |
| SCN1A- | 166862821 | 166862839 | 354 | TGATCCAAACAA | 714 | UGAUCCAAACAA |
| IVS23 + 901 | | | | AAATTC | | AAAUUC |
| SCN1A- | 166862816 | 166862834 | 355 | ATTGGTGATCCA | 715 | AUUGGUGAUCCA |
| IVS23 + 906 | | | | AACAAA | | AACAAA |
| SCN1A- | 166862811 | 166862829 | 356 | GATATATTGGTG | 716 | GAUAUAUUGGUG |
| IVS23 + 911 | | | | ATCCAA | | AUCCAA |
| SCN1A- | 166862806 | 166862824 | 357 | GCTATGATATATT | 717 | GCUAUGAUAUAU |
| IVS23 + 916 | | | | GGTGA | | UGGUGA |
| SCN1A- | 166862801 | 166862819 | 358 | TGTAAGCTATGA | 718 | UGUAAGCUAUGA |
| IVS23 + 921 | | | | TATATT | | UAUAUU |
| SCN1A- | 166862796 | 166862814 | 359 | TTTTTTGTAAGCT | 719 | UUUUUUGUAAGC |
| IVS23 + 926 | | | | ATGAT | | UAUGAU |
| SCN1A- | 166862791 | 166862809 | 360 | ACAGTTTTTTTGT | 720 | ACAGUUUUUUUG |
| IVS23 + 931 | | | | AAGCT | | UAAGCU |
| SCN1A- | 166862786 | 166862804 | 361 | TTAAGACAGTTTT | 721 | UUAAGACAGUUU |
| IVS23 + 936 | | | | TTTGT | | UUUUGU |
| SCN1A- | 166862781 | 166862799 | 362 | TTTAATTAAGAC | 722 | UUUAAUUAAGAC |
| IVS23 + 941 | | | | AGTTTT | | AGUUUU |
| SCN1A- | 166862776 | 166862794 | 363 | TGGGTTTTAATTA | 723 | UGGGUUUUAAUU |
| IVS23 + 946 | | | | AGACA | | AAGACA |
| SCN1A- | 166862771 | 166862789 | 364 | TGTTGTGGGTTTT | 724 | UGUUGUGGGUUU |
| IVS23 + 951 | | | | AATTA | | UAAUUA |
| SCN1A- | 166862766 | 166862784 | 365 | AATTATGTTGTG | 725 | AAUUAUGUUGUG |
| IVS23 + 956 | | | | GGTTTT | | GGUUUU |
| SCN1A- | 166862761 | 166862779 | 366 | AAAAAAATTATG | 726 | AAAAAAAUUAUG |
| IVS23 + 961 | | | | TTGTGG | | UUGUGG |
| SCN1A- | 166862756 | 166862774 | 367 | AATCTAAAAAAA | 727 | AAUCUAAAAAAA |
| IVS23 + 966 | | | | TTATGT | | UUAUGU |
| SCN1A- | 166862751 | 166862769 | 368 | TTAAAAATCTAA | 728 | UUAAAAAUCUAA |
| IVS23 + 971 | | | | AAAAAT | | AAAAAU |
| SCN1A- | 166862746 | 166862764 | 369 | CTTTCTTAAAAAT | 729 | CUUUCUUAAAAA |
| IVS23 + 976 | | | | CTAAA | | UCUAAA |
| SCN1A- | 166862741 | 166862759 | 370 | AGAATCTTTCTTA | 730 | AGAAUCUUUCUU |
| IVS23 + 981 | | | | AAAAT | | AAAAAU |
| SCN1A- | 166862736 | 166862754 | 371 | ATAATAGAATCT | 731 | AUAAUAGAAUCU |
| IVS23 + 986 | | | | TTCTTA | | UUCUUA |

### Example 4: SCN1A Exon 23 (Exon 20x) Region Extended ASO Walk Evaluated by RT-PCR

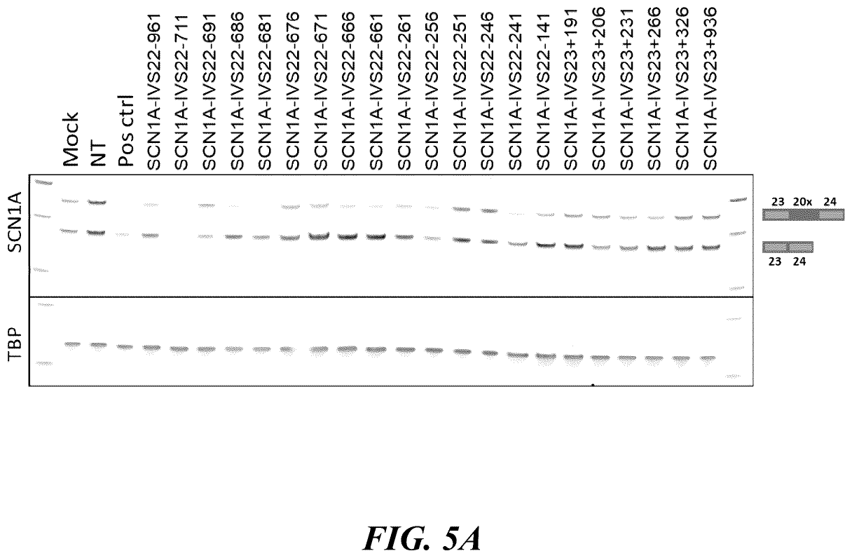

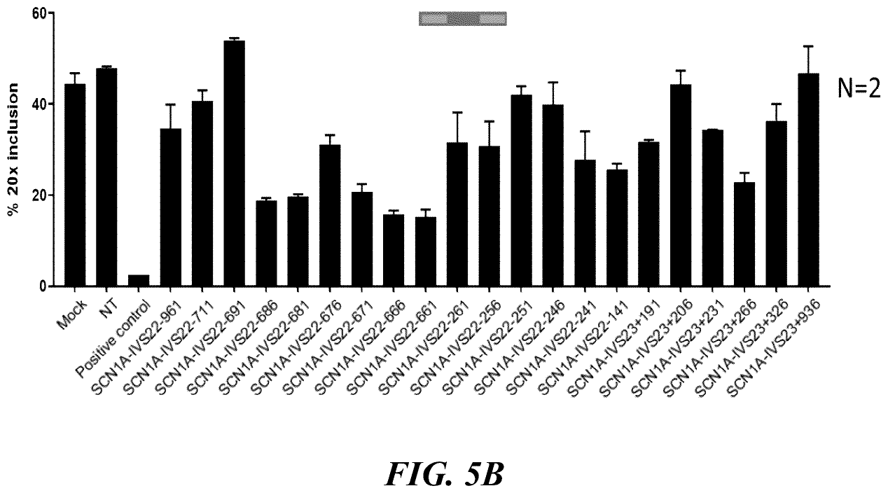

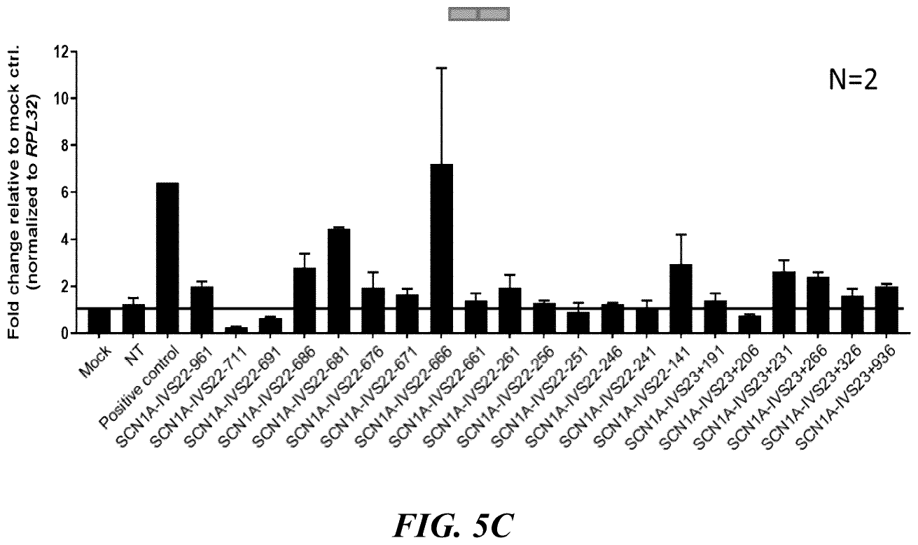

ASO walk sequences can be evaluated by for example RT-PCR. In FIG. 5A, a representative PAGE shows SYBR-safe-stained RT-PCR products of SCN1A mock-treated, control ASO treated, targeting the exon 20x region as described herein in the Example 3 and in the description of FIG. 3, at 1 μM concentration in RenCells by nucleofection. Two products, one comprising the exon 20x and one excluding exon 20x were quantified and the percent exon 20x inclusion is plotted in the bar graph (FIG. 5B). Taqman q PCR products were also normalized to RPL32 internal control and fold-change relative to mock is plotted in the bar graph (FIG. 5C).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-32. (canceled)

33. A pharmaceutical composition comprising a therapeutic agent or a vector encoding the therapeutic agent, wherein the therapeutic agent binds to a targeted portion of a pre-mRNA that contains a nonsense mediated RNA decay-inducing exon (NMD exon) and that encodes a target protein, whereby the therapeutic agent modulates exclusion of the NMD exon from the pre-mRNA encoding the target protein, thereby modulating a level of processed mRNA encoding the target protein, and wherein the targeted portion is:
from about 1,000 nucleotides upstream from the 5′ end of the NMD exon to about 100 nucleotides upstream of the 5′ end of the NMD exon; or
from about 100 nucleotides downstream of the 3′ end of the NMD exon to about 1,000 nucleotides downstream of the 3′ end of the NMD exon.

34. The pharmaceutical composition of claim 33, wherein the target protein is Nav1.1 protein.

35. The pharmaceutical composition of claim 33, wherein the therapeutic agent interferes with binding of a factor involved in splicing of the NMD exon from a region of the targeted portion.

36. The pharmaceutical composition of claim 33, wherein the targeted region is at most 500 nucleotides upstream of the 5′ end of the NMD exon.

37. The pharmaceutical composition of claim 33, wherein the targeted region is at least 500 nucleotides upstream of the 5′ end of the NMD exon.

38. The pharmaceutical composition of claim 33, wherein the targeted region is at most 500 nucleotides downstream of the 3′ end of the NMD exon.

39. The pharmaceutical composition of claim 33, wherein the targeted region is at least 500 nucleotides downstream of the 3′ end of the NMD exon.

40. The pharmaceutical composition of claim 33, wherein the NMD exon comprises a sequence with at least 95% sequence identity to SEQ ID NO: 7.

41. The pharmaceutical composition of claim 33, wherein the targeted region is within an intronic sequence flanking the NMD exon.

42. The pharmaceutical composition of claim 33, wherein the targeted region is comprised within a sequence with at least 95% sequence identity to SEQ ID NO: 6.

43. The pharmaceutical composition of claim 33, wherein the therapeutic agent is an antisense oligomer (ASO).

44. The pharmaceutical composition of claim 43, wherein the ASO comprises a sequence that is at least about 95% identical to any one of SEQ ID NOs: 12-731.

45. The pharmaceutical composition of claim 44, wherein the ASO consists of a sequence selected from the group consisting of SEQ ID NOs: 72, 73, 76, 181, 220, 432, 433, 436, 541, and 580.

46. The pharmaceutical composition of claim 43, wherein the ASO comprises at least one or more backbone modifications, one or more sugar moiety modifications, or both.

47. The pharmaceutical composition of claim 46, wherein the ASO comprises a 2′-O-methyl moiety, a 2′-fluoro moiety, or a 2′-O-methoxyethyl moiety.

48. The pharmaceutical composition of claim 46, wherein the ASO comprises a phosphorothioate linkage or a phosphorodiamidate linkage.

49. The pharmaceutical composition of claim 43, wherein the ASO is from 18 to 50 nucleobases in length.

50. The pharmaceutical composition of claim 33, wherein the therapeutic agent promotes exclusion of the NMD exon from the pre-mRNA.

51. The pharmaceutical composition of claim 34, wherein the therapeutic agent increases the level of the processed mRNA encoding Nav1.1 protein in a cell contacted with the pharmaceutical composition.

52. An antisense oligomer (ASO) comprising a sequence that is at least 80%, identical to any one of SEQ ID NOs: 12-731.


SEQUENCE LISTING (732 sequences)

SEQ ID NO: 1 - skipped

SEQ ID NO: 2 - RNA, 8579 nt, Homo sapiens
  source 1..8579: mol_type = mRNA
ttgctgatttgtattaggtaccatagagtgaggcgaggatgaagccgagaggatactgcagaggtctctggtgcatgtgtgtatgtgtgcgtttgtgtgtgtttgtgtgtctgtgtgttctgccccagtgagactgcagcccttgtaaatactttgacaccttttgcaagaaggaatctgaacaattgcaactgaaggcacattgttatcatctcgtctttgggtgatgctgttcctcactgcagatggataattttccttttaatcagaacagcataagaattatttctgagtggaggtgaggcttgtccaaatgtctttgctatcatggatttcctgactcctacctgtttgaggtttgggcaattatgaataaggctgctgtatacatccgtgtgcaggattttgtgtggacataagttttcaactcctttggttaaatcctaaggaatttcatatgcagaataaatggtaattaaaatgtgcaggatgacaagatggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagagggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataaatgaaaataaataaaaataattgggtgacaaattgtttacagcctgtgaaggtgatgtatttttatcaacaggactcctttaggaggtcaatgccaaactgactgtttttacacaaatctccttaaggtcagtgcctacaataagacagtgaccccttgtcagcaaactgtgactctgtgtaaaggggagatgaccttgacaggaggttactgttctcactaccagctgacactgctgaagataagatgcacaatggctagtcagactgtagggaccagtttcaaggggtgcaaacctgtgattttggggttgtttaacatgaaacactttagtgtagtaattgtatccactgtttgcatttcaactgccacatttgtcacatttttatggaatctgttagtggattcatctttttgttaatccatgtgtttattatatgtgactatttttgtaaacgaagtttctgttgagaaataggctaaggacctctataacaggtatgccacctggggggtatggcaaccacatggccctcccagctacacaaagtcgtggtttgcatgagggcatgctgcacttagagatcatgcatgagaaaaagtcacaagaaaaacaaattcttaaatttcaccatatttctgggaggggtaattgggtgataagtggaggtgctttgttgatcttgttttgcgaaatccagcccctagaccaagtagattatttgtgggtaggccagtaaatcttagcaggtgcaaacttcattcaaatgtttggagtcataaatgttatgtttctttttgttgtattaaaaaaaaaacctgaatagtgaatattgcccctcaccctccaccgccagaagactgaattgaccaaaattactctttataaatttctgctttttcctgcactttgtttagccatcttcggctctcagcaaggttgacactgtatatgttaatgaaatgctatttattatgtaaatagtcattttaccctgtggtgcacgtttgagcaaacaaataatgacctaagcacagtatttattgcatcaaatatgtaccacaagaaatgtagagtgcaagctttacacaggtaataaaatgtattctgtaccatttatagatagtttggatgctatcaatgcatgtttatattaccatgctgctgtatctggtttctctcactgctcagaatctcatttatgagaaaccatatgtcagtggtaaagtcaaggaaattgttcaacagatctcatttatttaagtcattaagcaatagtttgcagcactttaacagctttttggttatttttacattttaagtggataacatatggtatatagccagactgtacagacatgtttaaaaaaacacactgcttaacctattaaatatgtgtttagaattttataagcaaatataaatactgtaaaaagtcactttattttatttttcagcattatgtacataaatatgaagaggaaattatcttcaggttgatatcacaatcacttttcttactttctgtccatagtactttttcatgaaagaaatttgctaaataagacatgaaaacaagactgggtagttgtagatttctgctttttaaattacatttgctaattttagattatttcacaattttaaggagcaaaataggttcacgattcatatccaaattatgctttgcaattggaaaagggtttaaaattttatttatatttctggtagtacctgcactaactgaattgaaggtagtgcttatgttatttttgttctttttttctgacttcggtttatgttttcatttctttggagtaatgctgctctagattgttctaaatagaatgtgggcttcataatttttttttccacaaaaacagagtagtcaacttatatagtcaattacatcaggacattttgtgtttcttacagaagcaaaccataggctcctcttttccttaaaactacttagataaactgtattcgtgaactgcatgctggaaaatgctactattatgctaaataatgctaaccaacatttaaaatgtgcaaaactaataaagattacattttttatttta

SEQ ID NO: 3 - DNA, 2425 bp, Homo sapiens
  source 1..2425: mol_type = genomic DNA
gtaagaaaaatgaaagaacctgaagtattgtatatagccaaaattaaactaaattaaatttagaaaaaggaaaatctatgcatgcaaaaggaatggcaaattcttgcaaaattgctactttattgttttatctgttgcatatttacttctaggtgatatgcaagagaaataggcctctcttgaaatgatataatatcatttatctgctgtgcttatttaaatgactttatttcctaatccatcttgggagtttccttacaaatctatatacaaaaaaaagctgatgcattattaaagtactatgtgtaatgatataatggtaatctaaagtaaattctatatcaggtacttattctttgtgatgatatactgtacttaacgagttttcctgaaaataatgtgaatcacacatgtgcctaagtatgagtgttaagaaaaaaatgaaaggagttgttaaaacttttgtctgtataatgccaaagtttgcattatttgaatatattcaagattagatggttagatattaagtgttgactgaatttataaaactagtaatactaacttaaagattacatacaaatccacatcatttttataacaataaagtaaaacacttataatgaacagaaaatataattttgactcattactataggtaatttatacattaaccttaacttgcatcttattggtcagagtcacacaaaatgttattttatccttttcaaagatgcaataatcattttccatcatgcataacagattagaaattttgccattattgacttattttccatgcctttttttacggcatgaagcattagtttatagatatataatataaaaaattagttctgcttttttttaaaaaaaaatattatcaaaacaaaacactgaattgtgtgattccaatagaaaaacactgctctttcacctcctaaggtgtagttacttttatggaaactaagctgtattgtagacttccatttgcactttgtagattgtttatagccttatgttctcttctcaagtcttattataaatgtcactttgtaagaacgtaggacttgtcttcgatttccctaacatatatgaaaactttgtcctcattatcgacaactcagaacaatataatacaagtagtcctcttttatttctcacagagagcctcaaattttcaccaaaatgttaacagaaattatctctggggtgtataagaattaagtctgttttccaattaaatgtcactttgttttgtttcagactggcagtttcagttctggagaaaaaaaatgtcatttgtgtacattctacttgaaaacatgttgcctgaatcaaaataatatattttatatggcttgtgaaatctgaacaatgctaaacatttgaaaatattataaaccttttacatttgaccatttgaaagtttattaaattcattggtcaagtgctcagatatttccatacattacacttcatttctataaaaaagctgatcttatcggtatacttttaattttctcagaaataaccatatctataattattaatcaataatgccttttatattaaaagaggttagtttttgaaacttggagttttagacataaaatccttataaatgctgatagtgatataactaatagtttaaatggtcagatttatgaatatggctctattcctcataatgacaacatacacacagcactaaaatgactaatctcttcaatacgtgtttggcattgtagagtcaaaataacgttataattgattctattttttatacttctagtgtttggatattttattttgtaaaaatataatcatgaatgatggtgaggttggatataagaatgatgattatgattgggaagtgagatttgaacatgctcagaaactctcatttaattctttgccctagcagcataaaatcacaatagctgcgtcaaagcgtaactcaggcactcattttatttttgttgttctgttattttttcaaagcatgtgcttttatgcaacattactgaataaagcatgttgtacagtgcttgataagaagttagaaagtaacaaataaattatcatcacgttgcactttgtgttttgcatgttttatgcacatttctggctgacagcttttaaacatttattgtatttcaaatttccagtccaaatttttcaacttgtaaaattaaactgagtgaattgatgtcgtgaatatctagggtaaaataaaatttgtgtttaaatttgtatttttaatttcctaacctaggaaatcttaaataccttctttttcaaaagaactcaagtcttaatggatagggaaacagacggagagcatcatgaacaaaaagtaacaccaaatgttctgtcatatcagatttctaactaataacaaactatatatttctattttgtatag

SEQ ID NO: 4 - DNA, 64 bp, Homo sapiens
  source 1..64: mol_type = genomic DNA
gataatcttgctccaacttggatggggtggagcgctggttcctcccctgagccctttattatgg

SEQ ID NO: 5 - DNA, 4476 bp, Homo sapiens
  source 1..4476: mol_type = genomic DNA
gtactgtattaccccttttgctacctttaatccttgcactgtgacttatgtgtagtggggtgagggagggattgggaagggtactattattgcaccacagtagggaaaatacattatttacatcctaatcccctcttttcaattgtcttaaatttcatttgaaaaaaaaaaaaacctttatgaatttaccctctgtggattttaaccccaatggttgatatctttattaagtttcattgaatatgatttagttatgtgtatatggagttatccatctttggggagattactggattggtgagggcgggggaccctggtgtagaatgattatgtgaaaaaacaatttaacttgttaagctcatgatactgtttgaggcatacagcccctgctgtttagtacattggtctgggtcctgaaaattaccagttagataccatcagttgattattgatatgtatgagcagatactagggtgcaatatttcaggtttcataagactggtattgattgtgaccactctcattttttattgtgtaagttcatatggggttattttcaaaatgttaacaaggcaaaaatatattaagaaatagttgaataagcacatgtgaattgtgttgtaaacaaaaagttagaataaaaaaatccacttatttgaattatgcagaatagaatacatacctagaaataaaacaaaaacgtcttatcatgagtattaagataaaatttaaggcataaactcacttcttagaataagtaactcccaactaactttctaggattttaaaacataacacagtgaaaacatacataaacataactctacattttatttattcttaaagtttaagtgtattatacaagaagaagagtttatattcgagagacagaaaaagtcagaatttttgtttggatcaccaatatatcatagcttacaaaaaaactgtcttaattaaaacccacaacataatttttttagatttttaagaaagattctattattcttctttatacttaaaaatggatgattcctactttgcccacttttatttttattcacatagattttctttatttctattagagaagcactagaattcatgaatagtgttgatttgaagttcaaagtaattaattcagataaaaagacatttctgcatgtatgaaaatttctaatgtgaatttgcatatttaattatcaatccttcatttagtgtagacttatttttaaaaatgcaggtaatgaaccagaaatagaattggttgtgctagagtagagaaactttatttgatgattgttttgaaaaaaaagcttctgagaagaaacaacctctagtacagtattaattcattaagatagctcctttctcagacatttcctttcatgtagcctgaaagttcaatttgaaatttgttctttccaatttattcagactaattctgcctactttcttccccccataagaaccaattactgcagctttattgagactgaaaaaagttaatacacctccttctttgctgaaccaaggaatggcttggaactcttgggaaaagacaatcttttctatgatctttcattgtctaatttaatacatcataaatatgactatagctttgtataataaactccccaatactgtgccagatgttttctaagataaagttattttatgttcacaaaaaaaataaaacttttctctgggccaaatgtatgccaactttgcaaatcatatcctgaagtgcactgctgcagagtacatgcttgcgtcataaattccatagagttcgctttaactctaaatcaatccccagtttcaaagtaaacctctcaaacatattacctaagcacaaacttctccctgtgctcagttccttaattattctcatcccatattcagaaataacatttaaaaattatgctttgatcaataaatactaatctaaactttgcttcattaacccattcatttttgtcaaccattattttattcctatattcaaagctctctggtatgttcttatattcaagacactcaaggccctggaagattcacgaacatatgtttgcatcttaaatttttagaaaatcttacaatctgtcaggattacactgaactctagtacagagtaatatgggtaccagataagtgggagcaactcttccacgtagactggaaacagcactaaatgctatttataggctactttctgaacttaacttgttttaacctcatttttctcatatgccaaatgagaacgcaatactgaattatctgtacagttctgttcagtactagaattctgattcttgaattcaaaggggaaaacattcctctttattttggaggctaaactgggggacaaagttaggctccatgaaagaagtgctatttgaactaaagcctttaagaggggagagtatttcagaagaggagctattagacaaggaatttcaatgtaaatggcatctcaatcacctggcaattatattagcacacggttattatattaattgaagtggcatgaagtatagatgaccagggaagttaaaactggaaatatagattgtggagtgatgtgaataccaaggtaagaaaaatatttgttagttaccagagagccaataaataactttcaagtgggacttggggaagattaattcatcttacatagattaaatgaaggagaaggttaggagacagatgacagtgcaagtatgaaataacagagggcagttctaggtggtgactgtgagaatggaaaagaggtggcaaagctgagaaacgtttcaaagaaaaaatgtgagacaggtaatgtgaaaagaaaatcgagaaataggtatagataatcagtgttctgctcatactctaaattgggtgttgaaggcaaaatacgtattttaattagtactctgtgtatacacactagaaacagcattgtaatctggatagtggacaaaatattcagaaaagaagggaaatagtaacttgatttcaatttccaaatctctaatctgaaagaaatctaattctattcatccatttaaaataaattatataacgagaatttatgaagtccattgtattaatgcagacagtcagatgagataaggcaaagtgtcacgtgtcagcttggtagttgcatcggccacatcatttggttctgcctggataactcaaccaaattaatttttcatactcatcccctccacctttgtcattactggtattcttattttctttggcccacttatcacactgttttatgttccccagaaggcctagagttctttacaggctttaaacagggatcagaagtataagaaattggctcatgtatttttttttcagacaggcagttaaaaaaaattgttctaaaaatacactggcatcaaatggcaaatagaagatgttttgacgactacttccattggatcagactgacaagaataatacaagcacataggtggaattaaacttagctattaatgtccaagtttgaggcagctgccccttataagcattttagggtctgtttttagcttccctcttagccactcctgtgcagctccagtgggaggtatggaggaaaaagcaaggaagccatccctatgttgtttccaaacatgaacactcaagatttttaactagtggtccagaagtaaagagggggaaaacatccttctatagaaaaaaaaaaaagtagataataattgaacacagaacttcatgtgatcacatcagatttgagaactatgtatggcatccctctttttcttattttcctaagaaatgatttctattatgtttcatttgaaataagttttttgaattaaactcagtaaatgaaacaactgacatgactggagcttgaaataaacgatgtgatgatctaatgaaatacataatgcaaattgtcttgcttcttatgcaaaaattattagtcatagcaatgcatgaataattaaagacaattatattaggtatttaataatattttttatatttatcatctgaatttttaagttattttaaaaatatattggtcaaatcaactcaggtccaaatgttttagttttgttctttaatatattgcctttttaaaatgagttaaacttctgtataggctttttaacttttctttattctgataacacaattctgacttcatctggcagcaagttcctctgattttccttttcctttaaccttttaatgcttctccctcccttttttttaaaaacatttttgtttcatttcttggttatattgcctatagttgttttcctaagtgtattgcttaagaaaaaaaaatgaattttaagatttttttgaaccttgcttttacatatcctagaataaatagcattgatagaaaaaaagaatggaaagaccagagattactaggggaattttttttctttattaacagataagaattctgacttttctttttttccatttgtgtattag

SEQ ID NO: 6 - RNA, 6901 nt, Homo sapiens
  source 1..6901: mol_type = mRNA
gtaagaaaaatgaaagaacctgaagtattgtatatagccaaaattaaactaaattaaatttagaaaaaggaaaatctatgcatgcaaaaggaatggcaaattcttgcaaaattgctactttattgttttatctgttgcatatttacttctaggtgatatgcaagagaaataggcctctcttgaaatgatataatatcatttatctgctgtgcttatttaaatgactttatttcctaatccatcttgggagtttccttacaaatctatatacaaaaaaaagctgatgcattattaaagtactatgtgtaatgatataatggtaatctaaagtaaattctatatcaggtacttattctttgtgatgatatactgtacttaacgagttttcctgaaaataatgtgaatcacacatgtgcctaagtatgagtgttaagaaaaaaatgaaaggagttgttaaaacttttgtctgtataatgccaaagtttgcattatttgaatatattcaagattagatggttagatattaagtgttgactgaatttataaaactagtaatactaacttaaagattacatacaaatccacatcatttttataacaataaagtaaaacacttataatgaacagaaaatataattttgactcattactataggtaatttatacattaaccttaacttgcatcttattggtcagagtcacacaaaatgttattttatccttttcaaagatgcaataatcattttccatcatgcataacagattagaaattttgccattattgacttattttccatgcctttttttacggcatgaagcattagtttatagatatataatataaaaaattagttctgcttttttttaaaaaaaaatattatcaaaacaaaacactgaattgtgtgattccaatagaaaaacactgctctttcacctcctaaggtgtagttacttttatggaaactaagctgtattgtagacttccatttgcactttgtagattgtttatagccttatgttctcttctcaagtcttattataaatgtcactttgtaagaacgtaggacttgtcttcgatttccctaacatatatgaaaactttgtcctcattatcgacaactcagaacaatataatacaagtagtcctcttttatttctcacagagagcctcaaattttcaccaaaatgttaacagaaattatctctggggtgtataagaattaagtctgttttccaattaaatgtcactttgttttgtttcagactggcagtttcagttctggagaaaaaaaatgtcatttgtgtacattctacttgaaaacatgttgcctgaatcaaaataatatattttatatggcttgtgaaatctgaacaatgctaaacatttgaaaatattataaaccttttacatttgaccatttgaaagtttattaaattcattggtcaagtgctcagatatttccatacattacacttcatttctataaaaaagctgatcttatcggtatacttttaattttctcagaaataaccatatctataattattaatcaataatgccttttatattaaaagaggttagtttttgaaacttggagttttagacataaaatccttataaatgctgatagtgatataactaatagtttaaatggtcagatttatgaatatggctctattcctcataatgacaacatacacacagcactaaaatgactaatctcttcaatacgtgtttggcattgtagagtcaaaataacgttataattgattctattttttatacttctagtgtttggatattttattttgtaaaaatataatcatgaatgatggtgaggttggatataagaatgatgattatgattgggaagtgagatttgaacatgctcagaaactctcatttaattctttgccctagcagcataaaatcacaatagctgcgtcaaagcgtaactcaggcactcattttatttttgttgttctgttattttttcaaagcatgtgcttttatgcaacattactgaataaagcatgttgtacagtgcttgataagaagttagaaagtaacaaataaattatcatcacgttgcactttgtgttttgcatgttttatgcacatttctggctgacagcttttaaacatttattgtatttcaaatttccagtccaaatttttcaacttgtaaaattaaactgagtgaattgatgtcgtgaatatctagggtaaaataaaatttgtgtttaaatttgtatttttaatttcctaacctaggaaatcttaaataccttctttttcaaaagaactcaagtcttaatggatagggaaacagacggagagcatcatgaacaaaaagtaacaccaaatgttctgtcatatcagatttctaactaataacaaactatatatttctattttgtataggtactgtattaccccttttgctacctttaatccttgcactgtgacttatgtgtagtggggtgagggagggattgggaagggtactattattgcaccacagtagggaaaatacattatttacatcctaatcccctcttttcaattgtcttaaatttcatttgaaaaaaaaaaaaacctttatgaatttaccctctgtggattttaaccccaatggttgatatctttattaagtttcattgaatatgatttagttatgtgtatatggagttatccatctttggggagattactggattggtgagggcgggggaccctggtgtagaatgattatgtgaaaaaacaatttaacttgttaagctcatgatactgtttgaggcatacagcccctgctgtttagtacattggtctgggtcctgaaaattaccagttagataccatcagttgattattgatatgtatgagcagatactagggtgcaatatttcaggtttcataagactggtattgattgtgaccactctcattttttattgtgtaagttcatatggggttattttcaaaatgttaacaaggcaaaaatatattaagaaatagttgaataagcacatgtgaattgtgttgtaaacaaaaagttagaataaaaaaatccacttatttgaattatgcagaatagaatacatacctagaaataaaacaaaaacgtcttatcatgagtattaagataaaatttaaggcataaactcacttcttagaataagtaactcccaactaactttctaggattttaaaacataacacagtgaaaacatacataaacataactctacattttatttattcttaaagtttaagtgtattatacaagaagaagagtttatattcgagagacagaaaaagtcagaatttttgtttggatcaccaatatatcatagcttacaaaaaaactgtcttaattaaaacccacaacataatttttttagatttttaagaaagattctattattcttctttatacttaaaaatggatgattcctactttgcccacttttatttttattcacatagattttctttatttctattagagaagcactagaattcatgaatagtgttgatttgaagttcaaagtaattaattcagataaaaagacatttctgcatgtatgaaaatttctaatgtgaatttgcatatttaattatcaatccttcatttagtgtagacttatttttaaaaatgcaggtaatgaaccagaaatagaattggttgtgctagagtagagaaactttatttgatgattgttttgaaaaaaaagcttctgagaagaaacaacctctagtacagtattaattcattaagatagctcctttctcagacatttcctttcatgtagcctgaaagttcaatttgaaatttgttctttccaatttattcagactaattctgcctactttcttccccccataagaaccaattactgcagctttattgagactgaaaaaagttaatacacctccttctttgctgaaccaaggaatggcttggaactcttgggaaaagacaatcttttctatgatctttcattgtctaatttaatacatcataaatatgactatagctttgtataataaactccccaatactgtgccagatgttttctaagataaagttattttatgttcacaaaaaaaataaaacttttctctgggccaaatgtatgccaactttgcaaatcatatcctgaagtgcactgctgcagagtacatgcttgcgtcataaattccatagagttcgctttaactctaaatcaatccccagtttcaaagtaaacctctcaaacatattacctaagcacaaacttctccctgtgctcagttccttaattattctcatcccatattcagaaataacatttaaaaattatgctttgatcaataaatactaatctaaactttgcttcattaacccattcatttttgtcaaccattattttattcctatattcaaagctctctggtatgttcttatattcaagacactcaaggccctggaagattcacgaacatatgtttgcatcttaaatttttagaaaatcttacaatctgtcaggattacactgaactctagtacagagtaatatgggtaccagataagtgggagcaactcttccacgtagactggaaacagcactaaatgctatttataggctactttctgaacttaacttgttttaacctcatttttctcatatgccaaatgagaacgcaatactgaattatctgtacagttctgttcagtactagaattctgattcttgaattcaaaggggaaaacattcctctttattttggaggctaaactgggggacaaagttaggctccatgaaagaagtgctatttgaactaaagcctttaagaggggagagtatttcagaagaggagctattagacaaggaatttcaatgtaaatggcatctcaatcacctggcaattatattagcacacggttattatattaattgaagtggcatgaagtatagatgaccagggaagttaaaactggaaatatagattgtggagtgatgtgaataccaaggtaagaaaaatatttgttagttaccagagagccaataaataactttcaagtgggacttggggaagattaattcatcttacatagattaaatgaaggagaaggttaggagacagatgacagtgcaagtatgaaataacagagggcagttctaggtggtgactgtgagaatggaaaagaggtggcaaagctgagaaacgtttcaaagaaaaaatgtgagacaggtaatgtgaaaagaaaatcgagaaataggtatagataatcagtgttctgctcatactctaaattgggtgttgaaggcaaaatacgtattttaattagtactctgtgtatacacactagaaacagcattgtaatctggatagtggacaaaatattcagaaaagaagggaaatagtaacttgatttcaatttccaaatctctaatctgaaagaaatctaattctattcatccatttaaaataaattatataacgagaatttatgaagtccattgtattaatgcagacagtcagatgagataaggcaaagtgtcacgtgtcagcttggtagttgcatcggccacatcatttggttctgcctggataactcaaccaaattaatttttcatactcatcccctccacctttgtcattactggtattcttattttctttggcccacttatcacactgttttatgttccccagaaggcctagagttctttacaggctttaaacagggatcagaagtataagaaattggctcatgtatttttttttcagacaggcagttaaaaaaaattgttctaaaaatacactggcatcaaatggcaaatagaagatgttttgacgactacttccattggatcagactgacaagaataatacaagcacataggtggaattaaacttagctattaatgtccaagtttgaggcagctgccccttataagcattttagggtctgtttttagcttccctcttagccactcctgtgcagctccagtgggaggtatggaggaaaaagcaaggaagccatccctatgttgtttccaaacatgaacactcaagatttttaactagtggtccagaagtaaagagggggaaaacatccttctatagaaaaaaaaaaaagtagataataattgaacacagaacttcatgtgatcacatcagatttgagaactatgtatggcatccctctttttcttattttcctaagaaatgatttctattatgtttcatttgaaataagttttttgaattaaactcagtaaatgaaacaactgacatgactggagcttgaaataaacgatgtgatgatctaatgaaatacataatgcaaattgtcttgcttcttatgcaaaaattattagtcatagcaatgcatgaataattaaagacaattatattaggtatttaataatattttttatatttatcatctgaatttttaagttattttaaaaatatattggtcaaatcaactcaggtccaaatgttttagttttgttctttaatatattgcctttttaaaatgagttaaacttctgtataggctttttaacttttctttattctgataacacaattctgacttcatctggcagcaagttcctctgattttccttttcctttaaccttttaatgcttctccctcccttttttttaaaaacatttttgtttcatttcttggttatattgcctatagttgttttcctaagtgtattgcttaagaaaaaaaaatgaattttaagatttttttgaaccttgcttttacatatcctagaataaatagcattgatagaaaaaaagaatggaaagaccagagattactaggggaattttttttctttattaacagataagaattctgacttttctttttttccatttgtgtattag

SEQ ID NO: 7 - RNA, 64 nt, Homo sapiens
  source 1..64: mol_type = genomic RNA
gataatcttgctccaacttggatggggtggagcgctggttcctcccctgagccctttattatgg

SEQ ID NO: 8 - skipped

SEQ ID NO: 9 - RNA, 8320 nt, Mus musculus
  source 1..8320: mol_type = mRNA
atgtaagtctgtctaggtcaagtgtaggagactcactaccggcctgtggaaactcatggaactgtttcttcagattaacacttcaggggctatcgaggctgcaggaagctgagctttaactacatacatctttcgggggaatctcacatgaagagtaaagagtaattaaaatgtgcaggatgacaagatggagcaaacagtgcttgtaccaccaggacctgacagcttcaacttcttcaccagagaatcccttgcagctattgaaaggcgcattgcagaagagaaggctaagaatcccaagccagacaaaaaagatgatgatgaaaatggcccaaagccaaacagtgacttggaagctgggaagaaccttccatttatctatggagacattcctccagagatggtgtcggagcctctggaggacctggacccctactatatcaataagaagacttttatagtattgaataaagggaaggccatcttccggttcagtgccacctccgccctgtacattttaacacccttcaatcctcttaggaaaatagctattaagattttggtacactcattattcagcatgttaatcatgtgcactattttgacaaactgtgtatttatgacaatgagtaaccctcccgactggacaaagaatgtggagtacaccttcacaggaatatatacttttgaatcactaataaaaattattgcaaggggcttctgtttagaagattttactttccttcgcgacccatggaactggctggacttcactgtcattacattcgcatatgtgacggagtttgtggacctgggcaatgtctcagcattgagaacattcagagttcttcgagcattgaaaactatttcagtcattccaggcctgaagaccatcgtgggggccctgatccagtcggtgaagaagctgtctgacgtcatgatactcactgtgttctgtctcagtgtgttcgcactcatcgggttgcagctcttcatgggcaacctgaggaataaatgtgtacagtggcctcccaccaacgcttcccttgaggaacatagcatagagaagaatataactatggattacaatggcacacttgtaaatgaaaccgtgttcgagtttgactggaaatcatacattcaagactcaagatatcattatttcctggagggtgttttagatgcactgctgtgtggaaatagctctgatgcaggccaatgtccagaaggatatatgtgtgtaaaagctggtagaaaccctaattatggttacacaagctttgataccttcagttgggcatttttgtccctgtttcgactgatgactcaggacttctgggaaaatctataccaactgacattgcgtgctgctggcaaaacctacatgatattttttgtgctggtcattttcttgggctcattctacctgataaacttgatcctggctgtggtggccatggcctatgaggagcagaatcaggccacactggaggaggctgaacagaaagaggcagaatttcagcagatgttggagcaacttaagaagcagcaagaggctgcacagcaggcagcggctacaacagcctcagaacattccagggagcccagtgcagcaggcaggctctcagatagctcttcagaagcctctaagttgagttcgaagagtgctaaagaaagacgaaatcggaggaaaaaaaggaaacagaaagagcagtctggaggagaagagaaagatgatgatgaattccacaagtctgagtctgaagacagcatcaggaggaaggggtttcgcttctccatagaagggaatagactgacatatgaaaagaggtactcttccccgcatcagtctctgttaagcattcgtggttccctgttctccccaagacgcaatagcagaacaagtcttttcagctttagagggcgagccaaggatgtggggtctgagaatgactttgctgatgatgaacacagcacctttgaggataatgagagccgtagagactcactgttcgttccccgaagacacggagagcgacgcaacagtaacctgagccagaccagcaggtcctcccgaatgctggcggtgtttccagccaatgggaagatgcacagcacggtggattgcaatggtgtggtttccttggttggtggaccctcagttcccacatcgccagttggacagcttctgccagaggtgataatagataagccagctactgatgacaatggaacaaccactgaaactgagatgagaaagaggaggtcgagctctttccatgtttccatggactttctagaagatccttcccagaggcaaagggcaatgagcatagccagcatcttaacaaatacagtagaagaactagaagaatccaggcagaaatgtccaccctgttggtataaattttccaacatattcttaatttgggactgttctccatattggctgaaagttaaacatattgtcaacctggtggtgatggacccatttgttgatctggccattaccatctgcattgtgttaaatacgctcttcatggctatggagcactaccccatgactgaacatttcaaccatgttcttacagtgggaaacttggtcttcactgggattttcacagcagaaatgttcctgaaaatcatcgcaatggatccttactattacttccaagaaggctggaatatctttgatggtttcattgtgacactcagcctggtagaacttggccttgccaatgtggaaggattgtcagttctccgttcatttcgactgctccgagtgttcaagttggcaaagtcttggcccacactgaatatgctcattaagatcattggtaactcggtgggagcactgggcaacctgactctggtgttggccatcattgtctttatttttgccgtggttggcatgcagctgtttggaaaaagttacaaagattgtgtctgcaaaattgccactgactgcaaactcccacgttggcacatgaacgacttcttccactcgttcctgatcgtgttccgcgtgctgtgtggggagtggatagagaccatgtgggactgcatggaggtggcaggacaagctatgtgccttactgtcttcatgatggtcatggtgattgggaaccttgtggtcttgaacctctttctggccttgcttctgagctcatttagtgcagacaaccttgcagccactgatgatgacaatgagatgaacaacctgcagattgctgtggacaggatgcacaaaggaatagcttatgtaaaaagaaaaatatatgaattcattcaacaatcctttgttaagaaacagaagattctagatgaaattaagccacttgatgatctaaacaacagaaaagacaattgtatctctaaccacacaacagaaattgggaaagatctggactgtctgaaagatgtgaatggaaccacaagtggcatagggacgggcagcagtgtggagaagtacatcattgatgagagtgattatatgtcattcataaacaaccccagcctcactgtgactgtgcccattgctgtgggagagtctgactttgagaacttaaacacagaagactttagcagtgaatcagatctagaagaaagcaaagagaaactcaacgaaagcagtagctcctcagagggaagcacagtagacattggggcgcctgcagaggaacagcctgtcattgaaccagaagaaacccttgagcccgaagcttgcttcactgaaggctgtgtccagagattcaagtgctgtcaaatcagcgtggaagaaggaagagggaaacagtggtggaacctacggaggacgtgcttccgaatagttgaacacaactggtttgagaccttcattgtgttcatgattctcctgagtagtggtgccctggcctttgaggatatatatattgatcagcgaaagacgatcaaaaccatgctggagtatgctgacaaagtcttcacttacattttcatcctggagatgctcctcaaatgggtggcctatggctatcaaacatacttcaccaatgcctggtgttggctagacttcttaattgttgatgtttcattggtcagtttaacagcaaatgccttgggttactctgaactcggggccatcaaatccctaaggacactaagagctctgagacccctaagagccttatcacgatttgaagggatgagggtggttgtgaatgccctgttaggagcaattccatccatcatgaatgtgcttctggtttgccttatattctggctaattttcagcatcatgggcgtaaatttgtttgctggcaaattctaccactgtgttaacaccacaactggtgacatatttgagatcagcgaagtcaataatcattctgattgcctaaaactaatagaaagaaatgagaccgcccggtggaaaaatgtgaaagtaaactttgataatgtaggatttgggtatctttctttgcttcaagttgccacatttaagggctggatggatatcatgtatgctgcagttgattccagaaatgttgaactacagcctaagtatgaggaaagcctgtacatgtatttgtacttcgtcatcttcatcatcttcgggtccttctttaccctgaacctgtttattggtgtcattatcgacaatttcaaccagcaaaagaagaagtttggaggtcaagacatctttatgacagaagaacagaagaaatactataatgcaatgaagaaattaggatcaaaaaagccacaaaagcctatccctcgacctggaaacaaatttcaaggaatggtttttgactttgtaaccagacaagtgtttgatatcagcatcatgatcctcatctgtctgaacatggtgaccatgatggtggaaacggatgaccagagcgattatgtgacaagcattttgtcacgcatcaacctggtgttcatcgtcctgttcaccggcgagtgtgtgctcaagctcatctcgctccgccattattatttcaccattggatggaacattttcgattttgtggtggtcatcctctccattgtagggatgtttcttgcggagctaatagaaaagtattttgtgtctcctaccctgttccgagtcatccgcctggccaggattggacgaatcctacgcctgatcaaaggtgccaaggggatccgcacgctgctctttgctctgatgatgtcccttcctgcgctgtttaacatcggcctcctgctttttctcgtcatgttcatctacgccatctttgggatgtccaactttgcctatgttaagagggaagttgggattgatgacatgttcaactttgagaccttcggcaacagcatgatctgcctgttccaaatcaccacctctgcgggctgggatggactgctggcccccatcctcaacagcaaaccccctgactgtgaccctaataaagttaaccctggaagctcggtgaagggagactgtgggaacccatctgtggggattttcttttttgtcagctacatcatcatatccttcctggttgtggtgaacatgtacattgctgtcatcctggagaacttcagcgttgccacagaagaaagtgcagagcctctgagtgaggacgactttgagatgttctacgaggtctgggagaagttcgaccctgacgccacccagttcatggaatttgaaaaattatctcagtttgcagctgctctagaaccccctctcaatttgccacaaccaaacaaacttcagctcattgccatggacctgcccatggtgagtggagaccgcatccactgcctggacatcttatttgcttttacaaagcgggtgttgggtgagagtggagagatggatgctcttcgaatccagatggaagagcggttcatggcttccaacccctccaaggtctcttatcagcccatcactactacattaaaacgcaaacaagaggaggtgtcagctgttatcattcagcgagcttataggcgccaccttttgaagcgaacagtaaaacaagcttcattcacatacaataagaacaaactcaaaggtggggctaatcttcttgtaaaagaagacatgctcattgacagaataaacgaaaactctattacggagaaaactgacctgacaatgtccacagcagcttgtccgccctcctacgatcgggtgacaaagccaatcgtggagaaacacgagcaggaagggaaggatgaaaaagccaaagggaaatgaacaaaaacaacaaaaaaaaataataaattgggtgacaaattgtttacagcctgtgaagtcaaaaggactcctttaagaagtcaatgccaaactgactgtttttacacaaattcactttaaggtcagtgcctcaataagacagtgacccccttgtcggcaaactctgtgactgtgtaaaggggagataaccttgacaggagggtactgttctaactaccagctgacactgctgaagacaagagacaaatggctatgcagactgtagggatcagtctaaaggggtgcagagctataatttgggggttaatgtgaaacactttagtgtagtaactctatccaccgtttgcatttcaactgccacatttgtcaggttttttacaaactctgttagtggattcatcttttttttaatccatgtgttgtttgttacatgtgactatttttgtaagcagggtttctgttgggaaatagactaaaggaactcttttcccggtgtgtcacctgggagtgttgacatccttatggccctcccgtctgcacaacggtgtggtttcgcatgagggcatgttgcaccttaggatcatgcatgagaaaaagtcgaaaggaaagcaaagacagttcttcaacagttctatgtcatctgtgtttctgcggggggagggggcggtacggaggtgatcgctagatcgtgggctttgctgagctgggtttgtcaaaatctagtccttagtccaaggagattgttcagacaggtcatcaagtcttaggaggtgcaaacttcattcaaatgtctggagtcacgctacatttctgttcactgtctctgaaaggtctctaaattatgactctgactccctacccccaaccctagcagagcgaattgaccaaaagaaccctttataaatccccgcttcctcctgcactttgtttagccatcttcagctctcagcaaggttgacactgtatgtgttaatgaaatgctatttattatgtaaatagtcattttactctgtggtgcacgtttgagcaaacaaataatgacgtaagcacagtatttattgcatcaaatatgtaccacgagaaatgtagagtgcaagctgtccaccggaaatatactgtactctgtaccattgtagatagtgtggatgctgccaatgcatgtcgatattaccacgttgcggtatctagtttctctcagcactcagaatctcactcacgagaaaccataggtcagtggctaagtcaaggaaactgttcagcagacttcatttctttaagtcattaagcaatagtttgcagcactttaacaatgtttgggttgtttttaaaatttaagtggataacgttcaatgtatagccagactgtacatacgtgttcaaaagacaacactactgcttaacctgttaaaaacatgtttagagttttataagcaaatataaatactgtacaaagtcactttcttttacttttcagcattatgtacatatgaagaggaaattatcttcaggttgatatcacaatcacttttcttactttctgtccacagcagtttttcatggaagatatttgctaactaagaaatgaaactatgactgggtagtggtagatctctgcttttttattacaattgttaattttggattatttcataattttaaggggcaaaatacactcacaacttcacatccaaattatgatttgcaattggaaaaggtataacatttgatttatatttattgtagtgtttgtgctaacagatgaccagagtgcttttttttctcatttttttctttatttccccaatttctgtttatttttctttgctttgaaatcatgcttctttagattgttctaaatataaatgtgggcttcagaatttcttttcctcaaaatcagagaggattacacttatatagtcaattacatcaagatatttgtgtttcttacagaagcagaccgtaggcacctctttttcttaaaaaaaaaatatttaggcaaaccgtattcatgaactgcatgttggaaaatgctactattagcccaaaatgatgctaatcacaattaaaaaatgtgcaaatctaataaaattaattttttatttt

SEQ ID NO: 10 - RNA, 7597 nt, Mus musculus
  source 1..7597: mol_type = unassigned RNA
gtaagaaaaaggaaaactctgcagcgttgtatattgtcaaagctaggctgagttcaacttaactaacgaaaaacacgtgcatgcaaaaggaatggcaaccctttgcaaacttgctactttacccttttctctgttgcatatttacttcttggtgatatgcaagagaaaatcggcctctttgaaaatgatttaatatcatttatctgctttgctaattaaaatgaccttagttcataatcgatcttgggagtttccttataattcctaatacaaagggggaggggcagatactctcttaaagaactaagttgagtcatgtaataattacctagagataattttgtttcatatcgttctcctctatgacagcccatcagtacttaagggatcctatggaaagtaatgtgaatcacaaatgtgtatgaatacaaaggaaaaaatgaagaattgttaaatgttttgtctttacaatgccaaaattctcattatttgaatatatccaagggcagatattaaccattgactggagtataataatactgcctcaactgtaactaaattaatgacattgaataagtaagacactaatttaattactataaatacatacacatcttatgacaatacagctgataaggaaaaagaacatgtatttttattcattgccatacatggctcgtcaaccttaacttaaacctcggttctcagttacacagagttttatgttgctcttttgagcaaagcattattcccctctccataattcaacacgtatcagatttttgcatttattggctcattgttatgatgattatttcaaagcattatacaatcatttatagaagatgtgccgtgtgaaaattatttttttattaagatccaaaatttacgctctttaaaccaatcagatgaaatgtataaggcaaagagtgctcattgtcctgacacttacaaaccaaggctccaacaaacaggctcctctctgcaccacatagagggctttcagcctgtgtccccccataaaaacctattataaattttattatactatactgtaagaaacctgtccaatttttaatttctctagcacatatgaaaacttctcttcagtagatccaagtaagcacaaaggagctttgattctcacacaagaaatcacatttgtattaaaaatgtatcataaattttctcccaattatgcaaaacttaaatgcttttccaattaaaagagcactttcgtttcagaatagcaatttcagttgtcaaggaaaacattgtttttatacattttatataaaaatacatgagctaaatttaaattcacatttttcaactttttatggtttttttatgtttctttttctttcgcattttttaacaatccagccattacctctcctccctgtccccctcctatagtttctcatctcattcctcctcccccttgcctctgagaggatgctccctccctcactaggcctccctcttccccagggcttcaagttcctcaaggattatacacatcttctcccactgagaccaggccaggcagtcctctgcttctgcccagcctgtgtatgttcctgcttggtagctcagtctctggaagctccctggggtctgggttagttgggactgctggtcttcctatggagttaccctccccttcaacttcttcaatccttcccctaattcaaccacaggtatccagacttcagtccaatggttgagtgtaaatattggcatctgtctctgtcagctgttgttaggggctcagaggacagccatgctaggctcctgcctacaagcagcacaccataacatcagtaatagtgtcaggcctttaatgaataatgctacgtatataaggttgttagattatacttcaactttgatcttttagaatattattaaatccagtcgtttattttttatatgtaatattgactttccataacaaatgagtctattttccttttgtgtagaaataactttatcaattattgttaataatgcttttgtcaattattgttgatatgcttctcttttttaaaactggagtcatcaaaacaaaaattctgggtagatattacgaagctgactccttggtcagcttggcacatagtgagaccacaaatatctcaaggtcacggcaattcctcaccaccagtttggcattgtgaagtcaaaaccaaccttctgttgattctggtttttgtatttctagtatgagacattttctactttgtaagagtatataactgtggatggcggcgaggttgggcatgatgatgattgtaagcgggaagtgagctagagtatcttcagaaactctcactttatcctccttggcagcatagaaccgcaatcgctgtgtccgagtgtcaaccaggcagtcattttgttttgggttttttgtcactctttcaaagcatgtgcttctacgcaacactaccaaacacagcatgctgcatagtgcttgagaaggagttagaaagtaacaaacgagttatcatcacgttgccctttgtgttttgcatgtcttatgcacacttttggctgacagcttttgaacatttattgtatttcaaatttccagtccaaatttttttttcaacttgtgaaattgaacggaatgaaccgatgtcgtgaatacctagggtcaaataaaacttgtatttaaattcgtagttttaatttcccaagctgggaaaatcgtaaaaaccttttccaaagaactcaagtcttagttgctagggaaacaggcagtgagcatcatatacaaaaagtaacaccaaatgttctgtcatatcagctttctaactaataataaactatatatttctattttatataggataatcttgctccaacttggatggggtggagcggtggttcctcccctcagccctttattatgggtactgtattaccccttttgctacctttaatccttgcactgtgacttatgtgtagtgggattgagggagggagtgggaagggtacaattgcaccacagtagggacaatacaggatttatttccaaatccactacttttaatgagcttaaacttcttttgggaaaaaaaaagttatctctgacttaccatctgtggattataaccccagaagtacatatctttattacgtttcactgaatatgatttagctatttatacttcattgtccatttatggggaaattactcaattggtgagggtgggggaccctggtgtaggatgctgatgaaaacgttttcatttgtcaagctcatggtagtgacagagcatatagtccttattttttcaacacactgctctggtccctcaatgggccagtcacattccatcagttgatcgttgatgtgtgcgagcagtggctaaaggtacaacaggccaggtatctcagggttgccaatggttatgatcattctcatctttattgcataaaaatgtgttatttgcagaaagtagcaaggcaagatccctgtgaaacaagggaatacaaaaaaaaaaaaagatgtgctttaagttataaaaccaaaacatgtgaaaagtcaacttcattgaagtataaagaataggatatgcatgaaaaacaaaaaaatcatgagcactaagaaattggtgtataagccaactccttgtaagctaccccaattaacttcccagaatcttagaaggcatcacagtgcaccccaaaataaaaagccaaactgacacttctgcttcctcttaaaatgtaggagtcttggataagaaagataatttttattgtttggaagaaaaaaaaattgtttggataattgaggcatttatctatcaaaaatatttatcttaataaaatttcacaacactgatttagttgttggcttttctaaaaattttttatatttcatattaagaactcatgatttttactttccattttttaaattcttattcacatatggtttttctctatttcttagaaaagctatagaacccatggtttccggtgacttaaaaaactaatctaaatgtcttcacttagatgatactttcaaatgcactgaaatttctaatatcaacaagaatatttgcctggtcctaatttttcactgatttaataaaaagtatgaaccctaaagaagaaatagacttgaagaactggttgtgtgacaatacagaaattctgctgggagatgtccttttaaaacattgttagaagagacaacctctacaatccacccattaagcatacttctctcttagacatctccttttatgtaccttataacctcaatgtgttctttccaattgacttagaccaacacttcccagcgcatcccacatgggagccaattactgactctccctagagactgcaaagaattaatattgtagaaccaagggatggttgggctcttgggagaggcaatccgtttgtgatcttttgctctggatgttaatgaaatcgcaactttaagtggattttcagtggcaaatcctctgatcctatgccaaatgttctctaagacaaacatccttgtaaaataaatgtctcactgggccaaatctatggcaaatttgcacgttttcctgaactgcattcctatatagtatttgccatcctgaattcactaatgggcattacttttaattcaaaaccagtcccttcttcaaaggaaatctctcccatttattacattatgcaaactgctttcttatgcagtggttaaatccttagccaggcaagtatgaggactggaatttggatatccagaaccctcagaaatgtcggatgggcatagtagcttacatgtaattccagagctagaaagatgaaactagccctgtcctcaagctctgaattcagttggtgcaataaataagaaagaatccccccccccgacccctacatctctttcctctacatctatacatgtatttcccatacagctctatgcttccacatatatgctcacatatgtgcatgcacacttgcacacatatgtacacttgacacattgaagcatcataatctaacaattggaatataagaaaatatttaactttcacacagagcagtcagagaaaaccataagaggttgaaactcttgaaaaaacttgcaggataaacagaaaagagtatgagatgccaattctggtctactttctgaacctaacttgttttaccttcattagtctaattttccaaatgaaccccaagcaccaaattgtccttattgctctttccagtactaaattataattcctcaattcgaaggcaaacactctcatctttgttaaggatgtgtagagttagactcaataaacgacatgtatatttgagctaaagcctggaggagggagattatttcaagggcaagtacttggcagggagtttcaaagaaagaaggctctcaattctcagactaacaccttagcgtggagtgactgtcacagaggagggtgaagtgtatgtcaccagttagggaagctgaaaggggaaatgtatcaggcttgtgagaatccttcagcagccaagtctagcacctgagcacaatccccagaactgaccccacatggtggaaaggagaggaataattcctgcaaattttcctgtgacctccacccaagtgctatagaaaatgcatgcatgcccacatgcacacacaaataaacatagttgcaaactgttttgaggaaaataacctcacaaactgtcgagtgatgtaaatgccaaagaaagagaaatgttttctaatggctagagaaccattaaggaatttttcaaaatgggacatgggatagataaatttagtatccatactgaaagaaggcaagcaaataaaatctgataagaatgtaattcttagtaaccgaggacagagcgaagatagagaacagggccaatggccaaggtggaaaggtttgaaggaagcagcatgaaacctacagacttgtaccaaaatgttcagtgtcatgagtgttaaaagtgaaaagcttgcatgttagtatggatttcatatcctcggcagacagagcacctcactgtgagtgggagatgaagtattcagaaatgagaaacaactactcaatttcagtgttcatatctcaaatccaataaacaactttaggggtacaattttttaaaaaattacattaaaatgtttttaaatctcttcttaataatttaaaaattaaattgaaaataactttaaaaagtaatatatacaggaaagcctgtgtgctaattttttagggaggccataaagggagatagttgctcattaatttctacacatcagcctatctttggcttctgccttgatagcgcactctgaattatcttcttcatgttcatccctcatctttattgttactggtttcatttcccttggccacatagcccactattttgtattccccaatggatattgttccttacaaagttcagccagggctcagaagtacaaggaattggctcttatacttctgtcagacaggcaaaaacttctaaaattatactataataaaaatcaaagagatgatattcataattaaactaacaaaagtggcaggccccccctcccccaacatgagtagaattaatctgacgtccatgttcaagtctgaaacacacttgccaattaagagcacattagggccagcctttatctccctcttagttactaatgtgcagttcaatggtgagctatagagaaggaagccaagactaccatatgtcaaatataaaaaaaaaaaatcccattttaaatctgtagtcccgaattaaggacaagagagagggaaatatctttgacattagaaaatggagaaaatattttagcacaggactttactcagtcacatcagagttgataagtacgtatgacatccctctttttcctgttttcctgagaaaatgatctctctagtgtttcatttaagataagtttattgaattaaactcagtaaatgaaacaactgacatgactggagcttgaaataaacgatgtgatgatctactgaaatacatgatgctaaattgtcttgcttcttatgcaaaaactactattagttatagcaatgcatggataattaaggccaaaaatatattagatgttaaaaatagttttatatttatacatctgaattttaatttatatttaaagtatattggtccaatcaattcatgcccaaatgttttagttctattctttgagatactgttttgttttgggatttttttttatgagctaatctcttgcctaggagttcctacttctctctcctccttttattttttctaataaactacacatgtgtcttcatccaggagctaacttctcccattttgcttttcctttagcaccttttttatattagatttctttcttttctccatctctttgcatattgcctatatttcttttcctaagcataatatttaaaaaagactgagttttatgttaagattatttctgctttgctcttacacagataggataagtagtcttgatagaaaataaatcaatgattcctagggggatgtctttttgcttttaatcaataaggattctgacttctctttctctccatttgtgtattag

SEQ ID NO: 11 - RNA, 64 nt, Mus musculus
  source 1..64: mol_type = unassigned RNA
gataatcttgctccaacttggatggggtggagcggtggttcctcccctcagccctttattatgg

SEQ ID NO: 12 - DNA, 18 bp, synthetic construct
  source 1..18: mol_type = other DNA
atgaatttaataaacttt

SEQ ID NO: 13 - DNA, 18 bp, synthetic construct
  source 1..18: mol_type = other DNA
gaccaatgaatttaataa

SEQ ID NO: 14 - DNA, 18 bp, synthetic construct
  source 1..18: mol_type = other DNA
cacttgaccaatgaattt

SEQ ID NO: 15 - DNA, 18 bp, synthetic construct
  source 1..18: mol_type = other DNA
ctgagcacttgaccaatg

SEQ ID NO: 16 - DNA, 18 bp, synthetic construct
  source 1..18: mol_type = other DNA
aatatctgagcacttgac

SEQ ID NO: 17 - DNA, 18 bp, synthetic construct
  source 1..18: mol_type = other DNA
atggaaatatctgagcac

SEQ ID NO: 18 - DNA, 18 bp, synthetic construct
  source 1..18: mol_type = other DNA
aatgtatggaaatatctg

SEQ ID NO: 19 - DNA, 18 bp, synthetic construct
  source 1..18: mol_type = other DNA
agtgtaatgtatggaaat

SEQ ID NO: 20 - DNA, 18 bp, synthetic construct
  source 1..18: mol_type = other DNA
aatgaagtgtaatgtatg

SEQ ID NO: 21 - DNA, 18 bp, synthetic construct
  source 1..18: mol_type = other DNA
atagaaatgaagtgtaat

SEQ ID NO: 22 - DNA, 18 bp, synthetic construct
  source 1..18: mol_type = other DNA
tttttatagaaatgaagt

SEQ ID NO: 23 - DNA, 18 bp, synthetic construct
  source 1..18: mol_type = other DNA
cagcttttttatagaaat

SEQ ID NO: 24 - DNA, 18 bp, synthetic construct
  source 1..18: mol_type = other DNA
aagatcagcttttttata

SEQ ID NO: 25 - DNA, 18 bp, synthetic construct
  source 1..18: mol_type = other DNA
ccgataagatcagctttt

SEQ ID NO: 26 - DNA, 18 bp, synthetic construct
  source 1..18: mol_type = other DNA
gtataccgataagatcag

SEQ ID NO: 27 - DNA, 18 bp, synthetic construct
  source 1..18: mol_type = other DNA
taaaagtataccgataag

SEQ ID NO: 28 - DNA, 18 bp, synthetic construct
  source 1..18: mol_type = other DNA
aaaattaaaagtataccg

SEQ ID NO: 29 - DNA, 18 bp, synthetic construct
  source 1..18: mol_type = other DNA
ctgagaaaattaaaagta

SEQ ID NO: 30 - DNA, 18 bp, synthetic construct
  source 1..18: mol_type = other DNA
tatttctgagaaaattaa

SEQ ID NO: 31 - DNA, 18 bp, synthetic construct
  source 1..18: mol_type = other DNA
atggttatttctgagaaa

SEQ ID NO: 32 - DNA, 18 bp, synthetic construct
  source 1..18: mol_type = other DNA
tagatatggttatttctg

SEQ ID NO: 33 - DNA, 18 bp, synthetic construct
  source 1..18: mol_type = other DNA
aattatagatatggttat

SEQ ID NO: 34 - DNA, 18 bp, synthetic construct
  source 1..18: mol_type = other DNA
ttaataattatagatatg

SEQ ID NO: 35 - DNA, 18 bp, synthetic construct
  source 1..18: mol_type = other DNA
attgattaataattatag

SEQ ID NO: 36 - DNA, 18 bp, synthetic construct
  source 1..18: mol_type = other DNA
gcattattgattaataat

SEQ ID NO: 37 - DNA, 18 bp, synthetic construct
  source 1..18: mol_type = other DNA
aaaaggcattattgatta

SEQ ID NO: 38 - DNA, 18 bp, synthetic construct
  source 1..18: mol_type = other DNA
aatataaaaggcattatt

SEQ ID NO: 39 - DNA, 18 bp, synthetic construct
  source 1..18: mol_type = other DNA
cttttaatataaaaggca

SEQ ID NO: 40 - DNA, 18 bp, synthetic construct
  source 1..18: mol_type = other DNA
aacctcttttaatataaa

SEQ ID NO: 41 - DNA, 18 bp, synthetic construct
  source 1..18: mol_type = other DNA
aaactaacctcttttaat

SEQ ID NO: 42 - DNA, 18 bp, synthetic construct
  source 1..18: mol_type = other DNA
ttcaaaaactaacctctt

SEQ ID NO: 43 - DNA, 18 bp, synthetic construct
  source 1..18: mol_type = other DNA
caagtttcaaaaactaac

SEQ ID NO: 44 - DNA, 18 bp, synthetic construct
  source 1..18: mol_type = other DNA
aactccaagtttcaaaaa

SEQ ID NO: 45 - DNA, 18 bp, synthetic construct
  source 1..18: mol_type = other DNA
tctaaaactccaagtttc

SEQ ID NO: 46 - DNA, 18 bp, synthetic construct
  source 1..18: mol_type = other DNA
ttatgtctaaaactccaa

SEQ ID NO: 47 - DNA, 18 bp, synthetic construct
  source 1..18: mol_type = other DNA
ggattttatgtctaaaac

SEQ ID NO: 48 - DNA, 18 bp, synthetic construct
  source 1..18: mol_type = other DNA
tataaggattttatgtct

SEQ ID NO: 49 - DNA, 18 bp, synthetic construct
  source 1..18: mol_type = other DNA
gcatttataaggatttta

SEQ ID NO: 50 - DNA, 18 bp, synthetic construct
  source 1..18: mol_type = other DNA
tatcagcatttataagga

SEQ ID NO: 51 - DNA, 18 bp, synthetic construct
  source 1..18: mol_type = other DNA
atcactatcagcatttat

SEQ ID NO: 52 - DNA, 18 bp, synthetic construct
  source 1..18: mol_type = other DNA
gttatatcactatcagca

SEQ ID NO: 53 - DNA, 18 bp, synthetic construct
  source 1..18: mol_type = other DNA
tattagttatatcactat

SEQ ID NO: 54 - DNA, 18 bp, synthetic construct
  source 1..18: mol_type = other DNA
taaactattagttatatc

SEQ ID NO: 55 - DNA, 18 bp, synthetic construct
  source 1..18: mol_type = other DNA
ccatttaaactattagtt

SEQ ID NO: 56 - DNA, 18 bp, synthetic construct
  source 1..18: mol_type = other DNA
tctgaccatttaaactat

SEQ ID NO: 57 - DNA, 18 bp, synthetic construct
  source 1..18: mol_type = other DNA
ataaatctgaccatttaa

SEQ ID NO: 58 - DNA, 18 bp, synthetic construct
  source 1..18: mol_type = other DNA
tattcataaatctgacca

SEQ ID NO: 59 - DNA, 18 bp, synthetic construct
  source 1..18: mol_type = other DNA
agccatattcataaatct

SEQ ID NO: 60 - DNA, 18 bp, synthetic construct
  source 1..18: mol_type = other DNA
aatagagccatattcata

SEQ ID NO: 61 - DNA, 18 bp, synthetic construct
  source 1..18: mol_type = other DNA
tgaggaatagagccatat

SEQ ID NO: 62 - DNA, 18 bp, synthetic construct
  source 1..18: mol_type = other DNA
cattatgaggaatagagc

SEQ ID NO: 63 - DNA, 18 bp, synthetic construct
  source 1..18: mol_type = other DNA
gttgtcattatgaggaat

SEQ ID NO: 64 - DNA, 18 bp, synthetic construct
  source 1..18: mol_type = other DNA
tgtatgttgtcattatga

SEQ ID NO: 65 - DNA, 18 bp, synthetic construct
  source 1..18: mol_type = other DNA
ctgtgtgtatgttgtcat

SEQ ID NO: 66 - DNA, 18 bp, synthetic construct
  source 1..18: mol_type = other DNA
tagtgctgtgtgtatgtt

SEQ ID NO: 67 - DNA, 18 bp, synthetic construct
  source 1..18: mol_type = other DNA
cattttagtgctgtgtgt

SEQ ID NO: 68 - DNA, 18 bp, synthetic construct
  source 1..18: mol_type = other DNA
ttagtcattttagtgctg

SEQ ID NO: 69 - DNA, 18 bp, synthetic construct
  source 1..18: mol_type = other DNA
agagattagtcattttag

SEQ ID NO: 70 - DNA, 18 bp, synthetic construct
  source 1..18: mol_type = other DNA
attgaagagattagtcat

SEQ ID NO: 71 - DNA, 18 bp, synthetic construct
  source 1..18: mol_type = other DNA
cacgtattgaagagatta

SEQ ID NO: 72 - DNA, 18 bp, synthetic construct
  source 1..18: mol_type = other DNA
ccaaacacgtattgaaga

SEQ ID NO: 73 - DNA, 18 bp, synthetic construct
  source 1..18: mol_type = other DNA
caatgccaaacacgtatt

SEQ ID NO: 74 - DNA, 18 bp, synthetic construct
  source 1..18: mol_type = other DNA
ctctacaatgccaaacac

SEQ ID NO: 75 - DNA, 18 bp, synthetic construct
  source 1..18: mol_type = other DNA
tttgactctacaatgcca

SEQ ID NO: 76 - DNA, 18 bp, synthetic construct
  source 1..18: mol_type = other DNA
gttattttgactctacaa

SEQ ID NO: 77 - DNA, 18 bp, synthetic construct
  source 1..18: mol_type = other DNA
ataacgttattttgactc

SEQ ID NO: 78 - DNA, 18 bp, synthetic construct
  source 1..18: mol_type = other DNA
caattataacgttatttt

SEQ ID NO: 79 - DNA, 18 bp, synthetic construct
  source 1..18: mol_type = other DNA
agaatcaattataacgtt

SEQ ID NO: 80 - DNA, 18 bp, synthetic construct
  source 1..18: mol_type = other DNA
aaaatagaatcaattata

SEQ ID NO: 81 - DNA, 18 bp, synthetic construct
  source 1..18: mol_type = other DNA
tataaaaaatagaatcaa

SEQ ID NO: 82 - DNA, 18 bp, synthetic construct
  source 1..18: mol_type = other DNA
agaagtataaaaaataga

SEQ ID NO: 83 - DNA, 18 bp, synthetic construct
  source 1..18: mol_type = other DNA
acactagaagtataaaaa

SEQ ID NO: 84 - DNA, 18 bp, synthetic construct
  source 1..18: mol_type = other DNA
tccaaacactagaagtat

SEQ ID NO: 85 - DNA, 18 bp, synthetic construct
  source 1..18: mol_type = other DNA
aaatatccaaacactaga

SEQ ID NO: 86 - DNA, 18 bp, synthetic construct
  source 1..18: mol_type = other DNA
aaataaaatatccaaaca

SEQ ID NO: 87 - DNA, 18 bp, synthetic construct
  source 1..18: mol_type = other DNA
ttacaaaataaaatatcc

SEQ ID NO: 88 - DNA, 18 bp, synthetic construct
  source 1..18: mol_type = other DNA
tatttttacaaaataaaa

SEQ ID NO: 89 - DNA, 18 bp, synthetic construct
  source 1..18: mol_type = other DNA
gattatatttttacaaaa

SEQ ID NO: 90 - DNA, 18 bp, synthetic construct
  source 1..18: mol_type = other DNA
ttcatgattatattttta

SEQ ID NO: 91 - DNA, 18 bp, synthetic construct
  source 1..18: mol_type = other DNA
catcattcatgattatat

SEQ ID NO: 92 - DNA, 18 bp, synthetic construct
  source 1..18: mol_type = other DNA
ctcaccatcattcatgat

SEQ ID NO: 93 - DNA, 18 bp, synthetic construct
  source 1..18: mol_type = other DNA
ccaacctcaccatcattc

SEQ ID NO: 94 - DNA, 18 bp, synthetic construct
  source 1..18: mol_type = other DNA
tatatccaacctcaccat

SEQ ID NO: 95 - DNA, 18 bp, synthetic construct
  source 1..18: mol_type = other DNA
attcttatatccaacctc

SEQ ID NO: 96 - DNA, 18 bp, synthetic construct
  source 1..18: mol_type = other DNA
tcatcattcttatatcca

SEQ ID NO: 97 - DNA, 18 bp, synthetic construct
  source 1..18: mol_type = other DNA
cataatcatcattcttat

SEQ ID NO: 98 - DNA, 18 bp, synthetic construct
  source 1..18: mol_type = other DNA
ccaatcataatcatcatt

SEQ ID NO: 99 - DNA, 18 bp, synthetic construct
  source 1..18: mol_type = other DNA
acttcccaatcataatca

SEQ ID NO: 100 - DNA, 18 bp, synthetic construct
  source 1..18: mol_type = other DNA
atctcacttcccaatcat

SEQ ID NO: 101 - DNA, 18 bp, synthetic construct
  source 1..18: mol_type = other DNA
ttcaaatctcacttccca

SEQ ID NO: 102 - DNA, 18 bp, synthetic construct
  source 1..18: mol_type = other DNA
gcatgttcaaatctcact

SEQ ID NO: 103 - DNA, 18 bp, synthetic construct
  source 1..18: mol_type = other DNA
tctgagcatgttcaaatc

SEQ ID NO: 104 - DNA, 18 bp, synthetic construct
  source 1..18: mol_type = other DNA
gagtttctgagcatgttc

SEQ ID NO: 105 - DNA, 18 bp, synthetic construct
  source 1..18: mol_type = other DNA
aatgagagtttctgagca

SEQ ID NO: 106 - DNA, 18 bp, synthetic construct
  source 1..18: mol_type = other DNA
aattaaatgagagtttct

SEQ ID NO: 107 - DNA, 18 bp, synthetic construct
  source 1..18: mol_type = other DNA
caaagaattaaatgagag

SEQ ID NO: 108 - DNA, 18 bp, synthetic construct
  source 1..18: mol_type = other DNA
tagggcaaagaattaaat

SEQ ID NO: 109 - DNA, 18 bp, synthetic construct
  source 1..18: mol_type = other DNA
gctgctagggcaaagaat

SEQ ID NO: 110 - DNA, 18 bp, synthetic construct
  source 1..18: mol_type = other DNA
tttatgctgctagggcaa

SEQ ID NO: 111 - DNA, 18 bp, synthetic construct
  source 1..18: mol_type = other DNA
gtgattttatgctgctag

SEQ ID NO: 112 - DNA, 18 bp, synthetic construct
  source 1..18: mol_type = other DNA
ctattgtgattttatgct

SEQ ID NO: 113 - DNA, 18 bp, synthetic construct
  source 1..18: mol_type = other DNA
cgcagctattgtgatttt

SEQ ID NO: 114 - DNA, 18 bp, synthetic construct
  source 1..18: mol_type = other DNA
tttgacgcagctattgtg

SEQ ID NO: 115 - DNA, 18 bp, synthetic construct
  source 1..18: mol_type = other DNA
tacgctttgacgcagcta

SEQ ID NO: 116 - DNA, 18 bp, synthetic construct
  source 1..18: mol_type = other DNA
tgagttacgctttgacgc

SEQ ID NO: 117 - DNA, 18 bp, synthetic construct
  source 1..18: mol_type = other DNA
gtgcctgagttacgcttt

SEQ ID NO: 118 - DNA, 18 bp, synthetic construct
  source 1..18: mol_type = other DNA
aatgagtgcctgagttac

SEQ ID NO: 119 - DNA, 18 bp, synthetic construct
  source 1..18: mol_type = other DNA
aataaaatgagtgcctga

SEQ ID NO: 120 - DNA, 18 bp, synthetic construct
  source 1..18: mol_type = other DNA
acaaaaataaaatgagtg

SEQ ID NO: 121 - DNA, 18 bp, synthetic construct
  source 1..18: mol_type = other DNA
gaacaacaaaaataaaat

SEQ ID NO: 122 - DNA, 18 bp, synthetic construct
  source 1..18: mol_type = other DNA
taacagaacaacaaaaat

SEQ ID NO: 123 - DNA, 18 bp, synthetic construct
  source 1..18: mol_type = other DNA
aaaaataacagaacaaca

SEQ ID NO: 124 - DNA, 18 bp, synthetic construct
  source 1..18: mol_type = other DNA
tttgaaaaaataacagaa

SEQ ID NO: 125 - DNA, 18 bp, synthetic construct
  source 1..18: mol_type = other DNA
catgctttgaaaaaataa

SEQ ID NO: 126 - DNA, 18 bp, synthetic construct
  source 1..18: mol_type = other DNA
aagcacatgctttgaaaa

SEQ ID NO: 127 - DNA, 18 bp, synthetic construct
  source 1..18: mol_type = other DNA
cataaaagcacatgcttt

SEQ ID NO: 128 - DNA, 18 bp, synthetic construct
  source 1..18: mol_type = other DNA
tgttgcataaaagcacat

SEQ ID NO: 129 - DNA, 18 bp, synthetic construct
  source 1..18: mol_type = other DNA
agtaatgttgcataaaag

SEQ ID NO: 130 - DNA, 18 bp, synthetic construct
  source 1..18: mol_type = other DNA
tattcagtaatgttgcat

SEQ ID NO: 131 - DNA, 18 bp, synthetic construct
  source 1..18: mol_type = other DNA
tgctttattcagtaatgt

SEQ ID NO: 132 - DNA, 18 bp, synthetic construct
  source 1..18: mol_type = other DNA
caacatgctttattcagt

SEQ ID NO: 133 - DNA, 18 bp, synthetic construct
  source 1..18: mol_type = other DNA
ctgtacaacatgctttat

SEQ ID NO: 134 - DNA, 18 bp, synthetic construct
  source 1..18: mol_type = other DNA
aagcactgtacaacatgc

SEQ ID NO: 135 - DNA, 18 bp, synthetic construct
  source 1..18: mol_type = other DNA
ttatcaagcactgtacaa

SEQ ID NO: 136 - DNA, 18 bp, synthetic construct
  source 1..18: mol_type = other DNA
acttcttatcaagcactg

SEQ ID NO: 137 - DNA, 18 bp, synthetic construct
  source 1..18: mol_type = other DNA
ttctaacttcttatcaag

SEQ ID NO: 138 - DNA, 18 bp, synthetic construct
  source 1..18: mol_type = other DNA
ttactttctaacttctta

SEQ ID NO: 139 - DNA, 18 bp, synthetic construct
  source 1..18: mol_type = other DNA
atttgttactttctaact

SEQ ID NO: 140 - DNA, 18 bp, synthetic construct
  source 1..18: mol_type = other DNA
aatttatttgttactttc

SEQ ID NO: 141 - DNA, 18 bp, synthetic construct
  source 1..18: mol_type = other DNA
atgataatttatttgtta

SEQ ID NO: 142 - DNA, 18 bp, synthetic construct
  source 1..18: mol_type = other DNA
acgtgatgataatttatt

SEQ ID NO: 143 - DNA, 18 bp, synthetic construct
  source 1..18: mol_type = other DNA
gtgcaacgtgatgataat

SEQ ID NO: 144 - DNA, 18 bp, synthetic construct
  source 1..18: mol_type = other DNA
acaaagtgcaacgtgatg

SEQ ID NO: 145 - DNA, 18 bp, synthetic construct
  source 1..18: mol_type = other DNA
aaaacacaaagtgcaacg

SEQ ID NO: 146 - DNA, 18 bp, synthetic construct
  source 1..18: mol_type = other DNA
catgcaaaacacaaagtg

SEQ ID NO: 147 - DNA, 18 bp, synthetic construct
  source 1..18: mol_type = other DNA
taaaacatgcaaaacaca

SEQ ID NO: 148 - DNA, 18 bp, synthetic construct
  source 1..18: mol_type = other DNA
gtgcataaaacatgcaaa

SEQ ID NO: 149 - DNA, 18 bp, synthetic construct
  source 1..18: mol_type = other DNA
gaaatgtgcataaaacat

SEQ ID NO: 150 - DNA, 18 bp, synthetic construct
  source 1..18: mol_type = other DNA
agccagaaatgtgcataa

SEQ ID NO: 151 - DNA, 18 bp, synthetic construct
  source 1..18: mol_type = other DNA
ctgtcagccagaaatgtg

SEQ ID NO: 152 - DNA, 18 bp, synthetic construct
  source 1..18: mol_type = other DNA
aaaagctgtcagccagaa

SEQ ID NO: 153 - DNA, 18 bp, synthetic construct
  source 1..18: mol_type = other DNA
tgtttaaaagctgtcagc

SEQ ID NO: 154 - DNA, 18 bp, synthetic construct
  source 1..18: mol_type = other DNA
ataaatgtttaaaagctg

SEQ ID NO: 155 - DNA, 18 bp, synthetic construct
  source 1..18: mol_type = other DNA
atacaataaatgtttaaa

SEQ ID NO: 156 - DNA, 18 bp, synthetic construct
  source 1..18: mol_type = other DNA
ttgaaatacaataaatgt

SEQ ID NO: 157 - DNA, 18 bp, synthetic construct
  source 1..18: mol_type = other DNA
gaaatttgaaatacaata

SEQ ID NO: 158 - DNA, 18 bp, synthetic construct
  source 1..18: mol_type = other DNA
gactggaaatttgaaata

SEQ ID NO: 159 - DNA, 18 bp, synthetic construct
  source 1..18: mol_type = other DNA
atttggactggaaatttg

SEQ ID NO: 160 - DNA, 18 bp, synthetic construct
  source 1..18: mol_type = other DNA
gaaaaatttggactggaa

SEQ ID NO: 161 - DNA, 18 bp, synthetic construct
  source 1..18: mol_type = other DNA
aagttgaaaaatttggac

SEQ ID NO: 162 - DNA, 18 bp, synthetic construct
  source 1..18: mol_type = other DNA
tttacaagttgaaaaatt

SEQ ID NO: 163 - DNA, 18 bp, synthetic construct
  source 1..18: mol_type = other DNA
ttaattttacaagttgaa

SEQ ID NO: 164 - DNA, 18 bp, synthetic construct
  source 1..18: mol_type = other DNA
tcagtttaattttacaag

SEQ ID NO: 165 - DNA, 18 bp, synthetic construct
  source 1..18: mol_type = other DNA
ttcactcagtttaatttt

SEQ ID NO: 166 - DNA, 18 bp, synthetic construct
  source 1..18: mol_type = other DNA
atcaattcactcagttta

SEQ ID NO: 167 - DNA, 18 bp, synthetic construct
  source 1..18: mol_type = other DNA
acgacatcaattcactca

SEQ ID NO: 168 - DNA, 18 bp, synthetic construct
  source 1..18: mol_type = other DNA
tattcacgacatcaattc

SEQ ID NO: 169 - DNA, 18 bp, synthetic construct
  source 1..18: mol_type = other DNA
ctagatattcacgacatc

SEQ ID NO: 170 - DNA, 18 bp, synthetic construct
  source 1..18: mol_type = other DNA
ttaccctagatattcacg

SEQ ID NO: 171 - DNA, 18 bp, synthetic construct
  source 1..18: mol_type = other DNA
ttattttaccctagatat

SEQ ID NO: 172 - DNA, 18 bp, synthetic construct
  source 1..18: mol_type = other DNA
aaattttattttacccta

SEQ ID NO: 173 - DNA, 18 bp, synthetic construct
  source 1..18: mol_type = other DNA
aacacaaattttatttta

SEQ ID NO: 174 - DNA, 18 bp, synthetic construct
  source 1..18: mol_type = other DNA
atttaaacacaaatttta

SEQ ID NO: 175 - DNA, 18 bp, synthetic construct
  source 1..18: mol_type = other DNA
tacaaatttaaacacaaa

SEQ ID NO: 176 - DNA, 18 bp, synthetic construct
  source 1..18: mol_type = other DNA
aaaaatacaaatttaaac

SEQ ID NO: 177 - DNA, 18 bp, synthetic construct
  source 1..18: mol_type = other DNA
aaattaaaaatacaaatt

SEQ ID NO: 178 - DNA, 18 bp, synthetic construct
  source 1..18: mol_type = other DNA
ttaggaaattaaaaatac

SEQ ID NO: 179 - DNA, 18 bp, synthetic construct
  source 1..18: mol_type = other DNA
ctaggttaggaaattaaa

SEQ ID NO: 180 - DNA, 18 bp, synthetic construct
  source 1..18: mol_type = other DNA
atttcctaggttaggaaa

SEQ ID NO: 181 - DNA, 18 bp, synthetic construct
  source 1..18: mol_type = other DNA
ttaagatttcctaggtta

SEQ ID NO: 182 - DNA, 18 bp, synthetic construct
  source 1..18: mol_type = other DNA
ggtatttaagatttccta

SEQ ID NO: 183 - DNA, 18 bp, synthetic construct
  source 1..18: mol_type = other DNA
aagaaggtatttaagatt

SEQ ID NO: 184 - DNA, 18 bp, synthetic construct
  source 1..18: mol_type = other DNA
tgaaaaagaaggtattta

SEQ ID NO: 185 - DNA, 18 bp, synthetic construct
  source 1..18: mol_type = other DNA
tcttttgaaaaagaaggt

SEQ ID NO: 186 - DNA, 18 bp, synthetic construct
  source 1..18: mol_type = other DNA
tgagttcttttgaaaaag

SEQ ID NO: 187 - DNA, 18 bp, synthetic construct
  source 1..18: mol_type = other DNA
agacttgagttcttttga

SEQ ID NO: 188 - DNA, 18 bp, synthetic construct
  source 1..18: mol_type = other DNA
cattaagacttgagttct

SEQ ID NO: 189 - DNA, 18 bp, synthetic construct
  source 1..18: mol_type = other DNA
ctatccattaagacttga

SEQ ID NO: 190 - DNA, 18 bp, synthetic construct
  source 1..18: mol_type = other DNA
tttccctatccattaaga

SEQ ID NO: 191 - DNA, 18 bp, synthetic construct
  source 1..18: mol_type = other DNA
gtctgtttccctatccat

SEQ ID NO: 192 - DNA, 18 bp, synthetic construct
  source 1..18: mol_type = other DNA
tttccctactgtggtgca

SEQ ID NO: 193 - DNA, 18 bp, synthetic construct
  source 1..18: mol_type = other DNA
tgtattttccctactgtg

SEQ ID NO: 194 - DNA, 18 bp, synthetic construct
  source 1..18: mol_type = other DNA
aataatgtattttcccta

SEQ ID NO: 195 - DNA, 18 bp, synthetic construct
  source 1..18: mol_type = other DNA
atgtaaataatgtatttt

SEQ ID NO: 196 - DNA, 18 bp, synthetic construct
  source 1..18: mol_type = other DNA
ttaggatgtaaataatgt

SEQ ID NO: 197 - DNA, 18 bp, synthetic construct
  source 1..18: mol_type = other DNA
agggattaggatgtaaat

SEQ ID NO: 198 - DNA, 18 bp, synthetic construct
  source 1..18: mol_type = other DNA
aaagagggaattaggatg

SEQ ID NO: 199 - DNA, 18 bp, synthetic construct
  source 1..18: mol_type = other DNA
attgaaaagaagggatta

SEQ ID NO: 200 - DNA, 18 bp, synthetic construct
  source 1..18: mol_type = other DNA
agacaattgaaaagaggg

SEQ ID NO: 201 - DNA, 18 bp, synthetic construct
  source 1..18: mol_type = other DNA
atttaagacaattgaaaa

SEQ ID NO: 202 - DNA, 18 bp, synthetic construct
  source 1..18: mol_type = other DNA
atgaaatttaagacaatt

SEQ ID NO: 203 - DNA, 18 bp, synthetic construct
  source 1..18: mol_type = other DNA
ttcaaatgaaatttaaga

SEQ ID NO: 204 - DNA, 18 bp, synthetic construct
  source 1..18: mol_type = other DNA
tttttttcaaatgaaatt

SEQ ID NO: 205 - DNA, 18 bp, synthetic construct
  source 1..18: mol_type = other DNA
ttttttttttttcaaatg

SEQ ID NO: 206 - DNA, 18 bp, synthetic construct
  source 1..18: mol_type = other DNA
aaggtttttttttttttc

SEQ ID NO: 207 - DNA, 18 bp, synthetic construct
  source 1..18: mol_type = other DNA
tcataaaggttttttttt

SEQ ID NO: 208 - DNA, 18 bp, synthetic construct
  source 1..18: mol_type = other DNA
taaattcataaaggtttt

SEQ ID NO: 209 - DNA, 18 bp, synthetic construct
  source 1..18: mol_type = other DNA
gagggtaaattcataaag

SEQ ID NO: 210 - DNA, 18 bp, synthetic construct
  source 1..18: mol_type = other DNA
ccacagagggtaaattca

SEQ ID NO: 211 - DNA, 18 bp, synthetic construct
  source 1..18: mol_type = other DNA
aaaatccacagagggtaa

SEQ ID NO: 212 - DNA, 18 bp, synthetic construct
  source 1..18: mol_type = other DNA
gggttaaaatccacagag

SEQ ID NO: 213 - DNA, 18 bp, synthetic construct
  source 1..18: mol_type = other DNA
cattgggattaaaatcca

SEQ ID NO: 214 - DNA, 18 bp, synthetic construct
  source 1..18: mol_type = other DNA
tcaaccattagggttaaa

SEQ ID NO: 215 - DNA, 18 bp, synthetic construct
  source 1..18: mol_type = other DNA
agatatcaaccattggga

SEQ ID NO: 216 - DNA, 18 bp, synthetic construct
  source 1..18: mol_type = other DNA
aataaagatatcaaccat

SEQ ID NO: 217 - DNA, 18 bp, synthetic construct
  source 1..18: mol_type = other DNA
aacttaataaagatatca

SEQ ID NO: 218 - DNA, 18 bp, synthetic construct
  source 1..18: mol_type = other DNA
aatgaaacttaataaaga

SEQ ID NO: 219 - DNA, 18 bp, synthetic construct
  source 1..18: mol_type = other DNA
tattcaatgaaacttaat

SEQ ID NO: 220 - DNA, 18 bp, synthetic construct
  source 1..18: mol_type = other DNA
aatcatattcaatgaaac

SEQ ID NO: 221 - DNA, 18 bp, synthetic construct
  source 1..18: mol_type = other DNA
aactaaatcatattcaat

SEQ ID NO: 222 - DNA, 18 bp, synthetic construct
  source 1..18: mol_type = other DNA
cacataactaaatcatat

SEQ ID NO: 223 - DNA, 18 bp, synthetic construct
  source 1..18: mol_type = other DNA
atatacacataactaaat

SEQ ID NO: 224 - DNA, 18 bp, synthetic construct
  source 1..18: mol_type = other DNA
actccatatacacataac

SEQ ID NO: 225 - DNA, 18 bp, synthetic construct
  source 1..18: mol_type = other DNA
ggataactccatatacac

SEQ ID NO: 226 - DNA, 18 bp, synthetic construct
  source 1..18: mol_type = other DNA
aagatggataactccata

SEQ ID NO: 227 - DNA, 18 bp, synthetic construct
  source 1..18: mol_type = other DNA
ccccaaagatggataact

SEQ ID NO: 228 - DNA, 18 bp, synthetic construct
  source 1..18: mol_type = other DNA
aatctccccaaagatgga

SEQ ID NO: 229 - DNA, 18 bp, synthetic construct
  source 1..18: mol_type = other DNA
ccagtaatctccccaaag

SEQ ID NO: 230 - DNA, 18 bp, synthetic construct
  source 1..18: mol_type = other DNA
ccaatccagtaatctccc

SEQ ID NO: 231 - DNA, 18 bp, synthetic construct
  source 1..18: mol_type = other DNA
cctcaccaatccagtaat

SEQ ID NO: 232 - DNA, 18 bp, synthetic construct
  source 1..18: mol_type = other DNA
cccgccctcaccaatcca

SEQ ID NO: 233 - DNA, 18 bp, synthetic construct
  source 1..18: mol_type = other DNA
ggtcccccgccctcacca

SEQ ID NO: 234 - DNA, 18 bp, synthetic construct
  source 1..18: mol_type = other DNA
accagggtcccccgccct

SEQ ID NO: 235 - DNA, 18 bp, synthetic construct
  source 1..18: mol_type = other DNA
tctacaccagggtccccc

SEQ ID NO: 236 - DNA, 18 bp, synthetic construct
  source 1..18: mol_type = other DNA
atcattctacaccagggt

SEQ ID NO: 237 - DNA, 18 bp, synthetic construct
  source 1..18: mol_type = other DNA
acataatcattctacacc

SEQ ID NO: 238 - DNA, 18 bp, synthetic construct
  source 1..18: mol_type = other DNA
ttttcacataatcattct

SEQ ID NO: 239 - DNA, 18 bp, synthetic construct
  source 1..18: mol_type = other DNA
ttgttttttcacataatc

SEQ ID NO: 240 - DNA, 18 bp, synthetic construct
  source 1..18: mol_type = other DNA
ttaaattgttttttcaca

SEQ ID NO: 241 - DNA, 18 bp, synthetic construct
  source 1..18: mol_type = other DNA
acaagttaaattgttttt

SEQ ID NO: 242 - DNA, 18 bp, synthetic construct
  source 1..18: mol_type = other DNA
gcttaacaagttaaattg

SEQ ID NO: 243 - DNA, 18 bp, synthetic construct
  source 1..18: mol_type = other DNA
catgagcttaacaagtta

SEQ ID NO: 244 - DNA, 18 bp, synthetic construct
  source 1..18: mol_type = other DNA
agtatcatgagcttaaca

SEQ ID NO: 245 - DNA, 18 bp, synthetic construct
  source 1..18: mol_type = other DNA
caaacagtatcatgagct

SEQ ID NO: 246 - DNA, 18 bp, synthetic construct
  source 1..18: mol_type = other DNA
tgcctcaaacagtatcat

SEQ ID NO: 247 - DNA, 18 bp, synthetic construct
  source 1..18: mol_type = other DNA
ctgtatgcctcaaacagt

SEQ ID NO: 248 - DNA, 18 bp, synthetic construct
  source 1..18: mol_type = other DNA
agggactgtatgcctcaa

SEQ ID NO: 249 - DNA, 18 bp, synthetic construct
  source 1..18: mol_type = other DNA
acagcagggactgtatgc

SEQ ID NO: 250 - DNA, 18 bp, synthetic construct
  source 1..18: mol_type = other DNA
actaaacagcaagggctg

SEQ ID NO: 251 - DNA, 18 bp, synthetic construct
  source 1..18: mol_type = other DNA
aatgtactaaacagcagg

SEQ ID NO: 252 - DNA, 18 bp, synthetic construct
  source 1..18: mol_type = other DNA
agaccaatgtactaaaca

SEQ ID NO: 253 - DNA, 18 bp, synthetic construct
  source 1..18: mol_type = other DNA
gacccagaccaatgtact

SEQ ID NO: 254 - DNA, 18 bp, synthetic construct
  source 1..18: mol_type = other DNA
ttcaggacccagaccaat

SEQ ID NO: 255 - DNA, 18 bp, synthetic construct
  source 1..18: mol_type = other DNA
taattttcaggacccaga

SEQ ID NO: 256 - DNA, 18 bp, synthetic construct
  source 1..18: mol_type = other DNA
actggtaattttcaggac

SEQ ID NO: 257 - DNA, 18 bp, synthetic construct
  source 1..18: mol_type = other DNA
atctaactggtaattttc

SEQ ID NO: 258 - DNA, 18 bp, synthetic construct
  source 1..18: mol_type = other DNA
atggtatctaactggtaa

SEQ ID NO: 259 - DNA, 18 bp, synthetic construct
  source 1..18: mol_type = other DNA
aactgatggtatctaact

SEQ ID NO: 260 - DNA, 18 bp, synthetic construct
  source 1..18: mol_type = other DNA
taatcaactgatggtatc

SEQ ID NO: 261 - DNA, 18 bp, synthetic construct
  source 1..18: mol_type = other DNA
atcaataatcaactgatg

SEQ ID NO: 262 - DNA, 18 bp, synthetic construct
  source 1..18: mol_type = other DNA
tacatatcaataatcaac

SEQ ID NO: 263 - DNA, 18 bp, synthetic construct
  source 1..18: mol_type = other DNA
gctcatacatatcaataa

SEQ ID NO: 264 - DNA, 18 bp, synthetic construct
  source 1..18: mol_type = other DNA
tatctgctcatacatatc

SEQ ID NO: 265 - DNA, 18 bp, synthetic construct
  source 1..18: mol_type = other DNA
cctagtatctgctcatac

SEQ ID NO: 266 - DNA, 18 bp, synthetic construct
  source 1..18: mol_type = other DNA
tgcaccctagtatctgct

SEQ ID NO: 267 - DNA, 18 bp, synthetic construct
  source 1..18: mol_type = other DNA
aatattgcaccctagtat

SEQ ID NO: 268 - DNA, 18 bp, synthetic construct
  source 1..18: mol_type = other DNA
cctgaaatattgcaccct

SEQ ID NO: 269 - DNA, 18 bp, synthetic construct
  source 1..18: mol_type = other DNA
tgaaacctgaaatattgc

SEQ ID NO: 270 - DNA, 18 bp, synthetic construct
  source 1..18: mol_type = other DNA
tcttatgaaacctgaaat

SEQ ID NO: 271 - DNA, 18 bp, synthetic construct
  source 1..18: mol_type = other DNA
accagtcttatgaaacct

SEQ ID NO: 272 - DNA, 18 bp, synthetic construct
  source 1..18: mol_type = other DNA
tcaataccagtcttatga

SEQ ID NO: 273 - DNA, 18 bp, synthetic construct
  source 1..18: mol_type = other DNA
cacaatcaataccagtct

SEQ ID NO: 274 - DNA, 18 bp, synthetic construct
  source 1..18: mol_type = other DNA
gtggtcacaatcaatacc

SEQ ID NO: 275 - DNA, 18 bp, synthetic construct
  source 1..18: mol_type = other DNA
tgagagtggtcacaatca

SEQ ID NO: 276 - DNA, 18 bp, synthetic construct
  source 1..18: mol_type = other DNA
aaaaatgagagtggtcac

SEQ ID NO: 277 - DNA, 18 bp, synthetic construct
  source 1..18: mol_type = other DNA
caataaaaaatgagagtg

SEQ ID NO: 278 - DNA, 18 bp, synthetic construct
  source 1..18: mol_type = other DNA
ttacacaataaaaaatga

SEQ ID NO: 279 - DNA, 18 bp, synthetic construct
  source 1..18: mol_type = other DNA
tgaacttacacaataaaa

SEQ ID NO: 280 - DNA, 18 bp, synthetic construct
  source 1..18: mol_type = other DNA
ccatatgaacttacacaa

SEQ ID NO: 281 - DNA, 18 bp, synthetic construct
  source 1..18: mol_type = other DNA
taaccccatatgaactta

SEQ ID NO: 282 - DNA, 18 bp, synthetic construct
  source 1..18: mol_type = other DNA
gaaaataaccccatatga

SEQ ID NO: 283 - DNA, 18 bp, synthetic construct
  source 1..18: mol_type = other DNA
attttgaaaataacccca

SEQ ID NO: 284 - DNA, 18 bp, synthetic construct
  source 1..18: mol_type = other DNA
ttaacattttgaaaataa

SEQ ID NO: 285 - DNA, 18 bp, synthetic construct
  source 1..18: mol_type = other DNA
ccttgttaacattttgaa

SEQ ID NO: 286 - DNA, 18 bp, synthetic construct
  source 1..18: mol_type = other DNA
ttttgccttgttaacatt

SEQ ID NO: 287 - DNA, 18 bp, synthetic construct
  source 1..18: mol_type = other DNA
tatatttttgccttgtta

SEQ ID NO: 288 - DNA, 18 bp, synthetic construct
  source 1..18: mol_type = other DNA
cttaatatatttttgcct

SEQ ID NO: 289 - DNA, 18 bp, synthetic construct
  source 1..18: mol_type = other DNA
tatttcttaatatatttt

SEQ ID NO: 290 - DNA, 18 bp, synthetic construct
  source 1..18: mol_type = other DNA
tcaactatttcttaatat

SEQ ID NO: 291 - DNA, 18 bp, synthetic construct
  source 1..18: mol_type = other DNA
cttattcaactatttctt

SEQ ID NO: 292 - DNA, 18 bp, synthetic construct
  source 1..18: mol_type = other DNA
atgtgcttattcaactat

SEQ ID NO: 293 - DNA, 18 bp, synthetic construct
  source 1..18: mol_type = other DNA
ttcacatgtgcttattca

SEQ ID NO: 294 - DNA, 18 bp, synthetic construct
  source 1..18: mol_type = other DNA
cacaattcacatgtgctt

SEQ ID NO: 295 - DNA, 18 bp, synthetic construct
  source 1..18: mol_type = other DNA
tacaacacaattcacatg

SEQ ID NO: 296 - DNA, 18 bp, synthetic construct
  source 1..18: mol_type = other DNA
ttgtttacaacacaattc

SEQ ID NO: 297 - DNA, 18 bp, synthetic construct
  source 1..18: mol_type = other DNA
actttttgtttacaacac

SEQ ID NO: 298 - DNA, 18 bp, synthetic construct
  source 1..18: mol_type = other DNA
ttctaactttttgtttac

SEQ ID NO: 299 - DNA, 18 bp, synthetic construct
  source 1..18: mol_type = other DNA
ttttattctaactttttg

SEQ ID NO: 300 - DNA, 18 bp, synthetic construct
  source 1..18: mol_type = other DNA
gatttttttattctaact

SEQ ID NO: 301 - DNA, 18 bp, synthetic construct
  source 1..18: mol_type = other DNA
aagtggatttttttattc

SEQ ID NO: 302 - DNA, 18 bp, synthetic construct
  source 1..18: mol_type = other DNA
caaataagtggatttttt

SEQ ID NO: 303 - DNA, 18 bp, synthetic construct
  source 1..18: mol_type = other DNA
taattcaaataagtggat

SEQ ID NO: 304 - DNA, 18 bp, synthetic construct
  source 1..18: mol_type = other DNA
ctgcataattcaaataag

SEQ ID NO: 305 - DNA, 18 bp, synthetic construct
  source 1..18: mol_type = other DNA
ctattctgcataattcaa

SEQ ID NO: 306 - DNA, 18 bp, synthetic construct
  source 1..18: mol_type = other DNA
gtattctattctgcataa

SEQ ID NO: 307 - DNA, 18 bp, synthetic construct
  source 1..18: mol_type = other DNA
ggtatgtattctattctg

SEQ ID NO: 308 - DNA, 18 bp, synthetic construct
  source 1..18: mol_type = other DNA
ttctaggtatgtattcta

SEQ ID NO: 309 - DNA, 18 bp, synthetic construct
  source 1..18: mol_type = other DNA
tttatttctaggtatgta

SEQ ID NO: 310 - DNA, 18 bp, synthetic construct
  source 1..18: mol_type = other DNA
tttgttttatttctaggt

SEQ ID NO: 311 - DNA, 18 bp, synthetic construct
  source 1..18: mol_type = other DNA
acgtttttgttttatttc

SEQ ID NO: 312 - DNA, 18 bp, synthetic construct
  source 1..18: mol_type = other DNA
ataagacgtttttgtttt

SEQ ID NO: 313 - DNA, 18 bp, synthetic construct
  source 1..18: mol_type = other DNA
tcatgataagacgttttt

SEQ ID NO: 314 - DNA, 18 bp, synthetic construct
  source 1..18: mol_type = other DNA
aatactcatgataagacg

SEQ ID NO: 315 - DNA, 18 bp, synthetic construct
  source 1..18: mol_type = other DNA
atcttaatactcatgata

SEQ ID NO: 316 - DNA, 18 bp, synthetic construct
  source 1..18: mol_type = other DNA
attttatcttaatactca

SEQ ID NO: 317 - DNA, 18 bp, synthetic construct
  source 1..18: mol_type = other DNA
cttaaattttatcttaat

SEQ ID NO: 318 - DNA, 18 bp, synthetic construct
  source 1..18: mol_type = other DNA
tatgccttaaattttatc

SEQ ID NO: 319 - DNA, 18 bp, synthetic construct
  source 1..18: mol_type = other DNA
gagtttatgccttaaatt

SEQ ID NO: 320 - DNA, 18 bp, synthetic construct
  source 1..18: mol_type = other DNA
gaagtgagtttatgcctt

SEQ ID NO: 321 - DNA, 18 bp, synthetic construct
  source 1..18: mol_type = other DNA
tctaagaagtgagtttat

SEQ ID NO: 322 - DNA, 18 bp, synthetic construct
  source 1..18: mol_type = other DNA
cttattctaagaagtgag

SEQ ID NO: 323 - DNA, 18 bp, synthetic construct
  source 1..18: mol_type = other DNA
agttacttattctaagaa

SEQ ID NO: 324 - DNA, 18 bp, synthetic construct
  source 1..18: mol_type = other DNA
ttgggagttacttattct

SEQ ID NO: 325 - DNA, 18 bp, synthetic construct
  source 1..18: mol_type = other DNA
gttagttgggagttactt

SEQ ID NO: 326 - DNA, 18 bp, synthetic construct
  source 1..18: mol_type = other DNA
agaaagttagttgggagt

SEQ ID NO: 327 - DNA, 18 bp, synthetic construct
  source 1..18: mol_type = other DNA
atcctagaaagttagttg

SEQ ID NO: 328 - DNA, 18 bp, synthetic construct
  source 1..18: mol_type = other DNA
ttaaaatcctagaaagtt

SEQ ID NO: 329 - DNA, 18 bp, synthetic construct
  source 1..18: mol_type = other DNA
atgttttaaaatcctaga

SEQ ID NO: 330 - DNA, 18 bp, synthetic construct
  source 1..18: mol_type = other DNA
gtgttatgttttaaaatc

SEQ ID NO: 331 - DNA, 18 bp, synthetic construct
  source 1..18: mol_type = other DNA
tcactgtgttatgtttta

SEQ ID NO: 332 - DNA, 18 bp, synthetic construct
  source 1..18: mol_type = other DNA
tgttttcactgtgttatg

SEQ ID NO: 333 - DNA, 18 bp, synthetic construct
  source 1..18: mol_type = other DNA
atgtatgttttcactgtg

SEQ ID NO: 334 - DNA, 18 bp, synthetic construct
  source 1..18: mol_type = other DNA
tgtttatgtatgttttca

SEQ ID NO: 335 - DNA, 18 bp, synthetic construct
  source 1..18: mol_type = other DNA
agttatgtttatgtatgt

SEQ ID NO: 336 - DNA, 18 bp, synthetic construct
  source 1..18: mol_type = other DNA
tgtagagttatgtttatg

SEQ ID NO: 337 - DNA, 18 bp, synthetic construct
  source 1..18: mol_type = other DNA
taaaatgtagagttatgt

SEQ ID NO: 338 - DNA, 18 bp, synthetic construct
  source 1..18: mol_type = other DNA
ataaataaaatgtagagt

SEQ ID NO: 339 - DNA, 18 bp, synthetic construct
  source 1..18: mol_type = other DNA
taagaataaataaaatgt

SEQ ID NO: 340 - DNA, 18 bp, synthetic construct
  source 1..18: mol_type = other DNA
aactttaagaataaataa

SEQ ID NO: 341 - DNA, 18 bp, synthetic construct
  source 1..18: mol_type = other DNA
acttaaactttaagaata

SEQ ID NO: 342 - DNA, 18 bp, synthetic construct
  source 1..18: mol_type = other DNA
aatacacttaaactttaa

SEQ ID NO: 343 - DNA, 18 bp, synthetic construct
  source 1..18: mol_type = other DNA
tgtataatacacttaaac

SEQ ID NO: 344 - DNA, 18 bp, synthetic construct
  source 1..18: mol_type = other DNA
cttcttgtataatacact

SEQ ID NO: 345 - DNA, 18 bp, synthetic construct
  source 1..18: mol_type = other DNA
ctcttcttcttgtataat

SEQ ID NO: 346 - DNA, 18 bp, synthetic construct
  source 1..18: mol_type = other DNA
ataaactcttcttcttgt

SEQ ID NO: 347 - DNA, 18 bp, synthetic construct
  source 1..18: mol_type = other DNA
cgaatataaactcttctt

SEQ ID NO: 348 - DNA, 18 bp, synthetic construct
  source 1..18: mol_type = other DNA
tctctcgaatataaactc

SEQ ID NO: 349 - DNA, 18 bp, synthetic construct
  source 1..18: mol_type = other DNA
ttctgtctctcgaatata

SEQ ID NO: 350 - DNA, 18 bp, synthetic construct
  source 1..18: mol_type = other DNA
actttttctgtctctcga

SEQ ID NO: 351 - DNA, 18 bp, synthetic construct
  source 1..18: mol_type = other DNA
ttctgactttttctgtct

SEQ ID NO: 352 - DNA, 18 bp, synthetic construct
  source 1..18: mol_type = other DNA
aaaaattctgactttttc

SEQ ID NO: 353 - DNA, 18 bp, synthetic construct
  source 1..18: mol_type = other DNA
caaacaaaaattctgact

SEQ ID NO: 354 - DNA, 18 bp, synthetic construct
  source 1..18: mol_type = other DNA
tgatccaaacaaaaattc

SEQ ID NO: 355 - DNA, 18 bp, synthetic construct
  source 1..18: mol_type = other DNA
attggtgatccaaacaaa

SEQ ID NO: 356 - DNA, 18 bp, synthetic construct
  source 1..18: mol_type = other DNA
gatatattggtgatccaa

SEQ ID NO: 357 - DNA, 18 bp, synthetic construct
  source 1..18: mol_type = other DNA
gctatgatatattggtga

SEQ ID NO: 358 - DNA, 18 bp, synthetic construct
  source 1..18: mol_type = other DNA
tgtaagctatgatatatt

SEQ ID NO: 359 - DNA, 18 bp, synthetic construct
  source 1..18: mol_type = other DNA
ttttttgtaagctatgat

SEQ ID NO: 360 - DNA, 18 bp, synthetic construct
  source 1..18: mol_type = other DNA
acagtttttttgtaagct

SEQ ID NO: 361 - DNA, 18 bp, synthetic construct
  source 1..18: mol_type = other DNA
ttaagacagtttttttgt

SEQ ID NO: 362 - DNA, 18 bp, synthetic construct
  source 1..18: mol_type = other DNA
tttaattaagacagtttt

SEQ ID NO: 363 - DNA, 18 bp, synthetic construct
  source 1..18: mol_type = other DNA
tgggttttaattaagaca

SEQ ID NO: 364 - DNA, 18 bp, synthetic construct
  source 1..18: mol_type = other DNA
tgttgtgggttttaatta

SEQ ID NO: 365 - DNA, 18 bp, synthetic construct
  source 1..18: mol_type = other DNA
aattatgttgtgggtttt

SEQ ID NO: 366 - DNA, 18 bp, synthetic construct
  source 1..18: mol_type = other DNA
aaaaaaattatgttgtgg

SEQ ID NO: 367 - DNA, 18 bp, synthetic construct
  source 1..18: mol_type = other DNA
aatctaaaaaaattatgt

SEQ ID NO: 368 - DNA, 18 bp, synthetic construct
  source 1..18: mol_type = other DNA
ttaaaaatctaaaaaaat

SEQ ID NO: 369 - DNA, 18 bp, synthetic construct
  source 1..18: mol_type = other DNA
ctttcttaaaaatctaaa

SEQ ID NO: 370 - DNA, 18 bp, synthetic construct
  source 1..18: mol_type = other DNA
agaatctttcttaaaaat

SEQ ID NO: 371 - DNA, 18 bp, synthetic construct
  source 1..18: mol_type = other DNA
ataatagaatctttctta

SEQ ID NO: 372 - RNA, 18 nt, synthetic construct
  source 1..18: mol_type = other RNA
atgaatttaataaacttt

SEQ ID NO: 373 - RNA, 18 nt, synthetic construct
  source 1..18: mol_type = other RNA
gaccaatgaatttaataa

SEQ ID NO: 374 - RNA, 18 nt, synthetic construct
  source 1..18: mol_type = other RNA
cacttgaccaatgaattt

SEQ ID NO: 375 - RNA, 18 nt, synthetic construct
  source 1..18: mol_type = other RNA
ctgagcacttgaccaatg

SEQ ID NO: 376 - RNA, 18 nt, synthetic construct
  source 1..18: mol_type = other RNA
aatatctgagcacttgac

SEQ ID NO: 377 - RNA, 18 nt, synthetic construct
  source 1..18: mol_type = other RNA
atggaaatatctgagcac

SEQ ID NO: 378 - RNA, 18 nt, synthetic construct
  source 1..18: mol_type = other RNA
aatgtatggaaatatctg

SEQ ID NO: 379 - RNA, 18 nt, synthetic construct
  source 1..18: mol_type = other RNA
agtgtaatgtatggaaat

SEQ ID NO: 380 - RNA, 18 nt, synthetic construct
  source 1..18: mol_type = other RNA
aatgaagtgtaatgtatg

SEQ ID NO: 381 - RNA, 18 nt, synthetic construct
  source 1..18: mol_type = other RNA
atagaaatgaagtgtaat

SEQ ID NO: 382 - RNA, 18 nt, synthetic construct
  source 1..18: mol_type = other RNA
tttttatagaaatgaagt

SEQ ID NO: 383 - RNA, 18 nt, synthetic construct
  source 1..18: mol_type = other RNA
cagcttttttatagaaat

SEQ ID NO: 384 - RNA, 18 nt, synthetic construct
  source 1..18: mol_type = other RNA
aagatcagcttttttata

SEQ ID NO: 385 - RNA, 18 nt, synthetic construct
  source 1..18: mol_type = other RNA
ccgataagatcagctttt

SEQ ID NO: 386 - RNA, 18 nt, synthetic construct
  source 1..18: mol_type = other RNA
gtataccgataagatcag

SEQ ID NO: 387 - RNA, 18 nt, synthetic construct
  source 1..18: mol_type = other RNA
taaaagtataccgataag

SEQ ID NO: 388 - RNA, 18 nt, synthetic construct
  source 1..18: mol_type = other RNA
aaaattaaaagtataccg

SEQ ID NO: 389 - RNA, 18 nt, synthetic construct
  source 1..18: mol_type = other RNA
ctgagaaaattaaaagta

SEQ ID NO: 390 - RNA, 18 nt, synthetic construct
  source 1..18: mol_type = other RNA
tatttctgagaaaattaa

SEQ ID NO: 391 - RNA, 18 nt, synthetic construct
  source 1..18: mol_type = other RNA
atggttatttctgagaaa

SEQ ID NO: 392 - RNA, 18 nt, synthetic construct
  source 1..18: mol_type = other RNA
tagatatggttatttctg

SEQ ID NO: 393 - RNA, 18 nt, synthetic construct
  source 1..18: mol_type = other RNA
aattatagatatggttat

SEQ ID NO: 394 - RNA, 18 nt, synthetic construct
  source 1..18: mol_type = other RNA
ttaataattatagatatg

SEQ ID NO: 395 - RNA, 18 nt, synthetic construct
  source 1..18: mol_type = other RNA
attgattaataattatag

SEQ ID NO: 396 - RNA, 18 nt, synthetic construct
  source 1..18: mol_type = other RNA
gcattattgattaataat

SEQ ID NO: 397 - RNA, 18 nt, synthetic construct
  source 1..18: mol_type = other RNA
aaaaggcattattgatta

SEQ ID NO: 398 - RNA, 18 nt, synthetic construct
  source 1..18: mol_type = other RNA
aatataaaaggcattatt

SEQ ID NO: 399 - RNA, 18 nt, synthetic construct
  source 1..18: mol_type = other RNA
cttttaatataaaaggca

SEQ ID NO: 400 - RNA, 18 nt, synthetic construct
  source 1..18: mol_type = other RNA
aacctcttttaatataaa

SEQ ID NO: 401 - RNA, 18 nt, synthetic construct
  source 1..18: mol_type = other RNA
aaactaacctcttttaat

SEQ ID NO: 402 - RNA, 18 nt, synthetic construct
  source 1..18: mol_type = other RNA
ttcaaaaactaacctctt

SEQ ID NO: 403 - RNA, 18 nt, synthetic construct
  source 1..18: mol_type = other RNA
caagtttcaaaaactaac

SEQ ID NO: 404 - RNA, 18 nt, synthetic construct
  source 1..18: mol_type = other RNA
aactccaagtttcaaaaa

SEQ ID NO: 405 - RNA, 18 nt, synthetic construct
  source 1..18: mol_type = other RNA
tctaaaactccaagtttc

SEQ ID NO: 406 - RNA, 18 nt, synthetic construct
  source 1..18: mol_type = other RNA
ttatgtctaaaactccaa

SEQ ID NO: 407 - RNA, 18 nt, synthetic construct
  source 1..18: mol_type = other RNA
ggattttatgtctaaaac

SEQ ID NO: 408 - RNA, 18 nt, synthetic construct
  source 1..18: mol_type = other RNA
tataaggattttatgtct

SEQ ID NO: 409 - RNA, 18 nt, synthetic construct
  source 1..18: mol_type = other RNA
gcatttataaggatttta

SEQ ID NO: 410 - RNA, 18 nt, synthetic construct
  source 1..18: mol_type = other RNA
tatcagcatttataagga

SEQ ID NO: 411 - RNA, 18 nt, synthetic construct
  source 1..18: mol_type = other RNA
atcactatcagcatttat

SEQ ID NO: 412 - RNA, 18 nt, synthetic construct
  source 1..18: mol_type = other RNA
gttatatcactatcagca

SEQ ID NO: 413 - RNA, 18 nt, synthetic construct
  source 1..18: mol_type = other RNA
tattagttatatcactat

SEQ ID NO: 414 - RNA, 18 nt, synthetic construct
  source 1..18: mol_type = other RNA
taaactattagttatatc

SEQ ID NO: 415 - RNA, 18 nt, synthetic construct
  source 1..18: mol_type = other RNA
ccatttaaactattagtt

SEQ ID NO: 416 - RNA, 18 nt, synthetic construct
  source 1..18: mol_type = other RNA
tctgaccatttaaactat

SEQ ID NO: 417 - RNA, 18 nt, synthetic construct
  source 1..18: mol_type = other RNA
ataaatctgaccatttaa

SEQ ID NO: 418 - RNA, 18 nt, synthetic construct
  source 1..18: mol_type = other RNA
tattcataaatctgacca

SEQ ID NO: 419 - RNA, 18 nt, synthetic construct
  source 1..18: mol_type = other RNA
agccatattcataaatct

SEQ ID NO: 420 - RNA, 18 nt, synthetic construct
  source 1..18: mol_type = other RNA
aatagagccatattcata

SEQ ID NO: 421 - RNA, 18 nt, synthetic construct
  source 1..18: mol_type = other RNA
tgaggaatagagccatat

SEQ ID NO: 422 - RNA, 18 nt, synthetic construct
  source 1..18: mol_type = other RNA
cattatgaggaatagagc

SEQ ID NO: 423 - RNA, 18 nt, synthetic construct
  source 1..18: mol_type = other RNA
gttgtcattatgaggaat

SEQ ID NO: 424 - RNA, 18 nt, synthetic construct
  source 1..18: mol_type = other RNA
tgtatgttgtcattatga

SEQ ID NO: 425 - RNA, 18 nt, synthetic construct
  source 1..18: mol_type = other RNA
ctgtgtgtatgttgtcat

SEQ ID NO: 426 - RNA, 18 nt, synthetic construct
  source 1..18: mol_type = other RNA
tagtgctgtgtgtatgtt

SEQ ID NO: 427 - RNA, 18 nt, synthetic construct
  source 1..18: mol_type = other RNA
cattttagtgctgtgtgt

SEQ ID NO: 428 - RNA, 18 nt, synthetic construct
  source 1..18: mol_type = other RNA
ttagtcattttagtgctg

SEQ ID NO: 429 - RNA, 18 nt, synthetic construct
  source 1..18: mol_type = other RNA
agagattagtcattttag

SEQ ID NO: 430 - RNA, 18 nt, synthetic construct
  source 1..18: mol_type = other RNA
attgaagagattagtcat

SEQ ID NO: 431 - RNA, 18 nt, synthetic construct
  source 1..18: mol_type = other RNA
cacgtattgaagagatta

SEQ ID NO: 432 - RNA, 18 nt, synthetic construct
  source 1..18: mol_type = other RNA
ccaaacacgtattgaaga

SEQ ID NO: 433 - RNA, 18 nt, synthetic construct
  source 1..18: mol_type = other RNA
caatgccaaacacgtatt

SEQ ID NO: 434 - RNA, 18 nt, synthetic construct
  source 1..18: mol_type = other RNA
ctctacaatgccaaacac

SEQ ID NO: 435 - RNA, 18 nt, synthetic construct
  source 1..18: mol_type = other RNA
tttgactctacaatgcca

SEQ ID NO: 436 - RNA, 18 nt, synthetic construct
  source 1..18: mol_type = other RNA
gttattttgactctacaa

SEQ ID NO: 437 - RNA, 18 nt, synthetic construct
  source 1..18: mol_type = other RNA
ataacgttattttgactc

SEQ ID NO: 438 - RNA, 18 nt, synthetic construct
  source 1..18: mol_type = other RNA
caattataacgttatttt

SEQ ID NO: 439 - RNA, 18 nt, synthetic construct
  source 1..18: mol_type = other RNA
agaatcaattataacgtt

SEQ ID NO: 440 - RNA, 18 nt, synthetic construct
  source 1..18: mol_type = other RNA
aaaatagaatcaattata

SEQ ID NO: 441 - RNA, 18 nt, synthetic construct
  source 1..18: mol_type = other RNA
tataaaaaatagaatcaa

SEQ ID NO: 442 - RNA, 18 nt, synthetic construct
  source 1..18: mol_type = other RNA
agaagtataaaaaataga

SEQ ID NO: 443 - RNA, 18 nt, synthetic construct
  source 1..18: mol_type = other RNA
acactagaagtataaaaa

SEQ ID NO: 444 - RNA, 18 nt, synthetic construct
  source 1..18: mol_type = other RNA
tccaaacactagaagtat

SEQ ID NO: 445 - RNA, 18 nt, synthetic construct
  source 1..18: mol_type = other RNA
aaatatccaaacactaga

SEQ ID NO: 446 - RNA, 18 nt, synthetic construct
  source 1..18: mol_type = other RNA
aaataaaatatccaaaca

SEQ ID NO: 447 - RNA, 18 nt, synthetic construct
  source 1..18: mol_type = other RNA
ttacaaaataaaatatcc

SEQ ID NO: 448 - RNA, 18 nt, synthetic construct
  source 1..18: mol_type = other RNA
tatttttacaaaataaaa

SEQ ID NO: 449 - RNA, 18 nt, synthetic construct
  source 1..18: mol_type = other RNA
gattatatttttacaaaa

SEQ ID NO: 450 - RNA, 18 nt, synthetic construct
  source 1..18: mol_type = other RNA
ttcatgattatattttta

SEQ ID NO: 451 - RNA, 18 nt, synthetic construct
  source 1..18: mol_type = other RNA
catcattcatgattatat

SEQ ID NO: 452 - RNA, 18 nt, synthetic construct
  source 1..18: mol_type = other RNA
ctcaccatcattcatgat

SEQ ID NO: 453 - RNA, 18 nt, synthetic construct
  source 1..18: mol_type = other RNA
ccaacctcaccatcattc

SEQ ID NO: 454 - RNA, 18 nt, synthetic construct
  source 1..18: mol_type = other RNA
tatatccaacctcaccat

SEQ ID NO: 455 - RNA, 18 nt, synthetic construct
  source 1..18: mol_type = other RNA
attcttatatccaacctc

SEQ ID NO: 456 - RNA, 18 nt, synthetic construct
  source 1..18: mol_type = other RNA
tcatcattcttatatcca

SEQ ID NO: 457 - RNA, 18 nt, synthetic construct
  source 1..18: mol_type = other RNA
cataatcatcattcttat

SEQ ID NO: 458 - RNA, 18 nt, synthetic construct
  source 1..18: mol_type = other RNA
ccaatcataatcatcatt

SEQ ID NO: 459 - RNA, 18 nt, synthetic construct
  source 1..18: mol_type = other RNA
acttcccaatcataatca

SEQ ID NO: 460 - RNA, 18 nt, synthetic construct
  source 1..18: mol_type = other RNA
atctcacttcccaatcat

SEQ ID NO: 461 - RNA, 18 nt, synthetic construct
  source 1..18: mol_type = other RNA
ttcaaatctcacttccca

SEQ ID NO: 462 - RNA, 18 nt, synthetic construct
  source 1..18: mol_type = other RNA
gcatgttcaaatctcact

SEQ ID NO: 463 - RNA, 18 nt, synthetic construct
  source 1..18: mol_type = other RNA
tctgagcatgttcaaatc

SEQ ID NO: 464 - RNA, 18 nt, synthetic construct
  source 1..18: mol_type = other RNA
gagtttctgagcatgttc

SEQ ID NO: 465 - RNA, 18 nt, synthetic construct
  source 1..18: mol_type = other RNA
aatgagagtttctgagca

SEQ ID NO: 466 - RNA, 18 nt, synthetic construct
  source 1..18: mol_type = other RNA
aattaaatgagagtttct

SEQ ID NO: 467 - RNA, 18 nt, synthetic construct
  source 1..18: mol_type = other RNA
caaagaattaaatgagag

SEQ ID NO: 468 - RNA, 18 nt, synthetic construct
  source 1..18: mol_type = other RNA
tagggcaaagaattaaat

SEQ ID NO: 469 - RNA, 18 nt, synthetic construct
  source 1..18: mol_type = other RNA
gctgctagggcaaagaat

SEQ ID NO: 470 - RNA, 18 nt, synthetic construct
  source 1..18: mol_type = other RNA
tttatgctgctagggcaa

SEQ ID NO: 471 - RNA, 18 nt, synthetic construct
  source 1..18: mol_type = other RNA
gtgattttatgctgctag

SEQ ID NO: 472 - RNA, 18 nt, synthetic construct
  source 1..18: mol_type = other RNA
ctattgtgattttatgct

SEQ ID NO: 473 - RNA, 18 nt, synthetic construct
  source 1..18: mol_type = other RNA
cgcagctattgtgatttt

SEQ ID NO: 474 - RNA, 18 nt, synthetic construct
  source 1..18: mol_type = other RNA
tttgacgcagctattgtg

SEQ ID NO: 475 - RNA, 18 nt, synthetic construct
  source 1..18: mol_type = other RNA
tacgctttgacgcagcta

SEQ ID NO: 476 - RNA, 18 nt, synthetic construct
  source 1..18: mol_type = other RNA
tgagttacgctttgacgc

SEQ ID NO: 477 - RNA, 18 nt, synthetic construct
  source 1..18: mol_type = other RNA
gtgcctgagttacgcttt

SEQ ID NO: 478 - RNA, 18 nt, synthetic construct
  source 1..18: mol_type = other RNA
aatgagtgcctgagttac

SEQ ID NO: 479 - RNA, 18 nt, synthetic construct
  source 1..18: mol_type = other RNA
aataaaatgagtgcctga

SEQ ID NO: 480 - RNA, 18 nt, synthetic construct
  source 1..18: mol_type = other RNA
acaaaaataaaatgagtg

SEQ ID NO: 481 - RNA, 18 nt, synthetic construct
  source 1..18: mol_type = other RNA
gaacaacaaaaataaaat

SEQ ID NO: 482 - RNA, 18 nt, synthetic construct
  source 1..18: mol_type = other RNA
taacagaacaacaaaaat

SEQ ID NO: 483 - RNA, 18 nt, synthetic construct
  source 1..18: mol_type = other RNA
aaaaataacagaacaaca

SEQ ID NO: 484 - RNA, 18 nt, synthetic construct
  source 1..18: mol_type = other RNA
tttgaaaaaataacagaa

SEQ ID NO: 485 - RNA, 18 nt, synthetic construct
  source 1..18: mol_type = other RNA
catgctttgaaaaaataa

SEQ ID NO: 486 - RNA, 18 nt, synthetic construct
  source 1..18: mol_type = other RNA
aagcacatgctttgaaaa

SEQ ID NO: 487 - RNA, 18 nt, synthetic construct
  source 1..18: mol_type = other RNA
cataaaagcacatgcttt

SEQ ID NO: 488 - RNA, 18 nt, synthetic construct
  source 1..18: mol_type = other RNA
tgttgcataaaagcacat

SEQ ID NO: 489 - RNA, 18 nt, synthetic construct
  source 1..18: mol_type = other RNA
agtaatgttgcataaaag

SEQ ID NO: 490 - RNA, 18 nt, synthetic construct
  source 1..18: mol_type = other RNA
tattcagtaatgttgcat

SEQ ID NO: 491 - RNA, 18 nt, synthetic construct
  source 1..18: mol_type = other RNA
tgctttattcagtaatgt

SEQ ID NO: 492 - RNA, 18 nt, synthetic construct
  source 1..18: mol_type = other RNA
caacatgctttattcagt

SEQ ID NO: 493 - RNA, 18 nt, synthetic construct
  source 1..18: mol_type = other RNA
ctgtacaacatgctttat

SEQ ID NO: 494 - RNA, 18 nt, synthetic construct
  source 1..18: mol_type = other RNA
aagcactgtacaacatgc

SEQ ID NO: 495 - RNA, 18 nt, synthetic construct
  source 1..18: mol_type = other RNA
ttatcaagcactgtacaa

SEQ ID NO: 496 - RNA, 18 nt, synthetic construct
  source 1..18: mol_type = other RNA
acttcttatcaagcactg

SEQ ID NO: 497 - RNA, 18 nt, synthetic construct
  source 1..18: mol_type = other RNA
ttctaacttcttatcaag

SEQ ID NO: 498 - RNA, 18 nt, synthetic construct
  source 1..18: mol_type = other RNA
ttactttctaacttctta

SEQ ID NO: 499 - RNA, 18 nt, synthetic construct
  source 1..18: mol_type = other RNA
atttgttactttctaact

SEQ ID NO: 500 - RNA, 18 nt, synthetic construct
  source 1..18: mol_type = other RNA
aatttatttgttactttc

SEQ ID NO: 501 - RNA, 18 nt, synthetic construct
  source 1..18: mol_type = other RNA
atgataatttatttgtta

SEQ ID NO: 502 - RNA, 18 nt, synthetic construct
  source 1..18: mol_type = other RNA
acgtgatgataatttatt

SEQ ID NO: 503 - RNA, 18 nt, synthetic construct
  source 1..18: mol_type = other RNA
gtgcaacgtgatgataat

SEQ ID NO: 504 - RNA, 18 nt, synthetic construct
  source 1..18: mol_type = other RNA
acaaagtgcaacgtgatg

SEQ ID NO: 505 - RNA, 18 nt, synthetic construct
  source 1..18: mol_type = other RNA
aaaacacaaagtgcaacg

SEQ ID NO: 506 - RNA, 18 nt, synthetic construct
  source 1..18: mol_type = other RNA
catgcaaaacacaaagtg

SEQ ID NO: 507 - RNA, 18 nt, synthetic construct
  source 1..18: mol_type = other RNA
taaaacatgcaaaacaca

SEQ ID NO: 508 - RNA, 18 nt, synthetic construct
  source 1..18: mol_type = other RNA
gtgcataaaacatgcaaa

SEQ ID NO: 509 - RNA, 18 nt, synthetic construct
  source 1..18: mol_type = other RNA
gaaatgtgcataaaacat

SEQ ID NO: 510 - RNA, 18 nt, synthetic construct
  source 1..18: mol_type = other RNA
agccagaaatgtgcataa

SEQ ID NO: 511 - RNA, 18 nt, synthetic construct
  source 1..18: mol_type = other RNA
ctgtcagccagaaatgtg

SEQ ID NO: 512 - RNA, 18 nt, synthetic construct
  source 1..18: mol_type = other RNA
aaaagctgtcagccagaa

SEQ ID NO: 513 - RNA, 18 nt, synthetic construct
  source 1..18: mol_type = other RNA
tgtttaaaagctgtcagc

SEQ ID NO: 514 - RNA, 18 nt, synthetic construct
  source 1..18: mol_type = other RNA
ataaatgtttaaaagctg

SEQ ID NO: 515 - RNA, 18 nt, synthetic construct
  source 1..18: mol_type = other RNA
atacaataaatgtttaaa

SEQ ID NO: 516 - RNA, 18 nt, synthetic construct
  source 1..18: mol_type = other RNA
ttgaaatacaataaatgt

SEQ ID NO: 517 - RNA, 18 nt, synthetic construct
  source 1..18: mol_type = other RNA
gaaatttgaaatacaata

SEQ ID NO: 518 - RNA, 18 nt, synthetic construct
  source 1..18: mol_type = other RNA
gactggaaatttgaaata

SEQ ID NO: 519 - RNA, 18 nt, synthetic construct
  source 1..18: mol_type = other RNA
atttggactggaaatttg

SEQ ID NO: 520 - RNA, 18 nt, synthetic construct
  source 1..18: mol_type = other RNA
gaaaaatttggactggaa

SEQ ID NO: 521 - RNA, 18 nt, synthetic construct
  source 1..18: mol_type = other RNA
aagttgaaaaatttggac

SEQ ID NO: 522 - RNA, 18 nt, synthetic construct
  source 1..18: mol_type = other RNA
tttacaagttgaaaaatt

SEQ ID NO: 523 - RNA, 18 nt, synthetic construct
  source 1..18: mol_type = other RNA
ttaattttacaagttgaa

SEQ ID NO: 524 - RNA, 18 nt, synthetic construct
  source 1..18: mol_type = other RNA
tcagtttaattttacaag

SEQ ID NO: 525 - RNA, 18 nt, synthetic construct
  source 1..18: mol_type = other RNA
ttcactcagtttaatttt

SEQ ID NO: 526 - RNA, 18 nt, synthetic construct
  source 1..18: mol_type = other RNA
atcaattcactcagttta

SEQ ID NO: 527 - RNA, 18 nt, synthetic construct
  source 1..18: mol_type = other RNA
acgacatcaattcactca

SEQ ID NO: 528 - RNA, 18 nt, synthetic construct
  source 1..18: mol_type = other RNA
tattcacgacatcaattc

SEQ ID NO: 529 - RNA, 18 nt, synthetic construct
  source 1..18: mol_type = other RNA
ctagatattcacgacatc

SEQ ID NO: 530 - RNA, 18 nt, synthetic construct
  source 1..18: mol_type = other RNA
ttaccctagatattcacg

SEQ ID NO: 531 - RNA, 18 nt, synthetic construct
  source 1..18: mol_type = other RNA
ttattttaccctagatat

SEQ ID NO: 532 - RNA, 18 nt, synthetic construct
  source 1..18: mol_type = other RNA
aaattttattttacccta

SEQ ID NO: 533 - RNA, 18 nt, synthetic construct
  source 1..18: mol_type = other RNA
aacacaaattttatttta

SEQ ID NO: 534 - RNA, 18 nt, synthetic construct
  source 1..18: mol_type = other RNA
atttaaacacaaatttta

SEQ ID NO: 535 - RNA, 18 nt, synthetic construct
  source 1..18: mol_type = other RNA
tacaaatttaaacacaaa

SEQ ID NO: 536 - RNA, 18 nt, synthetic construct
  source 1..18: mol_type = other RNA
aaaaatacaaatttaaac

SEQ ID NO: 537 - RNA, 18 nt, synthetic construct
  source 1..18: mol_type = other RNA
aaattaaaaatacaaatt

SEQ ID NO: 538 - RNA, 18 nt, synthetic construct
  source 1..18: mol_type = other RNA
ttaggaaattaaaaatac

SEQ ID NO: 539 - RNA, 18 nt, synthetic construct
  source 1..18: mol_type = other RNA
ctaggttaggaaattaaa

SEQ ID NO: 540 - RNA, 18 nt, synthetic construct
  source 1..18: mol_type = other RNA
atttcctaggttaggaaa

SEQ ID NO: 541 - RNA, 18 nt, synthetic construct
  source 1..18: mol_type = other RNA
ttaagatttcctaggtta

SEQ ID NO: 542 - RNA, 18 nt, synthetic construct
  source 1..18: mol_type = other RNA
ggtatttaagatttccta

SEQ ID NO: 543 - RNA, 18 nt, synthetic construct
  source 1..18: mol_type = other RNA
aagaaggtatttaagatt

SEQ ID NO: 544 - RNA, 18 nt, synthetic construct
  source 1..18: mol_type = other RNA
tgaaaaagaaggtattta

SEQ ID NO: 545 - RNA, 18 nt, synthetic construct
  source 1..18: mol_type = other RNA
tcttttgaaaaagaaggt

SEQ ID NO: 546 - RNA, 18 nt, synthetic construct
  source 1..18: mol_type = other RNA
tgagttcttttgaaaaag

SEQ ID NO: 547 - RNA, 18 nt, synthetic construct
  source 1..18: mol_type = other RNA
agacttgagttcttttga

SEQ ID NO: 548 - RNA, 18 nt, synthetic construct
  source 1..18: mol_type = other RNA
cattaagacttgagttct

SEQ ID NO: 549 - RNA, 18 nt, synthetic construct
  source 1..18: mol_type = other RNA
ctatccattaagacttga

SEQ ID NO: 550 - RNA, 18 nt, synthetic construct
  source 1..18: mol_type = other RNA
tttccctatccattaaga

SEQ ID NO: 551 - RNA, 18 nt, synthetic construct
  source 1..18: mol_type = other RNA
gtctgtttccctatccat

SEQ ID NO: 552 - RNA, 18 nt, synthetic construct
  source 1..18: mol_type = other RNA
tttccctactgtggtgca

SEQ ID NO: 553 - RNA, 18 nt, synthetic construct
  source 1..18: mol_type = other RNA
tgtattttccctactgtg

SEQ ID NO: 554 - RNA, 18 nt, synthetic construct
  source 1..18: mol_type = other RNA
aataatgtattttcccta

SEQ ID NO: 555 - RNA, 18 nt, synthetic construct
  source 1..18: mol_type = other RNA
atgtaaataatgtatttt

SEQ ID NO: 556 - RNA, 18 nt, synthetic construct
  source 1..18: mol_type = other RNA
ttaggatgtaaataatgt

SEQ ID NO: 557 - RNA, 18 nt, synthetic construct
  source 1..18: mol_type = other RNA
agggattaggatgtaaat

SEQ ID NO: 558 - RNA, 18 nt, synthetic construct
  source 1..18: mol_type = other RNA
aaagagggaattaggatg

SEQ ID NO: 559 - RNA, 18 nt, synthetic construct
  source 1..18: mol_type = other RNA
attgaaaagaagggatta

SEQ ID NO: 560 - RNA, 18 nt, synthetic construct
  source 1..18: mol_type = other RNA
agacaattgaaaagaggg

SEQ ID NO: 561 - RNA, 18 nt, synthetic construct
  source 1..18: mol_type = other RNA
atttaagacaattgaaaa

SEQ ID NO: 562 - RNA, 18 nt, synthetic construct
  source 1..18: mol_type = other RNA
atgaaatttaagacaatt

SEQ ID NO: 563 - RNA, 18 nt, synthetic construct
  source 1..18: mol_type = other RNA
ttcaaatgaaatttaaga

SEQ ID NO: 564 - RNA, 18 nt, synthetic construct
  source 1..18: mol_type = other RNA
tttttttcaaatgaaatt

SEQ ID NO: 565 - RNA, 18 nt, synthetic construct
  source 1..18: mol_type = other RNA
ttttttttttttcaaatg

SEQ ID NO: 566 - RNA, 18 nt, synthetic construct
  source 1..18: mol_type = other RNA
aaggtttttttttttttc

SEQ ID NO: 567 - RNA, 18 nt, synthetic construct
  source 1..18: mol_type = other RNA
tcataaaggttttttttt

SEQ ID NO: 568 - RNA, 18 nt, synthetic construct
  source 1..18: mol_type = other RNA
taaattcataaaggtttt

SEQ ID NO: 569 - RNA, 18 nt, synthetic construct
  source 1..18: mol_type = other RNA
gagggtaaattcataaag

SEQ ID NO: 570 - RNA, 18 nt, synthetic construct
  source 1..18: mol_type = other RNA
ccacagagggtaaattca

SEQ ID NO: 571 - RNA, 18 nt, synthetic construct
  source 1..18: mol_type = other RNA
aaaatccacagagggtaa

SEQ ID NO: 572 - RNA, 18 nt, synthetic construct
  source 1..18: mol_type = other RNA
gggttaaaatccacagag

SEQ ID NO: 573 - RNA, 18 nt, synthetic construct
  source 1..18: mol_type = other RNA
cattgggattaaaatcca

SEQ ID NO: 574 - RNA, 18 nt, synthetic construct
  source 1..18: mol_type = other RNA
tcaaccattagggttaaa

SEQ ID NO: 575 - RNA, 18 nt, synthetic construct
  source 1..18: mol_type = other RNA
agatatcaaccattggga

SEQ ID NO: 576 - RNA, 18 nt, synthetic construct
  source 1..18: mol_type = other RNA
aataaagatatcaaccat

SEQ ID NO: 577 - RNA, 18 nt, synthetic construct
  source 1..18: mol_type = other RNA
aacttaataaagatatca

SEQ ID NO: 578 - RNA, 18 nt, synthetic construct
  source 1..18: mol_type = other RNA
aatgaaacttaataaaga

SEQ ID NO: 579 - RNA, 18 nt, synthetic construct
  source 1..18: mol_type = other RNA
tattcaatgaaacttaat

SEQ ID NO: 580 - RNA, 18 nt, synthetic construct
  source 1..18: mol_type = other RNA
aatcatattcaatgaaac

SEQ ID NO: 581 - RNA, 18 nt, synthetic construct
  source 1..18: mol_type = other RNA
aactaaatcatattcaat

SEQ ID NO: 582 - RNA, 18 nt, synthetic construct
  source 1..18: mol_type = other RNA
cacataactaaatcatat

SEQ ID NO: 583 - RNA, 18 nt, synthetic construct
  source 1..18: mol_type = other RNA
atatacacataactaaat

SEQ ID NO: 584 - RNA, 18 nt, synthetic construct
  source 1..18: mol_type = other RNA
actccatatacacataac

SEQ ID NO: 585 - RNA, 18 nt, synthetic construct
  source 1..18: mol_type = other RNA
ggataactccatatacac

SEQ ID NO: 586 - RNA, 18 nt, synthetic construct
  source 1..18: mol_type = other RNA
aagatggataactccata

SEQ ID NO: 587 - RNA, 18 nt, synthetic construct
  source 1..18: mol_type = other RNA
ccccaaagatggataact

SEQ ID NO: 588 - RNA, 18 nt, synthetic construct
  source 1..18: mol_type = other RNA
aatctccccaaagatgga

SEQ ID NO: 589 - RNA, 18 nt, synthetic construct
  source 1..18: mol_type = other RNA
ccagtaatctccccaaag

SEQ ID NO: 590 - RNA, 18 nt, synthetic construct
  source 1..18: mol_type = other RNA
ccaatccagtaatctccc

SEQ ID NO: 591 - RNA, 18 nt, synthetic construct
  source 1..18: mol_type = other RNA
cctcaccaatccagtaat

SEQ ID NO: 592 - RNA, 18 nt, synthetic construct
  source 1..18: mol_type = other RNA
cccgccctcaccaatcca

SEQ ID NO: 593 - RNA, 18 nt, synthetic construct
  source 1..18: mol_type = other RNA
ggtcccccgccctcacca

SEQ ID NO: 594 - RNA, 18 nt, synthetic construct
  source 1..18: mol_type = other RNA
accagggtcccccgccct

SEQ ID NO: 595 - RNA, 18 nt, synthetic construct
  source 1..18: mol_type = other RNA
tctacaccagggtccccc

SEQ ID NO: 596 - RNA, 18 nt, synthetic construct
  source 1..18: mol_type = other RNA
atcattctacaccagggt

SEQ ID NO: 597 - RNA, 18 nt, synthetic construct
  source 1..18: mol_type = other RNA
acataatcattctacacc

SEQ ID NO: 598 - RNA, 18 nt, synthetic construct
  source 1..18: mol_type = other RNA
ttttcacataatcattct

SEQ ID NO: 599 - RNA, 18 nt, synthetic construct
  source 1..18: mol_type = other RNA
ttgttttttcacataatc

SEQ ID NO: 600 - RNA, 18 nt, synthetic construct
  source 1..18: mol_type = other RNA
ttaaattgttttttcaca

SEQ ID NO: 601 - RNA, 18 nt, synthetic construct
  source 1..18: mol_type = other RNA
acaagttaaattgttttt

SEQ ID NO: 602 - RNA, 18 nt, synthetic construct
  source 1..18: mol_type = other RNA
gcttaacaagttaaattg

SEQ ID NO: 603 - RNA, 18 nt, synthetic construct
  source 1..18: mol_type = other RNA
catgagcttaacaagtta

SEQ ID NO: 604 - RNA, 18 nt, synthetic construct
  source 1..18: mol_type = other RNA
agtatcatgagcttaaca

SEQ ID NO: 605 - RNA, 18 nt, synthetic construct
  source 1..18: mol_type = other RNA
caaacagtatcatgagct

SEQ ID NO: 606 - RNA, 18 nt, synthetic construct
  source 1..18: mol_type = other RNA
tgcctcaaacagtatcat

SEQ ID NO: 607 - RNA, 18 nt, synthetic construct
  source 1..18: mol_type = other RNA
ctgtatgcctcaaacagt

SEQ ID NO: 608 - RNA, 18 nt, synthetic construct
  source 1..18: mol_type = other RNA
agggactgtatgcctcaa

SEQ ID NO: 609 - RNA, 18 nt, synthetic construct
  source 1..18: mol_type = other RNA
acagcagggactgtatgc

SEQ ID NO: 610 - RNA, 18 nt, synthetic construct
  source 1..18: mol_type = other RNA
actaaacagcaagggctg

SEQ ID NO: 611 - RNA, 18 nt, synthetic construct
  source 1..18: mol_type = other RNA
aatgtactaaacagcagg

SEQ ID NO: 612 - RNA, 18 nt, synthetic construct
  source 1..18: mol_type = other RNA
agaccaatgtactaaaca

SEQ ID NO: 613 - RNA, 18 nt, synthetic construct
  source 1..18: mol_type = other RNA
gacccagaccaatgtact

SEQ ID NO: 614 - RNA, 18 nt, synthetic construct
  source 1..18: mol_type = other RNA
ttcaggacccagaccaat

SEQ ID NO: 615 - RNA, 18 nt, synthetic construct
  source 1..18: mol_type = other RNA
taattttcaggacccaga

SEQ ID NO: 616 - RNA, 18 nt, synthetic construct
  source 1..18: mol_type = other RNA
actggtaattttcaggac

SEQ ID NO: 617 - RNA, 18 nt, synthetic construct
  source 1..18: mol_type = other RNA
atctaactggtaattttc

SEQ ID NO: 618 - RNA, 18 nt, synthetic construct
  source 1..18: mol_type = other RNA
atggtatctaactggtaa

SEQ ID NO: 619 - RNA, 18 nt, synthetic construct
  source 1..18: mol_type = other RNA
aactgatggtatctaact

SEQ ID NO: 620 - RNA, 18 nt, synthetic construct
  source 1..18: mol_type = other RNA
taatcaactgatggtatc

SEQ ID NO: 621 - RNA, 18 nt, synthetic construct
  source 1..18: mol_type = other RNA
atcaataatcaactgatg

SEQ ID NO: 622 - RNA, 18 nt, synthetic construct
  source 1..18: mol_type = other RNA
tacatatcaataatcaac

SEQ ID NO: 623 - RNA, 18 nt, synthetic construct
  source 1..18: mol_type = other RNA
gctcatacatatcaataa

SEQ ID NO: 624 - RNA, 18 nt, synthetic construct
  source 1..18: mol_type = other RNA
tatctgctcatacatatc

SEQ ID NO: 625 - RNA, 18 nt, synthetic construct
  source 1..18: mol_type = other RNA
cctagtatctgctcatac

SEQ ID NO: 626 - RNA, 18 nt, synthetic construct
  source 1..18: mol_type = other RNA
tgcaccctagtatctgct

SEQ ID NO: 627 - RNA, 18 nt, synthetic construct
  source 1..18: mol_type = other RNA
aatattgcaccctagtat

SEQ ID NO: 628 - RNA, 18 nt, synthetic construct
  source 1..18: mol_type = other RNA
cctgaaatattgcaccct

SEQ ID NO: 629 - RNA, 18 nt, synthetic construct
  source 1..18: mol_type = other RNA
tgaaacctgaaatattgc

SEQ ID NO: 630 - RNA, 18 nt, synthetic construct
  source 1..18: mol_type = other RNA
tcttatgaaacctgaaat

SEQ ID NO: 631 - RNA, 18 nt, synthetic construct
  source 1..18: mol_type = other RNA
accagtcttatgaaacct

SEQ ID NO: 632 - RNA, 18 nt, synthetic construct
  source 1..18: mol_type = other RNA
tcaataccagtcttatga

SEQ ID NO: 633 - RNA, 18 nt, synthetic construct
  source 1..18: mol_type = other RNA
cacaatcaataccagtct

SEQ ID NO: 634 - RNA, 18 nt, synthetic construct
  source 1..18: mol_type = other RNA
gtggtcacaatcaatacc

SEQ ID NO: 635 - RNA, 18 nt, synthetic construct
  source 1..18: mol_type = other RNA
tgagagtggtcacaatca

SEQ ID NO: 636 - RNA, 18 nt, synthetic construct
  source 1..18: mol_type = other RNA
aaaaatgagagtggtcac

SEQ ID NO: 637 - RNA, 18 nt, synthetic construct
  source 1..18: mol_type = other RNA
caataaaaaatgagagtg

SEQ ID NO: 638 - RNA, 18 nt, synthetic construct
  source 1..18: mol_type = other RNA
ttacacaataaaaaatga

SEQ ID NO: 639 - RNA, 18 nt, synthetic construct
  source 1..18: mol_type = other RNA
tgaacttacacaataaaa

SEQ ID NO: 640 - RNA, 18 nt, synthetic construct
  source 1..18: mol_type = other RNA
ccatatgaacttacacaa

SEQ ID NO: 641 - RNA, 18 nt, synthetic construct
  source 1..18: mol_type = other RNA
taaccccatatgaactta

SEQ ID NO: 642 - RNA, 18 nt, synthetic construct
  source 1..18: mol_type = other RNA
gaaaataaccccatatga

SEQ ID NO: 643 - RNA, 18 nt, synthetic construct
  source 1..18: mol_type = other RNA
attttgaaaataacccca

SEQ ID NO: 644 - RNA, 18 nt, synthetic construct
  source 1..18: mol_type = other RNA
ttaacattttgaaaataa

SEQ ID NO: 645 - RNA, 18 nt, synthetic construct
  source 1..18: mol_type = other RNA
ccttgttaacattttgaa

SEQ ID NO: 646 - RNA, 18 nt, synthetic construct
  source 1..18: mol_type = other RNA
ttttgccttgttaacatt

SEQ ID NO: 647 - RNA, 18 nt, synthetic construct
  source 1..18: mol_type = other RNA
tatatttttgccttgtta

SEQ ID NO: 648 - RNA, 18 nt, synthetic construct
  source 1..18: mol_type = other RNA
cttaatatatttttgcct

SEQ ID NO: 649 - RNA, 18 nt, synthetic construct
  source 1..18: mol_type = other RNA
tatttcttaatatatttt

SEQ ID NO: 650 - RNA, 18 nt, synthetic construct
  source 1..18: mol_type = other RNA
tcaactatttcttaatat

SEQ ID NO: 651 - RNA, 18 nt, synthetic construct
  source 1..18: mol_type = other RNA
cttattcaactatttctt

SEQ ID NO: 652 - RNA, 18 nt, synthetic construct
  source 1..18: mol_type = other RNA
atgtgcttattcaactat

SEQ ID NO: 653 - RNA, 18 nt, synthetic construct
  source 1..18: mol_type = other RNA
ttcacatgtgcttattca

SEQ ID NO: 654 - RNA, 18 nt, synthetic construct
  source 1..18: mol_type = other RNA
cacaattcacatgtgctt

SEQ ID NO: 655 - RNA, 18 nt, synthetic construct
  source 1..18: mol_type = other RNA
tacaacacaattcacatg

SEQ ID NO: 656 - RNA, 18 nt, synthetic construct
  source 1..18: mol_type = other RNA
ttgtttacaacacaattc

SEQ ID NO: 657 - RNA, 18 nt, synthetic construct
  source 1..18: mol_type = other RNA
actttttgtttacaacac

SEQ ID NO: 658 - RNA, 18 nt, synthetic construct
  source 1..18: mol_type = other RNA
ttctaactttttgtttac

SEQ ID NO: 659 - RNA, 18 nt, synthetic construct
  source 1..18: mol_type = other RNA
ttttattctaactttttg

SEQ ID NO: 660 - RNA, 18 nt, synthetic construct
  source 1..18: mol_type = other RNA
gatttttttattctaact

SEQ ID NO: 661 - RNA, 18 nt, synthetic construct
  source 1..18: mol_type = other RNA
aagtggatttttttattc

SEQ ID NO: 662 - RNA, 18 nt, synthetic construct
  source 1..18: mol_type = other RNA
caaataagtggatttttt

SEQ ID NO: 663 - RNA, 18 nt, synthetic construct
  source 1..18: mol_type = other RNA
taattcaaataagtggat

SEQ ID NO: 664 - RNA, 18 nt, synthetic construct
  source 1..18: mol_type = other RNA
ctgcataattcaaataag

SEQ ID NO: 665 - RNA, 18 nt, synthetic construct
  source 1..18: mol_type = other RNA
ctattctgcataattcaa

SEQ ID NO: 666 - RNA, 18 nt, synthetic construct
  source 1..18: mol_type = other RNA
gtattctattctgcataa

SEQ ID NO: 667 - RNA, 18 nt, synthetic construct
  source 1..18: mol_type = other RNA
ggtatgtattctattctg

SEQ ID NO: 668 - RNA, 18 nt, synthetic construct
  source 1..18: mol_type = other RNA
ttctaggtatgtattcta

SEQ ID NO: 669 - RNA, 18 nt, synthetic construct
  source 1..18: mol_type = other RNA
tttatttctaggtatgta

SEQ ID NO: 670 - RNA, 18 nt, synthetic construct
  source 1..18: mol_type = other RNA
tttgttttatttctaggt

SEQ ID NO: 671 - RNA, 18 nt, synthetic construct
  source 1..18: mol_type = other RNA
acgtttttgttttatttc

SEQ ID NO: 672 - RNA, 18 nt, synthetic construct
  source 1..18: mol_type = other RNA
ataagacgtttttgtttt

SEQ ID NO: 673 - RNA, 18 nt, synthetic construct
  source 1..18: mol_type = other RNA
tcatgataagacgttttt

SEQ ID NO: 674 - RNA, 18 nt, synthetic construct
  source 1..18: mol_type = other RNA
aatactcatgataagacg

SEQ ID NO: 675 - RNA, 18 nt, synthetic construct
  source 1..18: mol_type = other RNA
atcttaatactcatgata

SEQ ID NO: 676 - RNA, 18 nt, synthetic construct
  source 1..18: mol_type = other RNA
attttatcttaatactca

SEQ ID NO: 677 - RNA, 18 nt, synthetic construct
  source 1..18: mol_type = other RNA
cttaaattttatcttaat

SEQ ID NO: 678 - RNA, 18 nt, synthetic construct
  source 1..18: mol_type = other RNA
tatgccttaaattttatc

SEQ ID NO: 679 - RNA, 18 nt, synthetic construct
  source 1..18: mol_type = other RNA
gagtttatgccttaaatt

SEQ ID NO: 680 - RNA, 18 nt, synthetic construct
  source 1..18: mol_type = other RNA
gaagtgagtttatgcctt

SEQ ID NO: 681 - RNA, 18 nt, synthetic construct
  source 1..18: mol_type = other RNA
tctaagaagtgagtttat

SEQ ID NO: 682 - RNA, 18 nt, synthetic construct
  source 1..18: mol_type = other RNA
cttattctaagaagtgag

SEQ ID NO: 683 - RNA, 18 nt, synthetic construct
  source 1..18: mol_type = other RNA
agttacttattctaagaa

SEQ ID NO: 684 - RNA, 18 nt, synthetic construct
  source 1..18: mol_type = other RNA
ttgggagttacttattct

SEQ ID NO: 685 - RNA, 18 nt, synthetic construct
  source 1..18: mol_type = other RNA
gttagttgggagttactt

SEQ ID NO: 686 - RNA, 18 nt, synthetic construct
  source 1..18: mol_type = other RNA
agaaagttagttgggagt

SEQ ID NO: 687 - RNA, 18 nt, synthetic construct
  source 1..18: mol_type = other RNA
atcctagaaagttagttg

SEQ ID NO: 688 - RNA, 18 nt, synthetic construct
  source 1..18: mol_type = other RNA
ttaaaatcctagaaagtt

SEQ ID NO: 689 - RNA, 18 nt, synthetic construct
  source 1..18: mol_type = other RNA
atgttttaaaatcctaga

SEQ ID NO: 690 - RNA, 18 nt, synthetic construct
  source 1..18: mol_type = other RNA
gtgttatgttttaaaatc

SEQ ID NO: 691 - RNA, 18 nt, synthetic construct
  source 1..18: mol_type = other RNA
tcactgtgttatgtttta

SEQ ID NO: 692 - RNA, 18 nt, synthetic construct
  source 1..18: mol_type = other RNA
tgttttcactgtgttatg

SEQ ID NO: 693 - RNA, 18 nt, synthetic construct
  source 1..18: mol_type = other RNA
atgtatgttttcactgtg

SEQ ID NO: 694 - RNA, 18 nt, synthetic construct
  source 1..18: mol_type = other RNA
tgtttatgtatgttttca

SEQ ID NO: 695 - RNA, 18 nt, synthetic construct
  source 1..18: mol_type = other RNA
agttatgtttatgtatgt

SEQ ID NO: 696 - RNA, 18 nt, synthetic construct
  source 1..18: mol_type = other RNA
tgtagagttatgtttatg

SEQ ID NO: 697 - RNA, 18 nt, synthetic construct
  source 1..18: mol_type = other RNA
taaaatgtagagttatgt

SEQ ID NO: 698 - RNA, 18 nt, synthetic construct
  source 1..18: mol_type = other RNA
ataaataaaatgtagagt

SEQ ID NO: 699 - RNA, 18 nt, synthetic construct
  source 1..18: mol_type = other RNA
taagaataaataaaatgt

SEQ ID NO: 700 - RNA, 18 nt, synthetic construct
  source 1..18: mol_type = other RNA
aactttaagaataaataa

SEQ ID NO: 701 - RNA, 18 nt, synthetic construct
  source 1..18: mol_type = other RNA
acttaaactttaagaata

SEQ ID NO: 702 - RNA, 18 nt, synthetic construct
  source 1..18: mol_type = other RNA
aatacacttaaactttaa

SEQ ID NO: 703 - RNA, 18 nt, synthetic construct
  source 1..18: mol_type = other RNA
tgtataatacacttaaac

SEQ ID NO: 704 - RNA, 18 nt, synthetic construct
  source 1..18: mol_type = other RNA
cttcttgtataatacact

SEQ ID NO: 705 - RNA, 18 nt, synthetic construct
  source 1..18: mol_type = other RNA
ctcttcttcttgtataat

SEQ ID NO: 706 - RNA, 18 nt, synthetic construct
  source 1..18: mol_type = other RNA
ataaactcttcttcttgt

SEQ ID NO: 707 - RNA, 18 nt, synthetic construct
  source 1..18: mol_type = other RNA
cgaatataaactcttctt

SEQ ID NO: 708 - RNA, 18 nt, synthetic construct
  source 1..18: mol_type = other RNA
tctctcgaatataaactc

SEQ ID NO: 709 - RNA, 18 nt, synthetic construct
  source 1..18: mol_type = other RNA
ttctgtctctcgaatata

SEQ ID NO: 710 - RNA, 18 nt, synthetic construct
  source 1..18: mol_type = other RNA
actttttctgtctctcga

SEQ ID NO: 711 - RNA, 18 nt, synthetic construct
  source 1..18: mol_type = other RNA
ttctgactttttctgtct

SEQ ID NO: 712 - RNA, 18 nt, synthetic construct
  source 1..18: mol_type = other RNA
aaaaattctgactttttc

SEQ ID NO: 713 - RNA, 18 nt, synthetic construct
  source 1..18: mol_type = other RNA
caaacaaaaattctgact

SEQ ID NO: 714 - RNA, 18 nt, synthetic construct
  source 1..18: mol_type = other RNA
tgatccaaacaaaaattc

SEQ ID NO: 715 - RNA, 18 nt, synthetic construct
  source 1..18: mol_type = other RNA
attggtgatccaaacaaa

SEQ ID NO: 716 - RNA, 18 nt, synthetic construct
  source 1..18: mol_type = other RNA
gatatattggtgatccaa

SEQ ID NO: 717 - RNA, 18 nt, synthetic construct
  source 1..18: mol_type = other RNA
gctatgatatattggtga

SEQ ID NO: 718 - RNA, 18 nt, synthetic construct
  source 1..18: mol_type = other RNA
tgtaagctatgatatatt

SEQ ID NO: 719 - RNA, 18 nt, synthetic construct
  source 1..18: mol_type = other RNA
ttttttgtaagctatgat

SEQ ID NO: 720 - RNA, 18 nt, synthetic construct
  source 1..18: mol_type = other RNA
acagtttttttgtaagct

SEQ ID NO: 721 - RNA, 18 nt, synthetic construct
  source 1..18: mol_type = other RNA
ttaagacagtttttttgt

SEQ ID NO: 722 - RNA, 18 nt, synthetic construct
  source 1..18: mol_type = other RNA
tttaattaagacagtttt

SEQ ID NO: 723 - RNA, 18 nt, synthetic construct
  source 1..18: mol_type = other RNA
tgggttttaattaagaca

SEQ ID NO: 724 - RNA, 18 nt, synthetic construct
  source 1..18: mol_type = other RNA
tgttgtgggttttaatta

SEQ ID NO: 725 - RNA, 18 nt, synthetic construct
  source 1..18: mol_type = other RNA
aattatgttgtgggtttt

SEQ ID NO: 726 - RNA, 18 nt, synthetic construct
  source 1..18: mol_type = other RNA
aaaaaaattatgttgtgg

SEQ ID NO: 727 - RNA, 18 nt, synthetic construct
  source 1..18: mol_type = other RNA
aatctaaaaaaattatgt

SEQ ID NO: 728 - RNA, 18 nt, synthetic construct
  source 1..18: mol_type = other RNA
ttaaaaatctaaaaaaat

SEQ ID NO: 729 - RNA, 18 nt, synthetic construct
  source 1..18: mol_type = other RNA
ctttcttaaaaatctaaa

SEQ ID NO: 730 - RNA, 18 nt, synthetic construct
  source 1..18: mol_type = other RNA
agaatctttcttaaaaat

SEQ ID NO: 731 - RNA, 18 nt, synthetic construct
  source 1..18: mol_type = other RNA
ataatagaatctttctta

SEQ ID NO: 732 - DNA, 264 bp, Homo sapiens
  source 1..264: mol_type = genomic DNA
ggaaacagacggagagcatcatgaacaaaaagtaacaccaaatgttctgtcatatcagatttctaactaataacaaactatatatttctattttgtataggataatcttgctccaacttggatggggtggagcgctggttcctcccctgagccctttattatgggtactgtattaccccttttgctacctttaatccttgcactgtgacttatgtgtagtggggtgagggagggattgggaagggtactattattgcaccacag